(19) United States
(12) Patent Application Publication
(10) Pub. No.: US 2010/0209914 A1
(43) Pub. Date: Aug. 19, 2010

(54) METHODS, SYSTEMS, AND KITS FOR EVALUATING MULTIPLE SCLEROSIS

(75) Inventors: Douglas Bigwood, Medison, CT (US); Eric Eastman, Hihgland, MD (US); Eric Kaldjian, Ann Arbor, MI (US)

Correspondence Address: COOLEY LLP;ATTN: Patent Group, Suite 1100, 777 - 6th Street, NW, WASHINGTON, DC, 20001, US

(73) Assignee: ORE PHARMACEUTICALS , INC., Gaithersburg, MA (US)

(21) Appl. No.: 12/513,764

(22) PCT Filed: May 26, 2008
(86) PCT No.: PCT/US2008/064924

§ 371 Date: Apr. 26, 2010

Related U.S. Application Data
(60) Provisional application No. 60/924,671, filed on May 25, 2007.

(51) Int. Cl. C12Q 1/68

(52) U.S. Cl. 435/6

(57) ABSTRACT

The present invention provides a method for evaluating multiple sclerosis (MS), or excluding MS as a diagnosis for a patient The method comprises determining a gene expression profile for a sample from such a patient. The gene expression profile, which contains gene expression values for a plurality of genes that are differentially expressed in white blood cells of MS patients, is compared to an MS-profile and/or a non-MS profile, and classified. The invention also provides a method for monitoring treatment of an MS patient Pre-treatment and post-treatment gene expression profiles contain gene expression values for a plurality of genes that are differentially expressed upon treatment of MS patients. The expression profiles may then be compared, to identify differences between pre-treatment and post-treatment gene expression. These differences are indicative of the patient's response to treatment The invention further provides kits and systems for performing the methods of the invention.

### PRIORITY

This application claims priority to U.S. Provisional Application No. 60/924,671 filed May 25, 2007, which is hereby incorporated by reference in its entirety.

### FIELD OF THE INVENTION

The present invention relates to methods, systems, and kits for evaluating multiple sclerosis (MS), and for assisting in the diagnosis, prognosis, and/or treatment of MS.

### SEQUENCE LISTING

The contents of the accompanying Sequence Listing are hereby incorporated by reference.

### BACKGROUND OF THE INVENTION

Multiple sclerosis (MS) is a disease that affects the central nervous system, and can range from relatively benign to somewhat disabling to devastating. In MS, the myelin surrounding nerve cells is damaged or destroyed, impacting the ability of the nerves to conduct electrical impulses to and from the brain, and leaving scar tissue called sclerosis. These damaged areas are also known as “plaques” or “lesions.” According to the National Multiple Sclerosis Society, there are approximately 400,000 individuals with MS in the United States.

The first symptoms of MS typically appear between the ages of 20 and 40, and include blurred or double vision, red-green color distortion, or even blindness in one eye. Most MS patients experience muscle weakness in their extremities and difficulty with coordination and balance. In severe cases, MS can produce partial or complete paralysis. Paresthesias (numbness, prickling, or “pins and needles”), speech impediments, tremors, and dizziness are frequent symptoms of MS. Approximately half of MS patients experience cognitive impairments.

Diagnosing MS is complicated, because there is no single test that can confirm the presence of MS. The process of diagnosing MS typically involves criteria from the patient's history, a clinical examination, and one or more laboratory tests, with all three often being necessary to rule out other possible causes for symptoms and/or to gather facts sufficient for a diagnosis of MS.

Magnetic resonance imaging (MRI) is a preferred test. An MRI can detect plaques or scarring possibly caused by MS. However, an abnormal MRI does not necessarily indicate MS, as lesions in the brain may be associated with other disorders. Further, spots may also be found in healthy individuals, particularly in healthy older persons. These spots are called UBOs, for unidentified bright objects, and are not related to an ongoing disease process. In addition, a normal MRI does not absolutely rule out the presence MS. About 5% of individuals who are confirmed to have MS on the basis of other criteria, have no brain lesions detectable by MRI. These individuals may have lesions in the spinal cord or may have lesions that cannot be detected by MRI.

A diagnosis of MS might be made based on an evaluation of symptoms, signs, and the results of an MRI, but additional tests may be ordered as well. These include tests of evoked potential, cerebrospinal fluid, and blood.

For example, cerebrospinal fluid is sampled by a lumbar puncture, and is tested for levels of immune system proteins and for the presence of an antibody staining pattern called “oligoclonal bands.” Oligoclonal bands indicate an immune response within the central nervous system and are found in the spinal fluid of 90-95% of individuals with MS. However, oligoclonal bands are also associated with diseases other than MS, and therefore the presence of oligoclonal bands alone is not definitive of MS.

There is likewise no definitive blood test for MS, but blood tests can exclude other possible causes for various neurologic symptoms, such as Lyme disease, collagen-vascular diseases, rare hereditary disorders, and AIDS.

Diagnosing MS generally requires: (1) objective evidence of at least two areas of myelin loss, or demyelinating lesions, “separated in time and space” (lesions occurring in different places within the brain, spinal cord, or optic nerve-at different points in time); and (2) all other diseases that can cause similar neurologic symptoms have been objectively excluded. Until (1) and (2) have been satisfied, a physician does not make a definite diagnosis of MS.

Depending on the clinical problems present when an individual sees a physician, one or more of the tests described above might be performed. Sometimes tests are performed several times over a period of months to help gather the necessary information. A definite MS diagnosis must satisfy the McDonald criteria, named for the distinguished neurologist W. Ian McDonald who sparked society-supported efforts to make the diagnostic process for MS faster and more precise.

There are a few distinct clinical courses for MS, referred to as relapsing-remitting MS, secondary-progressive MS, progressive-relapsing MS, and primary progressive MS. Relapsing-remitting MS is characterized by clearly-defined, acute attacks (relapses), usually with full or partial recovery, and no disease progression between attacks. Secondary-progressive MS is initially relapsing-remitting but then becomes continuously progressive at a variable rate, with or without occasional relapses along the way. The disease-modifying medications are thought to provide benefit for those who continue to have relapses. Primary progressive MS may be characterized by disease progression from the beginning with few or no periods of remission. Progressive-relapsing MS is characterized by disease progression from the beginning, but with clear, acute relapses along the way.

There are several options available for treating individuals diagnosed with MS. Beta-interferon (Avonex, Betaseron, Rebif) has been approved to treat MS. Interferons are also made by the body, mainly to combat viral infections. Interferons have been shown to decrease the worsening or relapse of MS, however disease progression remains unaffected and the side effects of interferons are poorly tolerated. Glatiramer acetate (Copaxone) is a mixture of amino acids that has been shown to decrease the relapse rates of MS by 30%, and appears to also have a positive effect on the overall level of disability. Glatiramer acetate is better tolerated than the interferons and has fewer side effects. Glatiramer acts by binding to major histocompatibility complex class II molecules and competing with MBP and other myelin proteins for such binding and presentation to T cells. Natalizumab (Tysabri) is a monoclonal antibody that binds to alpha-4-integrin on white blood cells and interferes with their movement from the bloodstream into the brain and spinal cord.

An object of the present invention is to provide a blood-based diagnostic test for a more objective, definitive, and rapid diagnosis of MS. Another object of the invention is to provide a diagnostic test for monitoring MS progression, adequacy of treatment, and/or response to treatment.

### SUMMARY OF THE INVENTION

The present invention provides methods, systems, and kits for evaluating multiple sclerosis (MS) in a patient. Particularly, the invention provides convenient blood-based, gene-expression tests for evaluating MS, including for diagnosing MS, for excluding MS as a diagnosis, and for monitoring the course of disease or efficacy of treatment.

In one aspect, the invention provides a method for diagnosing MS or excluding MS as a diagnosis for a patient. In this aspect, the invention provides for a confident diagnosis of MS, or a confident exclusion of MS as a diagnosis, and in certain embodiments may help predict a clinical course of the patient's disease and/or a beneficial course of treatment.

In certain embodiments, the method comprises determining a gene expression profile for a blood sample (such as a whole blood sample) of a patient having or suspected of having MS. The gene expression profile contains gene transcript levels (or “expression levels”) for a plurality of genes that are differentially expressed in blood cells of MS patients, and such genes are listed in Tables 1 and 2. Tables 1 and 2 list genes that are differentially expressed in whole blood and PBMCs, respectively, of MS patients, and provide, inter alia, Mean-MS and Mean-control gene expression levels for these differentially expressed genes. In certain embodiments, the gene expression profile contains the expression level in the sample for at least one gene that is also listed in Table 6 (that is, in addition to being listed in Table 1 and/or 2).

In some embodiments, the method comprises determining a gene expression profile for a white blood cell sample (such as a PBMC sample) of a patient having or suspected of having MS. In these embodiments, the gene expression profile contains gene expression levels for a plurality of genes that are differentially expressed in white blood cells of MS patients, and such genes are listed in Table 2. In certain embodiments, the gene expression profile contains the expression level in the sample for at least one gene that is also listed in Table 6 (that is, in addition to being listed in Table 1 and/or 2).

The gene expression profile prepared according to this aspect of the invention is compared to an MS-profile and/or a non-MS profile, to classify the patient's gene expression profile as an MS profile or a non-MS profile. Generally, the non-MS profile is a healthy, non-disease, profile. In certain embodiments, the MS-profile is a relapsing-remitting MS profile.

In a second aspect, the invention provides a method for monitoring the course of disease or efficacy of treatment for an MS patient, to thereby provide accurate and objective criteria for determining disease progression and the efficacy of an MS treatment.

In this second aspect, the method comprises determining a gene expression profile, as described above, at various time points after an initial diagnosis of MS. Where the patient is undergoing treatment for MS, the gene expression profiles may include a pre-treatment (or early treatment) gene expression profile and at least one post-treatment gene expression profile of a patient having MS. In some embodiments, the MS patient is undergoing treatment with at least one of Beta-interferon, Glatiramer acetate, and Natalizumab. In certain embodiments, the pre-treatment and post-treatment gene expression profiles are determined for blood samples (such as a whole blood samples) of the patient, or white blood cell samples (such as PBMC samples). The gene expression profiles in accordance with this aspect contain gene expression levels for a plurality of genes that are differentially expressed in blood or in white blood cells of MS patients, and such genes are listed in Table 1 or 2, respectively. In certain embodiments, the gene expression profile contains the expression level in the sample for at least one gene that is also listed in Table 6.

Further, in embodiments where the patient is undergoing treatment with Beta-Interferon, such pre- and post-treatment gene expression profiles may contain gene expression levels for the genes listed in Tables 3 and/or 5, which list genes that are differentially expressed in PBMCs between pre- and post-treatment with Beta-interferon, and which may be correlative of a positive response to treatment. In embodiments where the patient is undergoing treatment with Glatiramer acetate (e.g., Copaxone), the pre- and post-treatment profiles may contain gene expression levels for genes listed in Table 4 and/or 5, which lists genes that are differentially expressed in PBMCs between pre- and post-treatment with Copaxone, and which may be correlative of a positive response to treatment. Table 5 lists genes that are differentially expressed between pre- and post-treatment with either or both of Beta-Interferon and Glatiramer acetate.

The pre-treatment gene expression profile may be compared with the post-treatment gene expression profile, to identify differences between pre-treatment and post-treatment gene expression (e.g., differences between pre-treatment and post-treatment blood transcript levels). These differences may be indicative of the patient's response (positive or negative) to treatment. Alternatively or in addition, the gene expression profiles determined over time, including the pre- and post-treatment profiles, may be compared to MS- and non-MS gene expression profiles (such as those shown in Tables 1 and 2), to determine whether, or to the extent, that the profile becomes more comparable to an MS or non-MS profile. For example, the post-treatment profile may become more or less indicative of an MS profile. Such may be indicative of a patient's positive or negative response to treatment.

In a third aspect, the invention provides a method for preparing a patient gene expression profile for evaluating MS. According to this aspect, the gene expression profile is useful for determining whether the patient has MS, as well as for monitoring the course of the disease, and predicting whether a particular treatment is or will be efficacious. In accordance with this aspect, the method generally comprises quantifying the level of expression, in a patient blood sample, for a plurality of genes listed in one of Tables 1-5.

For example, the gene expression profile may contain the levels of expression in the sample for a plurality of genes listed in Table 1 or Table 2. In some embodiments, at least one of these genes is also listed in Table 6. In accordance with this aspect, the patient may have, or be suspected to have, MS. Such gene expression profiles are useful for classifying samples as MS or non-MS samples in accordance with the first aspect of the invention, or for monitoring the course of the patient's MS over time.

Alternatively, the gene expression profile may contain the levels of expression in the sample for a plurality of genes listed in Table 3, 4, and/or 5. Preferably, at least one of these genes is also listed in Table 6. Such gene expression profiles are useful for predicting whether a particular treatment might be efficacious, or where treatment is already ongoing, determining whether the current treatment is effective.

In still other aspects, the invention provides kits and systems for performing the methods of the invention.

### DETAILED DESCRIPTION OF THE INVENTION

The present invention provides methods, systems, and kits for evaluating multiple sclerosis (MS). The invention provides convenient blood-based, gene-expression tests for evaluating MS in patients. Such patients may be known to have MS, may be suspected of having MS on the basis of one or more MS-like symptoms or results from one or more MS-related clinical exams, or may be beginning or undergoing treatment for MS. In the various aspects of the invention, the invention aids in diagnosing MS, or excluding MS as a diagnosis, monitoring the progression of disease, and predicting or determining efficacy of the various options for MS treatment and care.

### Methods For Diagnosing MS

In one aspect, the invention provides a method for diagnosing MS, or for excluding MS as a diagnosis for a patient. The method comprises determining a gene expression profile for a blood sample (such as a whole blood sample) or a white blood cell sample (such as a PBMC sample) of a patient having or suspected of having MS. The gene expression profile prepared according to this aspect of the invention is compared to an MS-profile and/or a non-MS profile, to classify the patient's gene expression profile as an MS profile or a non-MS profile. By classifying the profile as an MS or non-MS profile, a diagnosis of MS may be made, or MS may be excluded as a diagnosis.

Generally, the patient is suspected of having MS. For example, the patient may be suspected of having MS on the basis of neurologic and/or immunologic symptoms consistent with MS, e.g., after an initial physician's exam. The patient may, in some embodiments, be positive for the presence of oligoclonal bands. In these or other embodiments, the patient may have CNS lesions characteristic of MS, which are observable on an MRI. In certain embodiments, the patient has not undergone treatment for MS, but in some embodiments, the patient is already undergoing treatment, such as treatment with Beta-interferon, Glatiramer acetate, and Natalizumab.

Thus, the patient may have one or more presumptive signs of a multiple sclerosis. Presumptive signs of multiple sclerosis include for example, altered sensory, motor, visual or proprioceptive system with at least one of numbness or weakness in one or more limbs, often occurring on one side of the body at a time or the lower half of the body, partial or complete loss of vision, frequently in one eye at a time and often with pain during eye movement, double vision or blurring of vision, tingling or pain in numb areas of the body, electric-shock sensations that occur with certain head movements, tremor, lack of coordination or unsteady gait, fatigue, dizziness, muscle stiffness or spasticity, slurred speech, paralysis, problems with bladder, bowel or sexual function, and mental changes such as forgetfulness or difficulties with concentration, relative to medical standards.

The gene expression profile is determined for a blood sample, such as a whole blood sample or a white blood cell sample, of the patient. The white blood cell sample may be a Peripheral Blood Mononuclear Cell (PBMC) sample of the patient. PBMCs are a mixture of monocytes and lymphocytes. There are several known methods for isolating PBMCs from whole blood. While any suitable method may be employed, in one embodiment, PBMCs are isolated from whole blood samples using density gradient centrifugation. For example, anticoagulated whole blood is layered over a separating medium, and after centrifugation, the following layers are visually observed from top to bottom: plasma/platelets, PBMCs, separating medium and erythrocytes/granulocytes. The PBMC layer may then be removed for RNA isolation. Alternatively, the blood cell sample may be further isolated from whole blood or PBMCs to yield a cell subpopulation, such as a population of lymphocytes (e.g., T-lymphocytes or sub-population thereof). Examples for isolating such sub-populations are known in the art, and include cell sorting, or cell-capturing using antibodies to particular cell-specific markers.

In preparing the gene expression profile of the patient sample(s), RNA is extracted from the collected cells (e.g., using whole blood or PBMC samples) by any known method. For example, RNA may be purified from cells using a variety of standard procedures as described, for example, in RNA Methodologies, A laboratory guide for isolation and characterization, 2nd edition, 1998, Robert E. Farrell, Jr., Ed., Academic Press. In addition, there are various products commercially available for RNA isolation which may be used. Total RNA or polyA+ RNA may be used for preparing gene expression profiles in accordance with the invention.

The gene expression profile (or gene expression signature) is then generated for the samples using any of various techniques known in the art, and described in detail elsewhere herein. Such methods generally include, without limitation, polymerase-based assays, such as RT-PCR (e.g., Taqman™), hybridization-based assays, such as DNA microarray analysis, flap-endonuclease-based assays (e.g., Invader™), as well as direct mRNA capture with branched DNA (QuantiGene™) or Hybrid Capture™ (Digene).

The gene expression profile contains gene expression levels for a plurality of genes that are differentially expressed in blood samples of MS patients, and such genes are disclosed herein. For example, Tables 1 and 2 list genes that are differentially expressed in whole blood and PBMC samples, respectively, of MS patients. As used herein, the term “gene,” refers to a DNA sequence expressed in a sample as an RNA transcript, and may be a full-length gene (protein encoding or non-encoding) or an expressed fragment such as expressed sequence tags or “ESTs.” Thus, the sequences listed in the Tables and Sequence Listing are each independently a full-length gene sequence, whose expression product is present in samples, or is a partial expressed sequence detectable in samples, such as an EST sequence. As used herein, “differentially expressed” means that the level or abundance of an RNA transcript (or abundance of an RNA population sharing a common target sequence, such as splice variant RNAs) is significantly higher or lower in a sample as compared to a reference level. For example, the level of the RNA or RNA population may be higher or lower by at least two-fold as compared to a reference level. The reference level is the level of the same RNA or RNA population in a control sample or control population (e.g., a Mean control level).

Table 1A lists genes that are differentially expressed in whole blood of MS patients, and thus the expression level of these genes or subset thereof may be determined in patient samples to prepare a gene expression profile in accordance with this aspect of the invention. Table 1A refers to these genes by name (title and abbreviation), GeneBank Accession No., and sequence identifier as found in the accompanying Sequence Listing. Table 1A further provides Mean-MS and Mean-control gene expression levels as generated from an exemplary sample set and data set, as well as measures of the statistical association of each differential gene expression level with MS. Table 1B lists these same genes, and expresses the differential RNA levels as fold change (Control/MS), MeanRatio (Control/MS), and Mean Difference (Control-MS).

Thus, in accordance with this aspect, the patient's gene expression profile, which is generated from the patient's blood sample (e.g., a whole blood sample), may contain the levels of expression for at least about 3 genes listed in Table 1. In some embodiments, the patient's gene expression profile contains the levels of expression for at least about 5, 7, 10, 12, 15, 20, 25, 40, 50, 75, or 100 genes listed in Table 1, such genes being differentially expressed in blood of MS patients over non-MS individuals. In some embodiments, the gene expression profile may contain the levels of expression for all or substantially all genes listed in Table 1, such as at least about 200, 250, or 300 genes. In any of the embodiments described in this paragraph, the gene expression profile may comprise the level of expression of at least one gene that is also listed in Table 6. That is, at least one of the genes is listed in both Table 1 and Table 6.

Table 2A lists genes that are differentially expressed in PBMCs of MS patients, and thus the expression level of these genes or subset thereof may be determined in patient samples to prepare a gene expression profile in accordance with this aspect of the invention. Table 2A refers to these genes by name (title and abbreviation), GeneBank Accession No., and sequence identifier as found in the accompanying Sequence Listing. Table 2A further provides the fold change between control and MS samples as generated from an exemplary sample set and data set, as well as measures of the statistical association of each differential gene expression level with MS. Table 2B lists these same genes, and shows the mean control and MS data signals, and indicates the top 42 genes in terms of fold change.

Thus, in accordance with these embodiments, the patient's gene expression profile (generated from whole blood or a white blood cell sample, including a PBMC sample) may contain the levels of expression for at least about 3 genes listed in Table 2. In some embodiments, the patient's gene expression profile contains the levels of expression for at least about 5, 7, 10, 12, 15, 20, 25, 40, 50, 75 or 100 genes listed in Table 2, such genes being differentially expressed in blood, and particularly PBMCs, of MS patients over non-MS individuals (controls). In some embodiments, the gene expression profile may contain the levels of expression for all or substantially all genes listed in Table 2, such as at least about 200, 250, or 300 genes. In any of the embodiments described in this paragraph, the gene expression profile may comprise the level of expression of at least one gene that is also listed in Table 6. That is, at least one of the genes is listed in both Table 2 and Table 6. In some preferred embodiments, one or more, or all, of the genes in Table 2 that are included in the gene expression profile, are in the top 42 genes in terms of fold change between MS and controls, as shown in Table 2.

In certain embodiments, the gene expression profile contains a measure of expression levels for a plurality of genes that are each, independently, expressed in MS samples relative to control samples by a fold change magnitude (up or down) of at least 1.2. In some embodiments, the plurality of genes are differentially expressed in MS samples with respect to control samples (e.g., non-MS sample) by a fold change magnitude of at least 1.5, or at least about 1.7, or at least about 2, or at least about 2.5. Alternatively, the expression levels may differ by at least about 3- or 5-, 10-fold, or more. Tables 1 and 2 list genes by differential levels of expression in control versus MS samples, as determined in whole blood or PBMCs, respectively, and such levels may be used to select genes for profiling in accordance with this paragraph.

The gene expression profile prepared according to this aspect of the invention is compared to an MS-profile and/or a non-MS profile, to classify the patient's gene expression profile as an MS profile or a non-MS profile. In certain embodiments, the non-MS profile is a healthy profile. In these or other embodiments, the MS-profile may be a general MS-profile (e.g., not limited to a clinical MS subtype), or may be a relapsing-remitting MS profile. Tables 1 and 2 present exemplary MS and non-MS profiles, which may be used to classify patient samples. Of course, additional MS and non-MS profiles for classifying samples may be generated from additional MS and control sample sets, using the genes listed in Tables 1 and 2 as described above.

Various classification schemes are known for classifying samples between two or more classes or groups, and these include, without limitation: Naïve Bayes, Support Vector Machines, Nearest Neighbors, Decision Trees, Logistic, Artificial Neural Networks, and Rule-based schemes. In addition, the predictions from multiple models can be combined to generate an overall prediction. For example, a “majority rules” prediction may be generated from the outputs of a Naïve Bayes model, a Support Vector Machine model, and a Nearest Neighbor model.

Thus, a classification algorithm or “class predictor” may be constructed to classify samples. The process for preparing a suitable class predictor is reviewed in R. Simon, Diagnostic and prognostic prediction using gene expression profiles in high-dimensional microarray data, British Journal of Cancer (2003) 89, 1599-1604, which review is hereby incorporated by reference in its entirety.

Generally, the gene expression profile for the patient is compared to both a non-MS profile and an MS profile. Such MS and non-MS profiles may be assembled from gene expression data disclosed herein (Tables 1 and 2), which may be stored in a database and correlated to patient profiles. Thus, MS and non-MS profiles may be assembled from data and matched to a particular patient by, for example, age, race, gender, and/or clinical manifestations of MS. The MS profile may represent a particular clinical course of MS, such as relapsing-remitting MS.

After comparing the patient's gene expression profile to the MS and/or non-MS profile, the sample is classified as, or for example, given a probability of being, an MS profile or a non-MS profile. The classification may be determined computationally based upon known methods as described above. The result of the computation may be displayed on a computer screen or presented in a tangible form, for example, as a probability (e.g., from 0 to 100%) of the patient having MS. The report will aid a physician in diagnosis or treatment of the patient. For example, in certain embodiments of the invention, the patient's gene expression profile will be determined to be an MS profile on the basis of a probability, and the patient will be subsequently treated for MS as appropriate. In other embodiments, the patient's profile will be determined to be a non-MS profile, thereby allowing the physician to exclude MS as a diagnosis for the patient.

In various embodiments, the method according to this aspect of the invention distinguishes a MS-afflicted patient from a non-MS afflicted patient with at least about 50%, 75%, 80%, 85%, 90%, 95%, 97%, 98%, 99% or greater accuracy. In this respect, the method according to this aspect may lend additional or alternative predictive value over standard clinical methods of diagnosing MS, such as for example, absence or presence of lesions on an MRI, testing positive or negative for oligoclonal bands, or the absence or presence of other signs and symptoms of MS such as blurred vision, fatigue, and/or loss of balance.

### Methods For Determining Efficacy of Treatment

In a second aspect, the invention is a method for monitoring treatment of an MS patient. While any treatment program may be monitored, including test compounds, in certain embodiments the patient is undergoing treatment with one or more of Beta-Interferon, Glatiramer acetate, and Natalizumab. In this aspect, the invention comprises determining a pre-treatment (or early treatment) gene expression profile and at least one post-treatment gene expression profile for the patient, as already described. The pre-treatment profile may be determined from a sample taken prior to treatment, or may be an early treatment profile determined, for example, for a sample taken within the first six months of treatment. The post-treatment profile(s) may be determined for samples taken anytime after the start of treatment, such as after about three months, after about six months, after about twelve months of treatment, and/or later.

The pre-treatment and post-treatment gene expression profiles are prepared from blood samples (e.g., whole blood) or white blood cell samples (such as PBMC samples or subpopulation thereof), isolated from the patient at the selected pre- and post-treatment time points.

The pre- and post-treatment gene expression profiles contain gene expression levels for a plurality of genes that are differentially expressed in blood cells of MS patients, as described in the preceding section with respect to Tables 1, 2, and 6. Thus, in accordance with this aspect, the patient's gene expression profile, which is generated from the patient's blood sample (e.g., a whole blood sample), may contain the levels of expression for at least about 3 genes listed in Table 1. In some embodiments, the patient's gene expression profile contains the levels of expression for at least about 5, 7, 10, 12, 15, 20, 25, 40, 50, 75, or 100 genes listed in Table 1, such genes being differentially expressed in blood of MS patients over non-MS individuals. In some embodiments, the gene expression profile may contain the levels of expression for all or substantially all genes listed in Table 1, such as at least about 200, 250, or 300 genes. In any of the embodiments described in this paragraph, the gene expression profile may comprise the level of expression of at least one gene that is also listed in Table 6. That is, at least one of the genes is listed in both Table 1 and Table 6. Alternatively, the patient's gene expression profile (generated from whole blood or a white blood cell sample, including a PBMC sample) may contain the levels of expression for at least about 3 genes listed in Table 2. In some embodiments, the patient's gene expression profile contains the levels of expression for at least about 5, 7, 10, 12, 15, 20, 25, 40, 50, 75 or 100 genes listed in Table 2, such genes being differentially expressed in blood, and particularly PBMCs, of MS patients over non-MS individuals (controls). In some embodiments, the gene expression profile may contain the levels of expression for all or substantially all genes listed in Table 2, such as at least about 200, 250, or 300 genes. In any of the embodiments described in this paragraph, the gene expression profile may comprise the level of expression of at least one gene that is also listed in Table 6. That is, at least one of the genes is listed in both Table 2 and Table 6. In some preferred embodiments, one or more, or all, of the genes in Table 2 that are included in the gene expression profile, are in the top 42 genes in terms of fold change between MS and controls, as shown in Table 2.

Thus, such gene expression profiles may be useful for monitoring a patient's treatment, to determine whether the post-treatment sample classifies as an MS-sample, to the same, lesser, or greater extent as the pre-treatment sample. Such pre-treatment and post-treatment samples may be classified or scored as MS or non-MS samples as disclosed elsewhere herein.

Alternatively, or in addition, the pre-treatment and post-treatment gene expression profiles may be compared to identify differences in gene expression upon treatment with MS. For example, where the patient is being treated with Beta-interferon, gene expression values may be determined (pre- and post-treatment) for genes (e.g., 3, 5, 7, 10, 15, 20, or 40 genes) listed in Tables 3 and/or 5. In certain embodiments, at least one gene is also listed in Table 6, in addition to being listed in Tables 3 and/or 5. Or, where the patient is being treated with Glatiramer acetate, gene expression values may be determined (pre- and post-treatment) for genes (e.g., 3, 5, 7, 10, 15, 20, or 40 genes) listed in Tables 4 or 5. In certain embodiments, at least one gene is also listed in Table 6, in addition to being listed in Tables 3 and/or 5. Tables 3 and 5 list genes that are differentially expressed in PBMCs between pre- and post-treatment with Beta-interferon. Table 4 and 5 list genes that are differentially expressed in PBMCs between pre- and post-treatment with Copaxone. Table 5 lists genes that are differentially expressed between pre- and post-treatment with each of Beta-Interferon and Glatiramer acetate.

The pre-treatment gene expression profile may then be compared with the post-treatment gene expression profile, to identify differences between pre-treatment and post-treatment gene expression. These differences may be indicative of the patient's response (positive or negative) to treatment.

Many of the genes that are indicative of a patient's response to Beta-interferon may encode cell surface markers, e.g. cell surface markers on immune cells, and several of which are interferon-inducible genes (see Table 3). Accordingly, in some embodiments of the invention, at least one, or at least five, or at least 10 of the genes in the gene expression profile encode a cell-surface marker, some or all of which are interferon-inducible. Such genes are listed in Example 2, herein.

After comparison, the post-treatment gene expression profile may be classified as being indicative of MS, or not being indicative of MS (or being less indicative of MS than the pre-treatment sample), for example due to effective therapy. Alternatively, the post-treatment sample may be more indicative of MS, suggesting that an alternative therapy would be desirable. The analysis in accordance with this aspect may be performed computationally as described. The result of the analysis may be displayed or presented in tangible form to aid in considering further treatment options, such as adjusting or changing the treatment, if needed, and to track the clinical course of the patient's disease.

### Methods for Preparing Gene Expression Profiles

In a third aspect, the invention provides a method for preparing a patient gene expression profile for evaluating MS. According to this aspect, the gene expression profile is useful for determining whether the patient has MS, as well as for monitoring the course of the disease, and predicting whether a particular treatment is or will be efficacious. In accordance with this aspect, the method generally comprises quantifying the level of expression, in a patient blood sample, for a plurality of genes listed in one of Tables 1-5 as discussed above for the first and second aspects of the invention.

For example, the gene expression profile may contain the levels of expression in the sample for a plurality of genes listed in Table 1 or Table 2. In some embodiments, at least one of these genes is also listed in Table 6. In accordance with this aspect, the patient may have, or be suspected to have, MS. Such gene expression profiles are useful for classifying samples as MS or non-MS samples in accordance with the first aspect of the invention, or for monitoring the course of the patient's MS over time.

Specifically, in accordance with this aspect, the patient's gene expression profile, which is generated from the patient's blood sample (e.g., a whole blood sample), may contain the levels of expression for at least about 3 genes listed in Table 1. In some embodiments, the patient's gene expression profile contains the levels of expression for at least about 5, 7, 10, 12, 15, 20, 25, 40, 50, 75, or 100 genes listed in Table 1, such genes being differentially expressed in blood of MS patients over non-MS individuals. In some embodiments, the gene expression profile may contain the levels of expression for all or substantially all genes listed in Table 1, such as at least about 200, 250, or 300 genes. In any of the embodiments described in this paragraph, the gene expression profile may comprise the level of expression of at least one gene that is also listed in Table 6. That is, at least one of the genes is listed in both Table 1 and Table 6.

Alternatively, the patient's gene expression profile (generated from whole blood or a white blood cell sample, including a PBMC sample) may contain the levels of expression for at least about 3 genes listed in Table 2. In some embodiments, the patient's gene expression profile contains the levels of expression for at least about 5, 7, 10, 12, 15, 20, 25, 40, 50, 75 or 100 genes listed in Table 2, such genes being differentially expressed in blood, and particularly PBMCs, of MS patients over non-MS individuals (controls). In some embodiments, the gene expression profile may contain the levels of expression for all or substantially all genes listed in Table 2, such as at least about 200, 250, or 300 genes. In any of the embodiments described in this paragraph, the gene expression profile may comprise the level of expression of at least one gene that is also listed in Table 6. That is, at least one of the genes is listed in both Table 2 and Table 6. In some preferred embodiments, one or more, or all, of the genes in Table 2 that are included in the gene expression profile, are in the top 42 genes in terms of fold change between MS and controls, as shown in Table 2.

Alternatively, the gene expression profile may contain the levels of expression in the sample for a plurality of genes listed in Table 3, 4, and/or 5. Preferably, at least one of these genes is also listed in Table 6. Such gene expression profiles are useful for predicting whether a particular treatment might be efficacious, or where treatment is already ongoing, determining whether the current treatment is effective.

### Assay Formats

Gene expression profiles, including patient gene expression profiles and the MS and non-MS profiles as described herein, may be prepared according to any suitable method for measuring gene expression. That is, the profiles may be prepared using any quantitative or semi-quantitative method for determining RNA transcript levels in samples. Such methods include polymerase-based assays, such as RT-PCR, Taqman™, hybridization-based assays, for example using DNA microarrays or other solid support, nucleic acid sequence based amplification (NASBA), flap endonuclease-based assays, as well as direct mRNA capture with branched DNA (QuantiGene™) or Hybrid Capture™ (Digene). The assay format, in addition to determining the gene expression levels for a combination of genes listed in one or more of Tables 1-6, will also allow for the control of, inter alia, intrinsic signal intensity variation between tests. Such controls may include, for example, controls for background signal intensity and/or sample processing, and/or other desirable controls for gene expression quantification across samples. For example, expression levels between samples may be controlled by testing for the expression level of one or more genes that are not differentially expressed in MS patients, or which are generally expressed at similar levels across the population. Such genes may include constitutively expressed genes, many of which are known in the art. Exemplary assay formats for determining gene expression levels, and thus for preparing gene expression profiles and MS- and non-MS profiles are described in this section.

The nucleic acid sample is typically in the form of mRNA or reverse transcribed mRNA (cDNA) isolated from a blood sample, such as a whole blood sample, PBMC sample, or other subpopulation of blood cells (e.g., T-lymphocytes) isolated from the patient's blood. In some embodiments, the nucleic acids in the sample may be cloned or amplified, generally in a manner that does not bias the representation of the transcripts within a sample. In some embodiments, it may be preferable to use total RNA or polyA+ RNA as a source without cloning or amplification, to avoid additional processing steps.

As is apparent to one of skill in the art, nucleic acid samples used in the methods of the invention may be prepared by any available method or process. Methods of isolating total mRNA are well known to those of skill in the art. For example, methods of isolation and purification of nucleic acids are described in detail in Chapter 3 of Laboratory Techniques in Biochemistry and Molecular Biology, Vol. 24, Hybridization With Nucleic Acid Probes: Theory and Nucleic Acid Probes, P. Tijssen, Ed., Elsevier Press, New York, 1993. Such samples include RNA samples, but also include cDNA synthesized from a mRNA sample isolated from a cell or tissue of interest (e.g., whole blood or PBMC sample). Such samples also include DNA amplified from the cDNA, and RNA transcribed from the amplified DNA.

In determining a patient's gene expression profile, or in determining an MS- or non-MS profile in accordance with the invention, a hybridization-based assay may be employed. Nucleic acid hybridization involves contacting a probe and a target sample under conditions where the probe and its complementary target sequence (if present) in the sample can form stable hybrid duplexes through complementary base pairing. The nucleic acids that do not form hybrid duplexes may be washed away leaving the hybridized nucleic acids to be detected, typically through detection of an attached detectable label. It is generally recognized that nucleic acids may be denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In certain embodiments, hybridization is performed at low stringency, such as 6×SSPET at 37° C. (0.005% Triton X-100), to ensure hybridization, and then subsequent washes are performed at higher stringency (e.g., 1×SSPET at 37° C.) to eliminate mismatched hybrid duplexes. Successive washes may be performed at increasingly higher stringency (e.g., down to as low as 0.25×SSPET at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that may be present, as described below (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. The hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

The hybridized nucleic acids are typically detected by detecting one or more labels attached to the sample nucleic acids. The labels may be incorporated by any of a number of means well known to those of skill in the art. See WO 99/32660.

Numerous hybridization assay formats are known, and which may be used in accordance with the invention. Such hybridization-based formats include solution-based and solid support-based assay formats. Solid supports containing oligonucleotide probes designed to detect differentially expressed genes (e.g., listed in Tables 1-5) can be filters, polyvinyl chloride dishes, particles, beads, microparticles or silicon or glass based chips, etc. Any solid surface to which oligonucleotides can be bound, either directly or indirectly, either covalently or non-covalently, may be used. Bead-based assays are described, for example, in U.S. Pat. Nos. 6,355,431, 6,396,995, and 6,429,027, which are hereby incorporated by reference. Other chip-based assays are described in U.S. Pat. Nos. 6,673,579, 6,733,977, and 6,576,424, which are hereby incorporated by reference.

An exemplary solid support is a high density array or DNA chip, which may contain a particular oligonucleotide probes at predetermined locations on the array. Each predetermined location may contain more than one molecule of the probe, but each molecule within the predetermined location has an identical probe sequence. Such predetermined locations are termed features. Probes corresponding to the genes of Tables 1-5 may be attached to single or multiple solid support structures, e.g., the probes may be attached to a single chip or to multiple chips to comprise a chip set.

Oligonucleotide probe arrays for expression monitoring can be made and used according to any techniques known in the art (see for example, Lockhart et al (1996), Nat Biotechnol 14:1675-1680; McGall et al. (1996), Proc Nat Acad Sci USA 93:13555-13460). Such probe arrays may contain the oligonucleotide probes necessary for determining a patient's gene expression profile, or for preparing MS- and non-MS profiles with population samples. Thus, such arrays may contain oligonucleotide designed to hybridize to at least 2, 3, 4, 5, 6, 7, 8, 9, 10, 20, 30, 50, 70, 100, 200, 300 or more of the genes described herein (e.g., as described in one of Tables 1-5, or as described in any of Tables 1-5). In some embodiments, the array contains probes designed to hybridize to all or nearly all of the genes listed in Tables 1-5. In still other embodiments, arrays are constructed that contain oligonucleotides designed to detect all or nearly all of the genes in Table 1-5 on a single solid support substrate, such as a chip or a set of beads.

Probes based on the sequences of the genes described herein for preparing expression profiles may be prepared by any suitable method. Oligonucleotide probes, for hybridization-based assays, will be of sufficient length or composition (including nucleotide analogs) to specifically hybridize only to appropriate, complementary nucleic acids (e.g., exactly or substantially complementary RNA transcripts or cDNA). Typically the oligonucleotide probes will be at least about 10, 12, 14, 16, 18, 20 or 25 nucleotides in length. In some cases, longer probes of at least 30, 40, or 50 nucleotides may be desirable. In some embodiments, complementary hybridization between a probe nucleic acid and a target nucleic acid embraces minor mismatches (e.g., one, two, or three mismatches) that can be accommodated by reducing the stringency of the hybridization media to achieve the desired detection of the target polynucleotide sequence. Of course, the probes may be perfect matches with the intended target probe sequence, for example, the probes may each have a probe sequence that is perfectly complementary to a target sequence (e.g., a sequence of a gene listed in Tables 1-5).

A probe is a nucleic acid capable of binding to a target nucleic acid of complementary sequence through one or more types of chemical bonds, usually through complementary base pairing, usually through hydrogen bond formation. A probe may include natural (i.e., A, G, U, C, or T) or modified bases (7-deazaguanosine, inosine, etc.), or locked nucleic acid (LNA). In addition, the nucleotide bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages.

When using hybridization-based assays, in may be necessary to control for background signals. The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each location of the array. In an exemplary embodiment, background is calculated as the average hybridization signal intensity for the lowest 5% to 10% of the probes in the array. Alternatively, background may be calculated as the average hybridization signal intensity produced by hybridization to probes that are not complementary to any sequence found in the sample (e.g. probes directed to nucleic acids of the opposite sense or to genes not found in the sample such as bacterial genes where the sample is mammalian nucleic acids). Background can also be calculated as the average signal intensity produced by regions of the array that lack any probes at all. Of course, one of skill in the art will appreciate that hybridization signals may be controlled for background using one or a combination of known approached, including one or a combination of approaches described in this paragraph.

The hybridization-based assay will be generally conducted under conditions in which the probe(s) will hybridize to their intended target subsequence, but with only insubstantial hybridization to other sequences or to other sequences, such that the difference may be identified. Such conditions are sometimes called “stringent conditions.” Stringent conditions are sequence-dependent and can vary under different circumstances. For example, longer probe sequences generally hybridize to perfectly complementary sequences (over less than fully complementary sequences) at higher temperatures. Generally, stringent conditions may be selected to be about 5° C. lower than the thermal melting point (Tm) for the specific sequence at a defined ionic strength and pH. Exemplary stringent conditions may include those in which the salt concentration is at least about 0.01 to 1.0 M Na⁺ ion concentration (or other salts) at pH 7.0 to 8.3 and the temperature is at least about 30° C. for short probes (e.g., 10 to 50 nucleotides). Desired hybridization conditions may also be achieved with the addition of agents such as formamide or tetramethyl ammonium chloride (TMAC).

When using an array, one of skill in the art will appreciate that an enormous number of array designs are suitable for the practice of this invention. The array will typically include a number of test probes that specifically hybridize to the sequences of interest. That is, the array will include probes designed to hybridize to any region of the genes listed in Tables 1-5, and the accompanying sequence listing. In instances where the gene reference in the Tables is an EST, probes may be designed from that sequence or from other regions of the corresponding full-length transcript that may be available in any of the public sequence databases, such as those herein described. See WO 99/32660 for methods of producing probes for a given gene or genes. In addition, software is commercially available for designing specific probe sequences. Typically, the array will also include one or more control probes, such as probes specific for a constitutively expressed gene, thereby allowing data from different arrays to be normalized or controlled.

The hybridization-based assays may include, in addition to “test probes” (e.g., that bind the target sequences of interest, which are listed in Tables 1-6), the assay may also test for hybridization to one or a combination of control probes. Exemplary control probes include: normalization controls, expression level controls, and mismatch controls. For example, when determining the levels of gene expression in patient or control samples, the expression values may be normalized to control between samples. That is, the levels of gene expression in each sample may be normalized by determining the level of expression of at least one constitutively expressed gene in each sample. In accordance with the invention, the constitutively expressed gene is generally not differentially expressed in samples (blood samples, including whole blood or PBMC samples) of MS patients.

Other useful controls are normalization controls, for example, using probes designed to be complementary to a labeled reference oligonucleotide added to the nucleic acid sample to be assayed. The signals obtained from the normalization controls after hybridization provide a control for variations in hybridization conditions, label intensity, “reading” efficiency and other factors that may cause the signal of a perfect hybridization to vary between arrays. In one embodiment, signals (e.g., fluorescence intensity) read from all other probes in the array are divided by the signal (e.g., fluorescence intensity) from the control probes thereby normalizing the measurements. Exemplary normalization probes are selected to reflect the average length of the other probes (e.g., test probes) present in the array, however, they may be selected to cover a range of lengths. The normalization control(s) may also be selected to reflect the (average) base composition of the other probes in the array. In some embodiments, the assay employs one or a few normalization probes, and they are selected such that they hybridize well (i.e., no secondary structure) and do not hybridize to any potential targets.

The hybridization-based assay may employ expression level controls, for example, probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the actin gene, the transferrin receptor gene, the GAPDH gene, and the like.

The hybridization-based assay may also employ mismatch controls for the target sequences, and/or for expression level controls or for normalization controls. Mismatch controls are probes designed to be identical to their corresponding test or control probes, except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a 20-mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. For example, if the target is present, the perfect match probes should provide a more intense signal than the mismatch probes. The difference in intensity between the perfect match and the mismatch probe helps to provide a good measure of the concentration of the hybridized material.

Methods of forming high density arrays of oligonucleotides with a minimal number of synthetic steps are known. The oligonucleotide analogue array can be synthesized on a single or on multiple solid substrates by a variety of methods, including, but not limited to, light-directed chemical coupling, and mechanically directed coupling (see Pirrung, U.S. Pat. No. 5,143,854). In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithographic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in PCT Publication Nos. WO 93/09668 and WO 01/23614. High density nucleic acid arrays can also be fabricated by depositing pre-made or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

The hybdridization-based assay may, as an alternative to purely passive hybridization, employ the methods described in U.S. Pat. No. 6,326,173, which is hereby incorporated by reference. For example, the assay may involve electronically concentrating and hybridizing the nucleic acid sample to the surface of a microchip (e.g., capture sites). This method may allow for rapid concentration and subsequent specific hybridization of template nucleic acid molecules to their complementary anchored amplification primers.

Alternatively, the invention may employ reverse transcription polymerase chain reaction (RT-PCR), which is a sensitive method for the detection of mRNA, including low abundant mRNAs present in clinical samples. The application of fluorescence techniques to RT-PCR combined with suitable instrumentation has led to quantitative RT-PCR methods that combine amplification, detection and quantification in a closed system. Two commonly used quantitative RT-PCR techniques are the Taqman RT-PCR assay (ABI, Foster City, USA) and the Lightcycler assay (Roche, USA).

Thus, in one embodiment of the present invention, the preparation of patient gene expression profiles or the preparation of MS- and non-MS profiles comprises conducting real-time quantitative PCR (TaqMan) with sample-derived RNA and control RNA. Holland, et al., PNAS 88:7276-7280 (1991) describe an assay known as a Taqman assay. The 5′ to 3′ exonuclease activity of Taq polymerase is employed in a polymerase chain reaction product detection system to generate a specific detectable signal concomitantly with amplification. An oligonucleotide probe, non-extendable at the 3′ end, labeled at the 5′ end, and designed to hybridize within the target sequence, is introduced into the polymerase chain reaction assay. Annealing of the probe to one of the polymerase chain reaction product strands during the course of amplification generates a substrate suitable for exonuclease activity. During amplification, the 5′ to 3′ exonuclease activity of Taq polymerase degrades the probe into smaller fragments that can be differentiated from undegraded probe. A version of this assay is also described in Gelfand et al., in U.S. Pat. No. 5,210,015, which is hereby incorporated by reference.

Further, U.S. Pat. No. 5,491,063 to Fisher, et al., which is hereby incorporated by reference, provides a Taqman-type assay. The method of Fisher et al. provides a reaction that results in the cleavage of single-stranded oligonucleotide probes labeled with a light-emitting label wherein the reaction is carried out in the presence of a DNA binding compound that interacts with the label to modify the light emission of the label. The method of Fisher uses the change in light emission of the labeled probe that results from degradation of the probe.

The TaqMan detection assays offer certain advantages. First, the methodology makes possible the handling of large numbers of samples efficiently and without cross-contamination and is therefore adaptable for robotic sampling. As a result, large numbers of test samples can be processed in a very short period of time using the TaqMan assay. Another advantage of the TaqMan system is the potential for multiplexing. Since different fluorescent reporter dyes can be used to construct probes, the expression of several different genes associated with MS could be assayed in the same PCR reaction, thereby reducing the labor costs that would be incurred if each of the tests were performed individually. Thus, the TaqMan assay format is preferred where the patient's gene expression profile, and the corresponding MS- and non-MS profiles comprise the expression levels of about 20 of fewer, or about 10 or fewer, or about 7 of fewer, or about 5 genes (e.g., genes listed in Tables 1-6).

Alternatively, the assay format may employ the methodologies described in Direct Multiplexed Measurement of Gene Expression with Color-Coded Probe Pairs, Nature Biotechnology (Mar. 7, 2008), which describes the nCounter™ Analysis System (nanoString Technologies). This system captures and counts individual mRNA transcripts by a molecular bar-coding technology, and is commercialized by Nanostring.

In other embodiments, the invention employs detection and quantification of RNA levels in real-time using nucleic acid sequence based amplification (NASBA) combined with molecular beacon detection molecules. NASBA is described for example, in Compton J., Nucleic acid sequence-based amplification, Nature 1991; 350(6313): 91-2. NASBA is a singe-step isothermal RNA-specific amplification method. Generally, the method involves the following steps: RNA template is provided to a reaction mixture, where the first primer attaches to its complementary site at the 3′ end of the template; reverse transcriptase synthesizes the opposite, complementary DNA strand; RNAse H destroys the RNA template (RNAse H only destroys RNA in RNA-DNA hybrids, but not single-stranded RNA); the second primer attaches to the 3′ end of the DNA strand, and reverse transcriptase synthesizes the second strand of DNA; and T7 RNA polymerase binds double-stranded DNA and produces a complementary RNA strand which can be used again in step 1, such that the reaction is cyclic.

In yet other embodiments, the assay format is a flap endonuclease-based format, such as the Invader™ assay (Third Wave Technologies). In the case of using the invader method, an invader probe containing a sequence specific to the region 3′ to a target site, and a primary probe containing a sequence specific to the region 5′ to the target site of a template and an unrelated flap sequence, are prepared. Cleavase is then allowed to act in the presence of these probes, the target molecule, as well as a FRET probe containing a sequence complementary to the flap sequence and an auto-complementary sequence that is labeled with both a fluorescent dye and a quencher. When the primary probe hybridizes with the template, the 3′ end of the invader probe penetrates the target site, and this structure is cleaved by the Cleavase resulting in dissociation of the flap. The flap binds to the FRET probe and the fluorescent dye portion is cleaved by the Cleavase resulting in emission of fluorescence.

In yet other embodiments, the assay format employs direct mRNA capture with branched DNA (QuantiGene™, Panomics) or Hybrid Capture™ (Digene).

The design of appropriate probes for hybridizing to a particular target nucleic acid, and as configured for any appropriate nucleic acid detection assay, is well known.

### Computer Systems

In another aspect, the invention is a computer system that contains a database, on a computer-readable medium, of mean gene expression values determined in an MS patient population and in a non-MS patient population. These gene expression values are determined in blood samples, such as whole blood cell samples or white blood cell samples (e.g., PBMC samples), and for genes selected from one or more of Tables 1-5. The database may include gene expression measurements for at least one or a plurality of genes that are also listed in Table 6. The database may include, for each gene, Mean-MS and Mean-Control (e.g., non-MS or healthy) gene expression levels, as well as various statistical measures, including measures of value dispersion (e.g., Standard Variation), fold change (e.g., between control and MS populations), and statistical significance (statistical association with MS). Various such measures are shown in the accompanying Tables, and such measures may be employed in the computer systems of the invention. Further, the MS patient population may include patients being treated with Beta-interferon, Glatiramer acetate, and/or Natalizumab, and such treatment and other clinical information may be included in the database such that an appropriate gene expression profile may be assembled for use with the diagnostic methods of the invention. Generally, profiles may be assembled based upon parameters to be selected and input by a user, with these parameters including one or more of age, race, gender, MS treatment, and clinical manifestation and course of MS.

In certain embodiments, the database contains mean gene expression values for at least about 5, 7, 10, 20, 40, 50, or 100 genes selected from any one, or a combination of, Tables 1-6. In some embodiments, the database may contain mean gene expression values for more than about 100 genes, or about 300 genes, or about 400 genes selected from Tables 1-6. In one embodiment, the database contains mean gene expression values for all or substantially all the genes listed in Tables 1-6. The database may include gene expression measurements for at least one or a plurality of genes that are also listed in Table 6.

The computer system of the invention may be programmed to compare (e.g., in response to user inputs) a gene expression profile to a non-MS gene expression profile and/or an MS-gene expression profile stored and/or generated from the database, to determine whether the gene expression profile is itself an MS-profile or a non-MS profile. For example, the computer system may be programmed to perform any of the known classification schemes for classifying gene expression profiles. Various classification schemes are known for classifying samples, and these include, without limitation: Naïve Bayes, Support Vector Machines, Nearest Neighbors, Decision Trees, Logistic, Artificial Neural Networks, and Rule-based schemes. The computer system may employ a classification algorithm or “class predictor” as described in R. Simon, Diagnostic and prognostic prediction using gene expression profiles in high-dimensional microarray data, British Journal of Cancer (2003) 89, 1599-1604, which is hereby incorporated by reference in its entirety.

The computer system of the invention may comprise a user interface, allowing a user to input gene expression values for comparison to an MS and/or non-MS gene expression profile, or gene expression profile previously generated for the patient. The patient's gene expression values may be input from a location remote from the database.

The computer system may further comprise a display, for presenting and/or displaying a result, such as a profile assembled from the database, or the result of a comparison (or classification) between input gene expression values and an MS and non-MS profiles. Such results may further be provided in a tangible form (e.g., as a printed report).

The computer system of the invention may further comprise relational databases containing sequence information, for instance, for the genes of Tables 1-5. For example, the database may contain information associated with a given gene, or patient sample, such as descriptive information about the gene associated with the sequence information, or descriptive information concerning the clinical status of the patient. The database may be designed to include different parts, for instance a sequence database and a gene expression database. Methods for the configuration and construction of such databases and computer-readable media to which such databases are saved are widely available, for instance, see U.S. Pat. No. 5,953,727, which is hereby incorporated by reference in its entirety.

The databases of the invention may be linked to an outside or external database (e.g., on the world wide web) such as GenBank (ncbi.nlm.nih.gov/entrez.index.html); KEGG (genome.ad.jp/kegg); SPAD (grt.kuyshu-u.ac.jp/spad/index.html); HUGO (gene.ucl.ac.uk/hugo); Swiss-Prot (expasy.ch.sprot); Prosite (expasy.ch/tools/scnpsitl.html); OMIM (ncbi.nlm.nih.gov/omim); and GDB (gdb.org). In certain embodiments, the external database is GenBank and the associated databases maintained by the National Center for Biotechnology Information (NCBI) (ncbi.nlm.nih.gov).

Any appropriate computer platform, user interface, etc. may be used to perform the necessary comparisons between sequence information, gene expression information (e.g., gene expression profiles) and any other information in the database or information provided as an input. For example, a large number of computer workstations are available from a variety of manufacturers, such has those available from Silicon Graphics. Client/server environments, database servers and networks are also widely available and appropriate platforms for the databases described herein.

The databases of the invention may be used to produce, among other things, electronic Northerns that allow the user to determine the samples in which a given gene is expressed and to allow determination of the abundance or expression level of the given gene.

### Diagnostic Kits

The invention further provides a kit or array containing nucleic acid primers and/or probes for determining the level of expression in a patient sample of a plurality of genes listed in Tables 1-5. The kit may consist essentially of primers and/or probes related to evaluating MS in a sample, and primers and/or probes related to necessary or meaningful assay controls (such as expression level controls and normalization controls, as described herein under “Assay Formats”). The kit for evaluating MS may comprise nucleic acid probes and/or primers designed to detect the expression level of ten or more genes associated with MS, such as the genes listed in Tables 1, 2, 3, 4, and/or 5. The kit may include a set of probes and/or primers designed to detect or quantify the expression levels of at least 5, 7, 10, or 20 genes listed in one or more of Tables 1, 2, 3, 4, and/or 5. In certain embodiments, the kit includes at least one probe and/or primer for quantifying expression of at least one or a plurality of genes that are also listed in Table 6. The primers and/or probes may be designed to detect gene expression levels in accordance with any assay format, including those described herein under the heading “Assay Format.” Exemplary assay formats include polymerase-based assays, such as RT-PCR, Taqman™, hybridization-based assays, for example using DNA microarrays or other solid support, nucleic acid sequence based amplification (NASBA), flap endonuclease-based assays.

In accordance with this aspect, the probes and primers may comprise antisense nucleic acids or oligonucleotides that are wholly or partially complementary to the diagnostic targets described herein (e.g., Tables 1-6). The probes and primers will be designed to detect the particular diagnostic target via an available nucleic acid detection assay format, which are well known in the art. The kits of the invention may comprise probes and/or primers designed to detect the diagnostic targets via detection methods that include amplification, endonuclease cleavage, and hybridization.

Without further description, it is believed that one of ordinary skill in the art can, using the preceding description and the following illustrative examples, make and utilize the compounds of the present invention and practice the claimed methods.

### EXAMPLES

MS and control (non-MS) gene expression profiles were identified by hybridization of RNA samples (from whole blood or PBMC samples) to microarrays.

Total RNA isolated from PBMC samples of 11 MS patients and 8 healthy controls was hybridized to a U133A/B chip set (Affymetrix).

Total RNA isolated from whole blood of 62 MS patients and 64 healthy controls was hybridized to a U133 Plus 2.0 chip (Affymetrix). These patients had at least one diagnosis of relapsing remitting MS.

Samples were processed as follows.

For PBMC isolation and subsequent RNA isolation, whole blood was collected into Vacutainer tubes (BD) containing an anticoagulant such as heparin. Peripheral blood mononuclear cells (PBMCs) were isolated by ficoll-hypaque density gradient centrifugation. Total RNA was isolated using standard RNA isolation kits (Qiagen).

For whole blood and subsequent RNA isolation, approximately 2.5 ml of whole blood was collected into each of 2-4 PAXgene tubes (BD/PreAnalytiX) using a blood collection set (BD Safety-Lok™ Blood Collection Set with butterfly needle), per PAXgene tube) following the protocol recommended by the manufacture. The PAXgene tubes were inverted 8-10 times to ensure proper mixture of blood with RNA stabilization solution. Total RNA was isolated using the PAXgene 96 Blood RNA Kit (Qiagen).

Following RNA isolation, the RNA was checked for quality, quantity and purity. Total RNA was evaluated for Quality by using the Agilent Bioanalyzer. RNA preps were quantified by measuring the absorbance at A260 and purity was assessed based on the ratio of the absorbance at A260/A280.

Hybridization of total RNA to microarrays, and following analysis of the data, produced the following MS and non-MS gene expression profiles (or “signatures”).

Table 1A lists genes that are differentially expressed in whole blood of MS patients, and provides an exemplary MS and non-MS profile for such genes. Table 1A refers to these genes by name (title and abbreviation), GeneBank Accession No., and sequence identifier as found in the accompanying Sequence Listing. Table 1A further provides Mean-MS and Mean-control gene expression levels as generated from the sample and data set, as well as measures of the statistical association of each differential gene expression level with MS. Table 1B lists these same genes, and expresses the differential RNA levels as fold change (Control/MS).

Table 2A lists genes that are differentially expressed in PBMCs of MS patients, and provides an exemplary MS and non-MS profile for such genes. Table 2A refers to these genes by name (title and abbreviation), GeneBank Accession No., and sequence identifier as found in the accompanying Sequence Listing. Table 2A further provides the fold change between control and MS samples as generated from this exemplary sample and data set, as well as measures of the statistical association of each differential gene expression level with MS. Table 2B lists these same genes, and shows the mean control and MS data signals, and indicates the top 42 genes in terms of fold change.

Tables 3 and 5 list genes that are differentially expressed in PBMCs between pre- and post-treatment with Beta-interferon. Table 4 and 5 list genes that are differentially expressed in PBMCs between pre- and post-treatment with Copaxone. Table 5 lists genes that are differentially expressed between pre- and post-treatment with each of Beta-Interferon and Glatiramer acetate.

The identification of such gene signatures, is illustrated in the following examples.

### Example 1

### Genes that are Differentially Expressed Between MS and Normal Samples Using a False Discovery Rate P-Value of 0.001.

The procedure to derive the list of differentially expressed genes in Table 2 was as follows.

A Genesis® (GeneLogic proprietary software) Comparative Analysis was performed using normal white blood cell (WBC) samples as the reference set and MS WBC samples as the experimental set. Parameters were as follows:
Log with Floor=0,
95% Confidence level,
Unequal Variance,
Exclude Expression with Call Values of “Unknown” or “No Value,”
Sample Set Expression Lower and Upper Percentiles of 25 and 75,
MAS5.0 Affymetrix Summarization,
Exclude gene fragments absent in all samples,

No other analysis filters were used. The analysis yielded 25,340 gene fragments. Fold Change, t-test p-value, and all sample expression values for each of these 25,340 fragments were exported from Genesis. All MAS5 logged expression values from Genesis for the 25,340 gene fragments for all 19 samples were uploaded into Partek Genomics Suite(R) software. A Benjamini and Hochberg False Discovery Rate correction was performed using a p-value cutoff of 0.001. This yielded a set of 339 non-control gene fragments listed in Table 2. Annotations for gene sets were based on UniGene assignments using the Gene Logic GeneIndex Build 33.

A correlation heatmap was calculated and visualized using Stat->Correlate->Similarity Matrix. Parameters=Pearson, All Combinations (19×19). The default heatmap showed the following. First, the normal (Control) group is cohesive and highly correlated. Second, the MS within-group correlations were higher than the Normal within-group correlations, except for three MS samples. These three MS samples were not as highly correlated to the remainder of the MS samples as the correlations for the majority of this group's intersample correlations. However, the three samples in question are also not highly correlated to each other, indicating absence of “batch” effect for contiguous GID's. Third, the Normal vs. MS correlations indicate robust differences even before gene selection, a sign that many genes are differentially expressed between the groups. Fourth, correlations are at levels consistent with what is expected. No outliers.

The data was transposed in Partek to allow for Principal Components Analysis (PCA). A categorical factor variable was added to the transposed data for coloring the PCA visual by terms “MS” and “Normal”. PCA Parameters: Correlation Dispersion Matrix, Normalized Eigenvector Scaling. Review of PCA showed: the variance explained by the plot is relatively larger than expected such that a robust response is expected upon hypothesis testing for individual genes. Even without prior gene selection, both groups separate well on the 1^{st} principal component, indicating a robust separation of samples. Further, the number of fragments p<0.01 (unadjusted) was 4,146, and the number of fragments p<0.01 (unadjusted) and with a fold change of >=2 was 386. This number of changing fragments indicates a high degree of regulation that is likely to be biologically real.

Comparison of the genes and gene fragments listed in Table 2 between baseline MS samples and control blood samples identifies a differentiating signature.

Thus, genes were further identified on the basis of stringent statistical differentiation thresholding, for example, using a fold-change cut-off to identify gene fragments with expression intensity in MS WBC that is either less than half or more than twice the expression intensity in normal WBC. This rationale is based on the consideration that it is more practical to detect changes in clinical samples when the range of the difference between disease and normal is large.

The procedure to derive the list of gene fragments with a fold change of at least 2 was as follows. A Genesis® Comparative Analysis was performed using normal WBC samples as the reference set and MS WBC samples as the experimental set. Parameters were as follows:
Log with Floor=0,
95% Confidence level,
Unequal Variance,
Exclude expression with call values of “unknown” or “no value,”
MAS5.0 Affymetrix Summarization,
Include only gene fragments that do not have Affymetrix “Present” Call in 100% of Samples in either sample set,
Sample set expression lower and upper percentiles of 25 and 75,
Fold-change magnitude of +/−2.0,
Raw p-value threshold of 0.05.

This analysis yielded a set of 182 genes, which are listed in Table 2. Annotations for gene sets were based on UniGene assignments using the Gene Logic GeneIndex Build 33.

Thus comparison of the genes listed in Table 2 between baseline MS samples and control blood samples identifies a differentiating signature.

### Example 2

### Genes that are Differentially Expressed Between Pre- and Post-Avonex Treatment MS Samples

Expression changes in WBC after treatment can be used as a pharmacodynamic measure of exposure to drugs, and thus as a measure of adequate dosing. They may also be a measure of therapeutic response. In the context of neutralizing antibodies to a biologic therapy, it can also be used as a functional measure activity of the drug. Presence of neutralizing antibodies to beta-interferons is known to inhibit response to therapy in patients with MS. Consequently, a Genesis® Comparative Analysis was performed using pre-Avonex treatment WBC samples as the reference set and post-Avonex treatment MS samples WBC samples as the experimental set. Parameters were as follows:
Log with Floor=0,
95% confidence level,
Unequal Variance,
Exclude expression with call values of “Unknown” or “No Value,”
MAS5.0 Affymetrix Summarization,
Include only gene fragments that do not have Affymetrix “Present” Call in 100% of samples in either sample set,
Sample set expression lower and upper percentiles of 25 and 75,
Fold-change magnitude of +/−1.3,
Raw p-value threshold of 0.05.

This analysis yielded the set of 111 genes listed in Table 3. Gene expression values for the genes listed in Table 3 provide a gene signature associated with drug exposure and response to treatment, or worsening of disease.

Further, many interferon-inducible genes were identified as markers for adequate treatment monitoring, and these include:

| | |
|---|---|
| IFI44L | interferon-induced protein 44-like |
| RSAD2 | radical S-adenosyl methionine domain containing 2 |
| RSAD2 | radical S-adenosyl methionine domain containing 2 |
| IFI44 | interferon-induced protein 44 |
| MX1 | myxovirus (influenza virus) resistance 1, |
| | interferon-inducible protein p78 (mouse |
| LOC650557 | similar to HLA class II histocompatibility |
| | antigen, DQ(W1.1) beta chain precursor |
| IFIT3 | interferon-induced protein with tetratricopeptide repeats 3 |
| LY6E | lymphocyte antigen 6 complex, locus E |
| ISG15 | ISG15 ubiquitin-like modifier |
| BIRC4BP | XIAP associated factor-1 |
| LOC129607 | hypothetical protein LOC129607 |
| IFI44 | interferon-induced protein 44 |
| APOBEC3A | apolipoprotein B mRNA editing enzyme, |
| | catalytic polypeptide-like 3A |
| MS4A4A | membrane-spanning 4-domains, subfamily A, member 4 |
| IFIT2 | interferon-induced protein with tetratricopeptide repeats 2 |
| IFIT3 | interferon-induced protein with tetratricopeptide repeats 3 |
| PARP14 | poly (ADP-ribose) polymerase family, member 14 |
| IFI6 | interferon, alpha-inducible protein 6 |

### Example 3

### Genes that are Differentially Expressed Between Pre- and Post-Copaxone Treatment MS Samples

Parameters were the same as for Example 2. This analysis yielded the set of 229 gene listed in Table 4. Annotations for gene sets were based on UniGene assignments using the Gene Logic GeneIndex Build 33. Gene expression values for the genes listed in Table 4 provide a gene signature associated with drug exposure and response to treatment, or worsening of disease.

### Example 4

### Genes that are Differentially Expressed Between Pre- and Post-Copaxone Treatment MS Samples and Between Pre- and Post-Avonex Treatment MS Samples

Avonex and Copaxone have distinct mechanisms of molecular action. Beta-interferon (Avonex) is a protein made by the body, thought primarily to combat viral infections. Glatiramer acetate (Copaxone) is a mixture of amino acids that bind to major histocompatibility complex class II molecules and competition with MBP and other myelin proteins for such binding and presentation to T cells. Genes that are altered in expression after treatment with both agents may represent a common signature of therapeutic benefit. Table 5 lists the genes that are common to both Table 3 and Table 4. Parameters were the same as for Example 2.

This analysis yielded the set of 36 gene fragments listed in Table 5. Annotations for gene sets were based on UniGene assignments using the Gene Logic GeneIndex Build 33.

Gene expression values for the genes listed in Table 5 provide a gene signature associated with drug exposure and response to treatment, or worsening of disease.

All patents or publications disclosed herein are incorporated by reference in their entireties.

TABLE 1A

| | | | SEQ | | | | | | |
|---|---|---|---|---|---|---|---|---|---|
| | | Genbank | ID | | Bonferroni | Stepup | | Mean | Mean |
| Gene Title | Gene Symbol | Acc. No. | NO. | p-value | (p-value) | (p-value) | T | (CONTROL) | (MS) |
| FK506 binding protein 5 | FKBP5 | NM_004117 | 0813 | 4.95E−20 | 2.71E−15 | 2.71E−15 | −10.9889 | 78.0882 | 190.055 |
| FK506 binding protein 5 | FKBP5 | W86302 | 1003 | 9.38E−18 | 5.13E−13 | 2.56E−13 | −10.0535 | 473.34 | 934.916 |
| FK506 binding protein 5 | FKBP5 | AI753747 | 0255 | 3.80E−17 | 2.08E−12 | 6.93E−13 | −9.80302 | 1184.25 | 2126.26 |
| cyclin D3 | CCND3 | NM_001760 | 0778 | 9.21E−13 | 5.04E−08 | 1.26E−08 | −7.96208 | 2021.6 | 2569.7 |
| tetraspanin 14 | TSPAN14 | NM_030927 | 0958 | 8.54E−12 | 4.67E−07 | 9.09E−08 | −7.54226 | 436.935 | 543.259 |
| chemokine (C—X—C motif) | CXCR4 | AF348491 | 0158 | 9.97E−12 | 5.45E−07 | 9.09E−08 | −7.51268 | 1798.6 | 2290.67 |
| receptor 4 | | | | | | | | | |
| Rho GTPase activating | ARHGAP27 | AI814329 | 0276 | 1.22E−11 | 6.65E−07 | 9.50E−08 | −7.47481 | 47.2712 | 91.2659 |
| protein 27 | | | | | | | | | |
| integrin, beta 2 | ITGB2 | L78790 | 0728 | 1.83E−11 | 1.00E−06 | 1.25E−07 | −7.39644 | 3519.18 | 4443.09 |
| (complement component 3 | | | | | | | | | |
| receptor 3 and 4 subunit) | | | | | | | | | |
| adrenergic, beta, receptor | ADRBK1 | M80776 | 0738 | 4.17E−11 | 2.28E−06 | 2.53E−07 | −7.23859 | 275.479 | 414.407 |
| kinase 1 | | | | | | | | | |
| solute carrier family 6 | SLC6A6 | NM_003043 | 0797 | 5.34E−11 | 2.92E−06 | 2.72E−07 | −7.1906 | 125.121 | 202.558 |
| (neurotransmitter | | | | | | | | | |
| transporter, taurine), | | | | | | | | | |
| member 6 | | | | | | | | | |
| Rho GTPase activating | ARHGAP18 | BE501862 | 0576 | 5.47E−11 | 2.99E−06 | 2.72E−07 | 7.18598 | 237.506 | 168.061 |
| protein 18 | | | | | | | | | |
| DNA-damage-inducible | DDIT4 | NM_019058 | 0928 | 6.90E−11 | 3.77E−06 | 3.14E−07 | −7.1411 | 247.135 | 427.375 |
| transcript 4 | | | | | | | | | |
| chromodomain helicase | CHD9 | AW300405 | 0505 | 7.89E−11 | 4.31E−06 | 3.32E−07 | 7.11505 | 241.321 | 194.754 |
| DNA binding protein 9 | | | | | | | | | |
| polypyrimidine tract | PTBP1 | AA679988 | 0041 | 9.27E−11 | 5.07E−06 | 3.54E−07 | −7.08362 | 140.67 | 188.645 |
| binding protein 1 | | | | | | | | | |
| DEAD (Asp-Glu-Ala-Asp) | DDX6 | NM_004397 | 0822 | 9.71E−11 | 5.31E−06 | 3.54E−07 | 7.07456 | 59.5517 | 43.1658 |
| box polypeptide 6 | | | | | | | | | |
| transmembrane 9 | TM9SF1 | BE899402 | 0606 | 1.17E−10 | 6.40E−06 | 4.00E−07 | −7.03808 | 95.4238 | 120.952 |
| superfamily member 1 | | | | | | | | | |
| structural maintenance of | SMC3 | AI373676 | 0193 | 1.62E−10 | 8.88E−06 | 4.76E−07 | 6.97411 | 277.737 | 200.67 |
| chromosomes 3 | | | | | | | | | |
| chromosome 6 open | C6orf166 | AI742378 | 0253 | 1.71E−10 | 9.35E−06 | 4.76E−07 | −6.96395 | 84.0063 | 129.545 |
| reading frame 166 | | | | | | | | | |
| salvador homolog 1 | SAV1 | AJ292969 | 0315 | 1.74E−10 | 9.52E−06 | 4.76E−07 | 6.96043 | 122.391 | 92.8185 |
| (Drosophila) | | | | | | | | | |
| heat shock 70 kDa protein | HSPA5 | AF216292 | 0145 | 1.74E−10 | 9.52E−06 | 4.76E−07 | −6.96042 | 1512.26 | 1749.33 |
| 5 (glucose-regulated | | | | | | | | | |
| protein, 78 kDa) | | | | | | | | | |
| MFNG O-fucosylpeptide | MFNG | AI738965 | 0249 | 2.02E−10 | 1.10E−05 | 5.26E−07 | −6.93133 | 171.014 | 235.075 |
| 3-beta-N- | | | | | | | | | |
| acetylglucosaminyltransferase | | | | | | | | | |
| ankyrin repeat and BTB | ABTB1 | BF115480 | 0622 | 2.50E−10 | 1.37E−05 | 6.20E−07 | −6.88974 | 39.5212 | 65.1926 |
| (POZ) domain containing 1 | | | | | | | | | |
| leukotriene B4 receptor | LTB4R | U33448 | 0980 | 4.64E−10 | 2.54E−05 | 1.06E−06 | −6.76716 | 127.687 | 245.457 |
| myeloid cell leukemia | MCL1 | BF594446 | 0650 | 4.65E−10 | 2.54E−05 | 1.06E−06 | −6.76677 | 182.812 | 295.014 |
| sequence 1 (BCL2- | | | | | | | | | |
| related) | | | | | | | | | |
| Friend leukemia virus | FLI1 | M93255 | 0741 | 5.83E−10 | 3.19E−05 | 1.27E−06 | −6.72202 | 620.183 | 755.664 |
| integration 1 | | | | | | | | | |
| unc-93 homolog B1 | UNC93B1 | AW001274 | 0460 | 7.24E−10 | 3.96E−05 | 1.52E−06 | −6.67888 | 130.118 | 217.625 |
| (C. elegans) | | | | | | | | | |
| mindbomb homolog 1 | MIB1 | W80418 | 1001 | 8.62E−10 | 4.71E−05 | 1.71E−06 | 6.64405 | 230.686 | 181.53 |
| (Drosophila) | | | | | | | | | |
| period homolog 1 | PER1 | NM_002616 | 0790 | 8.86E−10 | 4.85E−05 | 1.71E−06 | −6.63854 | 54.7084 | 86.7084 |
| (Drosophila) | | | | | | | | | |
| splicing factor, | SFRS17A | M99578 | 0743 | 9.08E−10 | 4.96E−05 | 1.71E−06 | −6.63372 | 89.447 | 111.808 |
| arginine/serine-rich 17A | | | | | | | | | |
| zinc finger and BTB | ZBTB16 | NM_006006 | 0849 | 9.49E−10 | 5.19E−05 | 1.73E−06 | −6.62487 | 122.114 | 211.501 |
| domain containing 16 | | | | | | | | | |
| ER lipid raft associated 1 | ERLIN1 | NM_006459 | 0862 | 9.95E−10 | 5.44E−05 | 1.76E−06 | −6.61532 | 49.7838 | 68.0357 |
| PHD finger protein 3 | PHF3 | AW189430 | 0482 | 1.05E−09 | 5.75E−05 | 1.80E−06 | 6.60417 | 170.33 | 132.083 |
| toll-like receptor 2 | TLR2 | NM_003264 | 0801 | 1.15E−09 | 6.29E−05 | 1.85E−06 | −6.58638 | 1897.07 | 2567.42 |
| paxillin | PXN | D86862 | 0710 | 1.17E−09 | 6.39E−05 | 1.85E−06 | −6.58298 | 92.9515 | 193.438 |
| RAB GTPase activating | RABGAP1L | AB019490 | 0087 | 1.21E−09 | 6.62E−05 | 1.85E−06 | 6.57615 | 286.939 | 191.152 |
| protein 1-like | | | | | | | | | |
| proline rich 14 | PRR14 | BE788667 | 0599 | 1.25E−09 | 6.82E−05 | 1.85E−06 | −6.57015 | 122.602 | 235.313 |
| Fc fragment of IgA, | FCAR | D87858 | 0712 | 1.25E−09 | 6.86E−05 | 1.85E−06 | −6.56901 | 97.3093 | 155.905 |
| receptor for | | | | | | | | | |
| tripeptidyl peptidase I | TPP1 | AA602532 | 0032 | 1.76E−09 | 9.62E−05 | 2.53E−06 | −6.50075 | 200.439 | 264.395 |
| signal-regulatory protein | SIRPA | D86043 | 0709 | 1.85E−09 | 0.000101 | 2.59E−06 | −6.49062 | 69.319 | 119.932 |
| alpha | | | | | | | | | |
| insulin-like growth factor 2 | IGF2BP3 | AU160004 | 0441 | 2.08E−09 | 0.000114 | 2.84E−06 | 6.46737 | 35.8267 | 21.3437 |
| mRNA binding protein 3 | | | | | | | | | |
| RAN binding protein 3 | RANBP3 | AI689052 | 0244 | 2.52E−09 | 0.000138 | 3.36E−06 | −6.42824 | 128.056 | 158.814 |
| tankyrase, TRF1- | TNKS2 | BF060683 | 0615 | 2.84E−09 | 0.000155 | 3.69E−06 | 6.4041 | 346.06 | 256.529 |
| interacting ankyrin-related | | | | | | | | | |
| ADP-ribose polymerase 2 | | | | | | | | | |
| transmembrane 9 | TM9SF4 | AI418892 | 0200 | 3.11E−09 | 0.00017 | 3.87E−06 | −6.38564 | 167.867 | 208.305 |
| superfamily protein | | | | | | | | | |
| member 4 | | | | | | | | | |
| CCR4-NOT transcription | CNOT1 | BC040523 | 0561 | 3.12E−09 | 0.00017 | 3.87E−06 | −6.38501 | 70.5108 | 91.1365 |
| complex, subunit 1 | | | | | | | | | |
| trophoblast-derived | TncRNA | AV659198 | 0443 | 3.26E−09 | 0.000178 | 3.93E−06 | −6.37561 | 80.5859 | 112.859 |
| noncoding RNA | | | | | | | | | |
| CD83 molecule | CD83 | NM_004233 | 0816 | 3.31E−09 | 0.000181 | 3.93E−06 | 6.37304 | 119.09 | 91.3124 |
| Transcribed locus | — | AI032730 | 0160 | 3.49E−09 | 0.000191 | 4.05E−06 | 6.36222 | 195.488 | 149.453 |
| TBC1 domain family, | TBC1D2B | BF195608 | 0625 | 3.90E−09 | 0.000213 | 4.23E−06 | −6.33953 | 288.317 | 340.68 |
| member 28 | | | | | | | | | |
| hypothetical protein | FLJ10038 | NM_017976 | 0918 | 3.91E−09 | 0.000214 | 4.23E−06 | 6.33873 | 86.1568 | 69.6291 |
| FLJ10038 | | | | | | | | | |
| procollagen-proline, 2- | P4HB | AK075503 | 0362 | 3.93E−09 | 0.000215 | 4.23E−06 | −6.33781 | 289.177 | 412.473 |
| oxoglutarate 4- | | | | | | | | | |
| dioxygenase (proline 4- | | | | | | | | | |
| hydroxylase), beta | | | | | | | | | |
| polypeptide | | | | | | | | | |
| Ewing sarcoma breakpoint | EWSR1 /// | AF327066 | 0156 | 3.95E−09 | 0.000216 | 4.23E−06 | −6.3368 | 331.762 | 417.378 |
| region 1 /// Friend | FLI1 | | | | | | | | |
| leukemia virus integration 1 | | | | | | | | | |
| Rab interacting lysosomal | RILPL2 | AI810244 | 0275 | 4.09E−09 | 0.000224 | 4.30E−06 | −6.32963 | 726.676 | 871.377 |
| protein-like 2 | | | | | | | | | |
| RAB5C, member RAS | RAB5C | AF141304 | 0136 | 4.41E−09 | 0.000241 | 4.54E−06 | −6.31423 | 663.073 | 848.044 |
| oncogene family | | | | | | | | | |
| SMAD specific E3 | SMURF2 | AY014180 | 0532 | 4.48E−09 | 0.000245 | 4.54E−06 | 6.31098 | 417.357 | 352.415 |
| ubiquitin protein ligase 2 | | | | | | | | | |
| diacylglycerol lipase, beta | DAGLB | BE795104 | 0600 | 5.78E−09 | 0.000316 | 5.75E−06 | −6.2587 | 76.1839 | 103.52 |
| — | — | AW275093 | 0495 | 6.77E−09 | 0.00037 | 6.61E−06 | 6.22634 | 37.8503 | 25.7363 |
| cisplatin resistance- | CROP | AW089673 | 0478 | 7.20E−09 | 0.000394 | 6.80E−06 | 6.21366 | 1253.96 | 1024.1 |
| associated overexpressed | | | | | | | | | |
| protein | | | | | | | | | |
| CUG triplet repeat, RNA | CUGBP2 | AI652861 | 0231 | 7.22E−09 | 0.000395 | 6.80E−06 | −6.21313 | 439.059 | 508.726 |
| binding protein 2 | | | | | | | | | |
| cleft lip and palate | CLPTM1 | BC004865 | 0551 | 7.52E−09 | 0.000411 | 6.86E−06 | −6.20462 | 269.664 | 340.303 |
| associated | | | | | | | | | |
| transmembrane protein 1 | | | | | | | | | |
| plasminogen activator, | PLAUR | AY029180 | 0533 | 7.53E−09 | 0.000412 | 6.86E−06 | −6.20442 | 407.032 | 590.904 |
| urokinase receptor | | | | | | | | | |
| SH3 domain and | SH3TC1 | NM_018986 | 0926 | 7.68E−09 | 0.00042 | 6.89E−06 | −6.20022 | 156.867 | 208.68 |
| tetratricopeptide repeats 1 | | | | | | | | | |
| CCAAT/enhancer binding | CEBPD | AV655640 | 0442 | 7.96E−09 | 0.000435 | 7.02E−06 | −6.19304 | 66.2588 | 101.6 |
| protein (C/EBP), delta | | | | | | | | | |
| myotrophin | MTPN | AL533334 | 0410 | 8.33E−09 | 0.000455 | 7.23E−06 | 6.18357 | 2922.38 | 2651.39 |
| diacylglycerol kinase, zeta | DGKZ | NM_003646 | 0806 | 8.88E−09 | 0.000486 | 7.50E−06 | −6.17031 | 495.581 | 742.687 |
| 104 kDa | | | | | | | | | |
| coiled-coil domain | CCDC95 | AA743390 | 0052 | 9.01E−09 | 0.000492 | 7.50E−06 | −6.16737 | 114.613 | 146.782 |
| containing 95 | | | | | | | | | |
| golgi autoantigen, golgin | GOLGA8B | AI829170 | 0286 | 9.05E−09 | 0.000495 | 7.50E−06 | 6.16637 | 85.2551 | 49.0105 |
| subfamily a, 8B | | | | | | | | | |
| O-linked N- | OGT | U77413 | 0990 | 9.95E−09 | 0.000544 | 8.02E−06 | −6.14688 | 276.547 | 320.452 |
| acetylglucosamine | | | | | | | | | |
| (GlcNAc) transferase | | | | | | | | | |
| (UDP-N- | | | | | | | | | |
| acetylgluco- | | | | | | | | | |
| samine:polypeptide-N- | | | | | | | | | |
| acetylglucosaminyl | | | | | | | | | |
| transferase) | | | | | | | | | |
| alpha thalassemia/mental | ATRX /// | AI650257 | 0227 | 1.01E−08 | 0.00055 | 8.02E−06 | 6.14467 | 408.475 | 331.005 |
| retardation syndrome X- | LOC728849 | | | | | | | | |
| linked (RAD54 homolog, | | | | | | | | | |
| S. cerevisiae) /// similar to | | | | | | | | | |
| transcriptional regulator | | | | | | | | | |
| ATRX isoform 1 | | | | | | | | | |
| mitogen-activated protein | MAP2K1 | AI571419 | 0217 | 1.01E−08 | 0.000554 | 8.02E−06 | −6.14319 | 404.667 | 463.791 |
| kinase kinase 1 | | | | | | | | | |
| Glutathione S-transferase | GSTK1 | AV722006 | 0451 | 1.16E−08 | 0.000636 | 9.08E−06 | 6.11454 | 19.2432 | 11.7156 |
| kappa 1 | | | | | | | | | |
| ATP-binding cassette, | ABCC1 | NM_004996 | 0833 | 1.27E−08 | 0.000694 | 9.77E−06 | −6.09625 | 116.754 | 144.306 |
| sub-family C | | | | | | | | | |
| (CFTR/MRP), member 1 | | | | | | | | | |
| chromosome 19 open | C19orf6 | AI805266 | 0270 | 1.32E−08 | 0.000719 | 9.99E−06 | −6.0888 | 83.2411 | 131.22 |
| reading frame 6 | | | | | | | | | |
| potassium channel | KCTD5 | AA872593 | 0073 | 1.34E−08 | 0.000732 | 1.00E−05 | −6.08503 | 69.8096 | 86.2762 |
| tetramerisation domain | | | | | | | | | |
| containing 5 | | | | | | | | | |
| Transcribed locus | — | AI767751 | 0265 | 1.60E−08 | 0.000873 | 1.18E−05 | 6.04842 | 71.4585 | 56.7931 |
| leukotriene B4 receptor | LTB4R | U41070 | 0982 | 1.65E−08 | 0.0009 | 1.20E−05 | −6.04214 | 138.048 | 192.488 |
| TSC22 domain family, | TSC22D3 | NM_004089 | 0811 | 1.75E−08 | 0.000955 | 1.26E−05 | −6.02976 | 544.464 | 834.605 |
| member 3 | | | | | | | | | |
| peroxiredoxin 6 | PRDX6 | NM_004905 | 0831 | 1.78E−08 | 0.000971 | 1.26E−05 | 6.02632 | 877.551 | 731.524 |
| 6-phosphofructo-2- | PFKFB2 | AB044805 | 0093 | 1.86E−08 | 0.00102 | 1.30E−05 | −6.01598 | 29.4293 | 42.6113 |
| kinase/fructose-2,6- | | | | | | | | | |
| biphosphatase 2 | | | | | | | | | |
| chromobox homolog 4 (Pc | CBX4 | NM_003655 | 0807 | 1.88E−08 | 0.001028 | 1.30E−05 | −6.01429 | 72.9469 | 113.789 |
| class homolog, | | | | | | | | | |
| Drosophila) | | | | | | | | | |
| GTPase, IMAP family | GIMAP5 | AL080068 | 0378 | 1.99E−08 | 0.001088 | 1.36E−05 | 6.00246 | 95.8797 | 62.4753 |
| member 5 | | | | | | | | | |
| surfeit 4 | SURF4 | AF078866 | 0123 | 2.03E−08 | 0.00111 | 1.37E−05 | −5.99814 | 226.218 | 267.996 |
| PHD finger protein 12 | PHF12 | AL161953 | 0399 | 2.12E−08 | 0.001158 | 1.41E−05 | −5.98937 | 30.8623 | 52.3356 |
| CCAAT/enhancer binding | CEBPD | NM_005195 | 0837 | 2.29E−08 | 0.001253 | 1.51E−05 | −5.97285 | 2487.99 | 3256.48 |
| protein (C/EBP), delta | | | | | | | | | |
| prion protein (p27-30) | PRNP | AV725328 | 0453 | 2.37E−08 | 0.001297 | 1.54E−05 | −5.96555 | 110.316 | 135.332 |
| (Creutzfeldt-Jakob | | | | | | | | | |
| disease, Gerstmann- | | | | | | | | | |
| Strausler-Scheinker | | | | | | | | | |
| syndrome, fatal familial | | | | | | | | | |
| insomnia) | | | | | | | | | |
| phosphoinositide-3- | PIK3R5 | NM_014308 | 0883 | 2.40E−08 | 0.001313 | 1.54E−05 | −5.96298 | 182.242 | 221.794 |
| kinase, regulatory subunit | | | | | | | | | |
| 5, p101 | | | | | | | | | |
| myotubularin related | MTMR1 | AK001816 | 0322 | 2.49E−08 | 0.001362 | 1.57E−05 | −5.9552 | 65.8114 | 89.0136 |
| protein 1 | | | | | | | | | |
| SLAIN motif family, | SLAIN2 | AI979301 | 0310 | 2.49E−08 | 0.001363 | 1.57E−05 | 5.95515 | 209.516 | 151.766 |
| member 2 | | | | | | | | | |
| C-type lectin domain | CLEC1B | NM_016509 | 0902 | 2.90E−08 | 0.001584 | 1.80E−05 | 5.92338 | 93.1775 | 59.0062 |
| family 1, member B | | | | | | | | | |
| tetratricopeptide repeat | TTC7A | BE205790 | 0568 | 3.05E−08 | 0.001666 | 1.87E−05 | −5.91278 | 37.0092 | 50.8386 |
| domain 7A | | | | | | | | | |
| suppressor of Ty 16 | SUPT16H | AK024072 | 0341 | 3.08E−08 | 0.001685 | 1.87E−05 | 5.91033 | 263.419 | 187.951 |
| homolog (S. cerevisiae) | | | | | | | | | |
| SERPINE1 mRNA binding | SERBP1 | BC003049 | 0547 | 3.25E−08 | 0.001778 | 1.92E−05 | 5.89903 | 1399.78 | 1212.41 |
| protein 1 | | | | | | | | | |
| RNA binding motif protein | RBM14 | AF315633 | 0153 | 3.26E−08 | 0.001782 | 1.92E−05 | −5.89857 | 39.9955 | 59.7803 |
| 14 | | | | | | | | | |
| T-cell acute lymphocytic | TAL1 | NM_003189 | 0800 | 3.28E−08 | 0.001794 | 1.92E−05 | 5.89718 | 206.228 | 138.234 |
| leukemia 1 | | | | | | | | | |
| Ras association | RAPH1 | AA194149 | 0014 | 3.30E−08 | 0.001805 | 1.92E−05 | 5.89586 | 48.0791 | 34.1444 |
| (RAlGDS/AF-6) and | | | | | | | | | |
| pleckstrin homology | | | | | | | | | |
| domains 1 | | | | | | | | | |
| disabled homolog 2, | DAB2 | N21202 | 0744 | 3.55E−08 | 0.001939 | 2.01E−05 | 5.88069 | 97.375 | 73.5883 |
| mitogen-responsive | | | | | | | | | |
| phosphoprotein | | | | | | | | | |
| (Drosophila) | | | | | | | | | |
| v-ets erythroblastosis virus | ETS1 | NM_005238 | 0840 | 3.55E−08 | 0.00194 | 2.01E−05 | −5.8806 | 52.905 | 74.8306 |
| E26 oncogene homolog 1 | | | | | | | | | |
| (avian) | | | | | | | | | |
| elastin microfibril | EMILIN2 | AL552384 | 0416 | 3.58E−08 | 0.001958 | 2.01E−05 | −5.87865 | 39.6291 | 58.0146 |
| interfacer 2 | | | | | | | | | |
| myosin IXB | MYO9B | NM_004145 | 0815 | 3.60E−08 | 0.001968 | 2.01E−05 | −5.87757 | 63.7389 | 105.347 |
| DnaJ (Hsp40) homolog, | DNAJC13 | BC043583 | 0562 | 3.70E−08 | 0.002021 | 2.04E−05 | 5.87194 | 25.7886 | 18.3726 |
| subfamily C, member 13 | | | | | | | | | |
| acyl-Coenzyme A binding | ACBD5 | BC025309 | 0560 | 3.93E−08 | 0.002151 | 2.15E−05 | 5.85869 | 84.7927 | 62.1417 |
| domain containing 5 | | | | | | | | | |
| Ras association | RAPH1 | AA194149 | 0014 | 4.25E−08 | 0.002321 | 2.30E−05 | 5.84254 | 31.6578 | 22.9448 |
| (RAlGDS/AF-6) and | | | | | | | | | |
| pleckstrin homology | | | | | | | | | |
| domains 1 | | | | | | | | | |
| C-type lectin domain | CLEC4E | BC000715 | 0538 | 4.41E−08 | 0.002412 | 2.35E−05 | −5.8344 | 829.005 | 1208.34 |
| family 4, member E | | | | | | | | | |
| Fc fragment of IgA, | FCAR | U43677 | 0983 | 4.43E−08 | 0.002424 | 2.35E−05 | −5.8333 | 63.8877 | 101.844 |
| receptor for | | | | | | | | | |
| ATPase, Ca++ | ATP2A3 | AA877910 | 0076 | 4.84E−08 | 0.002649 | 2.52E−05 | −5.81444 | 72.7871 | 103.656 |
| transporting, ubiquitous | | | | | | | | | |
| methionine | MAT2A | NM_005911 | 0848 | 4.90E−08 | 0.002681 | 2.52E−05 | −5.8119 | 97.8356 | 120.978 |
| adenosyltransferase II, | | | | | | | | | |
| alpha | | | | | | | | | |
| general transcription factor | GTF2A1 | NM_015859 | 0891 | 4.94E−08 | 0.002701 | 2.52E−05 | 5.81025 | 49.1129 | 30.3493 |
| IIA, 1, 19/37 kDa | | | | | | | | | |
| DAZ associated protein 2 | DAZAP2 | AL534321 | 0411 | 4.96E−08 | 0.002711 | 2.52E−05 | −5.80949 | 1687.13 | 2024.25 |
| chromosome 1 open | C1orf38 | AB035482 | 0091 | 4.98E−08 | 0.002723 | 2.52E−05 | −5.80855 | 1388.6 | 1760.08 |
| reading frame 38 | | | | | | | | | |
| arrestin domain containing 1 | ARRDC1 | AK001822 | 0323 | 5.37E−08 | 0.002936 | 2.69E−05 | −5.79252 | 226.097 | 291.039 |
| RAD23 homolog B | RAD23B | AL527365 | 0409 | 5.42E−08 | 0.002966 | 2.70E−05 | −5.79036 | 500.181 | 673.845 |
| (S. cerevisiae) | | | | | | | | | |
| chromosome 19 open | C19orf6 | AC004528 | 0097 | 5.59E−08 | 0.003057 | 2.75E−05 | −5.78388 | 95.6151 | 147.154 |
| reading frame 6 | | | | | | | | | |
| procollagen-proline, 2- | P4HB | J02783 | 0716 | 5.81E−08 | 0.003178 | 2.84E−05 | −5.77559 | 1504.99 | 1750.23 |
| oxoglutarate 4- | | | | | | | | | |
| dioxygenase (proline 4- | | | | | | | | | |
| hydroxylase), beta | | | | | | | | | |
| polypeptide | | | | | | | | | |
| period homolog 1 | PER1 | AF022991 | 0108 | 6.03E−08 | 0.003298 | 2.92E−05 | −5.76764 | 72.7414 | 94.1226 |
| (Drosophila) | | | | | | | | | |
| galactosylceramidase | GALC | D25284 | 0705 | 6.24E−08 | 0.003413 | 2.99E−05 | −5.76034 | 55.0178 | 65.2393 |
| CDNA FLJ33748 fis, clone | — | H75391 | 0715 | 6.38E−08 | 0.00349 | 3.03E−05 | 5.75554 | 58.2421 | 42.1421 |
| BRCAN2000148 | | | | | | | | | |
| glucosamine (N-acetyl)-6- | GNS | NM_002076 | 0782 | 6.49E−08 | 0.00355 | 3.06E−05 | −5.75189 | 94.4501 | 134.182 |
| sulfatase (Sanfilippo | | | | | | | | | |
| disease IIID) | | | | | | | | | |
| non-POU domain | NONO | L14599 | 0721 | 6.55E−08 | 0.00358 | 3.06E−05 | −5.75007 | 523.202 | 604.536 |
| containing, octamer- | | | | | | | | | |
| binding | | | | | | | | | |
| zinc finger protein 117 | ZNF117 | NM_024498 | 0946 | 6.86E−08 | 0.003749 | 3.18E−05 | 5.74018 | 140.903 | 101.407 |
| activin A receptor, type IB | ACVR1B | AL117643 | 0384 | 7.04E−08 | 0.003851 | 3.24E−05 | −5.73445 | 173.827 | 206.214 |
| minichromosome | MCM3AP | AK022303 | 0333 | 7.15E−08 | 0.00391 | 3.26E−05 | −5.73118 | 30.2496 | 41.9715 |
| maintenance complex | | | | | | | | | |
| component 3 associated | | | | | | | | | |
| protein | | | | | | | | | |
| natural killer-tumor | NKTR | AA732581 | 0048 | 7.29E−08 | 0.003984 | 3.28E−05 | 5.72714 | 151.814 | 110.341 |
| recognition sequence | | | | | | | | | |
| sorbin and SH3 domain | SORBS3 | MM_005775 | 0844 | 7.34E−08 | 0.004013 | 3.28E−05 | −5.72558 | 27.3042 | 44.7622 |
| containing 3 | | | | | | | | | |
| phosphoinositide-3-kinase | PIK3IP1 | BE042976 | 0563 | 7.42E−08 | 0.004058 | 3.28E−05 | −5.7232 | 535.676 | 693.49 |
| interacting protein 1 | | | | | | | | | |
| CDNA FLJ34061 fis, clone | — | BG430133 | 0695 | 7.44E−08 | 0.004065 | 3.28E−05 | 5.72283 | 40.264 | 27.4554 |
| FCBBF3000462 | | | | | | | | | |
| heterogeneous nuclear | HNRNPC | AV725195 | 0452 | 8.11E−08 | 0.004435 | 3.53E−05 | 5.7041 | 1949.64 | 1687.13 |
| ribonucleoprotein C | | | | | | | | | |
| (C1/C2) | | | | | | | | | |
| calnexin | CANX | AI761759 | 0258 | 8.14E−08 | 0.004453 | 3.53E−05 | −5.70324 | 426.358 | 492.788 |
| inositol polyphosphate-5- | INPP5D | U53470 | 0985 | 8.34E−08 | 0.004561 | 3.59E−05 | −5.69807 | 246.284 | 314.816 |
| phosphatase, 145 kDa | | | | | | | | | |
| hepatocellular carcinoma- | HCRP1 | AK025343 | 0356 | 8.40E−08 | 0.00459 | 3.59E−05 | −5.69672 | 56.4645 | 71.8341 |
| related HCRP1 | | | | | | | | | |
| solute carrier family 12 | SLC12A9 | BC000154 | 0535 | 9.12E−08 | 0.004986 | 3.83E−05 | −5.6789 | 66.7681 | 99.8859 |
| (potassium/chloride | | | | | | | | | |
| transporters), member 9 | | | | | | | | | |
| suppression of | ST14 | U20428 | 0979 | 9.16E−08 | 0.005011 | 3.83E−05 | −5.67782 | 22.546 | 32.7603 |
| tumorigenicity 14 (colon | | | | | | | | | |
| carcinoma) | | | | | | | | | |
| plasminogen activator, | PLAUR | U08839 | 0975 | 9.17E−08 | 0.005016 | 3.83E−05 | −5.67761 | 524.092 | 709.03 |
| urokinase receptor | | | | | | | | | |
| TAF8 RNA polymerase II, | TAF8 | BU618741 | 0703 | 9.40E−08 | 0.005138 | 3.89E−05 | −5.67241 | 59.63 | 73.0919 |
| TATA box binding protein | | | | | | | | | |
| (TBP)-associated factor, | | | | | | | | | |
| 43 kDa | | | | | | | | | |
| chemokine (C—X—C motif) | CXCR4 | L01639 | 0719 | 9.54E−08 | 0.005218 | 3.90E−05 | −5.66907 | 1512.58 | 1818.68 |
| receptor 4 | | | | | | | | | |
| N-acetylglucosamine-1- | GNPTG | AF302786 | 0151 | 9.56E−08 | 0.005226 | 3.90E−05 | −5.66876 | 178.91 | 206.84 |
| phosphate transferase, | | | | | | | | | |
| gamma subunit | | | | | | | | | |
| protein associated with | PATL1 | AA359612 | 0018 | 9.69E−08 | 0.005298 | 3.92E−05 | −5.66579 | 72.1086 | 87.181 |
| topoisomerase II homolog | | | | | | | | | |
| 1 (yeast) | | | | | | | | | |
| galactosidase, beta 1 | GLB1 | NM_000404 | 0758 | 1.04E−07 | 0.005679 | 4.18E−05 | −5.6508 | 511.828 | 593.578 |
| PDZ and LIM domain 2 | PDLIM2 | NM_021630 | 0934 | 1.05E−07 | 0.005739 | 4.19E−05 | −5.64853 | 541.251 | 633.673 |
| (mystique) | | | | | | | | | |
| (clone 1NIB-4) normalized | — | BQ002274 | 0702 | 1.08E−07 | 0.005882 | 4.26E−05 | −5.64319 | 20.177 | 30.5449 |
| cDNA library sequence | | | | | | | | | |
| transmembrane protein 43 | TMEM43 | W74580 | 1000 | 1.10E−07 | 0.006007 | 4.32E−05 | −5.63862 | 440.535 | 521.401 |
| exosome component 1 | EXOSC1 | BC012538 | 0559 | 1.11E−07 | 0.006085 | 4.35E−05 | 5.63584 | 38.3959 | 29.0411 |
| transducin-like enhancer | TLE4 | AL358975 | 0400 | 1.15E−07 | 0.006276 | 4.45E−05 | −5.62913 | 204.851 | 242.919 |
| of split 4 (E(sp1) homolog, | | | | | | | | | |
| Drosophila) | | | | | | | | | |
| plectin 1, intermediate | PLEC1 | Z54367 | 1015 | 1.20E−07 | 0.006555 | 4.62E−05 | −5.61971 | 95.9978 | 135.448 |
| filament binding protein | | | | | | | | | |
| 500 kDa | | | | | | | | | |
| FIP1 like 1 (S. cerevisiae) | FIP1L1 | NM_030917 | 0957 | 1.23E−07 | 0.006726 | 4.70E−05 | 5.61413 | 201.771 | 180.087 |
| blocked early in transport | BET1L | NM_016526 | 0903 | 1.25E−07 | 0.006853 | 4.74E−05 | −5.61006 | 12.5373 | 29.2921 |
| 1 homolog (S. cerevisiae)- | | | | | | | | | |
| like | | | | | | | | | |
| SMEK homolog 1, | SMEK1 | NM_017936 | 0917 | 1.26E−07 | 0.006873 | 4.74E−05 | 5.60944 | 91.0002 | 70.0335 |
| suppressor of mek1 | | | | | | | | | |
| (Dictyostelium) | | | | | | | | | |
| nuclear casein kinase and | NUCKS1 | AW515443 | 0516 | 1.28E−07 | 0.006977 | 4.78E−05 | 5.60618 | 1001.36 | 797.94 |
| cyclin-dependent kinase | | | | | | | | | |
| substrate 1 | | | | | | | | | |
| myosin IXB | MYO9B | AF143684 | 0137 | 1.29E−07 | 0.007064 | 4.81E−05 | −5.60349 | 301.367 | 426.587 |
| zinc finger RNA binding | ZFR | AI459274 | 0207 | 1.33E−07 | 0.007252 | 4.90E−05 | 5.59779 | 90.3366 | 66.8956 |
| protein | | | | | | | | | |
| chromosome 8 open | C8orf42 | AI632224 | 0222 | 1.35E−07 | 0.007362 | 4.94E−05 | 5.5945 | 19.8855 | 12.7616 |
| reading frame 42 | | | | | | | | | |
| retinol saturase (all-trans- | RETSAT | AK098125 | 0364 | 1.35E−07 | 0.007405 | 4.94E−05 | −5.59324 | 38.949 | 52.5054 |
| retinol 13,14-reductase) | | | | | | | | | |
| protein kinase, AMP- | PRKAB2 | NM_005399 | 0841 | 1.50E−07 | 0.008186 | 5.39E−05 | 5.57142 | 44.2835 | 33.7496 |
| activated, beta 2 non- | | | | | | | | | |
| catalytic subunit | | | | | | | | | |
| U2 small nuclear RNA | U2AF2 | NM_007279 | 0875 | 1.50E−07 | 0.008188 | 5.39E−05 | −5.57136 | 111.086 | 152.14 |
| auxiliary factor 2 | | | | | | | | | |
| WD repeat domain 1 | WDR1 | AF274954 | 0150 | 1.58E−07 | 0.008627 | 5.64E−05 | −5.55997 | 413.045 | 580.746 |
| PTK2 protein tyrosine | PTK2 | AL037339 | 0368 | 1.61E−07 | 0.008819 | 5.67E−05 | 5.55518 | 73.6126 | 59.6382 |
| kinase 2 | | | | | | | | | |
| solute carrier family 16, | SLC16A3 | AL513917 | 0404 | 1.62E−07 | 0.008831 | 5.67E−05 | −5.55488 | 372.042 | 569.693 |
| member 3 | | | | | | | | | |
| (monocarboxylic acid | | | | | | | | | |
| transporter 4) | | | | | | | | | |
| tripartite motif-containing 8 | TRIM8 | NM_030912 | 0956 | 1.62E−07 | 0.008848 | 5.67E−05 | 5.55447 | 781.843 | 658.669 |
| Full-length cDNA clone | — | AW953794 | 0525 | 1.74E−07 | 0.009534 | 6.05E−05 | −5.53815 | 27.1676 | 38.0869 |
| CS0DF032YA11 of Fetal | | | | | | | | | |
| brain of Homo sapiens | | | | | | | | | |
| (human) | | | | | | | | | |
| speckle-type POZ protein | SPOP | NM_003563 | 0805 | 1.75E−07 | 0.009565 | 6.05E−05 | 5.53745 | 917.406 | 826.599 |
| BCL2-associated X | BAX | U19599 | 0977 | 1.77E−07 | 0.009659 | 6.07E−05 | −5.53531 | 114.446 | 151.371 |
| protein | | | | | | | | | |
| high mobility group AT- | HMGA1 | AF176039 | 0141 | 1.80E−07 | 0.009863 | 6.16E−05 | −5.53073 | 22.5906 | 40.0994 |
| hook 1 | | | | | | | | | |
| Transcribed locus | — | BE348304 | 0572 | 1.84E−07 | 0.010055 | 6.25E−05 | 5.52651 | 55.2633 | 42.0568 |
| transmembrane and | TMCO3 | NM_017905 | 0914 | 1.89E−07 | 0.010358 | 6.39E−05 | −5.52001 | 68.6431 | 86.2412 |
| coiled-coil domains 3 | | | | | | | | | |
| C-type lectin domain | CLEC4E | NM_014358 | 0885 | 1.99E−07 | 0.010891 | 6.67E−05 | −5.50901 | 152.544 | 201.687 |
| family 4, member E | | | | | | | | | |
| chromosome 7 open | C7orf27 | AK024482 | 0345 | 2.00E−07 | 0.010939 | 6.67E−05 | −5.50806 | 101.071 | 132.536 |
| reading frame 27 | | | | | | | | | |
| suppressor of zeste 12 | SUZ12 /// | AI924660 | 0297 | 2.03E−07 | 0.011081 | 6.69E−05 | 5.50523 | 295.079 | 253.21 |
| homolog (Drosophila) /// | SUZ12P | | | | | | | | |
| suppressor of zeste 12 | | | | | | | | | |
| homolog pseudogene | | | | | | | | | |
| unc-93 homolog B1 | UNC93B1 | NM_030930 | 0959 | 2.03E−07 | 0.011114 | 6.69E−05 | −5.50458 | 89.4778 | 132.242 |
| (C. elegans) | | | | | | | | | |
| Transcribed locus, | — | AI738675 | 0248 | 2.09E−07 | 0.011409 | 6.83E−05 | −5.49882 | 394.226 | 504.553 |
| strongly similar to | | | | | | | | | |
| XP_529518.1 | | | | | | | | | |
| PREDICTED: hypothetical | | | | | | | | | |
| protein XP_529518 [Pan | | | | | | | | | |
| troglodytes] | | | | | | | | | |
| differentially expressed in | DEF6 | NM_022047 | 0937 | 2.17E−07 | 0.011852 | 7.06E−05 | −5.49046 | 287.062 | 338.604 |
| FDCP 6 homolog (mouse) | | | | | | | | | |
| KIAA0146 | KIAA0146 | AI363213 | 0191 | 2.21E−07 | 0.01207 | 7.14E−05 | −5.48646 | 80.4881 | 111.914 |
| BCL6 co-repressor | BCOR | AF317392 | 0154 | 2.23E−07 | 0.012214 | 7.16E−05 | 5.48386 | 111.082 | 75.8345 |
| KIAA0319-like | KIAA0319L | W58365 | 0999 | 2.24E−07 | 0.012251 | 7.16E−05 | −5.48318 | 368.026 | 488.272 |
| — | — | AK000677 | 0317 | 2.26E−07 | 0.012378 | 7.20E−05 | 5.48093 | 62.6473 | 48.142 |
| ADP-dependent | ADPGK | BC006112 | 0557 | 2.32E−07 | 0.012687 | 7.33E−05 | −5.47549 | 135.74 | 160.824 |
| glucokinase | | | | | | | | | |
| TSC22 domain family, | TSC22D3 | AL110191 | 0382 | 2.34E−07 | 0.012819 | 7.37E−05 | −5.47322 | 3412.5 | 4338.52 |
| member 3 | | | | | | | | | |
| nemo-like kinase | NLK | NM_016231 | 0897 | 2.37E−07 | 0.012956 | 7.40E−05 | 5.47089 | 86.344 | 73.4371 |
| amyloid beta (A4) | APLP2 | AW001847 | 0461 | 2.40E−07 | 0.013111 | 7.41E−05 | −5.46827 | 677.954 | 884.114 |
| precursor-like protein 2 | | | | | | | | | |
| phospholipase D family, | PLD3 | NM_012268 | 0878 | 2.41E−07 | 0.013179 | 7.41E−05 | −5.46712 | 190.559 | 273.424 |
| member 3 | | | | | | | | | |
| Transcribed locus | — | BF114745 | 0621 | 2.41E−07 | 0.01319 | 7.41E−05 | 5.46695 | 103.561 | 92.2368 |
| ER lipid raft associated 1 | ERLIN1 | AL568449 | 0419 | 2.47E−07 | 0.013486 | 7.53E−05 | −5.46206 | 340.054 | 398.739 |
| Transcribed locus | — | H24473 | 0713 | 2.58E−07 | 0.014086 | 7.83E−05 | 5.45246 | 26.6195 | 19.7937 |
| procollagen-proline, 2- | P4HB | NM_000918 | 0762 | 2.66E−07 | 0.014538 | 8.03E−05 | −5.44549 | 795.05 | 902.135 |
| oxoglutarate 4- | | | | | | | | | |
| dioxygenase (proline 4- | | | | | | | | | |
| hydroxylase), beta | | | | | | | | | |
| polypeptide | | | | | | | | | |
| calreticulin | CALR | BE251303 | 0570 | 2.71E−07 | 0.014816 | 8.14E−05 | −5.44132 | 356.88 | 465.235 |
| PTK2B protein tyrosine | PTK2B | NM_004103 | 0812 | 2.73E−07 | 0.014902 | 8.14E−05 | −5.44003 | 101.943 | 153.102 |
| kinase 2 beta | | | | | | | | | |
| KIAA0892 | KIAA0892 | AC003030 | 0096 | 2.77E−07 | 0.01513 | 8.19E−05 | −5.43668 | 15.0909 | 27.2329 |
| angiotensin II receptor- | AGTRAP | AF165187 | 0139 | 2.77E−07 | 0.015159 | 8.19E−05 | −5.43627 | 577.465 | 745.496 |
| associated protein | | | | | | | | | |
| Transcribed locus | — | AW244016 | 0490 | 2.85E−07 | 0.015562 | 8.37E−05 | −5.43047 | 108.711 | 161.89 |
| Full length insert cDNA | — | AF086041 | 0124 | 2.90E−07 | 0.015846 | 8.40E−05 | 5.42647 | 73.6103 | 50.4274 |
| clone YX53E08 | | | | | | | | | |
| suppression of | ST14 | NM_021978 | 0936 | 2.90E−07 | 0.015869 | 8.40E−05 | −5.42615 | 36.3457 | 56.3781 |
| tumorigenicity 14 (colon | | | | | | | | | |
| carcinoma) | | | | | | | | | |
| serologically defined colon | SDCCAG1 | BC006001 | 0556 | 2.92E−07 | 0.015938 | 8.40E−05 | 5.42519 | 413.226 | 349.695 |
| cancer antigen 1 | | | | | | | | | |
| Cbp/p300-interacting | CITED2 | NM_006079 | 0852 | 2.92E−07 | 0.015952 | 8.40E−05 | −5.425 | 256.843 | 305.525 |
| transactivator, with | | | | | | | | | |
| Glu/Asp-rich carboxy- | | | | | | | | | |
| terminal domain, 2 | | | | | | | | | |
| ankyrin repeat and FYVE | ANKFY1 | NM_016376 | 0901 | 2.94E−07 | 0.016093 | 8.43E−05 | −5.42305 | 86.1838 | 107.681 |
| domain containing 1 | | | | | | | | | |
| interleukin 13 receptor, | IL13RA1 | NM_001560 | 0773 | 2.97E−07 | 0.016254 | 8.43E−05 | −5.42085 | 640.164 | 787.654 |
| alpha 1 | | | | | | | | | |
| EH domain binding protein | EHBP1L1 | AA149545 | 0012 | 2.98E−07 | 0.016276 | 8.43E−05 | −5.42055 | 260.818 | 334.559 |
| 1-like 1 | | | | | | | | | |
| Rho GTPase activating | ARHGAP4 | NM_001666 | 0777 | 3.01E−07 | 0.016452 | 8.48E−05 | −5.41817 | 204.777 | 266.516 |
| protein 4 | | | | | | | | | |
| Transcribed locus | — | AW298731 | 0504 | 3.03E−07 | 0.016586 | 8.49E−05 | −5.41638 | 27.4913 | 49.2688 |
| Ras and Rab interactor 3 | RIN3 | AW027923 | 0470 | 3.07E−07 | 0.016787 | 8.49E−05 | −5.4137 | 162.083 | 222.874 |
| CDNA clone | — | N57510 | 0749 | 3.07E−07 | 0.016793 | 8.49E−05 | 5.41363 | 127.327 | 110.056 |
| IMAGE: 5313062 | | | | | | | | | |
| Homo sapiens, clone | — | AV735241 | 0455 | 3.08E−07 | 0.016816 | 8.49E−05 | 5.41333 | 171.32 | 145.168 |
| IMAGE: 3345917, mRNA | | | | | | | | | |
| pro-platelet basic protein | PPBP | R64130 | 0963 | 3.13E−07 | 0.017119 | 8.60E−05 | 5.40937 | 4143.9 | 2480.31 |
| (chemokine (C—X—C motif) | | | | | | | | | |
| ligand 7) | | | | | | | | | |
| cell division cycle 2-like 1 | CDC2L1 /// | NM_001787 | 0779 | 3.18E−07 | 0.017373 | 8.69E−05 | −5.40611 | 167.96 | 197.461 |
| (PITSLRE proteins) /// cell | CDC2L2 | | | | | | | | |
| division cycle 2-like 2 | | | | | | | | | |
| (PITSLRE proteins) | | | | | | | | | |
| cysteine conjugate-beta | CCBL2 | BC000819 | 0540 | 3.28E−07 | 0.017938 | 8.92E−05 | 5.39902 | 322.023 | 274.341 |
| lyase 2 | | | | | | | | | |
| sema domain, | SEMA4A | NM_022367 | 0939 | 3.36E−07 | 0.018372 | 9.09E−05 | −5.39372 | 362.638 | 494.889 |
| immunoglobulin domain | | | | | | | | | |
| (Ig), transmembrane | | | | | | | | | |
| domain (TM) and short | | | | | | | | | |
| cytoplasmic domain, | | | | | | | | | |
| (semaphorin) 4A | | | | | | | | | |
| Rho GDP dissociation | ARHGDIA | D13989 | 0704 | 3.41E−07 | 0.018624 | 9.14E−05 | −5.3907 | 98.9323 | 215.826 |
| inhibitor (GDI) alpha | | | | | | | | | |
| Wolf-Hirschhorn syndrome | WHSC1L1 | NM_017778 | 0912 | 3.41E−07 | 0.01864 | 9.14E−05 | 5.3905 | 68.4864 | 57.256 |
| candidate 1-like 1 | | | | | | | | | |
| tissue factor pathway | TFPI | AF021834 | 0107 | 3.46E−07 | 0.018898 | 9.22E−05 | 5.38745 | 21.9809 | 12.7738 |
| inhibitor (lipoprotein- | | | | | | | | | |
| associated coagulation | | | | | | | | | |
| inhibitor) | | | | | | | | | |
| zinc finger protein 271 | ZNF271 | AF159567 | 0138 | 3.56E−07 | 0.019471 | 9.43E−05 | 5.38082 | 179.018 | 149.631 |
| Wiskott-Aldrich syndrome | WAS | NM_000377 | 0757 | 3.57E−07 | 0.019512 | 9.43E−05 | −5.38036 | 184.107 | 410.974 |
| (eczema- | | | | | | | | | |
| thrombocytopenia) | | | | | | | | | |
| EH domain binding protein 1 | EHBP1 | BF116032 | 0624 | 3.63E−07 | 0.019842 | 9.54E−05 | 5.37662 | 96.9949 | 68.9836 |
| solute carrier family 6 | SLC6A6 | U16120 | 0976 | 3.71E−07 | 0.020295 | 9.65E−05 | −5.37161 | 60.7858 | 85.2317 |
| (neurotransmitter | | | | | | | | | |
| transporter, taurine), | | | | | | | | | |
| member 6 | | | | | | | | | |
| ADAM metallopeptidase | ADAM8 | AI814527 | 0277 | 3.72E−07 | 0.020315 | 9.65E−05 | −5.37139 | 120.008 | 221.72 |
| domain 8 | | | | | | | | | |
| zinc finger protein 36, C3H | ZFP36L2 | AI356398 | 0189 | 3.73E−07 | 0.020368 | 9.65E−05 | −5.37081 | 563.157 | 986.407 |
| type-like 2 | | | | | | | | | |
| RAB27B, member RAS | RAB27B | BF438386 | 0637 | 3.77E−07 | 0.020613 | 9.72E−05 | 5.36815 | 187.305 | 137.411 |
| oncogene family | | | | | | | | | |
| versican | VCAN | D32039 | 0706 | 3.79E−07 | 0.020739 | 9.74E−05 | −5.36679 | 1066.01 | 1372.33 |
| Interleukin 8 | IL8 | AJ239383 | 0313 | 4.33E−07 | 0.02365 | 0.000108 | −5.33753 | 75.0461 | 119.62 |
| leukocyte immunoglobulin- | LILRA2 | U82277 | 0993 | 4.81E−07 | 0.026298 | 0.000116 | −5.31382 | 349.215 | 498.426 |
| like receptor, subfamily A | | | | | | | | | |
| (with TM domain), | | | | | | | | | |
| member 2 | | | | | | | | | |
| immunoglobulin heavy | IGH@ /// | S55735 | 0965 | 5.11E−07 | 0.027913 | 0.000122 | −5.30048 | 2641.16 | 4723.74 |
| locus /// immunoglobulin | IGHA1 /// | | | | | | | | |
| heavy constant alpha 1 /// | IGHA2 /// | | | | | | | | |
| immunoglobulin heavy | LOC100126583 | | | | | | | | |
| constant alpha 2 (A2m | | | | | | | | | |
| marker) /// hypothetical | | | | | | | | | |
| LOC100126583 | | | | | | | | | |
| regulator of G-protein | RGS14 | AF037194 | 0109 | 5.12E−07 | 0.027979 | 0.000122 | −5.29995 | 80.6164 | 117.832 |
| signaling 14 | | | | | | | | | |
| F-box protein 9 | FBXO9 | AK095315 | 0363 | 5.15E−07 | 0.028176 | 0.000122 | 5.29838 | 1081.96 | 798.526 |
| septin 9 | 9-Sep | NM_006640 | 0865 | 5.17E−07 | 0.028263 | 0.000122 | −5.29769 | 87.4231 | 164.873 |
| myeloid cell leukemia | MCL1 | NM_021960 | 0935 | 5.40E−07 | 0.029511 | 0.000126 | −5.288 | 3409.06 | 4155.72 |
| sequence 1 (BCL2- | | | | | | | | | |
| related) | | | | | | | | | |
| poly(A) polymerase alpha | PAPOLA | BF797555 | 0662 | 5.74E−07 | 0.031378 | 0.00013 | 5.27423 | 1470.85 | 1208.87 |
| Transcribed locus | — | AI492388 | 0212 | 6.97E−07 | 0.038094 | 0.00015 | −5.23055 | 216.947 | 349.917 |
| Rho GDP dissociation | ARHGDIA | AI571798 | 0218 | 7.40E−07 | 0.040446 | 0.000155 | −5.21702 | 94.6544 | 217.268 |
| inhibitor (GDI) alpha | | | | | | | | | |
| guanine nucleotide | GNAZ | NM_002073 | 0781 | 7.49E−07 | 0.040958 | 0.000155 | 5.21417 | 138.429 | 84.1772 |
| binding protein (G | | | | | | | | | |
| protein), alpha z | | | | | | | | | |
| polypeptide | | | | | | | | | |
| ATPase, Ca++ | ATP2A3 | AF068220 | 0120 | 8.34E−07 | 0.04561 | 0.000168 | −5.18981 | 95.5902 | 142.894 |
| transporting, ubiquitous | | | | | | | | | |
| prostaglandin- | PTGS1 | BE613133 | 0580 | 9.28E−07 | 0.050719 | 0.000182 | 5.1657 | 276.922 | 176.715 |
| endoperoxide synthase 1 | | | | | | | | | |
| (prostaglandin G/H | | | | | | | | | |
| synthase and | | | | | | | | | |
| cyclooxygenase) | | | | | | | | | |
| ankyrin repeat and BTB | ABTB1 | AW511257 | 0514 | 9.52E−07 | 0.052074 | 0.000185 | −5.15971 | 39.4124 | 61.2487 |
| (POZ) domain containing 1 | | | | | | | | | |
| folate receptor 1 (adult) | FOLR1 | AF000381 | 0102 | 1.38E−06 | 0.075578 | 0.000238 | 5.07454 | 1958.77 | 1397.2 |
| Fc fragment of IgA, | FCAR | NM_002000 | 0780 | 1.45E−06 | 0.079201 | 0.000246 | −5.06377 | 177.357 | 246.89 |
| receptor for | | | | | | | | | |
| G-protein signaling | GPSM3 | BG111168 | 0671 | 1.76E−06 | 0.095967 | 0.00028 | −5.01948 | 477.969 | 796.568 |
| modulator 3 (AGS3-like, | | | | | | | | | |
| C. elegans) | | | | | | | | | |
| Spermatid perinuclear | STRBP | AK024960 | 0353 | 1.93E−06 | 0.105266 | 0.000299 | 4.99807 | 44.2908 | 29.796 |
| RNA binding protein | | | | | | | | | |
| enoyl Coenzyme A | ECHDC3 | NM_024693 | 0948 | 2.10E−06 | 0.114859 | 0.000316 | −4.97782 | 61.4091 | 98.8895 |
| hydratase domain | | | | | | | | | |
| containing 3 | | | | | | | | | |
| heterogeneous nuclear | HNRNPC | NM_004500 | 0824 | 2.17E−06 | 0.118742 | 0.000322 | 4.97009 | 1575.25 | 1401.51 |
| ribonucleoprotein C | | | | | | | | | |
| (C1/C2) | | | | | | | | | |
| colony stimulating factor 3 | CSF3R | NM_000760 | 0761 | 2.36E−06 | 0.128959 | 0.000344 | −4.95087 | 3038.28 | 4004.48 |
| receptor (granulocyte) | | | | | | | | | |
| myosin, light chain 6, | MYL6 | BE734356 | 0594 | 2.45E−06 | 0.134031 | 0.000356 | 4.94187 | 5732.33 | 4706.58 |
| alkali, smooth muscle and | | | | | | | | | |
| non-muscle | | | | | | | | | |
| strawberry notch homolog | SBNO2 | AC005390 | 0100 | 2.72E−06 | 0.148716 | 0.000383 | −4.91756 | 126.198 | 194.376 |
| 2 (Drosophila) | | | | | | | | | |
| DEAD (Asp-Glu-Ala-Asp) | DDX17 | AA521056 | 0025 | 2.79E−06 | 0.152295 | 0.000387 | 4.91199 | 3247.14 | 2585.45 |
| box polypeptide 17 | | | | | | | | | |
| amyotrophic lateral | ALS2CR12 | AI057333 | 0163 | 3.30E−06 | 0.180188 | 0.000423 | 4.8725 | 92.0138 | 46.3901 |
| sclerosis 2 (juvenile) | | | | | | | | | |
| chromosome region, | | | | | | | | | |
| candidate 12 | | | | | | | | | |
| egl nine homolog 2 | EGLN2 | AW057545 | 0472 | 3.41E−06 | 0.186485 | 0.000434 | −4.8644 | 46.7042 | 81.0448 |
| (C. elegans) | | | | | | | | | |
| WD and tetratricopeptide | WDTC1 | AK001734 | 0321 | 3.74E−06 | 0.204421 | 0.000464 | −4.84273 | 143.089 | 215.035 |
| repeats 1 | | | | | | | | | |
| CD74 molecule, major | CD74 | M28590 | 0733 | 3.99E−06 | 0.218102 | 0.000477 | −4.82741 | 393.178 | 654.678 |
| histocompatibility | | | | | | | | | |
| complex, class II invariant | | | | | | | | | |
| chain | | | | | | | | | |
| glycoprotein VI (platelet) | GP6 | AB043821 | 0092 | 4.78E−06 | 0.261363 | 0.000537 | 4.78445 | 95.1473 | 61.4523 |
| dual specificity | DUSP1 | AA530892 | 0027 | 4.79E−06 | 0.261693 | 0.000537 | −4.78415 | 107.394 | 209.5 |
| phosphatase 1 | | | | | | | | | |
| transcription factor binding | TFE3 | AY034078 | 0534 | 4.79E−06 | 0.262114 | 0.000537 | −4.78376 | 58.8061 | 97.4688 |
| to IGHM enhancer 3 | | | | | | | | | |
| gelsolin (amyloidosis, | GSN | BE675337 | 0587 | 4.83E−06 | 0.2639 | 0.000538 | −4.78215 | 77.2986 | 118.847 |
| Finnish type) | | | | | | | | | |
| guanine nucleotide | GNG11 | NM_004126 | 0814 | 4.83E−06 | 0.264071 | 0.000538 | 4.78199 | 548.288 | 339.78 |
| binding protein (G | | | | | | | | | |
| protein), gamma 11 | | | | | | | | | |
| CD6 molecule | CD6 | NM_006725 | 0867 | 5.60E−06 | 0.306414 | 0.000594 | −4.74649 | 92.2557 | 150.98 |
| nerve growth factor | NGFRAP1 | NM_014380 | 0886 | 6.87E−06 | 0.375513 | 0.000699 | 4.69768 | 691.606 | 498.72 |
| receptor (TNFRSF16) | | | | | | | | | |
| associated protein 1 | | | | | | | | | |
| selectin P ligand | SELPLG | AI741056 | 0250 | 7.68E−06 | 0.419967 | 0.000755 | −4.67069 | 2721.41 | 3268.22 |
| CD44 molecule (Indian | CD44 | AF098641 | 0127 | 7.70E−06 | 0.420973 | 0.000756 | −4.67012 | 371.069 | 433.064 |
| blood group) | | | | | | | | | |
| ORAI calcium release- | ORAI2 | BF939788 | 0663 | 7.87E−06 | 0.430403 | 0.000771 | −4.66476 | 59.2229 | 93.4356 |
| activated calcium | | | | | | | | | |
| modulator 2 | | | | | | | | | |
| prosaposin (variant | PSAP | M32221 | 0734 | 8.14E−06 | 0.444966 | 0.000787 | −4.65671 | 5511.12 | 6507.81 |
| Gaucher disease and | | | | | | | | | |
| variant metachromatic | | | | | | | | | |
| leukodystrophy) | | | | | | | | | |
| cortactin | CTTN | NM_005231 | 0839 | 8.54E−06 | 0.467172 | 0.000808 | 4.64491 | 182.112 | 121.169 |
| CD6 molecule | CD6 | U66145 | 0988 | 9.03E−06 | 0.493658 | 0.00084 | −4.63152 | 108.146 | 166.093 |
| potassium voltage-gated | KCNAB2 | AF044253 | 0112 | 9.05E−06 | 0.494828 | 0.00084 | −4.63095 | 32.1399 | 55.8352 |
| channel, shaker-related | | | | | | | | | |
| subfamily, beta member 2 | | | | | | | | | |
| tyrosine 3- | YWHAZ | NM_003406 | 0803 | 1.07E−05 | 0.585411 | 0.000955 | 4.58999 | 3462.95 | 2974.74 |
| monooxygenase/tryptophan | | | | | | | | | |
| 5-monooxygenase | | | | | | | | | |
| activation protein, zeta | | | | | | | | | |
| polypeptide | | | | | | | | | |
| prefoldin subunit 5 | PFDN5 | NM_002624 | 0793 | 1.11E−05 | 0.608358 | 0.000981 | 4.58059 | 1086.43 | 759.973 |
| zinc finger E-box binding | ZEB2 | AV739670 | 0456 | 1.26E−05 | 0.687237 | 0.00107 | 4.5507 | 90.1064 | 58.9124 |
| homeobox 2 | | | | | | | | | |
| lymphocyte-specific | LSP1 | NM_002339 | 0784 | 1.80E−05 | 0.983838 | 0.001356 | −4.46201 | 2289.02 | 2996.22 |
| protein 1 | | | | | | | | | |
| GM2 ganglioside activator | GM2A | AL513583 | 0403 | 1.94E−05 | 1 | 0.001433 | −4.44336 | 295.155 | 407.539 |
| B-cell CLL/lymphoma 3 | BCL3 | AI829875 | 0287 | 2.01E−05 | 1 | 0.001474 | −4.43457 | 73.2591 | 122.329 |
| signal-regulatory protein | SIRPA | AC004832 | 0099 | 2.15E−05 | 1 | 0.00155 | −4.41809 | 66.2175 | 114.978 |
| alpha | | | | | | | | | |
| chromosome 21 open | C21orf7 | NM_020152 | 0932 | 2.42E−05 | 1 | 0.00169 | 4.3876 | 280.123 | 180.541 |
| reading frame 7 | | | | | | | | | |
| arachidonate 12- | ALOX12 | NM_000697 | 0760 | 2.59E−05 | 1 | 0.001773 | 4.37069 | 142.898 | 92.6631 |
| lipoxygenase | | | | | | | | | |
| immunoglobulin heavy | IGHD | AJ275469 | 0314 | 2.75E−05 | 1 | 0.001841 | −4.35534 | 115.496 | 181.147 |
| constant delta | | | | | | | | | |
| signal-regulatory protein | SIRPA | NM_004648 | 0827 | 2.87E−05 | 1 | 0.001899 | −4.34524 | 277.486 | 340.134 |
| alpha | | | | | | | | | |
| talin 1 | TLN1 | NM_006289 | 0857 | 3.03E−05 | 1 | 0.001968 | −4.33118 | 677.305 | 962.298 |
| versican | VCAN | R94644 | 0964 | 3.18E−05 | 1 | 0.002032 | −4.31901 | 1312.01 | 1597.22 |
| leukocyte immunoglobulin- | LILRA4 | AF041261 | 0110 | 3.36E−05 | 1 | 0.002112 | −4.30455 | 50.1417 | 71.7018 |
| like receptor, subfamily A | | | | | | | | | |
| (with TM domain), | | | | | | | | | |
| member 4 | | | | | | | | | |
| dual specificity | DUSP1 | NM_004417 | 0823 | 3.50E−05 | 1 | 0.002177 | −4.2941 | 2510.99 | 3381.13 |
| phosphatase 1 | | | | | | | | | |
| leukocyte immunoglobulin- | LILRA2 | U82278 | 0994 | 3.51E−05 | 1 | 0.002177 | −4.29389 | 1033.51 | 1293.64 |
| like receptor, subfamily A | | | | | | | | | |
| (with TM domain), | | | | | | | | | |
| member 2 | | | | | | | | | |
| leukocyte immunoglobulin- | LILRA2 | U82276 | 0992 | 3.94E−05 | 1 | 0.002317 | −4.26416 | 899.494 | 1137.99 |
| like receptor, subfamily A | | | | | | | | | |
| (with TM domain), | | | | | | | | | |
| member 2 | | | | | | | | | |
| colony stimulating factor 3 | CSF3R | NM_172313 | 0960 | 4.66E−05 | 1 | 0.002581 | −4.22109 | 3895.82 | 4816.3 |
| receptor (granulocyte) | | | | | | | | | |
| immunoglobulin lambda | IGL@ | D87021 | 0711 | 4.82E−05 | 1 | 0.002642 | −4.21222 | 44.8257 | 78.8501 |
| locus | | | | | | | | | |
| opioid growth factor | OGFR | AF172449 | 0140 | 5.54E−05 | 1 | 0.002911 | −4.17619 | 54.9759 | 81.4231 |
| receptor | | | | | | | | | |
| BCL2-associated | BAG1 | NM_004323 | 0820 | 5.83E−05 | 1 | 0.003012 | 4.16308 | 3331.77 | 2495.07 |
| athanogene | | | | | | | | | |
| tetraspanin 5 | TSPAN5 | AF065389 | 0119 | 6.55E−05 | 1 | 0.003253 | 4.13256 | 1141.55 | 805.081 |
| platelet factor 4 | PF4 | NM_002619 | 0791 | 7.11E−05 | 1 | 0.003433 | 4.11123 | 2982.12 | 2052.36 |
| (chemokine (C—X—C motif) | | | | | | | | | |
| ligand 4) | | | | | | | | | |
| Fc fragment of IgA, | FCAR | U56237 | 0986 | 7.24E−05 | 1 | 0.003466 | −4.10642 | 22.6711 | 32.7601 |
| receptor for | | | | | | | | | |
| transketolase (Wernicke- | TKT | BF696840 | 0656 | 7.66E−05 | 1 | 0.003582 | −4.09169 | 2569.82 | 3111.72 |
| Korsakoff syndrome) | | | | | | | | | |
| heterogeneous nuclear | HNRNPC | AA664258 | 0039 | 8.17E−05 | 1 | 0.003762 | 4.07462 | 1778.78 | 1539.09 |
| ribonucleoprotein C | | | | | | | | | |
| (C1/C2) | | | | | | | | | |
| tumor necrosis factor | TNFRSF10C | NM_003841 | 0808 | 8.60E−05 | 1 | 0.003866 | −4.06101 | 1233.42 | 1735.81 |
| receptor superfamily, | | | | | | | | | |
| member 10c, decoy | | | | | | | | | |
| without an intracellular | | | | | | | | | |
| domain | | | | | | | | | |
| clusterin | CLU | M25915 | 0732 | 0.000109 | 1 | 0.00464 | 3.99777 | 917.982 | 586.775 |
| Interleukin 8 | IL8 | L34164 | 0723 | 0.000115 | 1 | 0.004824 | −3.98299 | 68.9562 | 113.762 |
| chromosome Y open | CYorf15A | AW468885 | 0510 | 0.000124 | 1 | 0.005062 | 3.96438 | 61.848 | 33.1275 |
| reading frame 15A | | | | | | | | | |
| insulin-like growth factor 1 | IGF1R | BF347362 | 0633 | 0.000127 | 1 | 0.005152 | −3.95765 | 22.2597 | 31.1978 |
| receptor | | | | | | | | | |
| Immunoglobulin heavy | IGHG1 | M24668 | 0731 | 0.000144 | 1 | 0.005654 | −3.92265 | 42.8722 | 66.9195 |
| constant gamma 1 (G1m | | | | | | | | | |
| marker) | | | | | | | | | |
| adducin 3 (gamma) | ADD3 | AI763123 | 0262 | 0.000155 | 1 | 0.005926 | 3.90373 | 2119.65 | 1920.44 |
| GM2 ganglioside activator | GM2A | M76477 | 0737 | 0.000176 | 1 | 0.006491 | −3.86822 | 21.724 | 39.5498 |
| small EDRK-rich factor 2 | SERF2 | BE568651 | 0579 | 0.000179 | 1 | 0.00656 | 3.86335 | 4954.6 | 4192.08 |
| neurofilament, light | NEFL | AL537457 | 0413 | 0.000179 | 1 | 0.006562 | 3.86307 | 31.9431 | 23.1309 |
| polypeptide 68 kDa | | | | | | | | | |
| hypothetical protein | LOC284701 | AL137733 | 0398 | 0.000218 | 1 | 0.007625 | −3.80903 | 64.9496 | 112.816 |
| LOC284701 | | | | | | | | | |
| interferon regulatory factor 7 | IRF7 | NM_004030 | 0810 | 0.000231 | 1 | 0.007884 | −3.79395 | 409.493 | 623.352 |
| CTD (carboxy-terminal | CTDSPL | NM_005808 | 0846 | 0.000235 | 1 | 0.00799 | 3.78905 | 93.671 | 62.2121 |
| domain, RNA polymerase | | | | | | | | | |
| II, polypeptide A) small | | | | | | | | | |
| phosphatase-like | | | | | | | | | |
| immunoglobulin heavy | IGHA1 /// | U92706 | 0997 | 0.000252 | 1 | 0.008417 | −3.76895 | 86.7125 | 132.558 |
| constant alpha 1 /// | IGHG1 /// | | | | | | | | |
| immunoglobulin heavy | IGHG3 /// | | | | | | | | |
| constant gamma 1 (G1m | IGHM /// | | | | | | | | |
| marker) /// immunoglobulin | IGHV4-31 | | | | | | | | |
| heavy constant gamma 3 | | | | | | | | | |
| (G3m marker) /// | | | | | | | | | |
| immunoglobulin heavy | | | | | | | | | |
| constant mu /// | | | | | | | | | |
| immunoglobulin heavy | | | | | | | | | |
| variable 4-31 | | | | | | | | | |
| calreticulin | CALR | NM_004343 | 0821 | 0.00026 | 1 | 0.008592 | −3.76047 | 79.4426 | 119.207 |
| profilin 1 | PFN1 | NM_005022 | 0834 | 0.000262 | 1 | 0.008638 | −3.75833 | 6953.19 | 7641.43 |
| GM2 ganglioside activator | GM2A | M76477 | 0737 | 0.000299 | 1 | 0.009457 | −3.72107 | 162.01 | 213.128 |
| Immunoglobulin heavy | IGHA1 | AW519168 | 0517 | 0.000324 | 1 | 0.009983 | −3.69913 | 152.921 | 219.57 |
| constant alpha 1 | | | | | | | | | |
| BCL2-associated | BAG1 | AF116273 | 0132 | 0.000352 | 1 | 0.010602 | 3.67548 | 3286.14 | 2540.18 |
| athanogene | | | | | | | | | |
| integrin, beta 2 | ITGB2 | NM_000211 | 0756 | 0.000355 | 1 | 0.010648 | −3.67283 | 5760.57 | 6367.25 |
| (complement component 3 | | | | | | | | | |
| receptor 3 and 4 subunit) | | | | | | | | | |
| integral membrane protein | ITM2B | AF092128 | 0126 | 0.000477 | 1 | 0.013182 | −3.58869 | 10431.6 | 11336.4 |
| 2B | | | | | | | | | |
| myeloid cell leukemia | MCL1 | AI275690 | 0180 | 0.000503 | 1 | 0.013715 | −3.5734 | 6463.22 | 7020.98 |
| sequence 1 (BCL2- | | | | | | | | | |
| related) | | | | | | | | | |
| GNAS complex locus | GNAS | AA650558 | 0038 | 0.000515 | 1 | 0.013953 | 3.56649 | 1501.58 | 1173 |
| tubulin, beta 1 | TUBB1 | N63244 | 0750 | 0.000816 | 1 | 0.019091 | 3.4314 | 2271.09 | 1745.37 |
| ribosomal protein L23 | RPL23 | NM_000978 | 0767 | 0.000818 | 1 | 0.019126 | 3.43065 | 2085.23 | 1502.85 |
| peroxiredoxin 6 | PRDX6 | BE869583 | 0604 | 0.000914 | 1 | 0.020772 | 3.39769 | 3442.93 | 2661.75 |
| neurofilament, light | NEFL | BF055311 | 0613 | 0.00094 | 1 | 0.021238 | 3.38918 | 82.1 | 61.5983 |
| polypeptide 68 kDa | | | | | | | | | |
| immunoglobulin kappa | IGK@ /// IGKC | AW575927 | 0518 | 0.001341 | 1 | 0.027148 | −3.28165 | 4837.67 | 5839.72 |
| constant /// | /// IGKV1-5 /// | | | | | | | | |
| immunoglobulin kappa | IGKV2-24 | | | | | | | | |
| variable 1-5 /// | | | | | | | | | |
| immunoglobulin kappa | | | | | | | | | |
| variable 2-24 /// | | | | | | | | | |
| immunoglobulin kappa | | | | | | | | | |
| locus | | | | | | | | | |
| cystatin C (amyloid | CST3 | NM_000099 | 0755 | 0.001346 | 1 | 0.027219 | −3.28039 | 3566.07 | 4248.09 |
| angiopathy and cerebral | | | | | | | | | |
| hemorrhage) | | | | | | | | | |
| ribosomal protein L7 | RPL7 | NM_000971 | 0766 | 0.001461 | 1 | 0.028666 | 3.25511 | 6070.42 | 4837.24 |
| interferon induced | IFITM1 | AA749101 | 0057 | 0.001797 | 1 | 0.033003 | −3.19088 | 7152.48 | 8178.72 |
| transmembrane protein 1 | | | | | | | | | |
| (9-27) | | | | | | | | | |
| ribosomal protein L36a /// | LOC729362 /// | NM_021029 | 0933 | 0.001813 | 1 | 0.03322 | 3.18807 | 4537.53 | 3218.25 |
| similar to large subunit | RPL36A | | | | | | | | |
| ribosomal protein L36a | | | | | | | | | |
| SUB1 homolog (S. cerevisiae) | SUB1 | BE784583 | 0598 | 0.001896 | 1 | 0.034265 | 3.17412 | 1392.33 | 1081.88 |
| guanylate kinase 1 | GUK1 | BC006249 | 0558 | 0.001983 | 1 | 0.035305 | 3.15989 | 4000.02 | 3171.59 |
| Pre-B-cell colony | PBEF1 | AA873350 | 0074 | 0.002161 | 1 | 0.037276 | −3.13274 | 3292.91 | 3867.52 |
| enhancing factor 1 | | | | | | | | | |
| prostaglandin- | PTGS1 | NM_000962 | 0764 | 0.002218 | 1 | 0.037809 | 3.12446 | 103.535 | 79.4279 |
| endoperoxide synthase 1 | | | | | | | | | |
| (prostaglandin G/H | | | | | | | | | |
| synthase and | | | | | | | | | |
| cyclooxygenase) | | | | | | | | | |
| F-box protein 7 | FBXO7 | NM_012179 | 0877 | 0.002317 | 1 | 0.038996 | 3.11068 | 6069.43 | 5014.48 |
| hemoglobin, alpha 1 /// | HBA1 /// HBA2 | AF105974 | 0128 | 0.002367 | 1 | 0.039561 | 3.10384 | 6124.77 | 4656.14 |
| hemoglobin, alpha 2 | | | | | | | | | |
| hemoglobin, alpha 1 | HBA1 | BC005931 | 0555 | 0.00239 | 1 | 0.039846 | 3.10074 | 6143.06 | 4645.89 |
| immunoglobulin lambda | IGL@ /// IGLJ3 | X57812 | 1008 | 0.002707 | 1 | 0.043327 | −3.06075 | 3476.47 | 4451 |
| locus /// immunoglobulin | /// IGLV2-14 /// | | | | | | | | |
| lambda variable 4-3 /// | IGLV3-25 /// | | | | | | | | |
| immunoglobulin lambda | IGLV4-3 | | | | | | | | |
| variable 3-25 /// | | | | | | | | | |
| immunoglobulin lambda | | | | | | | | | |
| variable 2-14 /// | | | | | | | | | |
| immunoglobulin lambda | | | | | | | | | |
| joining 3 | | | | | | | | | |
| major histocompatibility | HLA-G | M90686 | 0740 | 0.002992 | 1 | 0.046215 | −3.02834 | 5755.57 | 7193.31 |
| complex, class I, G | | | | | | | | | |
| SUB1 homolog (S. cerevisiae) | SUB1 | BG231551 | 0678 | 0.003023 | 1 | 0.046448 | 3.02497 | 2004.47 | 1599.13 |
| chemokine (C—X3—C motif) | CX3CR1 | U20350 | 0978 | 0.003112 | 1 | 0.04726 | 3.0156 | 2713.44 | 2373.65 |
| receptor 1 | | | | | | | | | |
| metastasis associated | MALAT1 | AF132202 | 0134 | 0.003436 | 1 | 0.050486 | −2.98318 | 6262.75 | 7016.01 |
| lung adenocarcinoma | | | | | | | | | |
| transcript 1 (non-protein | | | | | | | | | |
| coding) | | | | | | | | | |
| metastasis associated | MALAT1 | BG534952 | 0699 | 0.00728 | 1 | 0.08193 | 2.72885 | 6776.17 | 6030.81 |
| lung adenocarcinoma | | | | | | | | | |
| transcript 1 (non-protein | | | | | | | | | |
| coding) | | | | | | | | | |
| tubulin, beta 2A | TUBB2A | NM_001069 | 0768 | 0.017717 | 1 | 0.144043 | 2.40355 | 1238.52 | 689.685 |
| solute carrier family 25, | SLC25A37 | BG251467 | 0679 | 0.019789 | 1 | 0.154414 | 2.36091 | 7684.14 | 6998.66 |
| member 37 | | | | | | | | | |
| solute carrier family 25, | SLC25A37 | BG251467 | 0679 | 0.037867 | 1 | 0.228447 | 2.09875 | 4732.41 | 4047.49 |
| member 37 | | | | | | | | | |
| GNAS complex locus | GNAS | NM_016592 | 0905 | 0.060396 | 1 | 0.298237 | 1.89518 | 6053.88 | 5672.78 |
| metastasis associated | MALAT1 | BE708432 | 0592 | 0.097547 | 1 | 0.387215 | 1.66947 | 15502.2 | 14795.3 |
| lung adenocarcinoma | | | | | | | | | |
| transcript 1 (non-protein | | | | | | | | | |
| coding) | | | | | | | | | |

TABLE 1B

| | | | | | MeanDiff | | |
|---|---|---|---|---|---|---|---|
| | | Genbank | SEQ ID | MeanRatio | (CONTROL − | FoldChange | |
| Gene Title | Gene Symbol | Acc. No. | NO. | (CONTROL/MS) | MS) | (CONTROL/MS) | Probe Set ID |
| FK506 binding protein 5 | FKBP5 | NM_004117 | 0813 | 0.410872 | −111.966 | −2.43385 | 204560_at |
| FK506 binding protein 5 | FKBP5 | W86302 | 1003 | 0.506291 | −461.576 | −1.97515 | 224856_at |
| FK506 binding protein 5 | FKBP5 | AI753747 | 0255 | 0.556963 | −942.013 | −1.79545 | 224840_at |
| cyclin D3 | CCND3 | NM_001760 | 0778 | 0.786706 | −548.102 | −1.27112 | 201700_at |
| tetraspanin 14 | TSPAN14 | NM_030927 | 0958 | 0.804285 | −106.324 | −1.24334 | 221002_s_at |
| chemokine (C—X—C motif) | CXCR4 | AF348491 | 0158 | 0.785186 | −492.069 | −1.27358 | 211919_s_at |
| receptor 4 | | | | | | | |
| Rho GTPase activating | ARHGAP27 | AI814329 | 0276 | 0.51795 | −43.9948 | −1.93069 | 227057_at |
| protein 27 | | | | | | | |
| integrin, beta 2 | ITGB2 | L78790 | 0728 | 0.792057 | −923.911 | −1.26254 | 1555349_a_at |
| (complement component 3 | | | | | | | |
| receptor 3 and 4 subunit) | | | | | | | |
| adrenergic, beta, receptor | ADRBK1 | M80776 | 0738 | 0.664754 | −138.928 | −1.50432 | 201401_s_at |
| kinase 1 | | | | | | | |
| solute carrier family 6 | SLC6A6 | NM_003043 | 0797 | 0.617703 | −77.4371 | −1.6189 | 205920_at |
| (neurotransmitter | | | | | | | |
| transporter, taurine), | | | | | | | |
| member 6 | | | | | | | |
| Rho GTPase activating | ARHGAP18 | BE501862 | 0576 | 1.41321 | 69.4447 | 1.41321 | 225173_at |
| protein 18 | | | | | | | |
| DNA-damage-inducible | DDIT4 | NM_019058 | 0928 | 0.578263 | −180.24 | −1.72932 | 202887_s_at |
| transcript 4 | | | | | | | |
| chromodomain helicase | CHD9 | AW300405 | 0505 | 1.23911 | 46.5668 | 1.23911 | 229586_at |
| DNA binding protein 9 | | | | | | | |
| polypyrimidine tract | PTBP1 | AA679988 | 0041 | 0.745687 | −47.9749 | −1.34105 | 212016_s_at |
| binding protein 1 | | | | | | | |
| DEAD (Asp-Glu-Ala-Asp) | DDX6 | NM_004397 | 0822 | 1.3796 | 16.3859 | 1.3796 | 204909_at |
| box polypeptide 6 | | | | | | | |
| transmembrane 9 | TM9SF1 | BE899402 | 0606 | 0.788937 | −25.5286 | −1.26753 | 209149_s_at |
| superfamily member 1 | | | | | | | |
| structural maintenance of | SMC3 | AI373676 | 0193 | 1.38405 | 77.0678 | 1.38405 | 209258_s_at |
| chromosomes 3 | | | | | | | |
| chromosome 6 open | C6orf166 | AI742378 | 0253 | 0.64847 | −45.5391 | −1.54209 | 223143_s_at |
| reading frame 166 | | | | | | | |
| salvador homolog 1 | SAV1 | AJ292969 | 0315 | 1.3186 | 29.5723 | 1.3186 | 234491_s_at |
| (Drosophila) | | | | | | | |
| heat shock 70 kDa protein | HSPA5 | AF216292 | 0145 | 0.864477 | −237.075 | −1.15677 | 211936_at |
| 5 (glucose-regulated | | | | | | | |
| protein, 78 kDa) | | | | | | | |
| MFNG O-fucosylpeptide | MFNG | AI738965 | 0249 | 0.727485 | −64.0613 | −1.3746 | 204152_s_at |
| 3-beta-N- | | | | | | | |
| acetylglucosaminyltransferase | | | | | | | |
| ankyrin repeat and BTB | ABTB1 | BF115480 | 0622 | 0.606222 | −25.6714 | −1.64956 | 242567_at |
| (POZ) domain containing 1 | | | | | | | |
| leukotriene B4 receptor | LTB4R | U33448 | 0980 | 0.520201 | −117.77 | −1.92234 | 216388_s_at |
| myeloid cell leukemia | MCL1 | BF594446 | 0650 | 0.619672 | −112.202 | −1.61376 | 200796_s_at |
| sequence 1 (BCL2- | | | | | | | |
| related) | | | | | | | |
| Friend leukemia virus | FLI1 | M93255 | 0741 | 0.820712 | −135.481 | −1.21845 | 210786_s_at |
| integration 1 | | | | | | | |
| unc-93 homolog B1 (C. elegans) | UNC93B1 | AW001274 | 0460 | 0.597899 | −87.5072 | −1.67252 | 225869_s_at |
| mindbomb homolog 1 | MIB1 | W80418 | 1001 | 1.27079 | 49.1563 | 1.27079 | 224726_at |
| (Drosophila) | | | | | | | |
| period homolog 1 | PER1 | NM_002616 | 0790 | 0.630947 | −32 | −1.58492 | 202861_at |
| (Drosophila) | | | | | | | |
| splicing factor, | SFRS17A | M99578 | 0743 | 0.800003 | −22.3614 | −1.25 | 210269_s_at |
| arginine/serine-rich 17A | | | | | | | |
| zinc finger and BTB | ZBTB16 | NM_006006 | 0849 | 0.577369 | −89.3868 | −1.73199 | 205883_at |
| domain containing 16 | | | | | | | |
| ER lipid raft associated 1 | ERLIN1 | NM_006459 | 0862 | 0.73173 | −18.2519 | −1.36662 | 202444_s_at |
| PHD finger protein 3 | PHF3 | AW189430 | 0482 | 1.28957 | 38.2468 | 1.28957 | 217951_s_at |
| toll-like receptor 2 | TLR2 | NM_003264 | 0801 | 0.738901 | −670.351 | −1.35336 | 204924_at |
| paxillin | PXN | D86862 | 0710 | 0.480522 | −100.487 | −2.08107 | 211823_s_at |
| RAB GTPase activating | RABGAP1L | AB019490 | 0087 | 1.5011 | 95.7868 | 1.5011 | 215342_s_at |
| protein 1-like | | | | | | | |
| proline rich 14 | PRR14 | BE788667 | 0599 | 0.521016 | −112.711 | −1.91933 | 1559397_s_at |
| Fc fragment of IgA, | FCAR | D87858 | 0712 | 0.62416 | −58.5952 | −1.60215 | 211816_x_at |
| receptor for | | | | | | | |
| tripeptidyl peptidase I | TPP1 | AA602532 | 0032 | 0.758102 | −63.9567 | −1.31908 | 214196_s_at |
| signal-regulatory protein | SIRPA | D86043 | 0709 | 0.577984 | −50.6133 | −1.73015 | 202895_s_at |
| alpha | | | | | | | |
| insulin-like growth factor 2 | IGF2BP3 | AU160004 | 0441 | 1.67856 | 14.483 | 1.67856 | 203819_s_at |
| mRNA binding protein 3 | | | | | | | |
| RAN binding protein 3 | RANBP3 | AI689052 | 0244 | 0.806327 | −30.7581 | −1.24019 | 202639_s_at |
| tankyrase, TRF1- | TNKS2 | BF060683 | 0615 | 1.34901 | 89.5309 | 1.34901 | 222562_s_at |
| interacting ankyrin-related | | | | | | | |
| ADP-ribose polymerase 2 | | | | | | | |
| transmembrane 9 | TM9SF4 | AI418892 | 0200 | 0.805871 | −40.4381 | −1.24089 | 212194_s_at |
| superfamily protein | | | | | | | |
| member 4 | | | | | | | |
| CCR4-NOT transcription | CNOT1 | BC040523 | 0561 | 0.773683 | −20.6257 | −1.29252 | 1554052_at |
| complex, subunit 1 | | | | | | | |
| trophoblast-derived | TncRNA | AV659198 | 0443 | 0.714038 | −32.2735 | −1.40049 | 238320_at |
| noncoding RNA | | | | | | | |
| CD83 molecule | CD83 | NM_004233 | 0816 | 1.30421 | 27.778 | 1.30421 | 204440_at |
| Transcribed locus | — | AI032730 | 0160 | 1.30802 | 46.0351 | 1.30802 | 244679_at |
| TBC1 domain family, | TBC1D2B | BF195608 | 0625 | 0.846298 | −52.3633 | −1.18162 | 212796_s_at |
| member 2B | | | | | | | |
| hypothetical protein | FLJ10038 | NM_017976 | 0918 | 1.23737 | 16.5277 | 1.23737 | 205511_at |
| FLJ10038 | | | | | | | |
| procollagen-proline, 2- | P4HB | AK075503 | 0362 | 0.70108 | −123.296 | −1.42637 | 1564494_s_at |
| oxoglutarate 4- | | | | | | | |
| dioxygenase (proline 4- | | | | | | | |
| hydroxylase), beta | | | | | | | |
| polypeptide | | | | | | | |
| Ewing sarcoma breakpoint | EWSR1 /// | AF327066 | 0156 | 0.794872 | −85.6159 | −1.25806 | 211825_s_at |
| region 1 /// Friend | FLI1 | | | | | | |
| leukemia virus integration 1 | | | | | | | |
| Rab interacting lysosomal | RILPL2 | AI810244 | 0275 | 0.833939 | −144.701 | −1.19913 | 227983_at |
| protein-like 2 | | | | | | | |
| RAB5C, member RAS | RAB5C | AF141304 | 0136 | 0.781885 | −184.971 | −1.27896 | 201156_s_at |
| oncogene family | | | | | | | |
| SMAD specific E3 | SMURF2 | AY014180 | 0532 | 1.18427 | 64.941 | 1.18427 | 205596_s_at |
| ubiquitin protein ligase 2 | | | | | | | |
| diacylglycerol lipase, beta | DAGLB | BE795104 | 0600 | 0.735937 | −27.3357 | −1.35881 | 225833_at |
| — | — | AW275093 | 0495 | 1.4707 | 12.114 | 1.4707 | 242929_at |
| cisplatin resistance- | CROP | AW089673 | 0478 | 1.22445 | 229.86 | 1.22445 | 208835_s_at |
| associated overexpressed | | | | | | | |
| protein | | | | | | | |
| CUG triplet repeat, RNA | CUGBP2 | AI652861 | 0231 | 0.863056 | −69.6671 | −1.15867 | 227178_at |
| binding protein 2 | | | | | | | |
| cleft lip and palate | CLPTM1 | BC004865 | 0551 | 0.792422 | −70.6394 | −1.26195 | 211136_s_at |
| associated | | | | | | | |
| transmembrane protein 1 | | | | | | | |
| plasminogen activator, | PLAUR | AY029180 | 0533 | 0.688829 | −183.872 | −1.45174 | 211924_s_at |
| urokinase receptor | | | | | | | |
| SH3 domain and | SH3TC1 | NM_018986 | 0926 | 0.751709 | −51.8135 | −1.3303 | 219256_s_at |
| tetratricopeptide repeats 1 | | | | | | | |
| CCAAT/enhancer binding | CEBPD | AV655640 | 0442 | 0.652156 | −35.3408 | −1.53337 | 213006_at |
| protein (C/EBP), delta | | | | | | | |
| myotrophin | MTPN | AL533334 | 0410 | 1.10221 | 270.99 | 1.10221 | 224656_s_at |
| diacylglycerol kinase, zeta | DGKZ | NM_003646 | 0806 | 0.667281 | −247.106 | −1.49862 | 207556_s_at |
| 104 kDa | | | | | | | |
| coiled-coil domain | CCDC95 | AA743390 | 0052 | 0.780837 | −32.1692 | −1.28068 | 227286_at |
| containing 95 | | | | | | | |
| golgi autoantigen, golgin | GOLGA8B | AI829170 | 0286 | 1.73953 | 36.2445 | 1.73953 | 208797_s_at |
| subfamily a, 8B | | | | | | | |
| O-linked N- | OGT | U77413 | 0990 | 0.86299 | −43.9052 | −1.15876 | 207563_s_at |
| acetylglucosamine | | | | | | | |
| (GlcNAc) transferase | | | | | | | |
| (UDP-N- | | | | | | | |
| acetylglucosamine:polypeptide- | | | | | | | |
| N- | | | | | | | |
| acetylglucosaminyl | | | | | | | |
| transferase) | | | | | | | |
| alpha thalassemia/mental | ATRX /// | AI650257 | 0227 | 1.23404 | 77.4692 | 1.23404 | 208859_s_at |
| retardation syndrome X- | LOC728849 | | | | | | |
| linked (RAD54 homolog, | | | | | | | |
| S. cerevisiae) /// similar to | | | | | | | |
| transcriptional regulator | | | | | | | |
| ATRX isoform 1 | | | | | | | |
| mitogen-activated protein | MAP2K1 | AI571419 | 0217 | 0.87252 | −59.124 | −1.14611 | 202670_at |
| kinase kinase 1 | | | | | | | |
| Glutathione S-transferase | GSTK1 | AV722006 | 0451 | 1.64253 | 7.52765 | 1.64253 | 243325_at |
| kappa 1 | | | | | | | |
| ATP-binding cassette, | ABCC1 | NM_004996 | 0833 | 0.809071 | −27.5522 | −1.23599 | 202805_s_at |
| sub-family C | | | | | | | |
| (CFTR/MRP), member 1 | | | | | | | |
| chromosome 19 open | C19orf6 | AI805266 | 0270 | 0.634364 | −47.9788 | −1.57638 | 213986_s_at |
| reading frame 6 | | | | | | | |
| potassium channel | KCTD5 | AA872593 | 0073 | 0.809141 | −16.4666 | −1.23588 | 222645_s_at |
| tetramerisation domain | | | | | | | |
| containing 5 | | | | | | | |
| Transcribed locus | — | AI767751 | 0265 | 1.25823 | 14.6655 | 1.25823 | 228084_at |
| leukotriene B4 receptor | LTB4R | U41070 | 0982 | 0.717178 | −54.4399 | −1.39435 | 210128_s_at |
| TSC22 domain family, | TSC22D3 | NM_004089 | 0811 | 0.652362 | −290.14 | −1.53289 | 207001_x_at |
| member 3 | | | | | | | |
| peroxiredoxin 6 | PRDX6 | NM_004905 | 0831 | 1.19962 | 146.027 | 1.19962 | 200845_s_at |
| 6-phosphofructo-2- | PFKFB2 | AB044805 | 0093 | 0.690646 | −13.182 | −1.44792 | 209992_at |
| kinase/fructose-2,6- | | | | | | | |
| biphosphatase 2 | | | | | | | |
| chromobox homolog 4 (Pc | CBX4 | NM_003655 | 0807 | 0.641073 | −40.8418 | −1.55988 | 206724_at |
| class homolog, | | | | | | | |
| Drosophila) | | | | | | | |
| GTPase, IMAP family | GIMAP5 | AL080068 | 0378 | 1.53468 | 33.4044 | 1.53468 | 215352_at |
| member 5 | | | | | | | |
| surfeit 4 | SURF4 | AF078866 | 0123 | 0.84411 | −41.7779 | −1.18468 | 222979_s_at |
| PHD finger protein 12 | PHF12 | AL161953 | 0399 | 0.5897 | −21.4733 | −1.69578 | 234939_s_at |
| CCAAT/enhancer binding | CEBPD | NM_005195 | 0837 | 0.764013 | −768.486 | −1.30888 | 203973_s_at |
| protein (C/EBP), delta | | | | | | | |
| prion protein (p27-30) | PRNP | AV725328 | 0453 | 0.815149 | −25.0163 | −1.22677 | 215707_s_at |
| (Creutzfeldt-Jakob | | | | | | | |
| disease, Gerstmann- | | | | | | | |
| Strausler-Scheinker | | | | | | | |
| syndrome, fatal familial | | | | | | | |
| insomnia) | | | | | | | |
| phosphoinositide-3- | PIK3R5 | NM_014308 | 0883 | 0.821673 | −39.5518 | −1.21703 | 220566_at |
| kinase, regulatory subunit | | | | | | | |
| 5, p101 | | | | | | | |
| myotubularin related | MTMR1 | AK001816 | 0322 | 0.739341 | −23.2022 | −1.35256 | 214975_s_at |
| protein 1 | | | | | | | |
| SLAIN motif family, | SLAIN2 | AI979301 | 0310 | 1.38052 | 57.75 | 1.38052 | 224854_s_at |
| member 2 | | | | | | | |
| C-type lectin domain | CLEC1B | NM_016509 | 0902 | 1.57911 | 34.1712 | 1.57911 | 220496_at |
| family 1, member B | | | | | | | |
| tetratricopeptide repeat | TTC7A | BE205790 | 0568 | 0.727973 | −13.8295 | −1.37368 | 224924_at |
| domain 7A | | | | | | | |
| suppressor of Ty 16 | SUPT16H | AK024072 | 0341 | 1.40153 | 75.4675 | 1.40153 | 233827_s_at |
| homolog (S. cerevisiae) | | | | | | | |
| SERPINE1 mRNA binding | SERBP1 | BC003049 | 0547 | 1.15454 | 187.371 | 1.15454 | 209669_s_at |
| protein 1 | | | | | | | |
| RNA binding motif protein | RBM14 | AF315633 | 0153 | 0.669042 | −19.7848 | −1.49467 | 1555639_a_at |
| 14 | | | | | | | |
| T-cell acute lymphocytic | TAL1 | NM_003189 | 0800 | 1.49187 | 67.9937 | 1.49187 | 206283_s_at |
| leukemia 1 | | | | | | | |
| Ras association | RAPH1 | AA194149 | 0014 | 1.40811 | 13.9347 | 1.40811 | 225188_at |
| (RalGDS/AF-6) and | | | | | | | |
| pleckstrin homology | | | | | | | |
| domains 1 | | | | | | | |
| disabled homolog 2, | DAB2 | N21202 | 0744 | 1.32324 | 23.7867 | 1.32324 | 201278_at |
| mitogen-responsive | | | | | | | |
| phosphoprotein | | | | | | | |
| (Drosophila) | | | | | | | |
| v-ets erythroblastosis virus | ETS1 | NM_005238 | 0840 | 0.706997 | −21.9256 | −1.41443 | 214447_at |
| E26 oncogene homolog 1 | | | | | | | |
| (avian) | | | | | | | |
| elastin microfibril | EMILIN2 | AL552384 | 0416 | 0.683088 | −18.3855 | −1.46394 | 242288_s_at |
| interfacer 2 | | | | | | | |
| myosin IXB | MYO9B | NM_004145 | 0815 | 0.605039 | −41.608 | −1.65279 | 208452_x_at |
| DnaJ (Hsp40) homolog, | DNAJC13 | BC043583 | 0562 | 1.40365 | 7.41601 | 1.40365 | 1560020_at |
| subfamily C, member 13 | | | | | | | |
| acyl-Coenzyme A binding | ACBD5 | BC025309 | 0560 | 1.36451 | 22.651 | 1.36451 | 1568877_a_at |
| domain containing 5 | | | | | | | |
| Ras association | RAPH1 | AA194149 | 0014 | 1.37974 | 8.71298 | 1.37974 | 225189_s_at |
| (RalGDS/AF-6) and | | | | | | | |
| pleckstrin homology | | | | | | | |
| domains 1 | | | | | | | |
| C-type lectin domain | CLEC4E | BC000715 | 0538 | 0.686069 | −379.336 | −1.45758 | 222934_s_at |
| family 4, member E | | | | | | | |
| Fc fragment of IgA, | FCAR | U43677 | 0983 | 0.627309 | −37.9565 | −1.59411 | 211307_s_at |
| receptor for | | | | | | | |
| ATPase, Ca++ | ATP2A3 | AA877910 | 0076 | 0.702202 | −30.8685 | −1.42409 | 213042_s_at |
| transporting, ubiquitous | | | | | | | |
| methionine | MAT2A | NM_005911 | 0848 | 0.808703 | −23.1428 | −1.23655 | 200769_s_at |
| adenosyltransferase II, | | | | | | | |
| alpha | | | | | | | |
| general transcription factor | GTF2A1 | NM_015859 | 0891 | 1.61825 | 18.7636 | 1.61825 | 206521_s_at |
| IIA, 1, 19/37 kDa | | | | | | | |
| DAZ associated protein 2 | DAZAP2 | AL534321 | 0411 | 0.83346 | −337.118 | −1.19982 | 212595_s_at |
| chromosome 1 open | C1orf38 | AB035482 | 0091 | 0.788942 | −371.48 | −1.26752 | 210785_s_at |
| reading frame 38 | | | | | | | |
| arrestin domain containing 1 | ARRDC1 | AK001822 | 0323 | 0.77686 | −64.9423 | −1.28723 | 226405_s_at |
| RAD23 homolog B | RAD23B | AL527365 | 0409 | 0.742279 | −173.664 | −1.3472 | 201222_s_at |
| (S. cerevisiae) | | | | | | | |
| chromosome 19 open | C19orf6 | AC004528 | 0097 | 0.649764 | −51.5385 | −1.53902 | 212574_x_at |
| reading frame 6 | | | | | | | |
| procollagen-proline, 2- | P4HB | J02783 | 0716 | 0.859883 | −245.237 | −1.16295 | 200654_at |
| oxoglutarate 4- | | | | | | | |
| dioxygenase (proline 4- | | | | | | | |
| hydroxylase), beta | | | | | | | |
| polypeptide | | | | | | | |
| period homolog 1 | PER1 | AF022991 | 0108 | 0.772837 | −21.3812 | −1.29393 | 36829_at |
| (Drosophila) | | | | | | | |
| galactosylceramidase | GALC | D25284 | 0705 | 0.843323 | −10.2215 | −1.18579 | 211810_s_at |
| CDNA FLJ33748 fis, clone | — | H75391 | 0715 | 1.38204 | 16.1 | 1.38204 | 1559515_at |
| BRCAN2000148 | | | | | | | |
| glucosamine (N-acetyl)-6- | GNS | NM_002076 | 0782 | 0.703894 | −39.7323 | −1.42067 | 203676_at |
| sulfatase (Sanfilippo | | | | | | | |
| disease IIID) | | | | | | | |
| non-POU domain | NONO | L14599 | 0721 | 0.865461 | −81.334 | −1.15545 | 208698_s_at |
| containing, octamer- | | | | | | | |
| binding | | | | | | | |
| zinc finger protein 117 | ZNF117 | NM_024498 | 0946 | 1.38949 | 39.4965 | 1.38949 | 207605_x_at |
| activin A receptor, type IB | ACVR1B | AL117643 | 0384 | 0.842946 | −32.3868 | −1.18632 | 213198_at |
| minichromosome | MCM3AP | AK022303 | 0333 | 0.720718 | −11.7219 | −1.38751 | 215581_s_at |
| maintenance complex | | | | | | | |
| component 3 associated | | | | | | | |
| protein | | | | | | | |
| natural killer-tumor | NKTR | AA732581 | 0048 | 1.37587 | 41.4737 | 1.37587 | 231235_at |
| recognition sequence | | | | | | | |
| sorbin and SH3 domain | SORBS3 | NM_005775 | 0844 | 0.609982 | −17.4581 | −1.63939 | 207788_s_at |
| containing 3 | | | | | | | |
| phosphoinositide-3-kinase | PIK3IP1 | BE042976 | 0563 | 0.772434 | −157.815 | −1.29461 | 221757_at |
| interacting protein 1 | | | | | | | |
| CDNA FLJ34061 fis, clone | — | BG430133 | 0695 | 1.46652 | 12.8086 | 1.46652 | 1565614_at |
| FCBBF3000462 | | | | | | | |
| heterogeneous nuclear | HNRNPC | AV725195 | 0452 | 1.15559 | 262.504 | 1.15559 | 214737_x_at |
| ribonucleoprotein C | | | | | | | |
| (C1/C2) | | | | | | | |
| calnexin | CANX | AI761759 | 0258 | 0.865197 | −66.4295 | −1.15581 | 208852_s_at |
| inositol polyphosphate-5- | INPP5D | U53470 | 0985 | 0.782311 | −68.532 | −1.27826 | 203331_s_at |
| phosphatase, 145 kDa | | | | | | | |
| hepatocellular carcinoma- | HCRP1 | AK025343 | 0356 | 0.78604 | −15.3697 | −1.2722 | 216174_at |
| related HCRP1 | | | | | | | |
| solute carrier family 12 | SLC12A9 | BC000154 | 0535 | 0.668443 | −33.1179 | −1.49601 | 223994_s_at |
| (potassium/chloride | | | | | | | |
| transporters), member 9 | | | | | | | |
| suppression of | ST14 | U20428 | 0979 | 0.688211 | −10.2143 | −1.45304 | 216905_s_at |
| tumorigenicity 14 (colon | | | | | | | |
| carcinoma) | | | | | | | |
| plasminogen activator, | PLAUR | U08839 | 0975 | 0.739167 | −184.938 | −1.35287 | 210845_s_at |
| urokinase receptor | | | | | | | |
| TAF8 RNA polymerase II, | TAF8 | BU618741 | 0703 | 0.815823 | −13.4618 | −1.22576 | 1556178_x_at |
| TATA box binding protein | | | | | | | |
| (TBP)-associated factor, | | | | | | | |
| 43 kDa | | | | | | | |
| chemokine (C—X—C motif) | CXCR4 | L01639 | 0719 | 0.831693 | −306.096 | −1.20237 | 209201_x_at |
| receptor 4 | | | | | | | |
| N-acetylglucosamine-1- | GNPTG | AF302786 | 0151 | 0.864971 | −27.9294 | −1.15611 | 224887_at |
| phosphate transferase, | | | | | | | |
| gamma subunit | | | | | | | |
| protein associated with | PATL1 | AA359612 | 0018 | 0.827113 | −15.0724 | −1.20902 | 235235_s_at |
| topoisomerase II homolog | | | | | | | |
| 1 (yeast) | | | | | | | |
| galactosidase, beta 1 | GLB1 | NM_000404 | 0758 | 0.862277 | −81.7493 | −1.15972 | 201576_s_at |
| PDZ and LIM domain 2 | PDLIM2 | NM_021630 | 0934 | 0.854149 | −92.4219 | −1.17076 | 219165_at |
| (mystique) | | | | | | | |
| (clone 1NIB-4) normalized | — | BQ002274 | 0702 | 0.660567 | −10.368 | −1.51385 | 1556314_a_at |
| cDNA library sequence | | | | | | | |
| transmembrane protein 43 | TMEM43 | W74580 | 1000 | 0.844907 | −80.8659 | −1.18356 | 217795_s_at |
| exosome component 1 | EXOSC1 | BC012538 | 0559 | 1.32212 | 9.35476 | 1.32212 | 1559044_at |
| transducin-like enhancer | TLE4 | AL358975 | 0400 | 0.843292 | −38.0674 | −1.18583 | 216997_x_at |
| of split 4 (E(sp1) homolog, | | | | | | | |
| Drosophila) | | | | | | | |
| plectin 1, intermediate | PLEC1 | Z54367 | 1015 | 0.708741 | −39.4505 | −1.41095 | 216971_s_at |
| filament binding protein | | | | | | | |
| 500 kDa | | | | | | | |
| FIP1 like 1 (S. cerevisiae) | FIP1L1 | NM_030917 | 0957 | 1.12041 | 21.6845 | 1.12041 | 221007_s_at |
| blocked early in transport | BET1L | NM_016526 | 0903 | 0.428008 | −16.7549 | −2.3364 | 220470_at |
| 1 homolog (S. cerevisiae)- | | | | | | | |
| like | | | | | | | |
| SMEK homolog 1, | SMEK1 | NM_017936 | 0917 | 1.29938 | 20.9668 | 1.29938 | 220369_at |
| suppressor of mek1 | | | | | | | |
| (Dictyostelium) | | | | | | | |
| nuclear casein kinase and | NUCKS1 | AW515443 | 0516 | 1.25494 | 203.425 | 1.25494 | 229353_s_at |
| cyclin-dependent kinase | | | | | | | |
| substrate 1 | | | | | | | |
| myosin IXB | MYO9B | AF143684 | 0137 | 0.706462 | −125.22 | −1.4155 | 217297_s_at |
| zinc finger RNA binding | ZFR | AI459274 | 0207 | 1.35041 | 23.441 | 1.35041 | 33148_at |
| protein | | | | | | | |
| chromosome 8 open | C8orf42 | AI632224 | 0222 | 1.55823 | 7.12392 | 1.55823 | 226778_at |
| reading frame 42 | | | | | | | |
| retinol saturase (all-trans- | RETSAT | AK098125 | 0364 | 0.74181 | −13.5564 | −1.34805 | 1566472_s_at |
| retinol 13,14-reductase) | | | | | | | |
| protein kinase, AMP- | PRKAB2 | NM_005399 | 0841 | 1.31212 | 10.5339 | 1.31212 | 214474_at |
| activated, beta 2 non- | | | | | | | |
| catalytic subunit | | | | | | | |
| U2 small nuclear RNA | U2AF2 | NM_007279 | 0875 | 0.730157 | −41.0539 | −1.36957 | 218382_s_at |
| auxiliary factor 2 | | | | | | | |
| WD repeat domain 1 | WDR1 | AF274954 | 0150 | 0.711232 | −167.701 | −1.40601 | 210935_s_at |
| PTK2 protein tyrosine | PTK2 | AL037339 | 0368 | 1.23432 | 13.9745 | 1.23432 | 208820_at |
| kinase 2 | | | | | | | |
| solute carrier family 16, | SLC16A3 | AL513917 | 0404 | 0.653057 | −197.651 | −1.53126 | 202855_s_at |
| member 3 | | | | | | | |
| (monocarboxylic acid | | | | | | | |
| transporter 4) | | | | | | | |
| tripartite motif-containing 8 | TRIM8 | NM_030912 | 0956 | 1.187 | 123.174 | 1.187 | 221012_s_at |
| Full-length cDNA clone | — | AW953794 | 0525 | 0.713306 | −10.9193 | −1.40192 | 230968_at |
| CS0DF032YA11 of Fetal | | | | | | | |
| brain of Homo sapiens | | | | | | | |
| (human) | | | | | | | |
| speckle-type POZ protein | SPOP | NM_003563 | 0805 | 1.10986 | 90.8075 | 1.10986 | 204640_s_at |
| BCL2-associated X | BAX | U19599 | 0977 | 0.756064 | −36.9248 | −1.32264 | 211833_s_at |
| protein | | | | | | | |
| high mobility group AT- | HMGA1 | AF176039 | 0141 | 0.563365 | −17.5088 | −1.77505 | 210457_x_at |
| hook 1 | | | | | | | |
| Transcribed locus | — | BE348304 | 0572 | 1.31402 | 13.2065 | 1.31402 | 237554_at |
| transmembrane and | TMCO3 | NM_017905 | 0914 | 0.795943 | −17.5981 | −1.25637 | 220240_s_at |
| coiled-coil domains 3 | | | | | | | |
| C-type lectin domain | CLEC4E | NM_014358 | 0885 | 0.75634 | −49.143 | −1.32216 | 219859_at |
| family 4, member E | | | | | | | |
| chromosome 7 open | C7orf27 | AK024482 | 0345 | 0.762591 | −31.4652 | −1.31132 | 225437_s_at |
| reading frame 27 | | | | | | | |
| suppressor of zeste 12 | SUZ12 /// | AI924660 | 0297 | 1.16535 | 41.8695 | 1.16535 | 213971_s_at |
| homolog (Drosophila) /// | SUZ12P | | | | | | |
| suppressor of zeste 12 | | | | | | | |
| homolog pseudogene | | | | | | | |
| unc-93 homolog B1 (C. elegans) | UNC93B1 | NM_030930 | 0959 | 0.676621 | −42.7644 | −1.47793 | 220998_s_at |
| Transcribed locus, | — | AI738675 | 0248 | 0.781337 | −110.327 | −1.27986 | 240064_at |
| strongly similar to | | | | | | | |
| XP_529518.1 | | | | | | | |
| PREDICTED: hypothetical | | | | | | | |
| protein XP_529518 [Pan | | | | | | | |
| troglodytes] | | | | | | | |
| differentially expressed in | DEF6 | NM_022047 | 0937 | 0.84778 | −51.5421 | −1.17955 | 221293_s_at |
| FDCP 6 homolog (mouse) | | | | | | | |
| KIAA0146 | KIAA0146 | AI363213 | 0191 | 0.719196 | −31.4259 | −1.39044 | 228325_at |
| BCL6 co-repressor | BCOR | AF317392 | 0154 | 1.4648 | 35.2478 | 1.4648 | 223916_s_at |
| KIAA0319-like | KIAA0319L | W58365 | 0999 | 0.753732 | −120.246 | −1.32673 | 222468_at |
| — | — | AK000677 | 0317 | 1.3013 | 14.5053 | 1.3013 | 233876_at |
| ADP-dependent | ADPGK | BC006112 | 0557 | 0.844025 | −25.0846 | −1.1848 | 224455_s_at |
| glucokinase | | | | | | | |
| TSC22 domain family, | TSC22D3 | AL110191 | 0382 | 0.786558 | −926.021 | −1.27136 | 208763_s_at |
| member 3 | | | | | | | |
| nemo-like kinase | NLK | NM_016231 | 0897 | 1.17576 | 12.9069 | 1.17576 | 218318_s_at |
| amyloid beta (A4) | APLP2 | AW001847 | 0461 | 0.766817 | −206.16 | −1.30409 | 214875_x_at |
| precursor-like protein 2 | | | | | | | |
| phospholipase D family, | PLD3 | NM_012268 | 0878 | 0.696935 | −82.8652 | −1.43485 | 201050_at |
| member 3 | | | | | | | |
| Transcribed locus | — | BF114745 | 0621 | 1.12277 | 11.3237 | 1.12277 | 235459_at |
| ER lipid raft associated 1 | ERLIN1 | AL568449 | 0419 | 0.852824 | −58.6846 | −1.17257 | 202441_at |
| Transcribed locus | — | H24473 | 0713 | 1.34485 | 6.82582 | 1.34485 | 229951_x_at |
| procollagen-proline, 2- | P4HB | NM_000918 | 0762 | 0.881299 | −107.084 | −1.13469 | 200656_s_at |
| oxoglutarate 4- | | | | | | | |
| dioxygenase (proline 4- | | | | | | | |
| hydroxylase), beta | | | | | | | |
| polypeptide | | | | | | | |
| calreticulin | CALR | BE251303 | 0570 | 0.767097 | −108.355 | −1.30362 | 212953_x_at |
| PTK2B protein tyrosine | PTK2B | NM_004103 | 0812 | 0.665846 | −51.1597 | −1.50185 | 203111_s_at |
| kinase 2 beta | | | | | | | |
| KIAA0892 | KIAA0892 | AC003030 | 0096 | 0.554142 | −12.142 | −1.80459 | 216926_s_at |
| angiotensin II receptor- | AGTRAP | AF165187 | 0139 | 0.774605 | −168.031 | −1.29098 | 1555736_a_at |
| associated protein | | | | | | | |
| Transcribed locus | — | AW244016 | 0490 | 0.671508 | −53.1796 | −1.48918 | 227762_at |
| Full length insert cDNA | — | AF086041 | 0124 | 1.45973 | 23.1829 | 1.45973 | 1556812_a_at |
| clone YX53E08 | | | | | | | |
| suppression of | ST14 | NM_021978 | 0936 | 0.644679 | −20.0323 | −1.55116 | 202005_at |
| tumorigenicity 14 (colon | | | | | | | |
| carcinoma) | | | | | | | |
| serologically defined colon | SDCCAG1 | BC006001 | 0556 | 1.18167 | 63.5301 | 1.18167 | 1569594_a_at |
| cancer antigen 1 | | | | | | | |
| Cbp/p300-interacting | CITED2 | NM_006079 | 0852 | 0.840662 | −48.6818 | −1.18954 | 207980_s_at |
| transactivator, with | | | | | | | |
| Glu/Asp-rich carboxy- | | | | | | | |
| terminal domain, 2 | | | | | | | |
| ankyrin repeat and FYVE | ANKFY1 | NM_016376 | 0901 | 0.800364 | −21.4969 | −1.24943 | 219868_s_at |
| domain containing 1 | | | | | | | |
| interleukin 13 receptor, | IL13RA1 | NM_001560 | 0773 | 0.812748 | −147.49 | −1.23039 | 201887_at |
| alpha 1 | | | | | | | |
| EH domain binding protein | EHBP1L1 | AA149545 | 0012 | 0.779588 | −73.7409 | −1.28273 | 91703_at |
| 1-like 1 | | | | | | | |
| Rho GTPase activating | ARHGAP4 | NM_001666 | 0777 | 0.768348 | −61.7389 | −1.30149 | 204425_at |
| protein 4 | | | | | | | |
| Transcribed locus | — | AW298731 | 0504 | 0.557986 | −21.7775 | −1.79216 | 239249_at |
| Ras and Rab interactor 3 | RIN3 | AW027923 | 0470 | 0.727238 | −60.7916 | −1.37507 | 219456_s_at |
| CDNA clone | — | N57510 | 0749 | 1.15693 | 17.2711 | 1.15693 | 234998_at |
| IMAGE: 5313062 | | | | | | | |
| Homo sapiens, clone | — | AV735241 | 0455 | 1.18015 | 26.1526 | 1.18015 | 225055_at |
| IMAGE: 3345917, mRNA | | | | | | | |
| pro-platelet basic protein | PPBP | R64130 | 0963 | 1.67072 | 1663.59 | 1.67072 | 214146_s_at |
| (chemokine (C—X—C motif) | | | | | | | |
| ligand 7) | | | | | | | |
| cell division cycle 2-like 1 | CDC2L1 /// | NM_001787 | 0779 | 0.850597 | −29.5012 | −1.17564 | 207428_x_at |
| (PITSLRE proteins) /// cell | CDC2L2 | | | | | | |
| division cycle 2-like 2 | | | | | | | |
| (PITSLRE proteins) | | | | | | | |
| cysteine conjugate-beta | CCBL2 | BC000819 | 0540 | 1.1738 | 47.6813 | 1.1738 | 209472_at |
| lyase 2 | | | | | | | |
| sema domain, | SEMA4A | NM_022367 | 0939 | 0.732766 | −132.251 | −1.36469 | 219259_at |
| immunoglobulin domain | | | | | | | |
| (Ig), transmembrane | | | | | | | |
| domain (TM) and short | | | | | | | |
| cytoplasmic domain, | | | | | | | |
| (semaphorin) 4A | | | | | | | |
| Rho GDP dissociation | ARHGDIA | D13989 | 0704 | 0.45839 | −116.893 | −2.18155 | 201167_x_at |
| inhibitor (GDI) alpha | | | | | | | |
| Wolf-Hirschhorn syndrome | WHSC1L1 | NM_017778 | 0912 | 1.19614 | 11.2303 | 1.19614 | 218173_s_at |
| candidate 1-like 1 | | | | | | | |
| tissue factor pathway | TFPI | AF021834 | 0107 | 1.72078 | 9.20714 | 1.72078 | 210664_s_at |
| inhibitor (lipoprotein- | | | | | | | |
| associated coagulation | | | | | | | |
| inhibitor) | | | | | | | |
| zinc finger protein 271 | ZNF271 | AF159567 | 0138 | 1.1964 | 29.3872 | 1.1964 | 211009_s_at |
| Wiskott-Aldrich syndrome | WAS | NM_000377 | 0757 | 0.447977 | −226.867 | −2.23226 | 205400_at |
| (eczema- | | | | | | | |
| thrombocytopenia) | | | | | | | |
| EH domain binding protein 1 | EHBP1 | BF116032 | 0624 | 1.40606 | 28.0113 | 1.40606 | 212650_at |
| solute carrier family 6 | SLC6A6 | U16120 | 0976 | 0.713183 | −24.4459 | −1.40216 | 205921_s_at |
| (neurotransmitter | | | | | | | |
| transporter, taurine), | | | | | | | |
| member 6 | | | | | | | |
| ADAM metallopeptidase | ADAM8 | AI814527 | 0277 | 0.541261 | −101.711 | −1.84754 | 205179_s_at |
| domain 8 | | | | | | | |
| zinc finger protein 36, C3H | ZFP36L2 | AI356398 | 0189 | 0.570918 | −423.25 | −1.75157 | 201367_s_at |
| type-like 2 | | | | | | | |
| RAB27B, member RAS | RAB27B | BF438386 | 0637 | 1.3631 | 49.8935 | 1.3631 | 228708_at |
| oncogene family | | | | | | | |
| versican | VCAN | D32039 | 0706 | 0.776785 | −306.325 | −1.28736 | 211571_s_at |
| Interleukin 8 | IL8 | AJ239383 | 0313 | 0.627373 | −44.5734 | −1.59395 | 217281_x_at |
| leukocyte immunoglobulin- | LILRA2 | U82277 | 0993 | 0.700637 | −149.21 | −1.42727 | 211102_s_at |
| like receptor, subfamily A | | | | | | | |
| (with TM domain), | | | | | | | |
| member 2 | | | | | | | |
| immunoglobulin heavy | IGH@ /// | S55735 | 0965 | 0.559124 | −2082.58 | −1.78851 | 217022_s_at |
| locus /// immunoglobulin | IGHA1 /// | | | | | | |
| heavy constant alpha 1 /// | IGHA2 /// | | | | | | |
| immunoglobulin heavy | LOC100126583 | | | | | | |
| constant alpha 2 (A2m | | | | | | | |
| marker) /// hypothetical | | | | | | | |
| LOC100126583 | | | | | | | |
| regulator of G-protein | RGS14 | AF037194 | 0109 | 0.684164 | −37.2156 | −1.46164 | 211021_s_at |
| signaling 14 | | | | | | | |
| F-box protein 9 | FBXO9 | AK095315 | 0363 | 1.35494 | 283.431 | 1.35494 | 1566509_s_at |
| septin 9 | 9-Sep | NM_006640 | 0865 | 0.530246 | −77.4496 | −1.88592 | 207425_s_at |
| myeloid cell leukemia | MCL1 | NM_021960 | 0935 | 0.820331 | −746.654 | −1.21902 | 200798_x_at |
| sequence 1 (BCL2- | | | | | | | |
| related) | | | | | | | |
| poly(A) polymerase alpha | PAPOLA | BF797555 | 0662 | 1.21671 | 261.98 | 1.21671 | 212718_at |
| Transcribed locus | — | AI492388 | 0212 | 0.619996 | −132.97 | −1.61291 | 228854_at |
| Rho GDP dissociation | ARHGDIA | AI571798 | 0218 | 0.435657 | −122.614 | −2.29538 | 213606_s_at |
| inhibitor (GDI) alpha | | | | | | | |
| guanine nucleotide | GNAZ | NM_002073 | 0781 | 1.6445 | 54.2522 | 1.6445 | 204993_at |
| binding protein (G | | | | | | | |
| protein), alpha z | | | | | | | |
| polypeptide | | | | | | | |
| ATPase, Ca++ | ATP2A3 | AF068220 | 0120 | 0.668956 | −47.3043 | −1.49487 | 207521_s_at |
| transporting, ubiquitous | | | | | | | |
| prostaglandin- | PTGS1 | BE613133 | 0580 | 1.56705 | 100.206 | 1.56705 | 238669_at |
| endoperoxide synthase 1 | | | | | | | |
| (prostaglandin G/H | | | | | | | |
| synthase and | | | | | | | |
| cyclooxygenase) | | | | | | | |
| ankyrin repeat and BTB | ABTB1 | AW511257 | 0514 | 0.643482 | −21.8363 | −1.55405 | 228848_at |
| (POZ) domain containing 1 | | | | | | | |
| folate receptor 1 (adult) | FOLR1 | AF000381 | 0102 | 1.40192 | 561.562 | 1.40192 | 211074_at |
| Fc fragment of IgA, | FCAR | NM_002000 | 0780 | 0.718362 | −69.5336 | −1.39206 | 207674_at |
| receptor for | | | | | | | |
| G-protein signaling | GPSM3 | BG111168 | 0671 | 0.600035 | −318.6 | −1.66657 | 214847_s_at |
| modulator 3 (AGS3-like, | | | | | | | |
| C. elegans) | | | | | | | |
| Spermatid perinuclear | STRBP | AK024960 | 0353 | 1.48647 | 14.4948 | 1.48647 | 233251_at |
| RNA binding protein | | | | | | | |
| enoyl Coenzyme A | ECHDC3 | NM_024693 | 0948 | 0.620987 | −37.4804 | −1.61034 | 219298_at |
| hydratase domain | | | | | | | |
| containing 3 | | | | | | | |
| heterogeneous nuclear | HNRNPC | NM_004500 | 0824 | 1.12397 | 173.739 | 1.12397 | 200014_s_at |
| ribonucleoprotein C | | | | | | | |
| (C1/C2) | | | | | | | |
| colony stimulating factor 3 | CSF3R | NM_000760 | 0761 | 0.75872 | −966.201 | −1.31801 | 203591_s_at |
| receptor (granulocyte) | | | | | | | |
| myosin, light chain 6, | MYL6 | BE734356 | 0594 | 1.21794 | 1025.74 | 1.21794 | 212082_s_at |
| alkali, smooth muscle and | | | | | | | |
| non-muscle | | | | | | | |
| strawberry notch homolog | SBNO2 | AC005390 | 0100 | 0.649244 | −68.1785 | −1.54025 | 215760_s_at |
| 2 (Drosophila) | | | | | | | |
| DEAD (Asp-Glu-Ala-Asp) | DDX17 | AA521056 | 0025 | 1.25593 | 661.69 | 1.25593 | 230180_at |
| box polypeptide 17 | | | | | | | |
| amyotrophic lateral | ALS2CR12 | AI057333 | 0163 | 1.98348 | 45.6236 | 1.98348 | 239458_at |
| sclerosis 2 (juvenile) | | | | | | | |
| chromosome region, | | | | | | | |
| candidate 12 | | | | | | | |
| egl nine homolog 2 (C. elegans) | EGLN2 | AW057545 | 0472 | 0.576276 | −34.3406 | −1.73528 | 223083_s_at |
| WD and tetratricopeptide | WDTC1 | AK001734 | 0321 | 0.665423 | −71.9458 | −1.5028 | 216036_x_at |
| repeats 1 | | | | | | | |
| CD74 molecule, major | CD74 | M28590 | 0733 | 0.600568 | −261.5 | −1.66509 | 1567628_at |
| histocompatibility | | | | | | | |
| complex, class II invariant | | | | | | | |
| chain | | | | | | | |
| glycoprotein VI (platelet) | GP6 | AB043821 | 0092 | 1.54831 | 33.695 | 1.54831 | 220336_s_at |
| dual specificity | DUSP1 | AA530892 | 0027 | 0.512623 | −102.105 | −1.95075 | 201044_x_at |
| phosphatase 1 | | | | | | | |
| transcription factor binding | TFE3 | AY034078 | 0534 | 0.603333 | −38.6627 | −1.65746 | 1565347_s_at |
| to IGHM enhancer 3 | | | | | | | |
| gelsolin (amyloidosis, | GSN | BE675337 | 0587 | 0.650402 | −41.5488 | −1.53751 | 214040_s_at |
| Finnish type) | | | | | | | |
| guanine nucleotide | GNG11 | NM_004126 | 0814 | 1.61366 | 208.508 | 1.61366 | 204115_at |
| binding protein (G | | | | | | | |
| protein), gamma 11 | | | | | | | |
| CD6 molecule | CD6 | NM_006725 | 0867 | 0.611045 | −58.7246 | −1.63654 | 208602_x_at |
| nerve growth factor | NGFRAP1 | NM_014380 | 0886 | 1.38676 | 192.886 | 1.38676 | 217963_s_at |
| receptor (TNFRSF16) | | | | | | | |
| associated protein 1 | | | | | | | |
| selectin P ligand | SELPLG | AI741056 | 0250 | 0.832688 | −546.812 | −1.20093 | 209879_at |
| CD44 molecule (Indian | CD44 | AF098641 | 0127 | 0.856847 | −61.9943 | −1.16707 | 210916_s_at |
| blood group) | | | | | | | |
| ORAI calcium release- | ORAI2 | BF939788 | 0663 | 0.633836 | −34.2127 | −1.57769 | 218811_at |
| activated calcium | | | | | | | |
| modulator 2 | | | | | | | |
| prosaposin (variant | PSAP | M32221 | 0734 | 0.846847 | −996.693 | −1.18085 | 200866_s_at |
| Gaucher disease and | | | | | | | |
| variant metachromatic | | | | | | | |
| leukodystrophy) | | | | | | | |
| cortactin | CTTN | NM_005231 | 0839 | 1.50296 | 60.9429 | 1.50296 | 201059_at |
| CD6 molecule | CD6 | U66145 | 0988 | 0.651121 | −57.9464 | −1.53581 | 211893_x_at |
| potassium voltage-gated | KCNAB2 | AF044253 | 0112 | 0.575621 | −23.6953 | −1.73725 | 211791_s_at |
| channel, shaker-related | | | | | | | |
| subfamily, beta member 2 | | | | | | | |
| tyrosine 3- | YWHAZ | NM_003406 | 0803 | 1.16412 | 488.217 | 1.16412 | 200639_s_at |
| monooxygenase/tryptophan | | | | | | | |
| 5-monooxygenase | | | | | | | |
| activation protein, zeta | | | | | | | |
| polypeptide | | | | | | | |
| prefoldin subunit 5 | PFDN5 | NM_002624 | 0793 | 1.42957 | 326.46 | 1.42957 | 207132_x_at |
| zinc finger E-box binding | ZEB2 | AV739670 | 0456 | 1.5295 | 31.1939 | 1.5295 | 233031_at |
| homeobox 2 | | | | | | | |
| lymphocyte-specific | LSP1 | NM_002339 | 0784 | 0.76397 | −707.2 | −1.30895 | 203523_at |
| protein 1 | | | | | | | |
| GM2 ganglioside activator | GM2A | AL513583 | 0403 | 0.724236 | −112.384 | −1.38076 | 212737_at |
| B-cell CLL/lymphoma 3 | BCL3 | AI829875 | 0287 | 0.59887 | −49.0698 | −1.66981 | 204907_s_at |
| signal-regulatory protein | SIRPA | AC004832 | 0099 | 0.575913 | −48.7608 | −1.73637 | 217024_x_at |
| alpha | | | | | | | |
| chromosome 21 open | C21orf7 | NM_020152 | 0932 | 1.55157 | 99.5819 | 1.55157 | 221211_s_at |
| reading frame 7 | | | | | | | |
| arachidonate 12- | ALOX12 | NM_000697 | 0760 | 1.54212 | 50.2344 | 1.54212 | 207206_s_at |
| lipoxygenase | | | | | | | |
| immunoglobulin heavy | IGHD | AJ275469 | 0314 | 0.637582 | −65.6509 | −1.56843 | 214973_x_at |
| constant delta | | | | | | | |
| signal-regulatory protein | SIRPA | NM_004648 | 0827 | 0.815813 | −62.6483 | −1.22577 | 202896_s_at |
| alpha | | | | | | | |
| talin 1 | TLN1 | NM_006289 | 0857 | 0.703842 | −284.992 | −1.42077 | 203254_s_at |
| versican | VCAN | R94644 | 0964 | 0.821433 | −285.21 | −1.21738 | 215646_s_at |
| leukocyte immunoglobulin- | LILRA4 | AF041261 | 0110 | 0.699309 | −21.5601 | −1.42998 | 210313_at |
| like receptor, subfamily A | | | | | | | |
| (with TM domain), | | | | | | | |
| member 4 | | | | | | | |
| dual specificity | DUSP1 | NM_004417 | 0823 | 0.742647 | −870.146 | −1.34654 | 201041_s_at |
| phosphatase 1 | | | | | | | |
| leukocyte immunoglobulin- | LILRA2 | U82278 | 0994 | 0.798914 | −260.133 | −1.2517 | 211100_x_at |
| like receptor, subfamily A | | | | | | | |
| (with TM domain), | | | | | | | |
| member 2 | | | | | | | |
| leukocyte immunoglobulin- | LILRA2 | U82276 | 0992 | 0.79042 | −238.501 | −1.26515 | 211101_x_at |
| like receptor, subfamily A | | | | | | | |
| (with TM domain), | | | | | | | |
| member 2 | | | | | | | |
| colony stimulating factor 3 | CSF3R | NM_172313 | 0960 | 0.808882 | −920.479 | −1.23627 | 1553297_a_at |
| receptor (granulocyte) | | | | | | | |
| immunoglobulin lambda | IGL@ | D87021 | 0711 | 0.568492 | −34.0245 | −1.75904 | 216560_x_at |
| locus | | | | | | | |
| opioid growth factor | OGFR | AF172449 | 0140 | 0.675188 | −26.4472 | −1.48107 | 211513_s_at |
| receptor | | | | | | | |
| BCL2-associated | BAG1 | NM_004323 | 0820 | 1.33534 | 836.698 | 1.33534 | 202387_at |
| athanogene | | | | | | | |
| tetraspanin 5 | TSPAN5 | AF065389 | 0119 | 1.41793 | 336.467 | 1.41793 | 209890_at |
| platelet factor 4 | PF4 | NM_002619 | 0791 | 1.45302 | 929.754 | 1.45302 | 206390_x_at |
| (chemokine (C—X—C motif) | | | | | | | |
| ligand 4) | | | | | | | |
| Fc fragment of IgA, | FCAR | U56237 | 0986 | 0.692035 | −10.089 | −1.44501 | 211306_s_at |
| receptor for | | | | | | | |
| transketolase (Wernicke- | TKT | BF696840 | 0656 | 0.825852 | −541.902 | −1.21087 | 208699_x_at |
| Korsakoff syndrome) | | | | | | | |
| heterogeneous nuclear | HNRNPC | AA664258 | 0039 | 1.15573 | 239.689 | 1.15573 | 212626_x_at |
| ribonucleoprotein C | | | | | | | |
| (C1/C2) | | | | | | | |
| tumor necrosis factor | TNFRSF10C | NM_003841 | 0808 | 0.710572 | −502.391 | −1.40732 | 206222_at |
| receptor superfamily, | | | | | | | |
| member 10c, decoy | | | | | | | |
| without an intracellular | | | | | | | |
| domain | | | | | | | |
| clusterin | CLU | M25915 | 0732 | 1.56445 | 331.206 | 1.56445 | 208792_s_at |
| Interleukin 8 | IL8 | L34164 | 0723 | 0.606145 | −44.8057 | −1.64977 | 211650_x_at |
| chromosome Y open | CYorf15A | AW468885 | 0510 | 1.86697 | 28.7205 | 1.86697 | 236694_at |
| reading frame 15A | | | | | | | |
| insulin-like growth factor 1 | IGF1R | BF347362 | 0633 | 0.713503 | −8.93807 | −1.40154 | 243358_at |
| receptor | | | | | | | |
| Immunoglobulin heavy | IGHG1 | M24668 | 0731 | 0.640653 | −24.0473 | −1.56091 | 211633_x_at |
| constant gamma 1 (G1m | | | | | | | |
| marker) | | | | | | | |
| adducin 3 (gamma) | ADD3 | AI763123 | 0262 | 1.10373 | 199.209 | 1.10373 | 201752_s_at |
| GM2 ganglioside activator | GM2A | M76477 | 0737 | 0.549282 | −17.8258 | −1.82056 | 209727_at |
| small EDRK-rich factor 2 | SERF2 | BE568651 | 0579 | 1.18189 | 762.518 | 1.18189 | 224625_x_at |
| neurofilament, light | NEFL | AL537457 | 0413 | 1.38097 | 8.81215 | 1.38097 | 221805_at |
| polypeptide 68 kDa | | | | | | | |
| hypothetical protein | LOC284701 | AL137733 | 0398 | 0.575711 | −47.8667 | −1.73698 | 234664_at |
| LOC284701 | | | | | | | |
| interferon regulatory factor 7 | IRF7 | NM_004030 | 0810 | 0.656921 | −213.859 | −1.52225 | 208436_s_at |
| CTD (carboxy-terminal | CTDSPL | NM_005808 | 0846 | 1.50567 | 31.4589 | 1.50567 | 201906_s_at |
| domain, RNA polymerase | | | | | | | |
| II, polypeptide A) small | | | | | | | |
| phosphatase-like | | | | | | | |
| immunoglobulin heavy | IGHA1 /// | U92706 | 0997 | 0.654147 | −45.8457 | −1.52871 | 216557_x_at |
| constant alpha 1 /// | IGHG1 /// | | | | | | |
| immunoglobulin heavy | IGHG3 /// | | | | | | |
| constant gamma 1 (G1m | IGHM /// | | | | | | |
| marker) /// immunoglobulin | IGHV4-31 | | | | | | |
| heavy constant gamma 3 | | | | | | | |
| (G3m marker) /// | | | | | | | |
| immunoglobulin heavy | | | | | | | |
| constant mu /// | | | | | | | |
| immunoglobulin heavy | | | | | | | |
| variable 4-31 | | | | | | | |
| calreticulin | CALR | NM_004343 | 0821 | 0.666427 | −39.7641 | −1.50054 | 200935_at |
| profilin 1 | PFN1 | NM_005022 | 0834 | 0.909933 | −688.243 | −1.09898 | 200634_at |
| GM2 ganglioside activator | GM2A | M76477 | 0737 | 0.760155 | −51.1178 | −1.31552 | 35820_at |
| Immunoglobulin heavy | IGHA1 | AW519168 | 0517 | 0.696459 | −66.6484 | −1.43583 | 215118_s_at |
| constant alpha 1 | | | | | | | |
| BCL2-associated | BAG1 | AF116273 | 0132 | 1.29366 | 745.959 | 1.29366 | 211475_s_at |
| athanogene | | | | | | | |
| integrin, beta 2 | ITGB2 | NM_000211 | 0756 | 0.904719 | −606.678 | −1.10532 | 202803_s_at |
| (complement component 3 | | | | | | | |
| receptor 3 and 4 subunit) | | | | | | | |
| integral membrane protein | ITM2B | AF092128 | 0126 | 0.920192 | −904.73 | −1.08673 | 217732_s_at |
| 2B | | | | | | | |
| myeloid cell leukemia | MCL1 | AI275690 | 0180 | 0.920558 | −557.764 | −1.0863 | 200797_s_at |
| sequence 1 (BCL2- | | | | | | | |
| related) | | | | | | | |
| GNAS complex locus | GNAS | AA650558 | 0038 | 1.28011 | 328.574 | 1.28011 | 217673_x_at |
| tubulin, beta 1 | TUBB1 | N63244 | 0750 | 1.30121 | 525.72 | 1.30121 | 230690_at |
| ribosomal protein L23 | RPL23 | NM_000978 | 0767 | 1.38751 | 582.376 | 1.38751 | 200888_s_at |
| peroxiredoxin 6 | PRDX6 | BE869583 | 0604 | 1.29349 | 781.185 | 1.29349 | 200844_s_at |
| neurofilament, light | NEFL | BF055311 | 0613 | 1.33283 | 20.5017 | 1.33283 | 221916_at |
| polypeptide 68 kDa | | | | | | | |
| immunoglobulin kappa | IGK@ /// IGKC | AW575927 | 0518 | 0.828408 | −1002.05 | −1.20713 | 224795_x_at |
| constant /// | /// IGKV1-5 /// | | | | | | |
| immunoglobulin kappa | IGKV2-24 | | | | | | |
| variable 1-5 /// | | | | | | | |
| immunoglobulin kappa | | | | | | | |
| variable 2-24 /// | | | | | | | |
| immunoglobulin kappa | | | | | | | |
| locus | | | | | | | |
| cystatin C (amyloid | CST3 | NM_000099 | 0755 | 0.839452 | −682.022 | −1.19125 | 201360_at |
| angiopathy and cerebral | | | | | | | |
| hemorrhage) | | | | | | | |
| ribosomal protein L7 | RPL7 | NM_000971 | 0766 | 1.25494 | 1233.19 | 1.25494 | 200717_x_at |
| interferon induced | IFITM1 | AA749101 | 0057 | 0.874523 | −1026.24 | −1.14348 | 214022_s_at |
| transmembrane protein 1 | | | | | | | |
| (9-27) | | | | | | | |
| ribosomal protein L36a /// | LOC729362 /// | NM_021029 | 0933 | 1.40994 | 1319.28 | 1.40994 | 201406_at |
| similar to large subunit | RPL36A | | | | | | |
| ribosomal protein L36a | | | | | | | |
| SUB1 homolog (S. cerevisiae) | SUB1 | BE784583 | 0598 | 1.28695 | 310.449 | 1.28695 | 224586_x_at |
| guanylate kinase 1 | GUK1 | BC006249 | 0558 | 1.2612 | 828.435 | 1.2612 | 200075_s_at |
| Pre-B-cell colony | PBEF1 | AA873350 | 0074 | 0.851427 | −574.61 | −1.1745 | 243296_at |
| enhancing factor 1 | | | | | | | |
| prostaglandin- | PTGS1 | NM_000962 | 0764 | 1.30351 | 24.1075 | 1.30351 | 205127_at |
| endoperoxide synthase 1 | | | | | | | |
| (prostaglandin G/H | | | | | | | |
| synthase and | | | | | | | |
| cyclooxygenase) | | | | | | | |
| F-box protein 7 | FBXO7 | NM_012179 | 0877 | 1.21038 | 1054.96 | 1.21038 | 201178_at |
| hemoglobin, alpha 1 /// | HBA1 /// HBA2 | AF105974 | 0128 | 1.31542 | 1468.63 | 1.31542 | 209458_x_at |
| hemoglobin, alpha 2 | | | | | | | |
| hemoglobin, alpha 1 | HBA1 | BC005931 | 0555 | 1.32226 | 1497.18 | 1.32226 | 211745_x_at |
| immunoglobulin lambda | IGL@ /// IGLJ3 | X57812 | 1008 | 0.781053 | −974.535 | −1.28032 | 214677_x_at |
| locus /// immunoglobulin | /// IGLV2-14 /// | | | | | | |
| lambda variable 4-3 /// | IGLV3-25 /// | | | | | | |
| immunoglobulin lambda | IGLV4-3 | | | | | | |
| variable 3-25 /// | | | | | | | |
| immunoglobulin lambda | | | | | | | |
| variable 2-14 /// | | | | | | | |
| immunoglobulin lambda | | | | | | | |
| joining 3 | | | | | | | |
| major histocompatibility | HLA-G | M90686 | 0740 | 0.800128 | −1437.74 | −1.2498 | 211530_x_at |
| complex, class I, G | | | | | | | |
| SUB1 homolog (S. cerevisiae) | SUB1 | BG231551 | 0678 | 1.25347 | 405.331 | 1.25347 | 212857_x_at |
| chemokine (C—X3—C motif) | CX3CR1 | U20350 | 0978 | 1.14315 | 339.788 | 1.14315 | 205898_at |
| receptor 1 | | | | | | | |
| metastasis associated | MALAT1 | AF132202 | 0134 | 0.892637 | −753.262 | −1.12028 | 223940_x_at |
| lung adenocarcinoma | | | | | | | |
| transcript 1 (non-protein | | | | | | | |
| coding) | | | | | | | |
| metastasis associated | MALAT1 | BG534952 | 0699 | 1.12359 | 745.361 | 1.12359 | 224567_x_at |
| lung adenocarcinoma | | | | | | | |
| transcript 1 (non-protein | | | | | | | |
| coding) | | | | | | | |
| tubulin, beta 2A | TUBB2A | NM_001069 | 0768 | 1.79577 | 548.832 | 1.79577 | 204141_at |
| solute carrier family 25, | SLC25A37 | BG251467 | 0679 | 1.09794 | 685.472 | 1.09794 | 222529_at |
| member 37 | | | | | | | |
| solute carrier family 25, | SLC25A37 | BG251467 | 0679 | 1.16922 | 684.917 | 1.16922 | 222528_s_at |
| member 37 | | | | | | | |
| GNAS complex locus | GNAS | NM_016592 | 0905 | 1.06718 | 381.104 | 1.06718 | 200981_x_at |
| metastasis associated | MALAT1 | BE708432 | 0592 | 1.04778 | 706.89 | 1.04778 | 1558678_s_at |
| lung adenocarcinoma | | | | | | | |
| transcript 1 (non-protein | | | | | | | |
| coding) | | | | | | | |

TABLE 2A

| | | | | | t-Test | Fold Change | | Log10 |
|---|---|---|---|---|---|---|---|---|
| | Known | Genbank | SEQ | | p-Value | Signed | | Mean |
| Fragment | Genes: Gene | Accession | ID | Sequence Clusters: | (MS vs | Magnitude (MS | FC | (Control |
| Name | Symbol | No. | NO. | Cluster Title | Control) | vs. Control) | Magnitude | Samples) |
| 214847_s_at | GPSM3 | BG111168 | 0671 | “G-protein signalling | 1.29E−11 | 1.82 | 1.82 | 2.2 |
| | | | | modulator 3 (AGS3-like, | | | | |
| | | | | C. elegans)” | | | | |
| 227510_x_at | | AL037917 | 0369 | Transcribed locus | 1.41E−10 | 6.2 | 6.2 | 2.29 |
| 223578_x_at | MALAT1 | AF113016 | 0131 | Metastasis associated | 1.55E−10 | 3.4 | 3.4 | 1.83 |
| | | | | lung adenocarcinoma | | | | |
| | | | | transcript 1 (non-coding | | | | |
| | | | | RNA) | | | | |
| 232431_at | | AI934556 | 0302 | “Glucocorticoid receptor | 4.46E−10 | −2.75 | 2.75 | 2.52 |
| | | | | alpha mRNA, variant 3′ | | | | |
| | | | | UTR” | | | | |
| 219878_s_at | KLF13 | NM_015995 | 0892 | Kruppel-like factor 13 | 8.23E−10 | 13.42 | 13.42 | 0.4 |
| 214352_s_at | KRAS | BF673699 | 0652 | V-Ki-ras2 Kirsten rat | 1.04E−09 | 2.25 | 2.25 | 2.06 |
| | | | | sarcoma viral oncogene | | | | |
| | | | | homolog | | | | |
| 228582_x_at | MALAT1 | AI475544 | 0211 | Metastasis associated | 1.06E−09 | 9.68 | 9.68 | 2.39 |
| | | | | lung adenocarcinoma | | | | |
| | | | | transcript 1 (non-coding | | | | |
| | | | | RNA) | | | | |
| 243981_at | | AI763206 | 0263 | Transcribed locus | 1.42E−09 | −2.43 | 2.43 | 2.5 |
| 201753_s_at | ADD3 | NM_019903 | 0931 | Adducin 3 (gamma) | 1.95E−09 | −1.85 | 1.85 | 3.16 |
| 211074_at | FOLR1 | AF000381 | 0102 | Folate receptor 1 (adult) | 3.38E−09 | 2.39 | 2.39 | 2.16 |
| 225956_at | LOC153222 | AL565238 | 0417 | Adult retina protein | 3.62E−09 | −1.48 | 1.48 | 2.87 |
| 223161_at | KIAA1147 | AA029331 | 0003 | KIAA1147 | 4.65E−09 | 2.34 | 2.34 | 2.27 |
| 218389_s_at | APH1A | NM_016022 | 0894 | Anterior pharynx defective | 4.69E−09 | 1.51 | 1.51 | 2.26 |
| | | | | 1 homolog A (C. elegans) | | | | |
| 236924_at | GLMN | AA814383 | 0065 | “Glomulin, FKBP | 7.55E−09 | −2.5 | 2.5 | 2.05 |
| | | | | associated protein” | | | | |
| 226148_at | ZBTB44 | AU144305 | 0422 | Zinc finger and BTB | 1.05E−08 | −1.78 | 1.78 | 2.96 |
| | | | | domain containing 44 | | | | |
| 219696_at | FLJ20054 | NM_019049 | 0927 | Hypothetical protein | 1.10E−08 | −1.47 | 1.47 | 2.18 |
| | | | | FLJ20054 | | | | |
| 223940_x_at | | AF132202 | 0134 | | 1.32E−08 | 2.92 | 2.92 | 2.45 |
| 225889_at | AEBP2 | BF475280 | 0639 | AE binding protein 2 | 1.37E−08 | −1.88 | 1.88 | 2.69 |
| 214697_s_at | ROD1 | AW190873 | 0485 | ROD1 regulator of | 1.71E−08 | 2.27 | 2.27 | 1.9 |
| | | | | differentiation 1 (S. pombe) | | | | |
| 208610_s_at | SRRM2 | AI655799 | 0233 | Serine/arginine repetitive | 2.18E−08 | 3.16 | 3.16 | 1.72 |
| | | | | matrix 2 | | | | |
| 241751_at | OFD1 | AW292752 | 0498 | Oral-facial-digital | 2.26E−08 | −2.4 | 2.4 | 2.17 |
| | | | | syndrome 1 | | | | |
| 204049_s_at | PHACTR2 | NM_014721 | 0889 | Phosphatase and actin | 3.26E−08 | −1.7 | 1.7 | 2.33 |
| | | | | regulator 2 | | | | |
| 225377_at | C9orf86 | BE783949 | 0597 | Chromosome 9 open | 3.78E−08 | 1.9 | 1.9 | 2.27 |
| | | | | reading frame 86 | | | | |
| 225139_at | RBM35B | AW070424 | 0473 | RNA binding motif protein | 3.81E−08 | −1.6 | 1.6 | 3 |
| | | | | 35B | | | | |
| 216177_at | LOC391132 | AW582267 | 0519 | Similar to 60S ribosomal | 3.88E−08 | −1.88 | 1.88 | 2.19 |
| | | | | protein L29 (P23) | | | | |
| 242232_at | | AI652864 | 0232 | | 4.50E−08 | 2.15 | 2.15 | 1.7 |
| 200734_s_at | ARF3 | BG341906 | 0689 | ADP-ribosylation factor 3 | 4.57E−08 | 1.78 | 1.78 | 2.25 |
| 211716_x_at | ARHGDIA | BC005851 | 0554 | Rho GDP dissociation | 4.64E−08 | 1.66 | 1.66 | 2.62 |
| | | | | inhibitor (GDI) alpha | | | | |
| 215460_x_at | BRD1 | AL080149 | 0380 | Bromodomain containing 1 | 4.65E−08 | −1.39 | 1.39 | 2.25 |
| 222576_s_at | EIF2C1 | AW071829 | 0475 | “Eukaryotic translation | 4.71E−08 | 1.88 | 1.88 | 2.14 |
| | | | | initiation factor 2C, 1” | | | | |
| 230141_at | ARID4A | AI640594 | 0226 | AT rich interactive domain | 5.62E−08 | −1.88 | 1.88 | 2.19 |
| | | | | 4A (RBP1-like) | | | | |
| 227740_at | UHMK1 | AW173222 | 0481 | U2AF homology motif | 5.74E−08 | 2.45 | 2.45 | 2.37 |
| | | | | (UHM) kinase 1 | | | | |
| 204805_s_at | H1FX | NM_006026 | 0851 | “H1 histone family, | 6.56E−08 | 2.3 | 2.3 | 2.26 |
| | | | | member X” | | | | |
| 208624_s_at | EIF4G1 | BE966878 | 0610 | “Eukaryotic translation | 6.81E−08 | 1.41 | 1.41 | 1.88 |
| | | | | initiation factor 4 gamma, | | | | |
| | | | | 1” | | | | |
| 243612_at | NSD1 | AL526448 | 0408 | Nuclear receptor binding | 7.09E−08 | −2.61 | 2.61 | 2.19 |
| | | | | SET domain protein 1 | | | | |
| 201167_x_at | ARHGDIA | D13989 | 0704 | Rho GDP dissociation | 7.24E−08 | 2.48 | 2.48 | 1.72 |
| | | | | inhibitor (GDI) alpha | | | | |
| 241786_at | | AI380514 | 0198 | Transcribed locus | 7.33E−08 | −2.3 | 2.3 | 2.61 |
| 238430_x_at | SLFN5 | AI923675 | 0295 | Schlafen family member 5 | 7.41E−08 | 2.67 | 2.67 | 2.57 |
| 202102_s_at | BRD4 | BF718610 | 0657 | Bromodomain containing 4 | 7.45E−08 | 1.54 | 1.54 | 2.32 |
| 201737_s_at | MARCH6 | NM_005885 | 0847 | Membrane-associated | 8.45E−08 | −1.77 | 1.77 | 2.53 |
| | | | | ring finger (C3HC4) 6 | | | | |
| 229926_at | | AI633738 | 0225 | Transcribed locus | 8.49E−08 | −1.61 | 1.61 | 2.04 |
| 230961_at | | BE856980 | 0603 | | 9.10E−08 | −2.05 | 2.05 | 2.07 |
| 201996_s_at | SPEN | AL524033 | 0406 | “Spen homolog, | 9.12E−08 | 2.59 | 2.59 | 1.55 |
| | | | | transcriptional regulator | | | | |
| | | | | (Drosophila)” | | | | |
| 231858_x_at | DKFZp761E198 | BC004895 | 0552 | DKFZp761E198 protein | 9.89E−08 | 1.83 | 1.83 | 2.02 |
| 200608_s_at | RAD21 | NM_006265 | 0855 | RAD21 homolog (S. pombe) | 9.99E−08 | −1.44 | 1.44 | 2.61 |
| 201168_x_at | ARHGDIA | NM_004309 | 0818 | Rho GDP dissociation | 1.00E−07 | 1.81 | 1.81 | 2.37 |
| | | | | inhibitor (GDI) alpha | | | | |
| 224631_at | ZFP91 | AA758013 | 0058 | Zinc finger protein 91 | 1.00E−07 | 1.92 | 1.92 | 2.02 |
| | | | | homolog (mouse) | | | | |
| 225563_at | PAN3 | AI970788 | 0309 | PAN3 polyA specific | 1.05E−07 | −1.62 | 1.62 | 3.03 |
| | | | | ribonuclease subunit | | | | |
| | | | | homolog (S. cerevisiae) | | | | |
| 222133_s_at | PHF20L1 | AK022280 | 0332 | PHD finger protein 20-like 1 | 1.09E−07 | −1.61 | 1.61 | 2.12 |
| 237768_x_at | | AA825925 | 0066 | | 1.10E−07 | −2.29 | 2.29 | 2.57 |
| 208677_s_at | BSG | AL550657 | 0415 | Basigin (Ok blood group) | 1.16E−07 | 2.21 | 2.21 | 1.92 |
| 224572_s_at | IRF2BP2 | BG485163 | 0697 | Interferon regulatory factor | 1.36E−07 | 2.35 | 2.35 | 2.63 |
| | | | | 2 binding protein 2 | | | | |
| 221899_at | PFAAP5 | AI809961 | 0274 | Phosphonoformate | 1.37E−07 | −1.92 | 1.92 | 2.5 |
| | | | | immuno-associated | | | | |
| | | | | protein 5 | | | | |
| 224969_at | ATXN7L3 | AL390158 | 0401 | Ataxin 7-like 3 | 1.45E−07 | 1.65 | 1.65 | 2.32 |
| 229389_at | ATG16L2 | AA741058 | 0050 | ATG16 autophagy related | 1.46E−07 | 3 | 3 | 2.32 |
| | | | | 16-like 2 (S. cerevisiae) | | | | |
| 242134_at | | AI733194 | 0247 | Transcribed locus | 1.50E−07 | 2.2 | 2.2 | 1.72 |
| 224676_at | TMED4 | AI472339 | 0208 | Transmembrane emp24 | 1.52E−07 | 2.09 | 2.09 | 2.45 |
| | | | | protein transport domain | | | | |
| | | | | containing 4 | | | | |
| 224568_x_at | MALAT1 | AW005982 | 0462 | Metastasis associated | 1.60E−07 | 3.14 | 3.14 | 2.28 |
| | | | | lung adenocarcinoma | | | | |
| | | | | transcript 1 (non-coding | | | | |
| | | | | RNA) | | | | |
| 218659_at | ASXL2 | NM_018263 | 0923 | Additional sex combs like | 1.65E−07 | −1.46 | 1.46 | 2.88 |
| | | | | 2 (Drosophila) | | | | |
| 203497_at | PPARBP | NM_004774 | 0829 | PPAR binding protein | 1.68E−07 | −1.57 | 1.57 | 2.4 |
| 204285_s_at | PMAIP1 | AI857639 | 0288 | Phorbol-12-myristate-13- | 1.74E−07 | 2.56 | 2.56 | 1.82 |
| | | | | acetate-induced protein 1 | | | | |
| 213015_at | | BF448315 | 0638 | “ARTC1 mRNA, complete | 1.93E−07 | 2.51 | 2.51 | 1.95 |
| | | | | sequence” | | | | |
| 224567_x_at | MALAT1 | BG534952 | 0699 | Metastasis associated | 1.95E−07 | 2.79 | 2.79 | 2.47 |
| | | | | lung adenocarcinoma | | | | |
| | | | | transcript 1 (non-coding | | | | |
| | | | | RNA) | | | | |
| 222790_s_at | RSBN1 | AK022166 | 0330 | Round spermatid basic | 2.00E−07 | −2.2 | 2.2 | 2.87 |
| | | | | protein 1 | | | | |
| 207057_at | SLC16A7 | NM_004731 | 0828 | “Solute carrier family 16, | 2.09E−07 | 4.01 | 4.01 | 1.41 |
| | | | | member 7 | | | | |
| | | | | (monocarboxylic acid | | | | |
| | | | | transporter 2)” | | | | |
| 212852_s_at | TROVE2 | AL538601 | 0414 | “TROVE domain family, | 2.09E−07 | −1.44 | 1.44 | 2.61 |
| | | | | member 2” | | | | |
| 238761_at | | BE645241 | 0581 | | 2.13E−07 | −2.49 | 2.49 | 2.86 |
| 214198_s_at | DGCR2 | AU150824 | 0430 | DiGeorge syndrome | 2.16E−07 | −1.66 | 1.66 | 2.25 |
| | | | | critical region gene 2 | | | | |
| 202809_s_at | INTS3 | NM_023015 | 0943 | Integrator complex subunit 3 | 2.18E−07 | −1.26 | 1.26 | 2.19 |
| 236072_at | | N64578 | 0751 | Transcribed locus | 2.23E−07 | −2.3 | 2.3 | 1.91 |
| 225827_at | EIF2C2 | AI832074 | 0288 | “Eukaryotic translation | 2.28E−07 | 2.51 | 2.51 | 2.14 |
| | | | | initiation factor 2C, 2” | | | | |
| 241775_at | | AW298119 | 0502 | “CDNA FLJ26437 fis, | 2.37E−07 | −2.2 | 2.2 | 2.5 |
| | | | | clone KDN02067” | | | | |
| 215706_x_at | ZYX | BC002323 | 0543 | Zyxin | 2.42E−07 | 1.68 | 1.68 | 2.6 |
| 201730_s_at | TPR | BF110993 | 0619 | Translocated promoter | 2.44E−07 | 1.77 | 1.77 | 2.29 |
| | | | | region (to activated MET | | | | |
| | | | | oncogene) | | | | |
| 226447_at | ASH1L | BG290742 | 0687 | “Ash1 (absent, small, or | 2.55E−07 | −1.52 | 1.52 | 2.6 |
| | | | | homeotic)-like | | | | |
| | | | | (Drosophila)” | | | | |
| 200623_s_at | CALM3 | MM_005184 | 0836 | “Calmodulin 3 | 2.82E−07 | 1.87 | 1.87 | 2.33 |
| | | | | (phosphorylase kinase, | | | | |
| | | | | delta)” | | | | |
| 215990_s_at | BCL6 | S67779 | 0966 | B-cell CLL/lymphoma 6 | 2.92E−07 | 1.96 | 1.96 | 1.8 |
| | | | | (zinc finger protein 51) | | | | |
| 212007_at | UBXD2 | AI927512 | 0301 | UBX domain containing 2 | 3.02E−07 | 1.5 | 1.5 | 2.05 |
| 225885_at | EEA1 | AI336848 | 0183 | Early endosome antigen 1 | 3.04E−07 | −1.63 | 1.63 | 2.5 |
| 212629_s_at | PKN2 | AI633689 | 0224 | Protein kinase N2 | 3.06E−07 | 1.66 | 1.66 | 1.83 |
| 216971_s_at | PLEC1 | Z54367 | 1015 | “Plectin 1, intermediate | 3.10E−07 | 3.34 | 3.34 | 1.55 |
| | | | | filament binding protein | | | | |
| | | | | 500 kDa” | | | | |
| 225289_at | STAT3 | AI139252 | 0174 | Signal transducer and | 3.15E−07 | 1.58 | 1.58 | 2.62 |
| | | | | activator of transcription 3 | | | | |
| | | | | (acute-phase response | | | | |
| | | | | factor) | | | | |
| 217713_x_at | | AA126763 | 0008 | | 3.42E−07 | −1.62 | 1.62 | 2.22 |
| 225361_x_at | FAM122B | AI348001 | 0187 | Family with sequence | 3.69E−07 | −1.44 | 1.44 | 2.8 |
| | | | | similarity 122B | | | | |
| 242920_at | | AW590838 | 0520 | | 3.89E−07 | −2.63 | 2.63 | 2.52 |
| 227754_at | | AV700815 | 0447 | “CDNA FLJ10417 fis, | 3.93E−07 | −2.01 | 2.01 | 2.24 |
| | | | | clone NT2RP1000112” | | | | |
| 226680_at | | BF056303 | 0614 | Transcribed locus | 4.00E−07 | −2.17 | 2.17 | 2.33 |
| 202157_s_at | CUGBP2 | U69546 | 0989 | “CUG triplet repeat, RNA | 4.02E−07 | −1.47 | 1.47 | 3.3 |
| | | | | binding protein 2” | | | | |
| 208988_at | | BE675843 | 0589 | | 4.05E−07 | −1.46 | 1.46 | 2.28 |
| 222243_s_at | TOB2 | AB051450 | 0094 | “Transducer of ERBB2, 2” | 4.21E−07 | −1.35 | 1.35 | 2.03 |
| 217862_at | | N24868 | 0746 | Transcribed locus | 4.42E−07 | 1.53 | 1.53 | 2.29 |
| 64486_at | CORO1B | AI341234 | 0185 | “Coronin, actin binding | 4.42E−07 | 1.38 | 1.38 | 2.35 |
| | | | | protein, 1B” | | | | |
| 230918_at | | BE856598 | 0601 | | 4.43E−07 | −1.77 | 1.77 | 2.38 |
| 204978_at | SFRS16 | NM_007056 | 0873 | “Splicing factor, | 4.48E−07 | 2.27 | 2.27 | 1.8 |
| | | | | arginine/serine-rich 16” | | | | |
| 232879_at | CRTC3 | AK024981 | 0354 | CREB regulated | 4.61E−07 | −2.01 | 2.01 | 1.96 |
| | | | | transcription coactivator 3 | | | | |
| 235032_at | DNAJA5 | BG112118 | 0672 | DnaJ homology subfamily | 4.73E−07 | −1.89 | 1.89 | 2.4 |
| | | | | A member 5 | | | | |
| 203591_s_at | CSF3R | NM_000760 | 0761 | Colony stimulating factor 3 | 5.00E−07 | 2.01 | 2.01 | 2.16 |
| | | | | receptor (granulocyte) | | | | |
| 228098_s_at | MYLIP | AW292746 | 0497 | Myosin regulatory light | 5.00E−07 | −1.57 | 1.57 | 3.02 |
| | | | | chain interacting protein | | | | |
| 234734_s_at | TNRC6A | AK025696 | 0358 | Trinucleotide repeat | 5.06E−07 | 1.67 | 1.67 | 2.26 |
| | | | | containing 6A | | | | |
| 244433_at | | AI950023 | 0307 | | 5.36E−07 | −2.03 | 2.03 | 2.63 |
| 226641_at | | AU157224 | 0438 | “CDNA FLJ11570 fis, | 5.42E−07 | −1.56 | 1.56 | 2.93 |
| | | | | clone HEMBA1003309” | | | | |
| 222791_at | RSBN1 | AK022166 | 0330 | Round spermatid basic | 5.54E−07 | −2.12 | 2.12 | 3.08 |
| | | | | protein 1 | | | | |
| 225706_at | GLCCI1 | AI761989 | 0259 | Glucocorticoid induced | 5.60E−07 | −1.86 | 1.86 | 3 |
| | | | | transcript 1 | | | | |
| 243790_at | ZNF585A | AA203136 | 0015 | Zinc finger protein 585A | 5.73E−07 | −2.21 | 2.21 | 2.19 |
| 244145_at | | BG260337 | 0683 | | 5.74E−07 | −1.62 | 1.62 | 2.4 |
| 203751_x_at | JUND | AI762296 | 0260 | Jun D proto-oncogene | 5.89E−07 | 1.97 | 1.97 | 1.9 |
| 225216_at | CXorf39 | AI590719 | 0220 | Chromosome X open | 5.91E−07 | −1.37 | 1.37 | 2.62 |
| | | | | reading frame 39 | | | | |
| 228853_at | “(LOC730432, | AI652546 | 0229 | Serine/threonine/tyrosine | 5.96E−07 | −1.49 | 1.49 | 2.5 |
| | STYX)” | | | interacting protein | | | | |
| 232909_s_at | “(BPTF, | AU146870 | 0426 | “(Bromodomain PHD | 6.00E−07 | −1.58 | 1.58 | 2.89 |
| | LOC646043)” | | | finger transcription factor, | | | | |
| | | | | Similar to Enhancer of | | | | |
| | | | | bithorax CG32346-PB, | | | | |
| | | | | isoform B)” | | | | |
| 36564_at | IBRDC3 | W27419 | 0998 | IBR domain containing 3 | 6.14E−07 | 1.89 | 1.89 | 2.03 |
| 200073_s_at | HNRPD | M94630 | 0742 | “Heterogeneous nuclear | 6.30E−07 | −1.43 | 1.43 | 3.24 |
| | | | | ribonucleoprotein D (AU- | | | | |
| | | | | rich element RNA binding | | | | |
| | | | | protein 1, 37 kDa)” | | | | |
| 200808_s_at | ZYX | NM_003461 | 0804 | Zyxin | 6.30E−07 | 1.78 | 1.78 | 2.6 |
| 235125_x_at | | AI078279 | 0167 | Transcribed locus | 6.45E−07 | −2.67 | 2.67 | 2.17 |
| 226712_at | | BF206389 | 0627 | “CDNA: FLJ22100 fis, | 6.45E−07 | 1.51 | 1.51 | 2.3 |
| | | | | clone HEP17127” | | | | |
| 208447_s_at | PRPS1 | NM_002764 | 0795 | Phosphoribosyl | 6.64E−07 | 1.93 | 1.93 | 1.83 |
| | | | | pyrophosphate synthetase 1 | | | | |
| 226426_at | | BG149849 | 0674 | Transcribed locus | 6.67E−07 | −1.5 | 1.5 | 2.66 |
| 239163_at | UBE2B | AW364833 | 0507 | Ubiquitin-conjugating | 6.80E−07 | 2.05 | 2.05 | 1.81 |
| | | | | enzyme E2B (RAD6 | | | | |
| | | | | homolog) | | | | |
| 219426_at | EIF2C3 | NM_024852 | 0949 | “Eukaryotic translation | 6.81E−07 | 1.65 | 1.65 | 1.64 |
| | | | | initiation factor 2C, 3” | | | | |
| 235341_at | LOC144871 | AL119957 | 0387 | Hypothetical protein | 7.43E−07 | 1.78 | 1.78 | 2.3 |
| | | | | LOC144871 | | | | |
| 242243_at | TMF1 | AI767435 | 0264 | TATA element modulatory | 7.88E−07 | −2.26 | 2.26 | 2.01 |
| | | | | factor 1 | | | | |
| 201236_s_at | BTG2 | NM_006763 | 0870 | “BTG family, member 2” | 8.07E−07 | 1.55 | 1.55 | 2.45 |
| 202577_s_at | DDX19A | BC005162 | 0553 | DEAD (Asp-Glu-Ala-As) | 8.20E−07 | −1.44 | 1.44 | 2.07 |
| | | | | box polypeptide 19A | | | | |
| 227772_at | | AV700849 | 0448 | | 9.74E−07 | −1.72 | 1.72 | 2.33 |
| 229897_at | ZNF641 | BF195808 | 0626 | Zinc finger protein 641 | 9.75E−07 | 1.86 | 1.86 | 2.15 |
| 218566_s_at | CHORDC1 | NM_012124 | 0876 | Cysteine and histidine-rich | 9.81E−07 | −1.61 | 1.61 | 2.33 |
| | | | | domain (CHORD)- | | | | |
| | | | | containing 1 | | | | |
| 203839_s_at | TNK2 | NM_005781 | 0845 | “Tyrosine kinase, non- | 9.95E−07 | 2.11 | 2.11 | 1.63 |
| | | | | receptor, 2” | | | | |
| 204361_s_at | SKAP2 | AB014486 | 0085 | Src kinase associated | 1.02E−06 | −1.79 | 1.79 | 2.09 |
| | | | | phosphoprotein 2 | | | | |
| 224250_s_at | SECISBP2 | BC001189 | 0541 | SECIS binding protein 2 | 1.02E−06 | 1.74 | 1.74 | 2.57 |
| 225350_s_at | ZYG11B | AV701229 | 0449 | Zyg-11 homolog B (C. elegans) | 1.03E−06 | −1.54 | 1.54 | 2.83 |
| 204516_at | | BG390306 | 0692 | Transcribed locus | 1.05E−06 | −2.17 | 2.17 | 2.56 |
| 225620_at | RAB35 | AL534848 | 0412 | “RAB35, member RAS | 1.05E−06 | 1.56 | 1.56 | 2.27 |
| | | | | oncogene family” | | | | |
| 226965_at | FAM116A | BF438017 | 0636 | “(Family with sequence | 1.07E−06 | −1.53 | 1.53 | 2.91 |
| | | | | similarity 116, member A, | | | | |
| | | | | Transcribed locus)” | | | | |
| 239808_at | | AI084489 | 0169 | Transcribed locus | 1.08E−06 | −1.87 | 1.87 | 2.47 |
| 227428_at | GABPA | BE876628 | 0605 | “GA binding protein | 1.20E−06 | −1.53 | 1.53 | 2.52 |
| | | | | transcription factor, alpha | | | | |
| | | | | subunit 60 kDa” | | | | |
| 218595_s_at | “(HEATR1, | NM_018072 | 0921 | “Lectin, galactoside- | 1.20E−06 | −1.43 | 1.43 | 2.3 |
| | LGALS8)” | | | binding, soluble, 8 | | | | |
| | | | | (galectin 8)” | | | | |
| 222266_at | C19orf2 | BF796940 | 0661 | Chromosome 19 open | 1.23E−06 | −1.72 | 1.72 | 2.17 |
| | | | | reading frame 2 | | | | |
| 239597_at | | AA993566 | 0080 | | 1.23E−06 | 2.33 | 2.33 | 2.06 |
| 203415_at | PDCD6 | NM_013232 | 0879 | Programmed cell death 6 | 1.23E−06 | 1.36 | 1.36 | 2.2 |
| 208866_at | | BF510713 | 0642 | CDNA clone | 1.23E−06 | −1.59 | 1.59 | 2.2 |
| | | | | IMAGE: 4044084 | | | | |
| 212575_at | C19orf6 | BF966155 | 0665 | Chromosome 19 open | 1.27E−06 | 12.23 | 12.23 | 0.38 |
| | | | | reading frame 6 | | | | |
| 225176_at | | AA156754 | 0013 | “CDNA FLJ42149 fis, | 1.30E−06 | −1.4 | 1.4 | 2.96 |
| | | | | clone THYMU1000692” | | | | |
| 227223_at | RBM39 | BE466173 | 0575 | RNA binding motif protein | 1.32E−06 | 2.29 | 2.29 | 2.22 |
| | | | | 39 | | | | |
| 41386_i_at | JMJD3 | AB002344 | 0082 | Jumonji domain | 1.33E−06 | 1.7 | 1.7 | 2.35 |
| | | | | containing 3 | | | | |
| 225995_x_at | “(FAM39DP, | BG178695 | 0675 | “(CXYorf1-related protein, | 1.38E−06 | 1.64 | 1.64 | 2.45 |
| | FLJ00038, | | | Family with sequence | | | | |
| | LOC376475, | | | similarity 39, member D | | | | |
| | LOC727741)” | | | pseudogene)” | | | | |
| 228328_at | KLHL28 | AI982758 | 0311 | Kelch-like 28 (Drosophila) | 1.41E−06 | −1.71 | 1.71 | 2.43 |
| 235409_at | MGA | AU149225 | 0429 | MAX gene associated | 1.41E−06 | 2.4 | 2.4 | 1.89 |
| 200649_at | NUCB1 | BC002356 | 0544 | Nucleobindin 1 | 1.42E−06 | 1.85 | 1.85 | 2.2 |
| 232615_at | | AA632758 | 0034 | Transcribed locus | 1.43E−06 | −2.01 | 2.01 | 3.06 |
| 205882_x_at | ADD3 | AI818488 | 0278 | Adducin 3 (gamma) | 1.44E−06 | −1.46 | 1.46 | 3.28 |
| 228496_s_at | CRIM1 | AW243081 | 0489 | Cysteine rich | 1.44E−06 | −2.14 | 2.14 | 2.18 |
| | | | | transmembrane BMP | | | | |
| | | | | regulator 1 (chordin-like) | | | | |
| 209189_at | FOS | BC004490 | 0549 | V-fos FBJ murine | 1.50E−06 | 3.42 | 3.42 | 2.61 |
| | | | | osteosarcoma viral | | | | |
| | | | | oncogene homolog | | | | |
| 239233_at | KIAA1212 | AA744613 | 0053 | KIAA1212 | 1.53E−06 | −2.29 | 2.29 | 1.4 |
| 224308_s_at | INTS2 | AL136800 | 0393 | Integrator complex subunit 2 | 1.61E−06 | −1.98 | 1.98 | 1.98 |
| 208705_s_at | EIF5 | BG481972 | 0696 | Eukaryotic translation | 1.62E−06 | 1.3 | 1.3 | 2.56 |
| | | | | initiation factor 5 | | | | |
| 218607_s_at | SDAD1 | NM_018115 | 0922 | SDA1 domain containing 1 | 1.63E−06 | 1.52 | 1.52 | 2.24 |
| 200041_s_at | BAT1 | NM_004640 | 0826 | HLA-B associated | 1.71E−06 | 1.29 | 1.29 | 2.94 |
| | | | | transcript 1 | | | | |
| 202850_at | ABCD3 | NM_002858 | 0796 | “ATP-binding cassette, | 1.71E−06 | −1.56 | 1.56 | 2.3 |
| | | | | sub-family D (ALD), | | | | |
| | | | | member 3” | | | | |
| 224562_at | “(LOC644739, | AK025566 | 0357 | “(Similar to Wiskott- | 1.73E−06 | 1.76 | 1.76 | 2.94 |
| | LOC647909, | | | Aldrich syndrome protein | | | | |
| | WASF2)” | | | family member 4 (WASP- | | | | |
| | | | | family protein member 4), | | | | |
| | | | | WAS protein family, | | | | |
| | | | | member 2)” | | | | |
| 222644_s_at | GLT25D1 | AI924150 | 0296 | Glycosyltransferase 25 | 1.80E−06 | 2.01 | 2.01 | 2.09 |
| | | | | domain containing 1 | | | | |
| 212536_at | ATP11B | AB023173 | 0088 | “ATPase, Class VI, type | 1.81E−06 | −1.52 | 1.52 | 2.17 |
| | | | | 11B” | | | | |
| 215269_at | TMEM1 | AI922538 | 0294 | Transmembrane protein 1 | 1.82E−06 | −1.96 | 1.96 | 2.01 |
| 243303_at | | AA811657 | 0064 | | 1.93E−06 | −1.92 | 1.92 | 2.34 |
| 201991_s_at | KIF5B | BF223224 | 0630 | Kinesin family member 5B | 1.95E−06 | −1.36 | 1.36 | 2.75 |
| 213327_s_at | USP12 | AI820101 | 0280 | Ubiquitin specific | 1.97E−06 | −1.58 | 1.58 | 1.97 |
| | | | | peptidase 12 | | | | |
| 213882_at | TM2D1 | AA012917 | 0001 | TM2 domain containing 1 | 1.97E−06 | −1.68 | 1.68 | 1.66 |
| 214715_x_at | ZNF160 | AK024789 | 0348 | Zinc finger protein 160 | 2.00E−06 | −1.65 | 1.65 | 2.37 |
| 225845_at | ZBTB44 | BG253884 | 0681 | Zinc finger and BTB | 2.01E−06 | −1.68 | 1.68 | 2.81 |
| | | | | domain containing 44 | | | | |
| 215529_x_at | DIP2A | AI590053 | 0219 | DIP2 disco-interacting | 2.01E−06 | −1.99 | 1.99 | 1.89 |
| | | | | protein 2 homolog A | | | | |
| | | | | (Drosophila) | | | | |
| 242539_at | LOC730069 | AW665509 | 0523 | Similar to nuclear receptor | 2.04E−06 | −2.21 | 2.21 | 2.33 |
| | | | | binding factor 2 | | | | |
| 203311_s_at | ARF6 | M57763 | 0735 | ADP-ribosylation factor 6 | 2.07E−06 | 1.9 | 1.9 | 2.23 |
| 205854_at | TULP3 | AK024246 | 0342 | Tubby like protein 3 | 2.08E−06 | −1.57 | 1.57 | 1.79 |
| 212042_x_at | “(LOC653949, | BG389744 | 0691 | Ribosomal protein L7 | 2.10E−06 | −1.37 | 1.37 | 3.51 |
| | RPL7, | | | | | | | |
| | hCG_2015956, | | | | | | | |
| | hCG_31916)” | | | | | | | |
| 233369_at | | AU146027 | 0424 | “CDNA FLJ11835 fis, | 2.23E−06 | −1.78 | 1.78 | 2.42 |
| | | | | clone HEMBA1006595” | | | | |
| 212513_s_at | USP33 | AB029020 | 0089 | Ubiquitin specific | 2.27E−06 | −1.31 | 1.31 | 2.52 |
| | | | | peptidase 33 | | | | |
| 218626_at | EIF4ENIF1 | NM_019843 | 0930 | Eukaryotic translation | 2.30E−06 | −1.33 | 1.33 | 2.1 |
| | | | | initiation factor 4E nuclear | | | | |
| | | | | import factor 1 | | | | |
| 208246_x_at | | NM_017618 | 0909 | | 2.31E−06 | −1.63 | 1.63 | 2.29 |
| 224559_at | MALAT1 | AF001540 | 0104 | Metastasis associated | 2.36E−06 | 4.55 | 4.55 | 2.49 |
| | | | | lung adenocarcinoma | | | | |
| | | | | transcript 1 (non-coding | | | | |
| | | | | RNA) | | | | |
| 205312_at | SPI1 | NM_003120 | 0798 | Spleen focus forming virus | 2.41E−06 | 2.77 | 2.77 | 1.91 |
| | | | | (SFFV) proviral integration | | | | |
| | | | | oncogene spi1 | | | | |
| 213318_s_at | BAT3 | BG028844 | 0669 | HLA-B associated | 2.45E−06 | 1.41 | 1.41 | 2.56 |
| | | | | transcript 3 | | | | |
| 212099_at | RHOB | AI263909 | 0179 | “Ras homolog gene | 2.50E−06 | 2.36 | 2.36 | 2.32 |
| | | | | family, member B” | | | | |
| 223131_s_at | TRIM8 | AI925572 | 0299 | Tripartite motif-containing 8 | 2.51E−06 | 1.74 | 1.74 | 1.9 |
| 212487_at | GPATCH8 | AI673812 | 0234 | G patch domain | 2.53E−06 | 2.33 | 2.33 | 1.57 |
| | | | | containing 8 | | | | |
| 200011_s_at | ARF3 | NM_001659 | 0775 | ADP-ribosylation factor 3 | 2.55E−06 | 1.43 | 1.43 | 2.95 |
| 210543_s_at | “(LOC731751, | U34994 | 0981 | “Protein kinase, DNA- | 2.61E−06 | −1.39 | 1.39 | 2.21 |
| | PRKDC)” | | | activated, catalytic | | | | |
| | | | | polypeptide” | | | | |
| 217313_at | | AC004692 | 0098 | | 2.63E−06 | −1.62 | 1.62 | 2.34 |
| 228106_at | C4orf30 | AA031528 | 0004 | Chromosome 4 open | 2.66E−06 | −1.57 | 1.57 | 2.42 |
| | | | | reading frame 30 | | | | |
| 218043_s_at | AZI2 | NM_022461 | 0940 | 5-azacytidine induced 2 | 2.67E−06 | −2.08 | 2.08 | 1.89 |
| 203514_at | MAP3K3 | BF971923 | 0666 | Mitogen-activated protein | 2.67E−06 | −1.52 | 1.52 | 2.21 |
| | | | | kinase kinase kinase 3 | | | | |
| 223295_s_at | LUC7L | BE049621 | 0566 | LUC7-like (S. cerevisiae) | 2.68E−06 | −1.57 | 1.57 | 2.67 |
| 235469_at | MGC40405 | AV744101 | 0457 | Hypothetical protein | 2.73E−06 | −2.48 | 2.48 | 1.95 |
| | | | | MGC40405 | | | | |
| 209440_at | PRPS1 | BC001605 | 0542 | Phosphoribosyl | 2.77E−06 | 1.42 | 1.42 | 2.22 |
| | | | | pyrophosphate synthetase 1 | | | | |
| 227039_at | AKAP13 | AI674926 | 0235 | A kinase (PRKA) anchor | 2.82E−06 | 1.92 | 1.92 | 2.46 |
| | | | | protein 13 | | | | |
| 218499_at | RP6-213H19.1 | NM_016542 | 0904 | Serine/threonine protein | 2.94E−06 | −1.52 | 1.52 | 2.53 |
| | | | | kinase MST4 | | | | |
| 232440_at | ZDHHC13 | AU155198 | 0433 | “Zinc finger, DHHC-type | 2.95E−06 | −1.75 | 1.75 | 2.26 |
| | | | | containing 13” | | | | |
| 209727_at | GM2A | M76477 | 0737 | GM2 ganglioside activator | 2.97E−06 | 5.7 | 5.7 | 1.01 |
| 241891_at | | T92908 | 0972 | | 2.97E−06 | −1.82 | 1.82 | 2.64 |
| 227525_at | GLCCI1 | AA058770 | 0006 | Glucocorticoid induced | 3.10E−06 | −1.75 | 1.75 | 2.66 |
| | | | | transcript 1 | | | | |
| 228088_at | | AI092265 | 0170 | “CDNA FLJ31513 fis, | 3.17E−06 | −1.49 | 1.49 | 2.11 |
| | | | | clone NT2RI1000127” | | | | |
| 242362_at | | AI797788 | 0269 | | 3.17E−06 | −2.27 | 2.27 | 2.69 |
| 34478_at | RAB11B | X79780 | 1009 | “RAB11B, member RAS | 3.23E−06 | 2.63 | 2.63 | 1.27 |
| | | | | oncogene family” | | | | |
| 223135_s_at | BBX | AL136769 | 0392 | Bobby sox homolog | 3.25E−06 | 1.41 | 1.41 | 2.47 |
| | | | | (Drosophila) | | | | |
| 225780_at | RSC1A1 | AL565415 | 0418 | “Regulatory solute carrier | 3.26E−06 | −1.59 | 1.59 | 2.5 |
| | | | | protein, family 1, member | | | | |
| | | | | 1” | | | | |
| 211791_s_at | KCNAB2 | AF044253 | 0112 | “Potassium voltage-gated | 3.27E−06 | 4.73 | 4.73 | 1.31 |
| | | | | channel, shaker-related | | | | |
| | | | | subfamily, beta member | | | | |
| | | | | 2” | | | | |
| 204252_at | CDK2 | M68520 | 0736 | Cyclin-dependent kinase 2 | 3.29E−06 | −1.38 | 1.38 | 2.06 |
| 202420_s_at | DHX9 | NM_001357 | 0771 | DEAH (Asp-Glu-Ala-His) | 3.31E−06 | −1.29 | 1.29 | 2.35 |
| | | | | box polypeptide 9 | | | | |
| 223138_s_at | DHX36 | AI937206 | 0304 | DEAH (Asp-Glu-Ala-His) | 3.31E−06 | 1.69 | 1.69 | 2.21 |
| | | | | box polypeptide 36 | | | | |
| 240452_at | GSPT1 | AA580082 | 0030 | G1 to S phase transition 1 | 3.38E−06 | −2.24 | 2.24 | 2.5 |
| 202082_s_at | SEC14L1 | AV748469 | 0458 | SEC14-like 1 (S. cerevisiae) | 3.38E−06 | 1.72 | 1.72 | 1.74 |
| 213038_at | IBRDC3 | AL031602 | 0365 | IBR domain containing 3 | 3.63E−06 | 1.55 | 1.55 | 1.95 |
| 232038_at | C6orf170 | AI052103 | 0162 | Chromosome 6 open | 3.76E−06 | −2 | 2 | 1.73 |
| | | | | reading frame 170 | | | | |
| 229036_at | TNRC6B | AI681177 | 0238 | “(Transcribed locus, | 3.80E−06 | 1.83 | 1.83 | 2.32 |
| | | | | Trinucleotide repeat | | | | |
| | | | | containing 6B)” | | | | |
| 229235_at | | AI983432 | 0312 | Transcribed locus | 3.81E−06 | −1.53 | 1.53 | 2.59 |
| 226412_at | C6orf111 | N66397 | 0752 | Chromosome 6 open | 3.84E−06 | −1.63 | 1.63 | 2.76 |
| | | | | reading frame 111 | | | | |
| 212637_s_at | WWP1 | AU155187 | 0432 | WW domain containing E3 | 3.95E−06 | −1.64 | 1.64 | 1.62 |
| | | | | ubiquitin protein ligase 1 | | | | |
| 220296_at | | NM_024564 | 0947 | | 3.99E−06 | −1.96 | 1.96 | 2 |
| 240695_at | | AA876138 | 0075 | | 4.03E−06 | 1.68 | 1.68 | 1.78 |
| 224759_s_at | C12orf23 | AK001731 | 0320 | Chromosome 12 open | 4.20E−06 | −1.63 | 1.63 | 2.48 |
| | | | | reading frame 23 | | | | |
| 222794_x_at | PAPD1 | AK022188 | 0331 | PAP associated domain | 4.32E−06 | −1.41 | 1.41 | 2.51 |
| | | | | containing 1 | | | | |
| 224974_at | SUDS3 | AK024460 | 0344 | Suppressor of defective | 4.36E−06 | −1.94 | 1.94 | 2.27 |
| | | | | silencing 3 homolog (S. cerevisiae) | | | | |
| 242650_at | | AW298590 | 0503 | Transcribed locus | 4.42E−06 | −2.21 | 2.21 | 1.84 |
| 212921_at | SMYD2 | AF070592 | 0121 | SET and MYND domain | 4.45E−06 | −1.74 | 1.74 | 1.88 |
| | | | | containing 2 | | | | |
| 217717_s_at | YWHAB | BF246499 | 0631 | “Tyrosine 3- | 4.47E−06 | 1.58 | 1.58 | 2.51 |
| | | | | monooxygenase/tryptophan | | | | |
| | | | | 5-monooxygenase | | | | |
| | | | | activation protein, beta | | | | |
| | | | | polypeptide” | | | | |
| 221419_s_at | | NM_013307 | 0880 | | 4.56E−06 | 2.08 | 2.08 | 2.03 |
| 204809_at | CLPX | NM_006660 | 0866 | ClpX caseinolytic | 4.61E−06 | −1.37 | 1.37 | 1.99 |
| | | | | peptidase X homolog (E. coli) | | | | |
| 201409_s_at | PPP1CB | NM_002709 | 0794 | “Protein phosphatase 1, | 4.64E−06 | −1.38 | 1.38 | 2.43 |
| | | | | catalytic subunit, beta | | | | |
| | | | | isoform” | | | | |
| 209829_at | C6orf32 | AB002384 | 0083 | Chromosome 6 open | 4.67E−06 | −1.61 | 1.61 | 2.89 |
| | | | | reading frame 32 | | | | |
| 225761_at | PAPD4 | AI434509 | 0203 | PAP associated domain | 4.74E−06 | −1.42 | 1.42 | 2.78 |
| | | | | containing 4 | | | | |
| 228471_at | ANKRD44 | AA744636 | 0054 | Ankyrin repeat domain 44 | 4.84E−06 | −1.71 | 1.71 | 2.75 |
| 201526_at | ARF5 | NM_001662 | 0776 | ADP-ribosylation factor 5 | 4.87E−06 | 1.49 | 1.49 | 2.21 |
| 241435_at | | AA702930 | 0044 | | 5.03E−06 | −2.16 | 2.16 | 3.23 |
| 227567_at | AMZ2 | AL524467 | 0407 | Archaemetzincins-2 | 5.11E−06 | −1.69 | 1.69 | 2.53 |
| 209563_x_at | CALM1 | BC000454 | 0537 | “Calmodulin 1 | 5.22E−06 | 1.68 | 1.68 | 2.84 |
| | | | | (phosphorylase kinase, | | | | |
| | | | | delta)” | | | | |
| 242518_at | | AA748423 | 0056 | | 5.26E−06 | 1.6 | 1.6 | 1.87 |
| 239049_at | | BF514509 | 0644 | | 5.31E−06 | −1.85 | 1.85 | 2.38 |
| 209024_s_at | SYNCRIP | AI472757 | 0209 | “Synaptotagmin binding, | 5.50E−06 | 1.48 | 1.48 | 2.14 |
| | | | | cytoplasmic RNA | | | | |
| | | | | interacting protein” | | | | |
| 211948_x_at | BAT2D1 | BG261071 | 0685 | BAT2 domain containing 1 | 5.57E−06 | 2.77 | 2.77 | 2.01 |
| 228446_at | KIAA2026 | BF062203 | 0616 | KIAA2026 | 5.67E−06 | −1.44 | 1.44 | 2.53 |
| 229541_at | | BE669703 | 0583 | “CDNA FLJ38918 fis, | 5.86E−06 | 1.96 | 1.96 | 1.97 |
| | | | | clone NT2NE2010633” | | | | |
| 237330_at | | AA603494 | 0033 | Transcribed locus | 5.89E−06 | −2.48 | 2.48 | 1.96 |
| 217842_at | LUC7L2 | NM_016019 | 0893 | LUC7-like 2 (S. cerevisiae) | 5.90E−06 | −1.51 | 1.51 | 1.79 |
| 201828_x_at | FAM127A | NM_003928 | 0809 | “Family with sequence | 5.96E−06 | 1.27 | 1.27 | 2.33 |
| | | | | similarity 127, member A” | | | | |
| 229366_at | | BG149765 | 0673 | Transcribed locus | 5.98E−06 | −1.33 | 1.33 | 2.34 |
| 201986_at | THRAP1 | AB011165 | 0084 | Thyroid hormone receptor | 6.02E−06 | −1.46 | 1.46 | 2.51 |
| | | | | associated protein 1 | | | | |
| 202373_s_at | RAB3GAP2 | AF255648 | 0148 | RAB3 GTPase activating | 6.03E−06 | −1.34 | 1.34 | 2.46 |
| | | | | protein subunit 2 (non- | | | | |
| | | | | catalytic) | | | | |
| 241906_at | | BF001773 | 0612 | | 6.07E−06 | −2.1 | 2.1 | 2.45 |
| 201318_s_at | “(MRCL3, | NM_006471 | 0863 | “(Myosin regulatory light | 6.16E−06 | −1.33 | 1.33 | 3.25 |
| | MRLC2)” | | | chain MRCL3, Myosin | | | | |
| | | | | regulatory light chain | | | | |
| | | | | MRLC2)” | | | | |
| 207941_s_at | RBM39 | NM_004902 | 0830 | RNA binding motif protein | 6.17E−06 | −1.34 | 1.34 | 2.85 |
| | | | | 39 | | | | |
| 229586_at | CHD9 | AW300405 | 0505 | Chromodomain helicase | 6.33E−06 | 1.64 | 1.64 | 2.01 |
| | | | | DNA binding protein 9 | | | | |
| 217336_at | | AL118510 | 0385 | | 6.34E−06 | −1.68 | 1.68 | 2.28 |
| 209648_x_at | SOCS5 | AL136896 | 0394 | Suppressor of cytokine | 6.36E−06 | −1.86 | 1.86 | 1.59 |
| | | | | signaling 5 | | | | |
| 226664_at | TBC1D20 | AL121747 | 0388 | “TBC1 domain family, | 6.40E−06 | 1.39 | 1.39 | 2.3 |
| | | | | member 20” | | | | |
| 225786_at | LOC284702 | AI440495 | 0204 | Hypothetical protein | 6.49E−06 | −1.6 | 1.6 | 2.45 |
| | | | | LOC284702 | | | | |
| 208635_x_at | NACA | BF976260 | 0667 | Nascent-polypeptide- | 6.53E−06 | −1.24 | 1.24 | 3.71 |
| | | | | associated complex alpha | | | | |
| | | | | polypeptide | | | | |
| 223444_at | SENP7 | AL136599 | 0391 | SUMO1/sentrin specific | 6.56E−06 | −1.88 | 1.88 | 2.36 |
| | | | | peptidase 7 | | | | |
| 208975_s_at | KPNB1 | L38951 | 0725 | Karyopherin (importin) | 6.58E−06 | −1.54 | 1.54 | 2.17 |
| | | | | beta 1 | | | | |
| 208935_s_at | LGALS8 | L78132 | 0727 | “Lectin, galactoside- | 6.59E−06 | −1.66 | 1.66 | 2.18 |
| | | | | binding, soluble, 8 | | | | |
| | | | | (galectin 8)” | | | | |
| 200847_s_at | TMEM66 | NM_016127 | 0895 | Transmembrane protein | 6.65E−06 | −1.29 | 1.29 | 3.1 |
| | | | | 66 | | | | |
| 218247_s_at | RKHD2 | NM_016626 | 0906 | Ring finger and KH | 6.67E−06 | −1.93 | 1.93 | 2.32 |
| | | | | domain containing 2 | | | | |
| 227121_at | | BF476076 | 0640 | MRNA; cDNA | 6.71E−06 | −1.51 | 1.51 | 2.61 |
| | | | | DKFZp586K1922 (from | | | | |
| | | | | clone DKFZp586K1922) | | | | |
| 235984_at | | AL036662 | 0367 | Transcribed locus | 6.72E−06 | 1.85 | 1.85 | 2 |
| 212069_s_at | KIAA0515 | AK026025 | 0360 | KIAA0515 | 6.72E−06 | 1.73 | 1.73 | 1.89 |
| 243649_at | | AI678692 | 0236 | | 6.73E−06 | −1.82 | 1.82 | 2.15 |
| 238549_at | | AI420611 | 0201 | Transcribed locus | 6.84E−06 | −1.43 | 1.43 | 2.5 |
| 233093_s_at | BIRC6 | AK023788 | 0339 | Baculoviral IAP repeat- | 6.88E−06 | −1.25 | 1.25 | 2.93 |
| | | | | containing 6 (apollon) | | | | |
| 221749_at | YTHDF3 | AU157915 | 0439 | “YTH domain family, | 6.98E−06 | −1.61 | 1.61 | 2.37 |
| | | | | member 3” | | | | |
| 242726_at | | BF221850 | 0629 | Transcribed locus | 7.03E−06 | −2 | 2 | 2.11 |
| 242195_x_at | NUMBL | BE350811 | 0573 | Numb homolog | 7.11E−06 | −1.48 | 1.48 | 2.77 |
| | | | | (Drosophila)-like | | | | |
| 204020_at | PURA | BF739943 | 0658 | Purine-rich element | 7.23E−06 | −1.4 | 1.4 | 2.5 |
| | | | | binding protein A | | | | |
| 208694_at | PRKDC | U47077 | 0984 | “Protein kinase, DNA- | 7.38E−06 | −1.5 | 1.5 | 2.12 |
| | | | | activated, catalytic | | | | |
| | | | | polypeptide” | | | | |
| 200047_s_at | YY1 | NM_003403 | 0802 | YY1 transcription factor | 7.42E−06 | −1.29 | 1.29 | 2.83 |
| 228097_at | MYLIP | AW292746 | 0497 | Myosin regulatory light | 7.43E−06 | −1.57 | 1.57 | 2.55 |
| | | | | chain interacting protein | | | | |
| 213605_s_at | | AL049987 | 0376 | MRNA; cDNA | 7.49E−06 | −2.47 | 2.47 | 2.16 |
| | | | | DKFZp564F112 (from | | | | |
| | | | | clone DKFZp564F112) | | | | |
| 218259_at | MKL2 | NM_014048 | 0881 | MKL/myocardin-like 2 | 7.81E−06 | −1.57 | 1.57 | 2.02 |
| 240338_at | LRAP | AI807341 | 0272 | Leukocyte-derived | 7.89E−06 | −2.06 | 2.06 | 1.76 |
| | | | | arginine aminopeptidase | | | | |
| 226366_at | SHPRH | AI828221 | 0285 | SNF2 histone linker PHD | 7.99E−06 | −1.32 | 1.32 | 2.24 |
| | | | | RING helicase | | | | |
| 218237_s_at | SLC38A1 | NM_030674 | 0955 | “Solute carrier family 38, | 8.02E−06 | −1.91 | 1.91 | 2.93 |
| | | | | member 1” | | | | |
| 201473_at | JUNB | NM_002229 | 0783 | Jun B proto-oncogene | 8.04E−06 | 1.97 | 1.97 | 2.54 |
| 226786_at | RFX1 | BF507952 | 0641 | “Regulatory factor X, 1 | 8.06E−06 | 1.46 | 1.46 | 2.56 |
| | | | | (influences HLA class II | | | | |
| | | | | expression)” | | | | |
| 214948_s_at | | AL050136 | 0377 | MRNA; cDNA | 8.09E−06 | −1.4 | 1.4 | 2.26 |
| | | | | DKFZp586L141 (from | | | | |
| | | | | clone DKFZp586L141) | | | | |
| 225672_at | GOLGA2 | AL514295 | 0405 | “Golgi autoantigen, golgin | 8.22E−06 | 1.32 | 1.32 | 2.53 |
| | | | | subfamily a, 2” | | | | |
| 229787_s_at | OGT | AI742039 | 0251 | O-linked N- | 8.27E−06 | −2.15 | 2.15 | 2.41 |
| | | | | acetylglucosamine | | | | |
| | | | | (GlcNAc) transferase | | | | |
| | | | | (UDP-N- | | | | |
| | | | | acetylglucosamine:polypeptide- | | | | |
| | | | | N- | | | | |
| | | | | acetylglucosaminyl | | | | |
| | | | | transferase) | | | | |
| 202253_s_at | DNM2 | NM_004945 | 0832 | Dynamin 2 | 8.34E−06 | 1.63 | 1.63 | 1.93 |
| 225564_at | SPATA13 | AW269397 | 0493 | Spermatogenesis | 8.50E−06 | −1.7 | 1.7 | 2.97 |
| | | | | associated 13 | | | | |
| 218967_s_at | PTER | NM_030664 | 0954 | Phosphotriesterase | 8.54E−06 | −1.53 | 1.53 | 2.11 |
| | | | | related | | | | |
| 217007_s_at | ADAM15 | AK000667 | 0316 | ADAM metallopeptidase | 8.61E−06 | 4.58 | 4.58 | 0.88 |
| | | | | domain 15 (metargidin) | | | | |
| 228250_at | FNIP1 | AW263086 | 0491 | Folliculin interacting | 8.65E−06 | 2.39 | 2.39 | 1.78 |
| | | | | protein 1 | | | | |
| 212291_at | HIPK1 | AI393355 | 0199 | Homeodomain interacting | 8.65E−06 | 1.96 | 1.96 | 2.07 |
| | | | | protein kinase 1 | | | | |
| 203203_s_at | KRR1 | NM_007043 | 0872 | “KRR1, small subunit | 8.67E−06 | −1.64 | 1.64 | 1.93 |
| | | | | (SSU) processome | | | | |
| | | | | component, homolog | | | | |
| | | | | (yeast)” | | | | |
| 229470_at | FAM105B | AW451634 | 0509 | “Family with sequence | 8.88E−06 | −1.53 | 1.53 | 2.41 |
| | | | | similarity 105, member B” | | | | |
| 224698_at | FAM62B | AB033054 | 0090 | Family with sequence | 8.90E−06 | −1.58 | 1.58 | 2.83 |
| | | | | similarity 62 (C2 domain | | | | |
| | | | | containing) member B | | | | |
| 244061_at | | AI510829 | 0213 | | 8.91E−06 | −2.35 | 2.35 | 2.44 |
| 204075_s_at | KIAA0562 | NM_014704 | 0887 | KIAA0562 | 9.05E−06 | −2.13 | 2.13 | 1.39 |
| 217019_at | | AL137162 | 0395 | | 9.06E−06 | −2.35 | 2.35 | 1.82 |
| 218600_at | LIMD2 | NM_030576 | 0953 | LIM domain containing 2 | 9.07E−06 | 1.85 | 1.85 | 2.01 |
| 213606_s_at | ARHGDIA | AI571798 | 0218 | Rho GDP dissociation | 9.08E−06 | 2.93 | 2.93 | 1.41 |
| | | | | inhibitor (GDI) alpha | | | | |
| 201260_s_at | SYPL1 | NM_006754 | 0869 | Synaptophysin-like 1 | 9.26E−06 | −1.5 | 1.5 | 2.48 |
| 234340_at | | AK021431 | 0324 | “CDNA FLJ11369 fis, | 9.44E−06 | −1.75 | 1.75 | 1.7 |
| | | | | clone HEMBA1000338” | | | | |
| 207686_s_at | CASP8 | NM_001228 | 0770 | “Caspase 8, apoptosis- | 9.51E−06 | 2.19 | 2.19 | 1.79 |
| | | | | related cysteine | | | | |
| | | | | peptidase” | | | | |
| 201332_s_at | STAT6 | NM_003153 | 0799 | “Signal transducer and | 9.57E−06 | 1.49 | 1.49 | 2.01 |
| | | | | activator of transcription 6, | | | | |
| | | | | interleukin-4 induced” | | | | |
| 203388_at | ARRB2 | NM_004313 | 0819 | “Arrestin, beta 2” | 9.59E−06 | 1.54 | 1.54 | 2.66 |
| 204275_at | SOLH | AI796687 | 0268 | Small optic lobes homolog | 9.61E−06 | 1.51 | 1.51 | 1.72 |
| | | | | (Drosophila) | | | | |
| 238057_at | USP45 | AW195800 | 0487 | Ubiquitin specific | 9.61E−06 | −2.28 | 2.28 | 1.91 |
| | | | | peptidase 45 | | | | |
| 242352_at | NIPBL | AW272262 | 0494 | Nipped-B homolog | 9.63E−06 | 2.68 | 2.68 | 1.78 |
| | | | | (Drosophila) | | | | |
| 212846_at | RRP1B | AA811192 | 0063 | KIAA0179 | 9.81E−06 | −1.28 | 1.28 | 2.51 |
| 212090_at | GRINA | AL571424 | 0420 | “Glutamate receptor, | 9.90E−06 | 1.94 | 1.94 | 1.99 |
| | | | | ionotropic, N-methyl D- | | | | |
| | | | | asparate-associated | | | | |
| | | | | protein 1 (glutamate | | | | |
| | | | | binding)” | | | | |
| 227636_at | THAP5 | BG500677 | 0698 | THAP domain containing 5 | 9.92E−06 | −1.73 | 1.73 | 2.29 |
| 209379_s_at | KIAA1128 | AF241785 | 0147 | KIAA1128 | 9.94E−06 | −1.5 | 1.5 | 2.33 |
| 230180_at | | AA521056 | 0025 | | 9.99E−06 | 2.56 | 2.56 | 1.96 |
| 217489_s_at | IL6R | S72848 | 0968 | Interleukin 6 receptor | 1.01E−05 | 2.12 | 2.12 | 1.44 |
| 200014_s_at | “(HNRPC, | NM_004500 | 0824 | Heterogeneous nuclear | 1.03E−05 | −1.21 | 1.21 | 2.71 |
| | hCG_1641229)” | | | ribonucleoprotein C | | | | |
| | | | | (C1/C2) | | | | |
| 221628_s_at | N-PAC | AF326966 | 0155 | Cytokine-like nuclear | 1.03E−05 | 2.06 | 2.06 | 1.63 |
| | | | | factor n-pac | | | | |
| 214594_x_at | ATP8B1 | BG252666 | 0680 | “ATPase, Class I, type 8B, | 1.05E−05 | −2.44 | 2.44 | 2.17 |
| | | | | member 1” | | | | |
| 218528_s_at | RNF38 | NM_022781 | 0941 | Ring finger protein 38 | 1.05E−05 | −1.27 | 1.27 | 2.49 |
| 223494_at | MGEA5 | AF307332 | 0152 | Meningioma expressed | 1.08E−05 | −1.49 | 1.49 | 3.22 |
| | | | | antigen 5 (hyaluronidase) | | | | |
| 234260_at | | AL122039 | 0390 | MRNA; cDNA | 1.09E−05 | −2.06 | 2.06 | 1.99 |
| | | | | DKFZp434E0572 (from | | | | |
| | | | | clone DKFZp434E0572) | | | | |
| 203104_at | CSF1R | NM_005211 | 0838 | “Colony stimulating factor | 1.09E−05 | 1.96 | 1.96 | 2.55 |
| | | | | 1 receptor, formerly | | | | |
| | | | | McDonough feline | | | | |
| | | | | sarcoma viral (v-fms) | | | | |
| | | | | oncogene homolog” | | | | |
| 221755_at | EHBP1L1 | BG334196 | 0688 | EH domain binding protein | 1.10E−05 | 1.88 | 1.88 | 1.68 |
| | | | | 1-like 1 | | | | |
| 200976_s_at | TAX1BP1 | NM_006024 | 0850 | Tax1 (human T-cell | 1.11E−05 | −1.26 | 1.26 | 2.84 |
| | | | | leukemia virus type I) | | | | |
| | | | | binding protein 1 | | | | |
| 212297_at | ATP13A3 | BF218804 | 0628 | ATPase type 13A3 | 1.12E−05 | −1.73 | 1.73 | 2 |
| 208841_s_at | G3BP2 | AB014560 | 0086 | GTPase activating protein | 1.12E−05 | −1.37 | 1.37 | 2.55 |
| | | | | (SH3 domain) binding | | | | |
| | | | | protein 2 | | | | |
| 200632_s_at | NDRG1 | NM_006096 | 0853 | N-myc downstream | 1.13E−05 | 1.38 | 1.38 | 2.35 |
| | | | | regulated gene 1 | | | | |
| 215383_x_at | SPG21 | AL137312 | 0397 | “Spastic paraplegia 21, | 1.13E−05 | −1.61 | 1.61 | 2.29 |
| | | | | maspardin (autosomal | | | | |
| | | | | recessive, Mast | | | | |
| | | | | syndrome)” | | | | |
| 235493_at | | AI927329 | 0300 | Transcribed locus | 1.15E−05 | −1.86 | 1.86 | 2.33 |
| 236614_at | LOC729683 | AW006288 | 0463 | Hypothetical protein | 1.15E−05 | 1.36 | 1.36 | 2.25 |
| | | | | LOC729683 | | | | |
| 209273_s_at | ISCA1 | BG387555 | 0690 | Iron-sulfur cluster | 1.16E−05 | −1.44 | 1.44 | 2.16 |
| | | | | assembly 1 homolog (S. cerevisiae) | | | | |
| 227621_at | WTAP | BE464729 | 0574 | Wilms tumor 1 associated | 1.18E−05 | −1.37 | 1.37 | 3.2 |
| | | | | protein | | | | |
| 225431_x_at | ACY1L2 | BE779764 | 0596 | Aminoacylase 1-like 2 | 1.18E−05 | −2.17 | 2.17 | 2.43 |
| 235879_at | | AI697540 | 0245 | Transcribed locus | 1.20E−05 | −2.13 | 2.13 | 2.94 |
| 226825_s_at | TMEM165 | AW665624 | 0524 | Transmembrane protein | 1.22E−05 | 1.67 | 1.67 | 2.76 |
| | | | | 165 | | | | |
| 218143_s_at | SCAMP2 | NM_005697 | 0843 | Secretory carrier | 1.22E−05 | 1.46 | 1.46 | 2.38 |
| | | | | membrane protein 2 | | | | |
| 203890_s_at | DAPK3 | BF686824 | 0655 | Death-associated protein | 1.24E−05 | 1.7 | 1.7 | 1.41 |
| | | | | kinase 3 | | | | |
| 208313_s_at | SF1 | NM_004630 | 0825 | Splicing factor 1 | 1.27E−05 | 1.4 | 1.4 | 2.67 |
| 211352_s_at | NCOA3 | U80737 | 0991 | Nuclear receptor | 1.28E−05 | 1.88 | 1.88 | 1.74 |
| | | | | coactivator 3 | | | | |
| 224579_at | SLC38A1 | BF247552 | 0632 | “Solute carrier family 38, | 1.28E−05 | −1.46 | 1.46 | 3.14 |
| | | | | member 1” | | | | |
| 201752_s_at | ADD3 | AI763123 | 0262 | Adducin 3 (gamma) | 1.28E−05 | −1.44 | 1.44 | 3.26 |
| 231747_at | CYSLTR1 | NM_006639 | 0864 | Cysteinyl leukotriene | 1.30E−05 | 1.73 | 1.73 | 2.57 |
| | | | | receptor 1 | | | | |
| 208319_s_at | RBM3 | NM_006743 | 0868 | “RNA binding motif | 1.30E−05 | −1.66 | 1.66 | 2.61 |
| | | | | (RNP1, RRM) protein 3” | | | | |
| 227159_at | GHDC | AI827015 | 0284 | GH3 domain containing | 1.33E−05 | 1.45 | 1.45 | 2.3 |
| 230387_at | | AL038450 | 0371 | Transcribed locus | 1.33E−05 | −1.75 | 1.75 | 2.34 |

TABLE 2B

| | Known | Genbank | SEQ | | Mean Signal | Log10 | Mean | Top 42 |
|---|---|---|---|---|---|---|---|---|
| Fragment | Genes: Gene | Accession | ID | Sequence Clusters: | Control | Mean (MS | Signal MS | (FC |
| Name | Symbol | No. | NO. | Cluster Title | Samples | Samples) | Samples | Filtered) |
| 214847_s_at | GPSM3 | BG111168 | 0671 | “G-protein signalling | 158.4893192 | 2.46 | 288.4031503 | |
| | | | | modulator 3 (AGS3-like, | | | | |
| | | | | C. elegans)” | | | | |
| 227510_x_at | | AL037917 | 0369 | Transcribed locus | 194.98446 | 3.09 | 1230.268771 | 1 |
| 223578_x_at | MALAT1 | AF113016 | 0131 | Metastasis associated | 67.60829754 | 2.36 | 229.0867653 | 2 |
| | | | | lung adenocarcinoma | | | | |
| | | | | transcript 1 (non-coding | | | | |
| | | | | RNA) | | | | |
| 232431_at | | AI934556 | 0302 | “Glucocorticoid receptor | 331.1311215 | 2.08 | 120.2264435 | 3 |
| | | | | alpha mRNA, variant 3′ | | | | |
| | | | | UTR” | | | | |
| 219878_s_at | KLF13 | NM_015995 | 0892 | Kruppel-like factor 13 | 2.511886432 | 1.53 | 33.88441561 | 4 |
| 214352_s_at | KRAS | BF673699 | 0652 | V-Ki-ras2 Kirsten rat | 114.8153621 | 2.41 | 257.0395783 | 5 |
| | | | | sarcoma viral oncogene | | | | |
| | | | | homolog | | | | |
| 228582_x_at | MALAT1 | AI475544 | 0211 | Metastasis associated | 245.4708916 | 3.37 | 2344.228815 | 6 |
| | | | | lung adenocarcinoma | | | | |
| | | | | transcript 1 (non-coding | | | | |
| | | | | RNA) | | | | |
| 243981_at | | AI763206 | 0263 | Transcribed locus | 316.227766 | 2.12 | 131.8256739 | 7 |
| 201753_s_at | ADD3 | NM_019903 | 0931 | Adducin 3 (gamma) | 1445.439771 | 2.89 | 776.2471166 | |
| 211074_at | FOLR1 | AF000381 | 0102 | Folate receptor 1 (adult) | 144.5439771 | 2.54 | 346.7368505 | 8 |
| 225956_at | LOC153222 | AL565238 | 0417 | Adult retina protein | 741.3102413 | 2.7 | 501.1872336 | |
| 223161_at | KIAA1147 | AA029331 | 0003 | KIAA1147 | 186.2087137 | 2.63 | 426.5795188 | 9 |
| 218389_s_at | APH1A | NM_016022 | 0894 | Anterior pharynx defective | 181.9700859 | 2.44 | 275.4228703 | |
| | | | | 1 homolog A (C. elegans) | | | | |
| 236924_at | GLMN | AA814383 | 0065 | “Glomulin, FKBP | 112.2018454 | 1.66 | 45.70881896 | 10 |
| | | | | associated protein” | | | | |
| 226148_at | ZBTB44 | AU144305 | 0422 | Zinc finger and BTB | 912.0108394 | 2.71 | 512.861384 | |
| | | | | domain containing 44 | | | | |
| 219696_at | FLJ20054 | NM_019049 | 0927 | Hypothetical protein | 151.3561248 | 2.02 | 104.7128548 | |
| | | | | FLJ20054 | | | | |
| 223940_x_at | | AF132202 | 0134 | | 281.8382931 | 2.92 | 831.7637711 | 11 |
| 225889_at | AEBP2 | BF475280 | 0639 | AE binding protein 2 | 489.7788194 | 2.41 | 257.0395783 | |
| 214697_s_at | ROD1 | AW190873 | 0485 | ROD1 regulator of | 79.43282347 | 2.26 | 181.9700859 | 12 |
| | | | | differentiation 1 (S. pombe) | | | | |
| 208610_s_at | SRRM2 | AI655799 | 0233 | Serine/arginine repetitive | 52.48074602 | 2.22 | 165.9586907 | 13 |
| | | | | matrix 2 | | | | |
| 241751_at | OFD1 | AW292752 | 0498 | Oral-facial-digital | 147.9108388 | 1.79 | 61.65950019 | 14 |
| | | | | syndrome 1 | | | | |
| 204049_s_at | PHACTR2 | NM_014721 | 0889 | Phosphatase and actin | 213.796209 | 2.1 | 125.8925412 | |
| | | | | regulator 2 | | | | |
| 225377_at | C9orf86 | BE783949 | 0597 | Chromosome 9 open | 186.2087137 | 2.55 | 354.8133892 | |
| | | | | reading frame 86 | | | | |
| 225139_at | RBM35B | AW070424 | 0473 | RNA binding motif protein | 1000 | 2.8 | 630.9573445 | |
| | | | | 35B | | | | |
| 216177_at | LOC391132 | AW582267 | 0519 | Similar to 60S ribosomal | 154.8816619 | 1.92 | 83.17637711 | |
| | | | | protein L29 (P23) | | | | |
| 242232_at | | AI652864 | 0232 | | 50.11872336 | 2.03 | 107.1519305 | 15 |
| 200734_s_at | ARF3 | BG341906 | 0689 | ADP-ribosylation factor 3 | 177.827941 | 2.5 | 316.227766 | |
| 211716_x_at | ARHGDIA | BC005851 | 0554 | Rho GDP dissociation | 416.8693835 | 2.84 | 691.8309709 | |
| | | | | inhibitor (GDI) alpha | | | | |
| 215460_x_at | BRD1 | AL080149 | 0380 | Bromodomain containing 1 | 177.827941 | 2.11 | 128.8249552 | |
| 222576_s_at | EIF2C1 | AW071829 | 0475 | “Eukaryotic translation | 138.0384265 | 2.42 | 263.0267992 | |
| | | | | initiation factor 2C, 1” | | | | |
| 230141_at | ARID4A | AI640594 | 0226 | AT rich interactive domain | 154.8816619 | 1.92 | 83.17637711 | |
| | | | | 4A (RBP1-like) | | | | |
| 227740_at | UHMK1 | AW173222 | 0481 | U2AF homology motif | 234.4228815 | 2.76 | 575.4399373 | 16 |
| | | | | (UHM) kinase 1 | | | | |
| 204805_s_at | H1FX | NM_006026 | 0851 | “H1 histone family, | 181.9700859 | 2.63 | 426.5795188 | 17 |
| | | | | member X” | | | | |
| 208624_s_at | EIF4G1 | BE966878 | 0610 | “Eukaryotic translation | 75.8577575 | 2.03 | 107.1519305 | |
| | | | | initiation factor 4 gamma, | | | | |
| | | | | 1” | | | | |
| 243612_at | NSD1 | AL526448 | 0408 | Nuclear receptor binding | 154.8816619 | 1.77 | 58.88436554 | 18 |
| | | | | SET domain protein 1 | | | | |
| 201167_x_at | ARHGDIA | D13989 | 0704 | Rho GDP dissociation | 52.48074602 | 2.11 | 128.8249552 | 19 |
| | | | | inhibitor (GDI) alpha | | | | |
| 241786_at | | AI380514 | 0198 | Transcribed locus | 407.3802778 | 2.25 | 177.827941 | 20 |
| 238430_x_at | SLFN5 | AI923675 | 0295 | Schlafen family member 5 | 371.5352291 | 3 | 1000 | 21 |
| 202102_s_at | BRD4 | BF718610 | 0657 | Bromodomain containing 4 | 208.9296131 | 2.51 | 323.5936569 | |
| 201737_s_at | MARCH6 | NM_005885 | 0847 | Membrane-associated | 338.8441561 | 2.28 | 190.5460718 | |
| | | | | ring finger (C3HC4) 6 | | | | |
| 229926_at | | AI633738 | 0225 | Transcribed locus | 109.6478196 | 1.83 | 67.60829754 | |
| 230961_at | | BE856980 | 0603 | | 117.4897555 | 1.76 | 57.54399373 | 22 |
| 201996_s_at | SPEN | AL524033 | 0406 | “Spen homolog, | 35.48133892 | 1.96 | 91.20108394 | 23 |
| | | | | transcriptional regulator | | | | |
| | | | | (Drosophila)” | | | | |
| 231858_x_at | DKFZp761E198 | BC004895 | 0552 | DKFZp761E198 protein | 104.7128548 | 2.28 | 190.5460718 | |
| 200608_s_at | RAD21 | NM_006265 | 0855 | RAD21 homolog (S. pombe) | 407.3802778 | 2.45 | 281.8382931 | |
| 201168_x_at | ARHGDIA | NM_004309 | 0818 | Rho GDP dissociation | 234.4228815 | 2.63 | 426.5795188 | |
| | | | | inhibitor (GDI) alpha | | | | |
| 224631_at | ZFP91 | AA758013 | 0058 | Zinc finger protein 91 | 104.7128548 | 2.31 | 204.1737945 | |
| | | | | homolog (mouse) | | | | |
| 225563_at | PAN3 | AI970788 | 0309 | PAN3 polyA specific | 1071.519305 | 2.82 | 660.693448 | |
| | | | | ribonuclease subunit | | | | |
| | | | | homolog (S. cerevisiae) | | | | |
| 222133_s_at | PHF20L1 | AK022280 | 0332 | PHD finger protein 20-like 1 | 131.8256739 | 1.91 | 81.28305162 | |
| 237768_x_at | | AA825925 | 0066 | | 371.5352291 | 2.21 | 162.1810097 | 24 |
| 208677_s_at | BSG | AL550657 | 0415 | Basigin (Ok blood group) | 83.17637711 | 2.27 | 186.2087137 | 25 |
| 224572_s_at | IRF2BP2 | BG485163 | 0697 | Interferon regulatory factor | 426.5795188 | 3 | 1000 | 26 |
| | | | | 2 binding protein 2 | | | | |
| 221899_at | PFAAP5 | AI809961 | 0274 | Phosphonoformate | 316.227766 | 2.22 | 165.9586907 | |
| | | | | immuno-associated | | | | |
| | | | | protein 5 | | | | |
| 224969_at | ATXN7L3 | AL390158 | 0401 | Ataxin 7-like 3 | 208.9296131 | 2.54 | 346.7368505 | |
| 229389_at | ATG16L2 | AA741058 | 0050 | ATG16 autophagy related | 208.9296131 | 2.8 | 630.9573445 | 27 |
| | | | | 16-like 2 (S. cerevisiae) | | | | |
| 242134_at | | AI733194 | 0247 | Transcribed locus | 52.48074602 | 2.06 | 114.8153621 | 28 |
| 224676_at | TMED4 | AI472339 | 0208 | Transmembrane emp24 | 281.8382931 | 2.77 | 588.8436554 | 29 |
| | | | | protein transport domain | | | | |
| | | | | containing 4 | | | | |
| 224568_x_at | MALAT1 | AW005982 | 0462 | Metastasis associated | 190.5460718 | 2.78 | 602.5595861 | 30 |
| | | | | lung adenocarcinoma | | | | |
| | | | | transcript 1 (non-coding | | | | |
| | | | | RNA) | | | | |
| 218659_at | ASXL2 | NM_018263 | 0923 | Additional sex combs like | 758.577575 | 2.72 | 524.8074602 | |
| | | | | 2 (Drosophila) | | | | |
| 203497_at | PPARBP | NM_004774 | 0829 | PPAR binding protein | 251.1886432 | 2.21 | 162.1810097 | |
| 204285_s_at | PMAIP1 | AI857639 | 0289 | Phorbol-12-myristate-13- | 66.0693448 | 2.23 | 169.8243652 | 31 |
| | | | | acetate-induced protein 1 | | | | |
| 213015_at | | BF448315 | 0638 | “ARTC1 mRNA, complete | 89.12509381 | 2.35 | 223.8721139 | 32 |
| | | | | sequence” | | | | |
| 224567_x_at | MALAT1 | BG534952 | 0699 | Metastasis associated | 295.1209227 | 2.91 | 812.8305162 | 33 |
| | | | | lung adenocarcinoma | | | | |
| | | | | transcript 1 (non-coding | | | | |
| | | | | RNA) | | | | |
| 222790_s_at | RSBN1 | AK022166 | 0330 | Round spermatid basic | 741.3102413 | 2.53 | 338.8441561 | 34 |
| | | | | protein 1 | | | | |
| 207057_at | SLC16A7 | NM_004731 | 0828 | “Solute carrier family 16, | 25.70395783 | 2.01 | 102.3292992 | 35 |
| | | | | member 7 | | | | |
| | | | | (monocarboxylic acid | | | | |
| | | | | transporter 2)” | | | | |
| 212852_s_at | TROVE2 | AL538601 | 0414 | “TROVE domain family, | 407.3802778 | 2.46 | 288.4031503 | |
| | | | | member 2” | | | | |
| 238761_at | | BE645241 | 0581 | | 724.4359601 | 2.46 | 288.4031503 | 36 |
| 214198_s_at | DGCR2 | AU150824 | 0430 | DiGeorge syndrome | 177.827941 | 2.03 | 107.1519305 | |
| | | | | critical region gene 2 | | | | |
| 202809_s_at | INTS3 | NM_023015 | 0943 | Integrator complex subunit 3 | 154.8816619 | 2.09 | 123.0268771 | |
| 236072_at | | N64578 | 0751 | Transcribed locus | 81.28305162 | 1.54 | 34.67368505 | 37 |
| 225827_at | EIF2C2 | AI832074 | 0288 | “Eukaryotic translation | 138.0384265 | 2.54 | 346.7368505 | 38 |
| | | | | initiation factor 2C, 2” | | | | |
| 241775_at | | AW298119 | 0502 | “CDNA FLJ26437 fis, | 316.227766 | 2.16 | 144.5439771 | 39 |
| | | | | clone KDN02067” | | | | |
| 215706_x_at | ZYX | BC002323 | 0543 | Zyxin | 398.1071706 | 2.83 | 676.0829754 | |
| 201730_s_at | TPR | BF110993 | 0619 | Translocated promoter | 194.98446 | 2.54 | 346.7368505 | |
| | | | | region (to activated MET | | | | |
| | | | | oncogene) | | | | |
| 226447_at | ASH1L | BG290742 | 0687 | “Ash1 (absent, small, or | 398.1071706 | 2.42 | 263.0267992 | |
| | | | | homeotic)-like | | | | |
| | | | | (Drosophila)” | | | | |
| 200623_s_at | CALM3 | NM_005184 | 0836 | “Calmodulin 3 | 213.796209 | 2.6 | 398.1071706 | |
| | | | | (phosphorylase kinase, | | | | |
| | | | | delta)” | | | | |
| 215990_s_at | BCL6 | S67779 | 0966 | B-cell CLL/lymphoma 6 | 63.09573445 | 2.1 | 125.8925412 | |
| | | | | (zinc finger protein 51) | | | | |
| 212007_at | UBXD2 | AI927512 | 0301 | UBX domain containing 2 | 112.2018454 | 2.23 | 169.8243652 | |
| 225885_at | EEA1 | AI336848 | 0183 | Early endosome antigen 1 | 316.227766 | 2.29 | 194.98446 | |
| 212629_s_at | PKN2 | AI633689 | 0224 | Protein kinase N2 | 67.60829754 | 2.06 | 114.8153621 | |
| 216971_s_at | PLEC1 | Z54367 | 1015 | “Plectin 1, intermediate | 35.48133892 | 2.07 | 117.4897555 | 40 |
| | | | | filament binding protein | | | | |
| | | | | 500 kDa” | | | | |
| 225289_at | STAT3 | AI139252 | 0174 | Signal transducer and | 416.8693835 | 2.82 | 660.693448 | |
| | | | | activator of transcription 3 | | | | |
| | | | | (acute-phase response | | | | |
| | | | | factor) | | | | |
| 217713_x_at | | AA126763 | 0008 | | 165.9586907 | 2.01 | 102.3292992 | |
| 225361_x_at | FAM122B | AI348001 | 0187 | Family with sequence | 630.9573445 | 2.64 | 436.5158322 | |
| | | | | similarity 122B | | | | |
| 242920_at | | AW590838 | 0520 | | 331.1311215 | 2.1 | 125.8925412 | 41 |
| 227754_at | | AV700815 | 0447 | “CDNA FLJ10417 fis, | 173.7800829 | 1.93 | 85.11380382 | 42 |
| | | | | clone NT2RP1000112” | | | | |
| 226680_at | | BF056303 | 0614 | Transcribed locus | 213.796209 | 1.99 | 97.7237221 | |
| 202157_s_at | CUGBP2 | U69546 | 0989 | “CUG triplet repeat, RNA | 1995.262315 | 3.14 | 1380.384265 | |
| | | | | binding protein 2” | | | | |
| 208988_at | | BE675843 | 0589 | | 190.5460718 | 2.11 | 128.8249552 | |
| 222243_s_at | TOB2 | AB051450 | 0094 | “Transducer of ERBB2, 2” | 107.1519305 | 1.9 | 79.43282347 | |
| 217862_at | | N24868 | 0746 | Transcribed locus | 194.98446 | 2.47 | 295.1209227 | |
| 64486_at | CORO1B | AI341234 | 0185 | “Coronin, actin binding | 223.8721139 | 2.49 | 309.0295433 | |
| | | | | protein, 1B” | | | | |
| 230918_at | | BE856598 | 0601 | | 239.8832919 | 2.14 | 138.0384265 | |
| 204978_at | SFRS16 | NM_007056 | 0873 | “Splicing factor, | 63.09573445 | 2.15 | 141.2537545 | |
| | | | | arginine/serine-rich 16” | | | | |
| 232879_at | CRTC3 | AK024981 | 0354 | CREB regulated | 91.20108394 | 1.65 | 44.66835922 | |
| | | | | transcription coactivator 3 | | | | |
| 235032_at | DNAJA5 | BG112118 | 0672 | DnaJ homology subfamily | 251.1886432 | 2.13 | 134.8962883 | |
| | | | | A member 5 | | | | |
| 203591_s_at | CSF3R | NM_000760 | 0761 | Colony stimulating factor 3 | 144.5439771 | 2.47 | 295.1209227 | |
| | | | | receptor (granulocyte) | | | | |
| 228098_s_at | MYLIP | AW292746 | 0497 | Myosin regulatory light | 1047.128548 | 2.83 | 676.0829754 | |
| | | | | chain interacting protein | | | | |
| 234734_s_at | TNRC6A | AK025696 | 0358 | Trinucleotide repeat | 181.9700859 | 2.48 | 301.995172 | |
| | | | | containing 6A | | | | |
| 244433_at | | AI950023 | 0307 | | 426.5795188 | 2.32 | 208.9296131 | |
| 226641_at | | AU157224 | 0438 | “CDNA FLJ11570 fis, | 851.1380382 | 2.73 | 537.0317964 | |
| | | | | clone HEMBA1003309” | | | | |
| 222791_at | RSBN1 | AK022166 | 0330 | Round spermatid basic | 1202.264435 | 2.75 | 562.3413252 | |
| | | | | protein 1 | | | | |
| 225706_at | GLCCI1 | AI761989 | 0259 | Glucocorticoid induced | 1000 | 2.73 | 537.0317964 | |
| | | | | transcript 1 | | | | |
| 243790_at | ZNF585A | AA203136 | 0015 | Zinc finger protein 585A | 154.8816619 | 1.85 | 70.79457844 | |
| 244145_at | | BG260337 | 0683 | | 251.1886432 | 2.19 | 154.8816619 | |
| 203751_x_at | JUND | AI762296 | 0260 | Jun D proto-oncogene | 79.43282347 | 2.19 | 154.8816619 | |
| 225216_at | CXorf39 | AI590719 | 0220 | Chromosome X open | 416.8693835 | 2.48 | 301.995172 | |
| | | | | reading frame 39 | | | | |
| 228853_at | “(LOC730432, | AI652546 | 0229 | Serine/threonine/tyrosine | 316.227766 | 2.33 | 213.796209 | |
| | STYX)” | | | interacting protein | | | | |
| 232909_s_at | “(BPTF, | AU146870 | 0426 | “(Bromodomain PHD | 776.2471166 | 2.69 | 489.7788194 | |
| | LOC646043)” | | | finger transcription factor, | | | | |
| | | | | Similar to Enhancer of | | | | |
| | | | | bithorax CG32346-PB, | | | | |
| | | | | isoform B)” | | | | |
| 36564_at | IBRDC3 | W27419 | 0998 | IBR domain containing 3 | 107.1519305 | 2.31 | 204.1737945 | |
| 200073_s_at | HNRPD | M94630 | 0742 | “Heterogeneous nuclear | 1737.800829 | 3.08 | 1202.264435 | |
| | | | | ribonucleoprotein D (AU- | | | | |
| | | | | rich element RNA binding | | | | |
| | | | | protein 1, 37 kDa)” | | | | |
| 200808_s_at | ZYX | NM_003461 | 0804 | Zyxin | 398.1071706 | 2.85 | 707.9457844 | |
| 235125_x_at | | AI078279 | 0167 | Transcribed locus | 147.9108388 | 1.75 | 56.23413252 | |
| 226712_at | | BF206389 | 0627 | “CDNA: FLJ22100 fis, | 199.5262315 | 2.48 | 301.995172 | |
| | | | | clone HEP17127” | | | | |
| 208447_s_at | PRPS1 | NM_002764 | 0795 | Phosphoribosyl | 67.60829754 | 2.12 | 131.8256739 | |
| | | | | pyrophosphate synthetase 1 | | | | |
| 226426_at | | BG149849 | 0674 | Transcribed locus | 457.0881896 | 2.49 | 309.0295433 | |
| 239163_at | UBE2B | AW364833 | 0507 | Ubiquitin-conjugating | 64.5654229 | 2.13 | 134.8962883 | |
| | | | | enzyme E2B (RAD6 | | | | |
| | | | | homolog) | | | | |
| 219426_at | EIF2C3 | NM_024852 | 0949 | “Eukaryotic translation | 43.65158322 | 1.86 | 72.44359601 | |
| | | | | initiation factor 2C, 3” | | | | |
| 235341_at | LOC144871 | AL119957 | 0387 | Hypothetical protein | 199.5262315 | 2.55 | 354.8133892 | |
| | | | | LOC144871 | | | | |
| 242243_at | TMF1 | AI767435 | 0264 | TATA element modulatory | 102.3292992 | 1.66 | 45.70881896 | |
| | | | | factor 1 | | | | |
| 201236_s_at | BTG2 | NM_006763 | 0870 | “BTG family, member 2” | 281.8382931 | 2.63 | 426.5795188 | |
| 202577_s_at | DDX19A | BC005162 | 0553 | DEAD (Asp-Glu-Ala-As) | 117.4897555 | 1.91 | 81.28305162 | |
| | | | | box polypeptide 19A | | | | |
| 227772_at | | AV700849 | 0448 | | 213.796209 | 2.09 | 123.0268771 | |
| 229897_at | ZNF641 | BF195808 | 0626 | Zinc finger protein 641 | 141.2537545 | 2.42 | 263.0267992 | |
| 218566_s_at | CHORDC1 | NM_012124 | 0876 | Cysteine and histidine-rich | 213.796209 | 2.12 | 131.8256739 | |
| | | | | domain (CHORD)- | | | | |
| | | | | containing 1 | | | | |
| 203839_s_at | TNK2 | NM_005781 | 0845 | “Tyrosine kinase, non- | 42.65795188 | 1.96 | 91.20108394 | |
| | | | | receptor, 2” | | | | |
| 204361_s_at | SKAP2 | AB014486 | 0085 | Src kinase associated | 123.0268771 | 1.84 | 69.18309709 | |
| | | | | phosphoprotein 2 | | | | |
| 224250_s_at | SECISBP2 | BC001189 | 0541 | SECIS binding protein 2 | 371.5352291 | 2.81 | 645.654229 | |
| 225350_s_at | ZYG11B | AV701229 | 0449 | Zyg-11 homolog B (C. elegans) | 676.0829754 | 2.64 | 436.5158322 | |
| 204516_at | | BG390306 | 0692 | Transcribed locus | 363.0780548 | 2.22 | 165.9586907 | |
| 225620_at | RAB35 | AL534848 | 0412 | “RAB35, member RAS | 186.2087137 | 2.47 | 295.1209227 | |
| | | | | oncogene family” | | | | |
| 226965_at | FAM116A | BF438017 | 0636 | “(Family with sequence | 812.8305162 | 2.72 | 524.8074602 | |
| | | | | similarity 116, member A, | | | | |
| | | | | Transcribed locus)” | | | | |
| 239808_at | | AI084489 | 0169 | Transcribed locus | 295.1209227 | 2.2 | 158.4893192 | |
| 227428_at | GABPA | BE876628 | 0605 | “GA binding protein | 331.1311215 | 2.33 | 213.796209 | |
| | | | | transcription factor, alpha | | | | |
| | | | | subunit 60 kDa” | | | | |
| 218595_s_at | “(HEATR1, | NM_018072 | 0921 | “Lectin, galactoside- | 199.5262315 | 2.15 | 141.2537545 | |
| | LGALS8)” | | | binding, soluble, 8 | | | | |
| | | | | (galectin 8)” | | | | |
| 222266_at | C19orf2 | BF796940 | 0661 | Chromosome 19 open | 147.9108388 | 1.93 | 85.11380382 | |
| | | | | reading frame 2 | | | | |
| 239597_at | | AA993566 | 0080 | | 114.8153621 | 2.43 | 269.1534804 | |
| 203415_at | PDCD6 | NM_013232 | 0879 | Programmed cell death 6 | 158.4893192 | 2.33 | 213.796209 | |
| 208866_at | | BF510713 | 0642 | CDNA clone | 158.4893192 | 2 | 100 | |
| | | | | IMAGE: 4044084 | | | | |
| 212575_at | C19orf6 | BF966155 | 0665 | Chromosome 19 open | 2.398832919 | 1.47 | 29.51209227 | |
| | | | | reading frame 6 | | | | |
| 225176_at | | AA156754 | 0013 | “CDNA FLJ42149 fis, | 912.0108394 | 2.81 | 645.654229 | |
| | | | | clone THYMU1000692” | | | | |
| 227223_at | RBM39 | BE466173 | 0575 | RNA binding motif protein | 165.9586907 | 2.58 | 380.1893963 | |
| | | | | 39 | | | | |
| 41386_i_at | JMJD3 | AB002344 | 0082 | Jumonji domain | 223.8721139 | 2.58 | 380.1893963 | |
| | | | | containing 3 | | | | |
| 225995_x_at | “(FAM39DP, | BG178695 | 0675 | “(CXYorf1-related protein, | 281.8382931 | 2.67 | 467.7351413 | |
| | FLJ00038, | | | Family with sequence | | | | |
| | LOC376475, | | | similarity 39, member D | | | | |
| | LOC727741)” | | | pseudogene)” | | | | |
| 228328_at | KLHL28 | AI982758 | 0311 | Kelch-like 28 (Drosophila) | 269.1534804 | 2.2 | 158.4893192 | |
| 235409_at | MGA | AU149225 | 0429 | MAX gene associated | 77.62471166 | 2.27 | 186.2087137 | |
| 200649_at | NUCB1 | BC002356 | 0544 | Nucleobindin 1 | 158.4893192 | 2.46 | 288.4031503 | |
| 232615_at | | AA632758 | 0034 | Transcribed locus | 1148.153621 | 2.75 | 562.3413252 | |
| 205882_x_at | ADD3 | AI818488 | 0278 | Adducin 3 (gamma) | 1905.460718 | 3.11 | 1288.249552 | |
| 228496_s_at | CRIM1 | AW243081 | 0489 | Cysteine rich | 151.3561248 | 1.85 | 70.79457844 | |
| | | | | transmembrane BMP | | | | |
| | | | | regulator 1 (chordin-like) | | | | |
| 209189_at | FOS | BC004490 | 0549 | V-fos FBJ murine | 407.3802778 | 3.14 | 1380.384265 | |
| | | | | osteosarcoma viral | | | | |
| | | | | oncogene homolog | | | | |
| 239233_at | KIAA1212 | AA744613 | 0053 | KIAA1212 | 25.11886432 | 1.04 | 10.96478196 | |
| 224308_s_at | INTS2 | AL136800 | 0393 | Integrator complex subunit 2 | 95.4992586 | 1.68 | 47.86300923 | |
| 208705_s_at | EIF5 | BG481972 | 0696 | Eukaryotic translation | 363.0780548 | 2.67 | 467.7351413 | |
| | | | | initiation factor 5 | | | | |
| 218607_s_at | SDAD1 | NM_018115 | 0922 | SDA1 domain containing 1 | 173.7800829 | 2.42 | 263.0267992 | |
| 200041_s_at | BAT1 | NM_004640 | 0826 | HLA-B associated | 870.96359 | 3.06 | 1148.153621 | |
| | | | | transcript 1 | | | | |
| 202850_at | ABCD3 | NM_002858 | 0796 | “ATP-binding cassette, | 199.5262315 | 2.11 | 128.8249552 | |
| | | | | sub-family D (ALD), | | | | |
| | | | | member 3” | | | | |
| 224562_at | “(LOC644739, | AK025566 | 0357 | “(Similar to Wiskott- | 870.96359 | 3.19 | 1548.816619 | |
| | LOC647909, | | | Aldrich syndrome protein | | | | |
| | WASF2)” | | | family member 4 (WASP- | | | | |
| | | | | family protein member 4), | | | | |
| | | | | WAS protein family, | | | | |
| | | | | member 2)” | | | | |
| 222644_s_at | GLT25D1 | AI924150 | 0296 | Glycosyltransferase 25 | 123.0268771 | 2.39 | 245.4708916 | |
| | | | | domain containing 1 | | | | |
| 212536_at | ATP11B | AB023173 | 0088 | “ATPase, Class VI, type | 147.9108388 | 1.99 | 97.7237221 | |
| | | | | 11B” | | | | |
| 215269_at | TMEM1 | AI922538 | 0294 | Transmembrane protein 1 | 102.3292992 | 1.72 | 52.48074602 | |
| 243303_at | | AA811657 | 0064 | | 218.7761624 | 2.06 | 114.8153621 | |
| 201991_s_at | KIF5B | BF223224 | 0630 | Kinesin family member 5B | 562.3413252 | 2.62 | 416.8693835 | |
| 213327_s_at | USP12 | AI820101 | 0280 | Ubiquitin specific | 93.32543008 | 1.77 | 58.88436554 | |
| | | | | peptidase 12 | | | | |
| 213882_at | TM2D1 | AA012917 | 0001 | TM2 domain containing 1 | 45.70881896 | 1.43 | 26.91534804 | |
| 214715_x_at | ZNF160 | AK024789 | 0348 | Zinc finger protein 160 | 234.4228815 | 2.15 | 141.2537545 | |
| 225845_at | ZBTB44 | BG253884 | 0681 | Zinc finger and BTB | 645.654229 | 2.58 | 380.1893963 | |
| | | | | domain containing 44 | | | | |
| 215529_x_at | DIP2A | AI590053 | 0219 | DIP2 disco-interacting | 77.62471166 | 1.59 | 38.9045145 | |
| | | | | protein 2 homolog A | | | | |
| | | | | (Drosophila) | | | | |
| 242539_at | LOC730069 | AW665509 | 0523 | Similar to nuclear receptor | 213.796209 | 1.99 | 97.7237221 | |
| | | | | binding factor 2 | | | | |
| 203311_s_at | ARF6 | M57763 | 0735 | ADP-ribosylation factor 6 | 169.8243652 | 2.51 | 323.5936569 | |
| 205854_at | TULP3 | AK024246 | 0342 | Tubby like protein 3 | 61.65950019 | 1.59 | 38.9045145 | |
| 212042_x_at | “(LOC653949, | BG389744 | 0691 | Ribosomal protein L7 | 3235.936569 | 3.37 | 2344.228815 | |
| | RPL7, | | | | | | | |
| | hCG_2015956, | | | | | | | |
| | hCG_31916)” | | | | | | | |
| 233369_at | | AU146027 | 0424 | “CDNA FLJ11835 fis, | 263.0267992 | 2.17 | 147.9108388 | |
| | | | | clone HEMBA1006595” | | | | |
| 212513_s_at | USP33 | AB029020 | 0089 | Ubiquitin specific | 331.1311215 | 2.4 | 251.1886432 | |
| | | | | peptidase 33 | | | | |
| 218626_at | EIF4ENIF1 | NM_019843 | 0930 | Eukaryotic translation | 125.8925412 | 1.98 | 95.4992586 | |
| | | | | initiation factor 4E nuclear | | | | |
| | | | | import factor 1 | | | | |
| 208246_x_at | | NM_017618 | 0909 | | 194.98446 | 2.08 | 120.2264435 | |
| 224559_at | MALAT1 | AF001540 | 0104 | Metastasis associated | 309.0295433 | 3.15 | 1412.537545 | |
| | | | | lung adenocarcinoma | | | | |
| | | | | transcript 1 (non-coding | | | | |
| | | | | RNA) | | | | |
| 205312_at | SPI1 | NM_003120 | 0798 | Spleen focus forming virus | 81.28305162 | 2.35 | 223.8721139 | |
| | | | | (SFFV) proviral integration | | | | |
| | | | | oncogene spi1 | | | | |
| 213318_s_at | BAT3 | BG028844 | 0669 | HLA-B associated | 363.0780548 | 2.71 | 512.861384 | |
| | | | | transcript 3 | | | | |
| 212099_at | RHOB | AI263909 | 0179 | “Ras homolog gene | 208.9296131 | 2.7 | 501.1872336 | |
| | | | | family, member B” | | | | |
| 223131_s_at | TRIM8 | AI925572 | 0299 | Tripartite motif-containing 8 | 79.43282347 | 2.15 | 141.2537545 | |
| 212487_at | GPATCH8 | AI673812 | 0234 | G patch domain | 37.15352291 | 1.94 | 87.096359 | |
| | | | | containing 8 | | | | |
| 200011_s_at | ARF3 | NM_001659 | 0775 | ADP-ribosylation factor 3 | 891.2509381 | 3.1 | 1258.925412 | |
| 210543_s_at | “(LOC731751, | U34994 | 0981 | “Protein kinase, DNA- | 162.1810097 | 2.07 | 117.4897555 | |
| | PRKDC)” | | | activated, catalytic | | | | |
| | | | | polypeptide” | | | | |
| 217313_at | | AC004692 | 0098 | | 218.7761624 | 2.13 | 134.8962883 | |
| 228106_at | C4orf30 | AA031528 | 0004 | Chromosome 4 open | 263.0267992 | 2.22 | 165.9586907 | |
| | | | | reading frame 30 | | | | |
| 218043_s_at | AZI2 | NM_022461 | 0940 | 5-azacytidine induced 2 | 77.62471166 | 1.57 | 37.15352291 | |
| 203514_at | MAP3K3 | BF971923 | 0666 | Mitogen-activated protein | 162.1810097 | 2.02 | 104.7128548 | |
| | | | | kinase kinase kinase 3 | | | | |
| 223295_s_at | LUC7L | BE049621 | 0566 | LUC7-like (S. cerevisiae) | 467.7351413 | 2.47 | 295.1209227 | |
| 235469_at | MGC40405 | AV744101 | 0457 | Hypothetical protein | 89.12509381 | 1.56 | 36.30780548 | |
| | | | | MGC40405 | | | | |
| 209440_at | PRPS1 | BC001605 | 0542 | Phosphoribosyl | 165.9586907 | 2.37 | 234.4228815 | |
| | | | | pyrophosphate synthetase 1 | | | | |
| 227039_at | AKAP13 | AI674926 | 0235 | A kinase (PRKA) anchor | 288.4031503 | 2.74 | 549.5408739 | |
| | | | | protein 13 | | | | |
| 218499_at | RP6-213H19.1 | NM_016542 | 0904 | Serine/threonine protein | 338.8441561 | 2.34 | 218.7761624 | |
| | | | | kinase MST4 | | | | |
| 232440_at | ZDHHC13 | AU155198 | 0433 | “Zinc finger, DHHC-type | 181.9700859 | 2.02 | 104.7128548 | |
| | | | | containing 13” | | | | |
| 209727_at | GM2A | M76477 | 0737 | GM2 ganglioside activator | 10.23292992 | 1.77 | 58.88436554 | |
| 241891_at | | T92908 | 0972 | | 436.5158322 | 2.38 | 239.8832919 | |
| 227525_at | GLCCI1 | AA058770 | 0006 | Glucocorticoid induced | 457.0881896 | 2.41 | 257.0395783 | |
| | | | | transcript 1 | | | | |
| 228088_at | | AI092265 | 0170 | “CDNA FLJ31513 fis, | 128.8249552 | 1.94 | 87.096359 | |
| | | | | clone NT2RI1000127” | | | | |
| 242362_at | | AI797788 | 0269 | | 489.7788194 | 2.33 | 213.796209 | |
| 34478_at | RAB11B | X79780 | 1009 | “RAB11B, member RAS | 18.62087137 | 1.69 | 48.97788194 | |
| | | | | oncogene family” | | | | |
| 223135_s_at | BBX | AL136769 | 0392 | Bobby sox homolog | 295.1209227 | 2.62 | 416.8693835 | |
| | | | | (Drosophila) | | | | |
| 225780_at | RSC1A1 | AL565415 | 0418 | “Regulatory solute carrier | 316.227766 | 2.3 | 199.5262315 | |
| | | | | protein, family 1, member | | | | |
| | | | | 1” | | | | |
| 211791_s_at | KCNAB2 | AF044253 | 0112 | “Potassium voltage-gated | 20.41737945 | 1.98 | 95.4992586 | |
| | | | | channel, shaker-related | | | | |
| | | | | subfamily, beta member | | | | |
| | | | | 2” | | | | |
| 204252_at | CDK2 | M68520 | 0736 | Cyclin-dependent kinase 2 | 114.8153621 | 1.92 | 83.17637711 | |
| 202420_s_at | DHX9 | NM_001357 | 0771 | DEAH (Asp-Glu-Ala-His) | 223.8721139 | 2.24 | 173.7800829 | |
| | | | | box polypeptide 9 | | | | |
| 223138_s_at | DHX36 | AI937206 | 0304 | DEAH (Asp-Glu-Ala-His) | 162.1810097 | 2.44 | 275.4228703 | |
| | | | | box polypeptide 36 | | | | |
| 240452_at | GSPT1 | AA580082 | 0030 | G1 to S phase transition 1 | 316.227766 | 2.15 | 141.2537545 | |
| 202082_s_at | SEC14L1 | AV748469 | 0458 | SEC14-like 1 (S. cerevisiae) | 54.95408739 | 1.98 | 95.4992586 | |
| 213038_at | IBRDC3 | AL031602 | 0365 | IBR domain containing 3 | 89.12509381 | 2.14 | 138.0384265 | |
| 232038_at | C6orf170 | AI052103 | 0162 | Chromosome 6 open | 53.70317964 | 1.43 | 26.91534804 | |
| | | | | reading frame 170 | | | | |
| 229036_at | TNRC6B | AI681177 | 0238 | “(Transcribed locus, | 208.9296131 | 2.58 | 380.1893963 | |
| | | | | Trinucleotide repeat | | | | |
| | | | | containing 6B)” | | | | |
| 229235_at | | AI983432 | 0312 | Transcribed locus | 389.045145 | 2.41 | 257.0395783 | |
| 226412_at | C6orf111 | N66397 | 0752 | Chromosome 6 open | 575.4399373 | 2.54 | 346.7368505 | |
| | | | | reading frame 111 | | | | |
| 212637_s_at | WWP1 | AU155187 | 0432 | WW domain containing E3 | 41.68693835 | 1.4 | 25.11886432 | |
| | | | | ubiquitin protein ligase 1 | | | | |
| 220296_at | | NM_024564 | 0947 | | 100 | 1.71 | 51.2861384 | |
| 240695_at | | AA876138 | 0075 | | 60.25595861 | 2.01 | 102.3292992 | |
| 224759_s_at | C12orf23 | AK001731 | 0320 | Chromosome 12 open | 301.995172 | 2.26 | 181.9700859 | |
| | | | | reading frame 23 | | | | |
| 222794_x_at | PAPD1 | AK022188 | 0331 | PAP associated domain | 323.5936569 | 2.36 | 229.0867653 | |
| | | | | containing 1 | | | | |
| 224974_at | SUDS3 | AK024460 | 0344 | Suppressor of defective | 186.2087137 | 1.98 | 95.4992586 | |
| | | | | silencing 3 homolog | | | | |
| | | | | (S. cerevisiae) | | | | |
| 242650_at | | AW298590 | 0503 | Transcribed locus | 69.18309709 | 1.5 | 31.6227766 | |
| 212921_at | SMYD2 | AF070592 | 0121 | SET and MYND domain | 75.8577575 | 1.64 | 43.65158322 | |
| | | | | containing 2 | | | | |
| 217717_s_at | YWHAB | BF246499 | 0631 | “Tyrosine 3- | 323.5936569 | 2.71 | 512.861384 | |
| | | | | monooxygenase/tryptophan | | | | |
| | | | | 5-monooxygenase | | | | |
| | | | | activation protein, beta | | | | |
| | | | | polypeptide” | | | | |
| 221419_s_at | | NM_013307 | 0880 | | 107.1519305 | 2.35 | 223.8721139 | |
| 204809_at | CLPX | NM_006660 | 0866 | ClpX caseinolytic | 97.7237221 | 1.85 | 70.79457844 | |
| | | | | peptidase X homolog (E. coli) | | | | |
| 201409_s_at | PPP1CB | NM_002709 | 0794 | “Protein phosphatase 1, | 269.1534804 | 2.29 | 194.98446 | |
| | | | | catalytic subunit, beta | | | | |
| | | | | isoform” | | | | |
| 209829_at | C6orf32 | AB002384 | 0083 | Chromosome 6 open | 776.2471166 | 2.68 | 478.6300923 | |
| | | | | reading frame 32 | | | | |
| 225761_at | PAPD4 | AI434509 | 0203 | PAP associated domain | 602.5595861 | 2.63 | 426.5795188 | |
| | | | | containing 4 | | | | |
| 228471_at | ANKRD44 | AA744636 | 0054 | Ankyrin repeat domain 44 | 562.3413252 | 2.51 | 323.5936569 | |
| 201526_at | ARF5 | NM_001662 | 0776 | ADP-ribosylation factor 5 | 162.1810097 | 2.39 | 245.4708916 | |
| 241435_at | | AA702930 | 0044 | | 1698.243652 | 2.9 | 794.3282347 | |
| 227567_at | AMZ2 | AL524467 | 0407 | Archaemetzincins-2 | 338.8441561 | 2.3 | 199.5262315 | |
| 209563_x_at | CALM1 | BC000454 | 0537 | “Calmodulin 1 | 691.8309709 | 3.07 | 1174.897555 | |
| | | | | (phosphorylase kinase, | | | | |
| | | | | delta)” | | | | |
| 242518_at | | AA748423 | 0056 | | 74.13102413 | 2.07 | 117.4897555 | |
| 239049_at | | BF514509 | 0644 | | 239.8832919 | 2.12 | 131.8256739 | |
| 209024_s_at | SYNCRIP | AI472757 | 0209 | “Synaptotagmin binding, | 138.0384265 | 2.31 | 204.1737945 | |
| | | | | cytoplasmic RNA | | | | |
| | | | | interacting protein” | | | | |
| 211948_x_at | BAT2D1 | BG261071 | 0685 | BAT2 domain containing 1 | 102.3292992 | 2.45 | 281.8382931 | |
| 228446_at | KIAA2026 | BF062203 | 0616 | KIAA2026 | 338.8441561 | 2.38 | 239.8832919 | |
| 229541_at | | BE669703 | 0583 | “CDNA FLJ38918 fis, | 93.32543008 | 2.27 | 186.2087137 | |
| | | | | clone NT2NE2010633” | | | | |
| 237330_at | | AA603494 | 0033 | Transcribed locus | 91.20108394 | 1.56 | 36.30780548 | |
| 217842_at | LUC7L2 | NM_016019 | 0893 | LUC7-like 2 (S. cerevisiae) | 61.65950019 | 1.61 | 40.73802778 | |
| 201828_x_at | FAM127A | NM_003928 | 0809 | “Family with sequence | 213.796209 | 2.43 | 269.1534804 | |
| | | | | similarity 127, member A” | | | | |
| 229366_at | | BG149765 | 0673 | Transcribed locus | 218.7761624 | 2.22 | 165.9586907 | |
| 201986_at | THRAP1 | AB011165 | 0084 | Thyroid hormone receptor | 323.5936569 | 2.35 | 223.8721139 | |
| | | | | associated protein 1 | | | | |
| 202373_s_at | RAB3GAP2 | AF255648 | 0148 | RAB3 GTPase activating | 288.4031503 | 2.33 | 213.796209 | |
| | | | | protein subunit 2 (non- | | | | |
| | | | | catalytic) | | | | |
| 241906_at | | BF001773 | 0612 | | 281.8382931 | 2.13 | 134.8962883 | |
| 201318_s_at | “(MRCL3, | NM_006471 | 0863 | “(Myosin regulatory light | 1778.27941 | 3.13 | 1348.962883 | |
| | MRLC2)” | | | chain MRCL3, Myosin | | | | |
| | | | | regulatory light chain | | | | |
| | | | | MRLC2)” | | | | |
| 207941_s_at | RBM39 | NM_004902 | 0830 | RNA binding motif protein | 707.9457844 | 2.72 | 524.8074602 | |
| | | | | 39 | | | | |
| 229586_at | CHD9 | AW300405 | 0505 | Chromodomain helicase | 102.3292992 | 2.23 | 169.8243652 | |
| | | | | DNA binding protein 9 | | | | |
| 217336_at | | AL118510 | 0385 | | 190.5460718 | 2.06 | 114.8153621 | |
| 209648_x_at | SOCS5 | AL136896 | 0394 | Suppressor of cytokine | 38.9045145 | 1.32 | 20.89296131 | |
| | | | | signaling 5 | | | | |
| 226664_at | TBC1D20 | AL121747 | 0388 | “TBC1 domain family, | 199.5262315 | 2.44 | 275.4228703 | |
| | | | | member 20” | | | | |
| 225786_at | LOC284702 | AI440495 | 0204 | Hypothetical protein | 281.8382931 | 2.25 | 177.827941 | |
| | | | | LOC284702 | | | | |
| 208635_x_at | NACA | BF976260 | 0667 | Nascent-polypeptide- | 5128.61384 | 3.62 | 4168.693835 | |
| | | | | associated complex alpha | | | | |
| | | | | polypeptide | | | | |
| 223444_at | SENP7 | AL136599 | 0391 | SUMO1/sentrin specific | 229.0867653 | 2.09 | 123.0268771 | |
| | | | | peptidase 7 | | | | |
| 208975_s_at | KPNB1 | L38951 | 0725 | Karyopherin (importin) | 147.9108388 | 1.98 | 95.4992586 | |
| | | | | beta 1 | | | | |
| 208935_s_at | LGALS8 | L78132 | 0727 | “Lectin, galactoside- | 151.3561248 | 1.96 | 91.20108394 | |
| | | | | binding, soluble, 8 | | | | |
| | | | | (galectin 8)” | | | | |
| 200847_s_at | TMEM66 | NM_016127 | 0895 | Transmembrane protein | 1258.925412 | 2.99 | 977.237221 | |
| | | | | 66 | | | | |
| 218247_s_at | RKHD2 | NM_016626 | 0906 | Ring finger and KH | 208.9296131 | 2.04 | 109.6478196 | |
| | | | | domain containing 2 | | | | |
| 227121_at | | BF476076 | 0640 | MRNA; cDNA | 407.3802778 | 2.43 | 269.1534804 | |
| | | | | DKFZp586K1922 (from | | | | |
| | | | | clone DKFZp586K1922) | | | | |
| 235984_at | | AL036662 | 0367 | Transcribed locus | 100 | 2.27 | 186.2087137 | |
| 212069_s_at | KIAA0515 | AK026025 | 0360 | KIAA0515 | 77.62471166 | 2.13 | 134.8962883 | |
| 243649_at | | AI678692 | 0236 | | 141.2537545 | 1.88 | 75.8577575 | |
| 238549_at | | AI420611 | 0201 | Transcribed locus | 316.227766 | 2.35 | 223.8721139 | |
| 233093_s_at | BIRC6 | AK023788 | 0339 | Baculoviral IAP repeat- | 851.1380382 | 2.83 | 676.0829754 | |
| | | | | containing 6 (apollon) | | | | |
| 221749_at | YTHDF3 | AU157915 | 0439 | “YTH domain family, | 234.4228815 | 2.16 | 144.5439771 | |
| | | | | member 3” | | | | |
| 242726_at | | BF221850 | 0629 | Transcribed locus | 128.8249552 | 1.81 | 64.5654229 | |
| 242195_x_at | NUMBL | BE350811 | 0573 | Numb homolog | 588.8436554 | 2.6 | 398.1071706 | |
| | | | | (Drosophila)-like | | | | |
| 204020_at | PURA | BF739943 | 0658 | Purine-rich element | 316.227766 | 2.36 | 229.0867653 | |
| | | | | binding protein A | | | | |
| 208694_at | PRKDC | U47077 | 0984 | “Protein kinase, DNA- | 131.8256739 | 1.94 | 87.096359 | |
| | | | | activated, catalytic | | | | |
| | | | | polypeptide” | | | | |
| 200047_s_at | YY1 | NM_003403 | 0802 | YY1 transcription factor | 676.0829754 | 2.72 | 524.8074602 | |
| 228097_at | MYLIP | AW292746 | 0497 | Myosin regulatory light | 354.8133892 | 2.35 | 223.8721139 | |
| | | | | chain interacting protein | | | | |
| 213605_s_at | | AL049987 | 0376 | MRNA; cDNA | 144.5439771 | 1.76 | 57.54399373 | |
| | | | | DKFZp564F112 (from | | | | |
| | | | | clone DKFZp564F112) | | | | |
| 218259_at | MKL2 | NM_014048 | 0881 | MKL/myocardin-like 2 | 104.7128548 | 1.83 | 67.60829754 | |
| 240338_at | LRAP | AI807341 | 0272 | Leukocyte-derived | 57.54399373 | 1.45 | 28.18382931 | |
| | | | | arginine aminopeptidase | | | | |
| 226366_at | SHPRH | AI828221 | 0285 | SNF2 histone linker PHD | 173.7800829 | 2.12 | 131.8256739 | |
| | | | | RING helicase | | | | |
| 218237_s_at | SLC38A1 | NM_030674 | 0955 | “Solute carrier family 38, | 851.1380382 | 2.65 | 446.6835922 | |
| | | | | member 1” | | | | |
| 201473_at | JUNB | NM_002229 | 0783 | Jun B proto-oncogene | 346.7368505 | 2.83 | 676.0829754 | |
| 226786_at | RFX1 | BF507952 | 0641 | “Regulatory factor X, 1 | 363.0780548 | 2.72 | 524.8074602 | |
| | | | | (influences HLA class II | | | | |
| | | | | expression)” | | | | |
| 214948_s_at | | AL050136 | 0377 | MRNA; cDNA | 181.9700859 | 2.11 | 128.8249552 | |
| | | | | DKFZp586L141 (from | | | | |
| | | | | clone DKFZp586L141) | | | | |
| 225672_at | GOLGA2 | AL514295 | 0405 | “Golgi autoantigen, golgin | 338.8441561 | 2.65 | 446.6835922 | |
| | | | | subfamily a, 2” | | | | |
| 229787_s_at | OGT | AI742039 | 0251 | O-linked N- | 257.0395783 | 2.08 | 120.2264435 | |
| | | | | acetylglucosamine | | | | |
| | | | | (GlcNAc) transferase | | | | |
| | | | | (UDP-N- | | | | |
| | | | | acetylglucosamine:polypeptide- | | | | |
| | | | | N- | | | | |
| | | | | acetylglucosaminyl | | | | |
| | | | | transferase) | | | | |
| 202253_s_at | DNM2 | NM_004945 | 0832 | Dynamin 2 | 85.11380382 | 2.15 | 141.2537545 | |
| 225564_at | SPATA13 | AW269397 | 0493 | Spermatogenesis | 933.2543008 | 2.74 | 549.5408739 | |
| | | | | associated 13 | | | | |
| 218967_s_at | PTER | NM_030664 | 0954 | Phosphotriesterase | 128.8249552 | 1.93 | 85.11380382 | |
| | | | | related | | | | |
| 217007_s_at | ADAM15 | AK000667 | 0316 | ADAM metallopeptidase | 7.58577575 | 1.54 | 34.67368505 | |
| | | | | domain 15 (metargidin) | | | | |
| 228250_at | FNIP1 | AW263086 | 0491 | Folliculin interacting | 60.25595861 | 2.16 | 144.5439771 | |
| | | | | protein 1 | | | | |
| 212291_at | HIPK1 | AI393355 | 0199 | Homeodomain interacting | 117.4897555 | 2.36 | 229.0867653 | |
| | | | | protein kinase 1 | | | | |
| 203203_s_at | KRR1 | NM_007043 | 0872 | “KRR1, small subunit | 85.11380382 | 1.71 | 51.2861384 | |
| | | | | (SSU) processome | | | | |
| | | | | component, homolog | | | | |
| | | | | (yeast)” | | | | |
| 229470_at | FAM105B | AW451634 | 0509 | “Family with sequence | 257.0395783 | 2.22 | 165.9586907 | |
| | | | | similarity 105, member B” | | | | |
| 224698_at | FAM62B | AB033054 | 0090 | Family with sequence | 676.0829754 | 2.63 | 426.5795188 | |
| | | | | similarity 62 (C2 domain | | | | |
| | | | | containing) member B | | | | |
| 244061_at | | AI510829 | 0213 | | 275.4228703 | 2.07 | 117.4897555 | |
| 204075_s_at | KIAA0562 | NM_014704 | 0887 | KIAA0562 | 24.54708916 | 1.07 | 11.74897555 | |
| 217019_at | | AL137162 | 0395 | | 66.0693448 | 1.45 | 28.18382931 | |
| 218600_at | LIMD2 | NM_030576 | 0953 | LIM domain containing 2 | 102.3292992 | 2.28 | 190.5460718 | |
| 213606_s_at | ARHGDIA | AI571798 | 0218 | Rho GDP dissociation | 25.70395783 | 1.88 | 75.8577575 | |
| | | | | inhibitor (GDI) alpha | | | | |
| 201260_s_at | SYPL1 | NM_006754 | 0869 | Synaptophysin-like 1 | 301.995172 | 2.3 | 199.5262315 | |
| 234340_at | | AK021431 | 0324 | “CDNA FLJ11369 fis, | 50.11872336 | 1.45 | 28.18382931 | |
| | | | | clone HEMBA1000338” | | | | |
| 207686_s_at | CASP8 | NM_001228 | 0770 | “Caspase 8, apoptosis- | 61.65950019 | 2.13 | 134.8962883 | |
| | | | | related cysteine | | | | |
| | | | | peptidase” | | | | |
| 201332_s_at | STAT6 | NM_003153 | 0799 | “Signal transducer and | 102.3292992 | 2.18 | 151.3561248 | |
| | | | | activator of transcription 6, | | | | |
| | | | | interleukin-4 induced” | | | | |
| 203388_at | ARRB2 | NM_004313 | 0819 | “Arrestin, beta 2” | 457.0881896 | 2.85 | 707.9457844 | |
| 204275_at | SOLH | AI796687 | 0268 | Small optic lobes homolog | 52.48074602 | 1.9 | 79.43282347 | |
| | | | | (Drosophila) | | | | |
| 238057_at | USP45 | AW195800 | 0487 | Ubiquitin specific | 81.28305162 | 1.55 | 35.48133892 | |
| | | | | peptidase 45 | | | | |
| 242352_at | NIPBL | AW272262 | 0494 | Nipped-B homolog | 60.25595861 | 2.21 | 162.1810097 | |
| | | | | (Drosophila) | | | | |
| 212846_at | RRP1B | AA811192 | 0063 | KIAA0179 | 323.5936569 | 2.41 | 257.0395783 | |
| 212090_at | GRINA | AL571424 | 0420 | “Glutamate receptor, | 97.7237221 | 2.27 | 186.2087137 | |
| | | | | ionotropic, N-methyl D- | | | | |
| | | | | asparate-associated | | | | |
| | | | | protein 1 (glutamate | | | | |
| | | | | binding)” | | | | |
| 227636_at | THAP5 | BG500677 | 0698 | THAP domain containing 5 | 194.98446 | 2.05 | 112.2018454 | |
| 209379_s_at | KIAA1128 | AF241785 | 0147 | KIAA1128 | 213.796209 | 2.16 | 144.5439771 | |
| 230180_at | | AA521056 | 0025 | | 91.20108394 | 2.37 | 234.4228815 | |
| 217489_s_at | IL6R | S72848 | 0968 | Interleukin 6 receptor | 27.54228703 | 1.77 | 58.88436554 | |
| 200014_s_at | “(HNRPC, | NM_004500 | 0824 | Heterogeneous nuclear | 512.861384 | 2.63 | 426.5795188 | |
| | hCG_1641229)” | | | ribonucleoprotein C | | | | |
| | | | | (C1/C2) | | | | |
| 221628_s_at | N-PAC | AF326966 | 0155 | Cytokine-like nuclear | 42.65795188 | 1.95 | 89.12509381 | |
| | | | | factor n-pac | | | | |
| 214594_x_at | ATP8B1 | BG252666 | 0680 | “ATPase, Class I, type 8B, | 147.9108388 | 1.79 | 61.65950019 | |
| | | | | member 1” | | | | |
| 218528_s_at | RNF38 | NM_022781 | 0941 | Ring finger protein 38 | 309.0295433 | 2.38 | 239.8832919 | |
| 223494_at | MGEA5 | AF307332 | 0152 | Meningioma expressed | 1659.586907 | 3.05 | 1122.018454 | |
| | | | | antigen 5 (hyaluronidase) | | | | |
| 234260_at | | AL122039 | 0390 | MRNA; cDNA | 97.7237221 | 1.68 | 47.86300923 | |
| | | | | DKFZp434E0572 (from | | | | |
| | | | | clone DKFZp434E0572) | | | | |
| 203104_at | CSF1R | NM_005211 | 0838 | “Colony stimulating factor | 354.8133892 | 2.84 | 691.8309709 | |
| | | | | 1 receptor, formerly | | | | |
| | | | | McDonough feline | | | | |
| | | | | sarcoma viral (v-fms) | | | | |
| | | | | oncogene homolog” | | | | |
| 221755_at | EHBP1L1 | BG334196 | 0688 | EH domain binding protein | 47.86300923 | 1.95 | 89.12509381 | |
| | | | | 1-like 1 | | | | |
| 200976_s_at | TAX1BP1 | NM_006024 | 0850 | Tax1 (human T-cell | 691.8309709 | 2.74 | 549.5408739 | |
| | | | | leukemia virus type I) | | | | |
| | | | | binding protein 1 | | | | |
| 212297_at | ATP13A3 | BF218804 | 0628 | ATPase type 13A3 | 100 | 1.77 | 58.88436554 | |
| 208841_s_at | G3BP2 | AB014560 | 0086 | GTPase activating protein | 354.8133892 | 2.41 | 257.0395783 | |
| | | | | (SH3 domain) binding | | | | |
| | | | | protein 2 | | | | |
| 200632_s_at | NDRG1 | NM_006096 | 0853 | N-myc downstream | 223.8721139 | 2.49 | 309.0295433 | |
| | | | | regulated gene 1 | | | | |
| 215383_x_at | SPG21 | AL137312 | 0397 | “Spastic paraplegia 21, | 194.98446 | 2.08 | 120.2264435 | |
| | | | | maspardin (autosomal | | | | |
| | | | | recessive, Mast | | | | |
| | | | | syndrome)” | | | | |
| 235493_at | | AI927329 | 0300 | Transcribed locus | 213.796209 | 2.05 | 112.2018454 | |
| 236614_at | LOC729683 | AW006288 | 0463 | Hypothetical protein | 177.827941 | 2.38 | 239.8832919 | |
| | | | | LOC729683 | | | | |
| 209273_s_at | ISCA1 | BG387555 | 0690 | Iron-sulfur cluster | 144.5439771 | 2 | 100 | |
| | | | | assembly 1 homolog | | | | |
| | | | | (S. cerevisiae) | | | | |
| 227621_at | WTAP | BE464729 | 0574 | Wilms tumor 1 associated | 1584.893192 | 3.07 | 1174.897555 | |
| | | | | protein | | | | |
| 225431_x_at | ACY1L2 | BE779764 | 0596 | Aminoacylase 1-like 2 | 269.1534804 | 2.09 | 123.0268771 | |
| 235879_at | | AI697540 | 0245 | Transcribed locus | 870.96359 | 2.61 | 407.3802778 | |
| 226825_s_at | TMEM165 | AW665624 | 0524 | Transmembrane protein | 575.4399373 | 2.98 | 954.992586 | |
| | | | | 165 | | | | |
| 218143_s_at | SCAMP2 | NM_005697 | 0843 | Secretory carrier | 239.8832919 | 2.54 | 346.7368505 | |
| | | | | membrane protein 2 | | | | |
| 203890_s_at | DAPK3 | BF686824 | 0655 | Death-associated protein | 25.70395783 | 1.64 | 43.65158322 | |
| | | | | kinase 3 | | | | |
| 208313_s_at | SF1 | NM_004630 | 0825 | Splicing factor 1 | 467.7351413 | 2.82 | 660.693448 | |
| 211352_s_at | NCOA3 | U80737 | 0991 | Nuclear receptor | 54.95408739 | 2.02 | 104.7128548 | |
| | | | | coactivator 3 | | | | |
| 224579_at | SLC38A1 | BF247552 | 0632 | “Solute carrier family 38, | 1380.384265 | 2.98 | 954.992586 | |
| | | | | member 1” | | | | |
| 201752_s_at | ADD3 | AI763123 | 0262 | Adducin 3 (gamma) | 1819.700859 | 3.1 | 1258.925412 | |
| 231747_at | CYSLTR1 | NM_006639 | 0864 | Cysteinyl leukotriene | 371.5352291 | 2.81 | 645.654229 | |
| | | | | receptor 1 | | | | |
| 208319_s_at | RBM3 | NM_006743 | 0868 | “RNA binding motif | 407.3802778 | 2.39 | 245.4708916 | |
| | | | | (RNP1, RRM) protein 3” | | | | |
| 227159_at | GHDC | AI827015 | 0284 | GH3 domain containing | 199.5262315 | 2.47 | 295.1209227 | |
| 230387_at | | AL038450 | 0371 | Transcribed locus | 218.7761624 | 2.1 | 125.8925412 | |

TABLE 3

| Fragment | | | | | |
|---|---|---|---|---|---|
| Id | Name | SEQ ID NO. | Accession | Symbol | Gene Name |
| 235101 | 204439_at | 0871 | NM_006820 | IFI44L | interferon-induced protein 44-like |
| 273089 | 242625_at | 0483 | AW189843 | RSAD2 | radical S-adenosyl methionine domain containing 2 |
| 244312 | 213797_at | 0184 | AI337069 | RSAD2 | radical S-adenosyl methionine domain containing 2 |
| 244967 | 214453_s_at | 0860 | NM_006417 | IFI44 | interferon-induced protein 44 |
| 272333 | 241869_at | 0469 | AW026509 | APOL6 | apolipoprotein L, 6 |
| 232750 | 202086_at | 0786 | NM_002462 | MX1 | myxovirus (influenza virus) resistance 1, interferon-inducible |
| | | | | | protein p78 (mouse) |
| 243517 | 212999_x_at | 0496 | AW276186 | LOC650557 | similar to HLA class II histocompatibility antigen, DQ(W1.1) beta |
| | | | | | chain precursor (DQB1*0501) |
| 259919 | 229450_at | 0166 | AI075407 | IFIT3 | interferon-induced protein with tetratricopeptide repeats 3 |
| 232809 | 202145_at | 0785 | NM_002346 | LY6E | lymphocyte antigen 6 complex, locus E |
| 236145 | 205483_s_at | 0835 | NM_005101 | ISG15 | ISG15 ubiquitin-like modifier |
| 262204 | 231735_s_at | 0882 | NM_014086 | | |
| 243866 | 213350_at | 0654 | BF680255 | RPS11 | ribosomal protein S11 |
| 261953 | 231484_at | 0202 | AI424825 | | |
| 245632 | 215123_at | 0375 | AL049250 | | |
| 236794 | 206133_at | 0908 | NM_017523 | XAF1 | XIAP associated factor-1 |
| 257172 | 226702_at | 0252 | AI742057 | LOC129607 | hypothetical protein LOC129607 |
| 244573 | 214059_at | 0565 | BE049439 | IFI44 | interferon-induced protein 44 |
| 241463 | 210873_x_at | 0973 | U03891 | APOBEC3A | apolipoprotein B mRNA editing enzyme, catalytic |
| | | | | | polypeptide-like 3A |
| 250106 | 219607_s_at | 0945 | NM_024021 | MS4A4A | membrane-spanning 4-domains, subfamily A, member 4 |
| 257227 | 226757_at | 0009 | AA131041 | IFIT2 | interferon-induced protein with tetratricopeptide repeats 2 |
| 269075 | 238611_at | 0292 | AI906424 | | |
| 235409 | 204747_at | 0772 | NM_001549 | IFIT3 | interferon-induced protein with tetratricopeptide repeats 3 |
| 268477 | 238013_at | 0634 | BF347859 | | |
| 265621 | 235157_at | 0501 | AW297731 | | |
| 247363 | 216858_x_at | 0379 | AL080112 | | |
| 235077 | 204415_at | 0942 | NM_022873 | IFI6 | interferon, alpha-inducible protein 6 |
| 235634 | 204972_at | 0907 | NM_016817 | OAS2 | 2′-5′-oligoadenylate synthetase 2, 69/71 kDa |
| 263006 | 232537_x_at | 0440 | AU159474 | MARK3 | MAP/microtubule affinity-regulating kinase 3 |
| 272288 | 241824_at | 0002 | AA019641 | | |
| 264429 | 233964_at | 0381 | AL110135 | | |
| 268332 | 237868_x_at | 0267 | AI791828 | | |
| 247318 | 216813_at | 0402 | AL512728 | | |
| 245901 | 215392_at | 0428 | AU148154 | | |
| 272081 | 241617_x_at | 0607 | BE961949 | | |
| 245771 | 215262_at | 0115 | AF052160 | | |
| 118805 | 44673_at | 0748 | N53555 | SIGLEC1 | sialic acid binding Ig-like lectin 1, sialoadhesin |
| 249851 | 219352_at | 0915 | NM_017912 | HERC6 | hect domain and RLD 6 |
| 245223 | 214712_at | 0340 | AK023827 | RUNDC2B | RUN domain containing 2B |
| 238391 | 207735_at | 0913 | NM_017831 | RNF125 | ring finger protein 125 |
| 236214 | 205552_s_at | 0787 | NM_002534 | OAS1 | 2′,5′-oligoadenylate synthetase 1, 40/46 kDa |
| 267022 | 236558_at | 0043 | AA699809 | | |
| 255042 | 224568_x_at | 0462 | AW005982 | MALAT1 | metastasis associated lung adenocarcinoma transcript 1 (non- |
| | | | | | coding RNA) |
| 273157 | 242693_at | 0522 | AW664859 | | |
| 233351 | 202687_s_at | 0987 | U57059 | TNFSF10 | tumor necrosis factor (ligand) superfamily, member 10 |
| 259086 | 228617_at | 0011 | AA142842 | XAF1 | XIAP associated factor-1 |
| 254068 | 223577_x_at | 0068 | AA827878 | MALAT1 | metastasis associated lung adenocarcinoma transcript 1 (non- |
| | | | | | coding RNA) |
| 268806 | 238342_at | 0653 | BF677084 | | |
| 273735 | 243271_at | 0164 | AI064690 | | |
| 258078 | 227609_at | 0035 | AA633203 | EPSTI1 | epithelial stromal interaction 1 (breast) |
| 274457 | 243993_at | 0020 | AA436887 | | |
| 250190 | 219691_at | 0911 | NM_017654 | SAMD9 | sterile alpha motif domain containing 9 |
| 231959 | 201295_s_at | 0620 | BF111821 | WSB1 | WD repeat and SOCS box-containing 1 |
| 235195 | 204533_at | 0774 | NM_001565 | CXCL10 | chemokine (C—X—C motif) ligand 10 |
| 247438 | 216933_x_at | 0967 | S67788 | APC | adenomatosis polyposis coli |
| 244919 | 214405_at | 1014 | Z39557 | | |
| 249708 | 219209_at | 0938 | NM_022168 | IFIH1 | interferon induced with helicase C domain 1 |
| 258895 | 228426_at | 0492 | AW268886 | CLEC2D | C-type lectin domain family 2, member D |
| 255711 | 225239_at | 0188 | AI355441 | | |
| 241858 | 211300_s_at | 0718 | K03199 | TP53 | tumor protein p53 (Li-Fraumeni syndrome) |
| 248900 | 218400_at | 0854 | NM_006187 | OAS3 | 2′-5′-oligoadenylate synthetase 3, 100 kDa |
| 260469 | 230000_at | 0214 | AI523817 | RNF213 | ring finger protein 213 |
| 254430 | 223940_x_at | 0134 | AF132202 | | |
| 250362 | 219863_at | 0899 | NM_016323 | HERC5 | hect domain and RLD 5 |
| 274878 | 244414_at | 0175 | AI148006 | | |
| 267794 | 237330_at | 0033 | AA603494 | | |
| 274252 | 243788_at | 0060 | AA789293 | | |
| 246704 | 216197_at | 0328 | AK021569 | ATF7IP | activating transcription factor 7 interacting protein |
| 263872 | 233406_at | 0329 | AK022100 | | |
| 241392 | 210797_s_at | 0117 | AF063612 | OASL | 2′-5′-oligoadenylate synthetase-like |
| 239530 | 208900_s_at | 0468 | AW025108 | TOP1 | topoisomerase (DNA) I |
| 266011 | 235547_at | 0701 | BG548427 | | |
| 270025 | 239561_at | 0059 | AA780679 | | |
| 263432 | 232964_at | 0396 | AL137266 | LOC441257 | hypothetical LOC441257 |
| | | | | LOC442609 | similar to Williams Beuren syndrome chromosome region 19 |
| | | | | WBSCR19 | Williams Beuren syndrome chromosome region 19 |
| 252807 | 222313_at | 0528 | AW972359 | | |
| 258499 | 228030_at | 0161 | AI041522 | RBM6 | RNA binding motif protein 6 |
| 252260 | 221765_at | 0196 | AI378044 | UGCG | UDP-glucose ceramide glucosyltransferase |
| 254110 | 223619_x_at | 0133 | AF119841 | PECR | peroxisomal trans-2-enoyl-CoA reductase |
| 265453 | 234989_at | 0445 | AV699657 | TncRNA | trophoblast-derived noncoding RNA |
| 252824 | 222330_at | 0530 | AW974995 | | |
| 242071 | 211530_x_at | 0740 | M90686 | HLA-G | HLA-G histocompatibility antigen, class I, G |
| 241567 | 210985_s_at | 0116 | AF056322 | SP100 | SP100 nuclear antigen |
| 246107 | 215599_at | 1012 | X83300 | GUSBP1 | glucuronidase, beta pseudogene 1 |
| | | | | LOC730390 | similar to SMA4 |
| | | | | SMA4 | SMA4 |
| 275143 | 244679_at | 0160 | AI032730 | | |
| 244135 | 213620_s_at | 0007 | AA126728 | ICAM2 | intercellular adhesion molecule 2 |
| 274272 | 243808_at | 0486 | AW193531 | | |
| 234059 | 203397_s_at | 0618 | BF063271 | GALNT3 | UDP-N-acetyl-alpha-D-galactosamine:polypeptide N- |
| | | | | | acetylgalactosaminyltransferase 3 |
| | | | | | (GalNAc-T3) |
| 272396 | 241932_at | 0165 | AI073803 | | |
| 251201 | 220702_at | 0925 | NM_018616 | | |
| 246681 | 216174_at | 0356 | AK025343 | HCRP1 | hepatocellular carcinoma-related HCRP1 |
| 247070 | 216565_x_at | 0389 | AL121994 | | |
| 239595 | 208965_s_at | 0682 | BG256677 | IFI16 | interferon, gamma-inducible protein 16 |
| 246029 | 215521_at | 0335 | AK023029 | PHC3 | polyhomeotic homolog 3 (Drosophila) |
| 272488 | 242024_at | 0971 | T90999 | | |
| 274112 | 243648_at | 0017 | AA280627 | ZC3H11A | zinc finger CCCH-type containing 11A |
| 264725 | 234260_at | 0390 | AL122039 | | |
| 271008 | 240544_at | 0745 | N23033 | | |
| 261578 | 231109_at | 0962 | R44974 | | |
| 244828 | 214314_s_at | 0567 | BE138647 | EIF5B | eukaryotic translation initiation factor 5B |
| 238481 | 207827_x_at | 0724 | L36675 | SNCA | synuclein, alpha (non A4 component of amyloid precursor) |
| 235128 | 204466_s_at | 0684 | BG260394 | SNCA | synuclein, alpha (non A4 component of amyloid precursor) |
| 265641 | 235177_at | 0221 | AI625022 | FAM119A | family with sequence similarity 119, member A |
| 233540 | 202876_s_at | 0789 | NM_002586 | PBX2 | pre-B-cell leukemia homeobox 2 |
| 263328 | 232860_x_at | 0337 | AK023732 | RBM41 | RNA binding motif protein 41 |
| 242745 | 212224_at | 0759 | NM_000689 | ALDH1A1 | aldehyde dehydrogenase 1 family, member A1 |
| 252300 | 221805_at | 0413 | AL537457 | NEFL | neurofilament, light polypeptide 68 kDa |
| 256137 | 225665_at | 0173 | AI129320 | ZAK | sterile alpha motif and leucine zipper containing kinase AZK |
| 252427 | 221932_s_at | 0010 | AA133341 | GLRX5 | glutaredoxin 5 homolog (S. cerevisiae) |
| 258398 | 227929_at | 0431 | AU151342 | | |
| 257281 | 226811_at | 0372 | AL046017 | FAM46C | family with sequence similarity 46, member C |
| 239330 | 208700_s_at | 0720 | L12711 | TKT | transketolase (Wernicke-Korsakoff syndrome) |
| 257126 | 226656_at | 0467 | AW024741 | CRTAP | cartilage associated protein |

TABLE 4

| Fragment Id | Name | SEQ ID NO. | Accession | Symbol | Gene Name |
|---|---|---|---|---|---|
| 255042 | 224568_x_at | 0462 | AW005982 | MALAT1 | metastasis associated lung adenocarcinoma transcript 1 (non-coding |
| | | | | | RNA) |
| 254430 | 223940_x_at | 0134 | AF132202 | | |
| 269238 | 238774_at | 0526 | AW960454 | | |
| 272333 | 241869_at | 0469 | AW026509 | APOL6 | apolipoprotein L, 6 |
| 255041 | 224567_x_at | 0699 | BG534952 | MALAT1 | metastasis associated lung adenocarcinoma transcript 1 (non-coding |
| | | | | | RNA) |
| 257145 | 226675_s_at | 1002 | W80468 | MALAT1 | metastasis associated lung adenocarcinoma transcript 1 (non-coding |
| | | | | | RNA) |
| 238469 | 207815_at | 0792 | NM_002620 | PF4V1 | platelet factor 4 variant 1 |
| 247363 | 216858_x_at | 0379 | AL080112 | | |
| 272488 | 242024_at | 0971 | T90999 | | |
| 245632 | 215123_at | 0375 | AL049250 | | |
| 238391 | 207735_at | 0913 | NM_017831 | RNF125 | ring finger protein 125 |
| 239530 | 208900_s_at | 0468 | AW025108 | TOP1 | topoisomerase (DNA) I |
| 265492 | 235028_at | 0686 | BG288330 | | |
| 235672 | 205010_at | 0929 | NM_019067 | GNL3L | guanine nucleotide binding protein-like 3 (nucleolar)-like |
| 268477 | 238013_at | 0634 | BF347859 | | |
| 240326 | 209703_x_at | 0550 | BC004492 | METTL7A | methyltransferase like 7A |
| 247318 | 216813_at | 0402 | AL512728 | | |
| 255711 | 225239_at | 0188 | AI355441 | | |
| 262881 | 232412_at | 0347 | AK024690 | | |
| 273238 | 242774_at | 0242 | AI684761 | SYNE2 | spectrin repeat containing, nuclear envelope 2 |
| 257532 | 227062_at | 0434 | AU155361 | TncRNA | trophoblast-derived noncoding RNA |
| 247055 | 216550_x_at | 1010 | X80821 | ANKRD12 | ankyrin repeat domain 12 |
| 241039 | 210426_x_at | 0974 | U04897 | RORA | RAR-related orphan receptor A |
| 263432 | 232964_at | 0396 | AL137266 | LOC441257 | hypothetical LOC441257 |
| | | | | LOC442609 | similar to Williams Beuren syndrome chromosome region 19 |
| | | | | WBSCR19 | Williams Beuren syndrome chromosome region 19 |
| 265852 | 235388_at | 0700 | BG538482 | CHD9 | chromodomain helicase DNA binding protein 9 |
| 268332 | 237868_x_at | 0267 | AI791828 | | |
| 264429 | 233964_at | 0381 | AL110135 | | |
| 137612 | 60815_at | 0031 | AA601208 | LOC441257 | hypothetical LOC441257 |
| 249611 | 219112_at | 0900 | NM_016340 | RAPGEF6 | Rap guanine nucleotide exchange factor (GEF) 6 |
| 272343 | 241879_at | 0513 | AW511222 | | |
| 245894 | 215385_at | 0334 | AK022473 | | |
| 251439 | 220940_at | 0952 | NM_025190 | KIAA1641 | KIAA1641 |
| 248290 | 217790_s_at | 0874 | NM_007107 | SSR3 | signal sequence receptor, gamma (translocon-associated protein |
| | | | | | gamma) |
| 232068 | 201404_x_at | 0536 | BC000268 | PSMB2 | proteasome (prosome, macropain) subunit, beta type, 2 |
| 246382 | 215874_at | 0361 | AK026820 | | |
| 247438 | 216933_x_at | 0967 | S67788 | APC | adenomatosis polyposis coli |
| 246107 | 215599_at | 1012 | X83300 | GUSBP1 | glucuronidase, beta pseudogene 1 |
| | | | | LOC730390 | similar to SMA4 |
| | | | | SMA4 | SMA4 |
| 270035 | 239571_at | 0172 | AI123399 | | |
| 260649 | 230180_at | 0025 | AA521056 | | |
| 273380 | 242916_at | 0037 | AA642477 | CEP110 | centrosomal protein 110 kDa |
| 272196 | 241732_at | 0643 | BF513820 | | |
| 274821 | 244357_at | 0970 | T90760 | | |
| 244165 | 213650_at | 0464 | AW006438 | GOLGA8A | golgi autoantigen, golgin subfamily a, 8A |
| 245771 | 215262_at | 0115 | AF052160 | | |
| 252807 | 222313_at | 0528 | AW972359 | | |
| 274112 | 243648_at | 0017 | AA280627 | ZC3H11A | zinc finger CCCH-type containing 11A |
| 248204 | 217704_x_at | 0281 | AI820796 | SUZ12P | suppressor of zeste 12 homolog pseudogene |
| 241090 | 210479_s_at | 0722 | L14611 | RORA | RAR-related orphan receptor A |
| 245901 | 215392_at | 0428 | AU148154 | | |
| 241969 | 211424_x_at | 0130 | AF113007 | METTL7A | methyltransferase like 7A |
| 251085 | 220586_at | 0951 | NM_025134 | CHD9 | chromodomain helicase DNA binding protein 9 |
| 257461 | 226991_at | 0023 | AA489681 | NFATC2 | nuclear factor of activated T-cells, cytoplasmic, calcineurin- |
| | | | | | dependent 2 |
| 268805 | 238341_at | 0653 | BF677084 | | |
| 255023 | 224549_x_at | 0142 | AF194537 | | |
| 243107 | 212587_s_at | 0273 | AI809341 | PTPRC | protein tyrosine phosphatase, receptor type, C |
| 270156 | 239692_at | 0168 | AI083578 | | |
| 262855 | 232386_at | 0450 | AV703288 | VPS13C | vacuolar protein sorting 13 homolog C (S. cerevisiae) |
| 262458 | 231989_s_at | 0095 | AC003007 | SMG1 | PI-3-kinase-related kinase SMG-1 |
| 252481 | 221986_s_at | 0465 | AW006750 | KLHL24 | kelch-like 24 (Drosophila) |
| 238601 | 207953_at | 0106 | AF010144 | AD7C-NTP | neuronal thread protein AD7c-NTP |
| 269722 | 239258_at | 0578 | BE551407 | | |
| 274495 | 244031_at | 0072 | AA868193 | | |
| 273155 | 242691_at | 0070 | AA829017 | | |
| 241975 | 211430_s_at | 0739 | M87789 | IGHG1 | immunoglobulin heavy constant gamma 1 (G1m marker) |
| 265453 | 234989_at | 0445 | AV699657 | TncRNA | trophoblast-derived noncoding RNA |
| 269566 | 239102_s_at | 0500 | AW293296 | | |
| 236769 | 206108_s_at | 0856 | NM_006275 | SFRS6 | splicing factor, arginine/serine-rich 6 |
| 274342 | 243878_at | 0177 | AI248610 | | |
| 242548 | 212027_at | 0298 | AI925305 | RBM25 | RNA binding motif protein 25 |
| 274316 | 243852_at | 0308 | AI963460 | | |
| 252824 | 222330_at | 0530 | AW974995 | | |
| 271008 | 240544_at | 0745 | N23033 | | |
| 272689 | 242225_at | 0216 | AI569482 | | |
| 245204 | 214693_x_at | 0593 | BE732345 | LOC727908 | hypothetical protein LOC727908 |
| | | | | LOC728936 | similar to CG10522-PA |
| | | | | LOC728980 | hypothetical protein LOC728980 |
| | | | | NBPF1 | neuroblastoma breakpoint family, member 1 |
| | | | | NBPF11 | neuroblastoma breakpoint family, member 11 |
| | | | | NBPF14 | neuroblastoma breakpoint family, member 14 |
| | | | | NBPF15 | neuroblastoma breakpoint family, member 15 |
| 259656 | 229187_at | 0159 | AI026708 | LOC283788 | hypothetical protein LOC283788 |
| 246029 | 215521_at | 0335 | AK023029 | PHC3 | polyhomeotic homolog 3 (Drosophila) |
| 272833 | 242369_x_at | 0215 | AI561070 | | |
| 239243 | 208610_s_at | 0233 | AI655799 | SRRM2 | serine/arginine repetitive matrix 2 |
| 243962 | 213446_s_at | 0237 | AI679073 | IQGAP1 | IQ motif containing GTPase activating protein 1 |
| 264616 | 234151_at | 0346 | AK024629 | | |
| 231152 | AFFX-r2- | 0729 | M10098 | | |
| | Hs18SrRNA- | | | | |
| | M_x_at | | | | |
| 261953 | 231484_at | 0202 | AI424825 | | |
| 274272 | 243808_at | 0486 | AW193531 | | |
| 264020 | 233554_at | 0157 | AF339764 | | |
| 245521 | 215012_at | 0423 | AU144775 | | |
| 247997 | 217497_at | 0521 | AW613387 | | |
| 273343 | 242879_x_at | 0305 | AI939442 | | |
| 265524 | 235060_at | 0373 | AL047052 | DKFZp547E087 | hypothetical gene LOC283846 |
| 251975 | 221477_s_at | 0647 | BF575213 | SOD2 | superoxide dismutase 2, mitochondrial |
| 248110 | 217610_at | 0374 | AL047879 | | |
| 244919 | 214405_at | 1014 | Z39557 | | |
| 252260 | 221765_at | 0196 | AI378044 | UGCG | UDP-glucose ceramide glucosyltransferase |
| 245538 | 215029_at | 0383 | AL117451 | | |
| 268267 | 237803_x_at | 0021 | AA455236 | | |
| 265068 | 234604_at | 0350 | AK024881 | | |
| 239886 | 209257_s_at | 0660 | BF795297 | SMC3 | structural maintenance of chromosomes 3 |
| 239489 | 208859_s_at | 0227 | AI650257 | ATRX | alpha thalassemia/mental retardation syndrome X-linked (RAD54 |
| | | | | LOC728849 | homolog, S. cerevisiae) |
| | | | | | similar to transcriptional regulator ATRX isoform 1 |
| 236256 | 205594_at | 0890 | NM_014897 | ZNF652 | zinc finger protein 652 |
| 242515 | 211993_at | 0266 | AI768512 | WNK1 | WNK lysine deficient protein kinase 1 |
| 268806 | 238342_at | 0653 | BF677084 | | |
| 254110 | 223619_x_at | 0133 | AF119841 | PECR | peroxisomal trans-2-enoyl-CoA reductase |
| 261578 | 231109_at | 0962 | R44974 | | |
| 263939 | 233473_x_at | 0437 | AU156202 | | |
| 266109 | 235645_at | 0512 | AW501507 | ESCO1 | establishment of cohesion 1 homolog 1 (S. cerevisiae) |
| 266011 | 235547_at | 0701 | BG548427 | | |
| 246096 | 215588_x_at | 0352 | AK024958 | RIOK3 | RIO kinase 3 (yeast) |
| 270605 | 240141_at | 0617 | BF062399 | | |
| 270275 | 239811_at | 0664 | BF954306 | | |
| 242529 | 212007_at | 0301 | AI927512 | UBXD2 | UBX domain containing 2 |
| 264725 | 234260_at | 0390 | AL122039 | | |
| 237626 | 206965_at | 0898 | NM_016285 | KLF12 | Kruppel-like factor 12 |
| 233810 | 203147_s_at | 0608 | BE962483 | TRIM14 | tripartite motif-containing 14 |
| 274340 | 243876_at | 0062 | AA806845 | | |
| 246372 | 215864_at | 0355 | AK025077 | | |
| 262735 | 232266_x_at | 0343 | AK024379 | CDC2L5 | cell division cycle 2-like 5 (cholinesterase-related cell division |
| | | | | | controller) |
| 267016 | 236552_at | 0261 | AI762475 | | |
| 265187 | 234723_x_at | 0350 | AK024881 | | |
| 239295 | 208663_s_at | 0230 | AI652848 | TTC3 | tetratricopeptide repeat domain 3 |
| 269628 | 239164_at | 0586 | BE674896 | | |
| 244440 | 213926_s_at | 0254 | AI742626 | HRB | HIV-1 Rev binding protein |
| 274283 | 243819_at | 0425 | AU146329 | | |
| 264641 | 234176_at | 0349 | AK024877 | | |
| 259455 | 228986_at | 0531 | AW978375 | OSBPL8 | oxysterol binding protein-like 8 |
| 239465 | 208835_s_at | 0478 | AW089673 | CROP | cisplatin resistance-associated overexpressed protein |
| 247068 | 216563_at | 1010 | X80821 | ANKRD12 | ankyrin repeat domain 12 |
| 246736 | 216229_x_at | 1011 | X81001 | HCG2P7 | HLA complex group 2 pseudogene 7 |
| 267786 | 237322_at | 0293 | AI914323 | MIAT | myocardial infarction associated transcript (non-protein coding) |
| 231366 | 200702_s_at | 0693 | BG421209 | DDX24 | DEAD (Asp-Glu-Ala-Asp) box polypeptide 24 |
| 262879 | 232410_at | 0694 | BG428861 | | |
| 243866 | 213350_at | 0654 | BF680255 | RPS11 | ribosomal protein S11 |
| 272288 | 241824_at | 0002 | AA019641 | | |
| 241872 | 211317_s_at | 0111 | AF041461 | CFLAR | CASP8 and FADD-like apoptosis regulator |
| 253530 | 223036_at | 0708 | D84430 | FARSB | phenylalanyl-tRNA synthetase, beta subunit |
| 240136 | 209508_x_at | 0105 | AF005774 | CFLAR | CASP8 and FADD-like apoptosis regulator |
| 251203 | 220704_at | 0924 | NM_018563 | | |
| 267085 | 236621_at | 0444 | AV683684 | | |
| 242600 | 212079_s_at | 0046 | AA715041 | MLL | myeloid/lymphoid or mixed-lineage leukemia (trithorax homolog, |
| | | | | | Drosophila) |
| 238023 | 207365_x_at | 0888 | NM_014709 | USP34 | ubiquitin specific peptidase 34 |
| 238386 | 207730_x_at | 0916 | NM_017932 | | |
| 243314 | 212794_s_at | 0319 | AK001728 | KIAA1033 | KIAA1033 |
| 266926 | 236462_at | 0051 | AA742310 | | |
| 265631 | 235167_at | 0611 | BE972419 | | |
| 266723 | 236259_at | 0635 | BF433725 | STK4 | serine/threonine kinase 4 |
| 240866 | 210251_s_at | 0129 | AF112221 | RUFY3 | RUN and FYVE domain containing 3 |
| 245168 | 214657_s_at | 0421 | AU134977 | TncRNA | trophoblast-derived noncoding RNA |
| 269022 | 238558_at | 0205 | AI445833 | | |
| 273291 | 242827_x_at | 0061 | AA806368 | | |
| 264284 | 233819_s_at | 0336 | AK023499 | ZNF294 | zinc finger protein 294 |
| 246694 | 216187_x_at | 0146 | AF222691 | | |
| 236717 | 206056_x_at | 1007 | X52075 | SPN | sialophorin (leukosialin, CD43) |
| 52 | AFFX- | 0729 | M10098 | | |
| | HUMRGE/ | | | | |
| | M10098_3_at | | | | |
| 243544 | 213026_at | 0609 | BE965998 | ATG12 | ATG12 autophagy related 12 homolog (S. cerevisiae) |
| 269715 | 239251_at | 0527 | AW963634 | | |
| 246037 | 215529_x_at | 0219 | AI590053 | DIP2A | DIP2 disco-interacting protein 2 homolog A (Drosophila) |
| 239887 | 209258_s_at | 0193 | AI373676 | SMC3 | structural maintenance of chromosomes 3 |
| 250837 | 220338_at | 0920 | NM_018037 | RALGPS2 | Ral GEF with PH domain and SH3 binding motif 2 |
| 244471 | 213957_s_at | 0036 | AA635523 | CEP350 | centrosomal protein 350 kDa |
| 234292 | 203630_s_at | 0859 | NM_006348 | COG5 | component of oligomeric golgi complex 5 |
| 252480 | 221985_at | 0465 | AW006750 | KLHL24 | kelch-like 24 (Drosophila) |
| 237212 | 206551_x_at | 0910 | NM_017644 | KLHL24 | kelch-like 24 (Drosophila) |
| 247346 | 216841_s_at | 1006 | X15132 | SOD2 | superoxide dismutase 2, mitochondrial |
| 232118 | 201454_s_at | 0471 | AW055008 | NPEPPS | aminopeptidase puromycin sensitive |
| 246112 | 215604_x_at | 0338 | AK023783 | | |
| 268974 | 238510_at | 0055 | AA744964 | ZNF720 | zinc finger protein 720 |
| 250731 | 220232_at | 0950 | NM_024906 | SCD5 | stearoyl-CoA desaturase 5 |
| 262594 | 232125_at | 0427 | AU147419 | | |
| 233043 | 202379_s_at | 0190 | AI361805 | NKTR | natural killer-tumor recognition sequence |
| 269738 | 239274_at | 0454 | AV729557 | | |
| 238885 | 208246_x_at | 0909 | NM_017618 | | |
| 271687 | 241223_x_at | 0282 | AI821721 | | |
| 231749 | 201085_s_at | 0040 | AA664291 | SON | SON DNA binding protein |
| 245712 | 215203_at | 0508 | AW438464 | | |
| 105867 | 39313_at | 0081 | AB002342 | WNK1 | WNK lysine deficient protein kinase 1 |
| 241338 | 210742_at | 0118 | AF064103 | CDC14A | CDC14 cell division cycle 14 homolog A (S. cerevisiae) |
| 248079 | 217579_x_at | 0506 | AW301806 | | |
| 258784 | 228315_at | 0223 | AI632728 | | |
| 262204 | 231735_s_at | 0882 | NM_014086 | | |
| 267022 | 236558_at | 0043 | AA699809 | | |
| 270061 | 239597_at | 0080 | AA993566 | | |
| 273495 | 243031_at | 0753 | N90377 | | |
| 266718 | 236254_at | 0564 | BE048857 | VPS13B | vacuolar protein sorting 13 homolog B (yeast) |
| 264495 | 234030_at | 0125 | AF090925 | | |
| 274975 | 244511_at | 0446 | AV700591 | | |
| 239717 | 209088_s_at | 0969 | T70262 | UBN1 | ubinuclein 1 |
| 236173 | 205511_at | 0918 | NM_017976 | | |
| 260682 | 230213_at | 0569 | BE220399 | C19orf43 | chromosome 19 open reading frame 43 |
| 253127 | 222633_at | 0149 | AF268193 | TBL1XR1 | transducin (beta)-like 1X-linked receptor 1 |
| 242364 | 211833_s_at | 0977 | U19599 | BAX | BCL2-associated X protein |
| 264377 | 233912_x_at | 0327 | AK021525 | | |
| 261710 | 231241_at | 0511 | AW469714 | LOC645744 | similar to PCAF associated factor 65 beta isoform b |
| 272801 | 242337_at | 0186 | AI347128 | | |
| 248179 | 217679_x_at | 0240 | AI683552 | | |
| 260844 | 230375_at | 0303 | AI936531 | C6orf111 | chromosome 6 open reading frame 111 |
| 271706 | 241242_at | 0577 | BE503118 | | |
| 272920 | 242456_at | 0079 | AA931565 | MRE11A | MRE11 meiotic recombination 11 homolog A (S. cerevisiae) |
| 268417 | 237953_at | 0171 | AI092511 | | |
| 271732 | 241268_x_at | 0306 | AI939447 | | |
| 262856 | 232387_at | 0359 | AK025700 | AP1GBP1 | AP1 gamma subunit binding protein 1 |
| 248249 | 217749_at | 0896 | NM_016128 | COPG | coatomer protein complex, subunit gamma |
| 269075 | 238611_at | 0292 | AI906424 | | |
| 273157 | 242693_at | 0522 | AW664859 | | |
| 246444 | 215936_s_at | 0318 | AK001657 | KIAA1033 | KIAA1033 |
| 274291 | 243827_at | 0370 | AL038125 | | |
| 237453 | 206792_x_at | 0763 | NM_000923 | PDE4C | phosphodiesterase 4C, cAMP-specific (phosphodiesterase E1 dunce |
| | | | | | homolog, Drosophila) |
| 269728 | 239264_at | 0529 | AW973078 | | |
| 275230 | 244766_at | 0676 | BG180003 | LOC440354 | PI-3-kinase-related kinase SMG-1 pseudogene |
| 253746 | 223254_s_at | 0077 | AA887053 | KIAA1333 | KIAA1333 |
| 274016 | 243552_at | 0466 | AW008914 | | |
| 273410 | 242946_at | 0499 | AW293276 | CD53 | CD53 molecule |
| 260055 | 229586_at | 0504 | AW300405 | CHD9 | chromodomain helicase DNA binding protein 9 |
| 239076 | 208442_s_at | 0754 | NM_000051 | ATM\|\|LOC651610 | ataxia telangiectasia mutated (includes complementation groups A, C |
| | | | | | and D) |
| | | | | | similar to Serine-protein kinase ATM (Ataxia telangiectasia mutated) |
| | | | | | (A-T, mutated) |
| 275010 | 244546_at | 0257 | AI760495 | CYCS | cytochrome c, somatic |
| 260997 | 230528_s_at | 0228 | AI651726 | MGC2752 | hypothetical protein MGC2752 |
| 273598 | 243134_at | 0484 | AW190862 | CHD2 | chromodomain helicase DNA binding protein 2 |
| 265990 | 235526_at | 0595 | BE748802 | C11orf58 | chromosome 11 open reading frame 58 |
| 256821 | 226350_at | 0435 | AU155565 | CHML | choroideremia-like (Rab escort protein 2) |
| 240298 | 209675_s_at | 0548 | BC004242 | HNRPUL1 | heterogeneous nuclear ribonucleoprotein U-like 1 |
| 253682 | 223189_x_at | 0476 | AW082219 | MLL5 | myeloid/lymphoid or mixed-lineage leukemia 5 (trithorax homolog, |
| | | | | | Drosophila) |
| 262364 | 231895_at | 0024 | AA501453 | SASS6 | spindle assembly 6 homolog (C. elegans) |
| 273868 | 243404_at | 0029 | AA553477 | | |
| 255450 | 224977_at | 0386 | AL119182 | C6orf89 | chromosome 6 open reading frame 89 |
| 254234 | 223743_s_at | 0539 | BC000756 | MRPL4 | mitochondrial ribosomal protein L4 |
| 232220 | 201556_s_at | 0546 | BC002737 | VAMP2 | vesicle-associated membrane protein 2 (synaptobrevin 2) |
| 255483 | 225010_at | 0351 | AK024913 | CCDC6 | coiled-coil domain containing 6 |
| 261662 | 231193_s_at | 0571 | BE326569 | | |
| 263781 | 233314_at | 0326 | AK021487 | PTEN | phosphatase and tensin homolog (mutated in multiple advanced |
| | | | | | cancers 1) |
| 233906 | 203243_s_at | 0861 | NM_006457 | PDLIM5 | PDZ and LIM domain 5 |
| 268932 | 238468_at | 0016 | AA214704 | TNRC6B | trinucleotide repeat containing 6B |
| 261248 | 230779_at | 0649 | BF594371 | TNRC6B | trinucleotide repeat containing 6B |

TABLE 5

| Fragment Id | Name | SEQ ID NO. | Accession | Symbol | Gene Name |
|---|---|---|---|---|---|
| 238391 | 207735_at | 0913 | NM_017831 | RNF125 | ring finger protein 125 |
| 239530 | 208900_s_at | 0468 | AW025108 | TOP1 | topoisomerase (DNA) I |
| 243866 | 213350_at | 0654 | BF680255 | RPS11 | ribosomal protein S11 |
| 244919 | 214405_at | 1014 | Z39557 | | |
| 245632 | 215123_at | 0375 | AL049250 | | |
| 245771 | 215262_at | 0115 | AF052160 | | |
| 245901 | 215392_at | 0428 | AU148154 | | |
| 246029 | 215521_at | 0335 | AK023029 | PHC3 | polyhomeotic homolog 3 (Drosophila) |
| 246107 | 215599_at | 1012 | X83300 | GUSBP1 | glucuronidase, beta pseudogene 1 |
| | | | | LOC730390 | similar to SMA4 |
| | | | | SMA4 | SMA4 |
| 247318 | 216813_at | 0402 | AL512728 | | |
| 247363 | 216858_x_at | 0379 | AL080112 | | |
| 247438 | 216933_x_at | 0967 | S67788 | APC | adenomatosis polyposis coli |
| 252260 | 221765_at | 0196 | AI378044 | UGCG | UDP-glucose ceramide glucosyltransferase |
| 252807 | 222313_at | 0528 | AW972359 | | |
| 252824 | 222330_at | 0530 | AW974995 | | |
| 254110 | 223619_x_at | 0133 | AF119841 | PECR | peroxisomal trans-2-enoyl-CoA reductase |
| 255042 | 224568_x_at | 0462 | AW005982 | MALAT1 | metastasis associated lung adenocarcinoma transcript 1 (non- |
| | | | | | coding RNA) |
| 255711 | 225239_at | 0188 | AI355441 | | |
| 261578 | 231109_at | 0962 | R44974 | | |
| 261953 | 231484_at | 0202 | AI424825 | | |
| 262204 | 231735_s_at | 0882 | NM_014086 | | |
| 264429 | 233964_at | 0381 | AL110135 | | |
| 264725 | 234260_at | 0390 | AL122039 | | |
| 265453 | 234989_at | 0445 | AV699657 | TncRNA | trophoblast-derived noncoding RNA |
| 266011 | 235547_at | 0701 | BG548427 | | |
| 267022 | 236558_at | 0043 | AA699809 | | |
| 268332 | 237868_x_at | 0267 | AI791828 | | |
| 268477 | 238013_at | 0634 | BF347859 | | |
| 268806 | 238342_at | 0653 | BF677084 | | |
| 269075 | 238611_at | 0292 | AI906424 | | |
| 271008 | 240544_at | 0745 | N23033 | | |
| 272288 | 241824_at | 0002 | AA019641 | | |
| 272333 | 241869_at | 0469 | AW026509 | APOL6 | apolipoprotein L, 6 |
| 273157 | 242693_at | 0522 | AW664859 | | |
| 274112 | 243648_at | 0017 | AA280627 | ZC3H11A | zinc finger CCCH-type containing 11A |
| 274272 | 243808_at | 0486 | AW193531 | | |

TABLE 6

| SEQ ID NO | Accession No. |
|---|---|
| 0001 | AA012917.1 |
| 0002 | AA019641.1 |
| 0003 | AA029331.1 |
| 0004 | AA031528.1 |
| 0005 | AA045257.1 |
| 0006 | AA058770.1 |
| 0007 | AA126728.1 |
| 0008 | AA126763.1 |
| 0009 | AA131041.1 |
| 0010 | AA133341.1 |
| 0011 | AA142842.1 |
| 0012 | AA149545.1 |
| 0013 | AA156754.1 |
| 0014 | AA194149.1 |
| 0015 | AA203136.1 |
| 0016 | AA214704.1 |
| 0017 | AA280627.1 |
| 0018 | AA359612.1 |
| 0019 | AA416756.1 |
| 0020 | AA436887.1 |
| 0021 | AA455236.1 |
| 0022 | AA468591.1 |
| 0023 | AA489681.1 |
| 0024 | AA501453.1 |
| 0025 | AA521056.1 |
| 0026 | AA521056.1 |
| 0027 | AA530892.1 |
| 0028 | AA530892.1 |
| 0030 | AA580082.1 |
| 0031 | AA601208.1 |
| 0032 | AA602532.1 |
| 0033 | AA603494.1 |
| 0034 | AA632758.1 |
| 0035 | AA633203.1 |
| 0036 | AA635523.1 |
| 0037 | AA642477.1 |
| 0038 | AA650558.1 |
| 0039 | AA664258.1 |
| 0040 | AA664291.1 |
| 0041 | AA679988.1 |
| 0042 | AA699443.1 |
| 0043 | AA699809.1 |
| 0044 | AA702930.1 |
| 0045 | AA703523.1 |
| 0046 | AA715041.1 |
| 0047 | AA721230.1 |
| 0048 | AA732581.1 |
| 0049 | AA732581.1 |
| 0050 | AA741058.1 |
| 0051 | AA742310.1 |
| 0052 | AA743390.1 |
| 0053 | AA744613.1 |
| 0054 | AA744636.1 |
| 0055 | AA744964.1 |
| 0056 | AA748423.1 |
| 0057 | AA749101.1 |
| 0058 | AA758013.1 |
| 0059 | AA780679.1 |
| 0060 | AA789293.1 |
| 0061 | AA806368.1 |
| 0062 | AA806845.1 |
| 0063 | AA811192.1 |
| 0064 | AA811657.1 |
| 0065 | AA814383.1 |
| 0066 | AA825925.1 |
| 0067 | AA827805.1 |
| 0068 | AA827878.1 |
| 0069 | AA828049.1 |
| 0070 | AA829017.1 |
| 0071 | AA843122.1 |
| 0072 | AA868193.1 |
| 0073 | AA872593.1 |
| 0074 | AA873350.1 |
| 0075 | AA876138.1 |
| 0076 | AA877910.1 |
| 0077 | AA887053.1 |
| 0078 | AA916568.1 |
| 0079 | AA931565.1 |
| 0080 | AA993566.1 |
| 0081 | AB002342.2 |
| 0084 | AB011165.2 |
| 0085 | AB014486.1 |
| 0086 | AB014560.1 |
| 0087 | AB019490.1 |
| 0088 | AB023173.1 |
| 0090 | AB033054.2 |
| 0091 | AB035482.1 |
| 0092 | AB043821.1 |
| 0093 | AB044805.1 |
| 0094 | AB051450.1 |
| 0095 | AC003007.1 |
| 0096 | AC003030.1 |
| 0097 | AC004528.1 |
| 0098 | AC004692.2 |
| 0099 | AC004832.3 |
| 0102 | AF000381.2 |
| 0103 | AF000381.2 |
| 0104 | AF001540.1 |
| 0106 | AF010144.1 |
| 0107 | AF021834.1 |
| 0108 | AF022991.1 |
| 0109 | AF037194.1 |
| 0110 | AF041261.1 |
| 0111 | AF041461.1 |
| 0112 | AF044253.1 |
| 0113 | AF044253.1 |
| 0114 | AF047002.1 |
| 0116 | AF056322.1 |
| 0117 | AF063612.1 |
| 0118 | AF064103.1 |
| 0119 | AF065389.1 |
| 0121 | AF070592.1 |
| 0122 | AF070620.1 |
| 0123 | AF078866.1 |
| 0124 | AF086041.1 |
| 0126 | AF092128.1 |
| 0129 | AF112221.1 |
| 0130 | AF113007.1 |
| 0131 | AF113016.1 |
| 0132 | AF116273.1 |
| 0133 | AF119841.1 |
| 0134 | AF132202.1 |
| 0135 | AF132202.1 |
| 0136 | AF141304.1 |
| 0137 | AF143684.1 |
| 0138 | AF159567.1 |
| 0139 | AF165187.1 |
| 0140 | AF172449.1 |
| 0141 | AF176039.1 |
| 0142 | AF194537.1 |
| 0143 | AF194973.1 |
| 0144 | AF212842.1 |
| 0146 | AF222691.1 |
| 0147 | AF241785.1 |
| 0148 | AF255648.1 |
| 0149 | AF268193.1 |
| 0150 | AF274954.1 |
| 0151 | AF302786.1 |
| 0152 | AF307332.1 |
| 0153 | AF315633.1 |
| 0154 | AF317392.1 |
| 0155 | AF326966.1 |
| 0156 | AF327066.1 |
| 0157 | AF339764.1 |
| 0159 | AI026708.1 |
| 0161 | AI041522.1 |
| 0162 | AI052103.1 |
| 0163 | AI057333.1 |
| 0164 | AI064690.1 |
| 0165 | AI073803.1 |
| 0166 | AI075407.1 |
| 0167 | AI078279.1 |
| 0168 | AI083578.1 |
| 0169 | AI084489.1 |
| 0170 | AI092265.1 |
| 0171 | AI092511.1 |
| 0172 | AI123399.1 |
| 0173 | AI129320.1 |
| 0174 | AI139252.1 |
| 0175 | AI148006.1 |
| 0176 | AI241945.1 |
| 0177 | AI248610.1 |
| 0178 | AI249980.1 |
| 0179 | AI263909.1 |
| 0181 | AI283051.1 |
| 0182 | AI332638.1 |
| 0183 | AI336848.1 |
| 0184 | AI337069.1 |
| 0185 | AI341234.1 |
| 0186 | AI347128.1 |
| 0187 | AI348001.1 |
| 0188 | AI355441.1 |
| 0190 | AI361805.1 |
| 0191 | AI363213.1 |
| 0192 | AI369073.1 |
| 0193 | AI373676.1 |
| 0194 | AI377324.1 |
| 0195 | AI378026.1 |
| 0196 | AI378044.1 |
| 0197 | AI379691.1 |
| 0198 | AI380514.1 |
| 0199 | AI393355.1 |
| 0200 | AI418892.1 |
| 0201 | AI420611.1 |
| 0202 | AI424825.1 |
| 0203 | AI434509.1 |
| 0204 | AI440495.1 |
| 0205 | AI445833.1 |
| 0206 | AI458949.1 |
| 0207 | AI459274.1 |
| 0208 | AI472339.1 |
| 0209 | AI472757.1 |
| 0210 | AI473796.1 |
| 0211 | AI475544.1 |
| 0212 | AI492388.1 |
| 0213 | AI510829.1 |
| 0214 | AI523817.1 |
| 0215 | AI561070.1 |
| 0216 | AI569482.1 |
| 0217 | AI571419.1 |
| 0218 | AI571798.1 |
| 0219 | AI590053.1 |
| 0220 | AI590719.1 |
| 0221 | AI625022.1 |
| 0222 | AI632224.1 |
| 0223 | AI632728.1 |
| 0225 | AI633738.1 |
| 0228 | AI651726.1 |
| 0229 | AI652546.1 |
| 0230 | AI652848.1 |
| 0231 | AI652861.1 |
| 0232 | AI652864.1 |
| 0233 | AI655799.1 |
| 0234 | AI673812.1 |
| 0235 | AI674926.1 |
| 0236 | AI678692.1 |
| 0238 | AI681177.1 |
| 0239 | AI681868.1 |
| 0240 | AI683552.1 |
| 0241 | AI683805.1 |
| 0242 | AI684761.1 |
| 0243 | AI688640.1 |
| 0244 | AI689052.1 |
| 0245 | AI697540.1 |
| 0246 | AI732802.1 |
| 0247 | AI733194.1 |
| 0248 | AI738675.1 |
| 0249 | AI738965.1 |
| 0250 | AI741056.1 |
| 0251 | AI742039.1 |
| 0252 | AI742057.1 |
| 0253 | AI742378.1 |
| 0254 | AI742626.1 |
| 0255 | AI753747.1 |
| 0256 | AI754928.1 |
| 0257 | AI760495.1 |
| 0259 | AI761989.1 |
| 0260 | AI762296.1 |
| 0261 | AI762475.1 |
| 0262 | AI763123.1 |
| 0263 | AI763206.1 |
| 0264 | AI767435.1 |
| 0265 | AI767751.1 |
| 0266 | AI768512.1 |
| 0267 | AI791828.1 |
| 0268 | AI796687.1 |
| 0269 | AI797788.1 |
| 0270 | AI805266.1 |
| 0271 | AI806984.1 |
| 0272 | AI807341.1 |
| 0274 | AI809961.1 |
| 0275 | AI810244.1 |
| 0276 | AI814329.1 |
| 0277 | AI814527.1 |
| 0278 | AI818488.1 |
| 0279 | AI819630.1 |
| 0281 | AI820796.1 |
| 0282 | AI821721.1 |
| 0284 | AI827015.1 |
| 0285 | AI828221.1 |
| 0286 | AI829170.1 |
| 0287 | AI829875.1 |
| 0288 | AI832074.1 |
| 0289 | AI857639.1 |
| 0290 | AI860647.1 |
| 0291 | AI871160.1 |
| 0292 | AI906424.1 |
| 0293 | AI914323.1 |
| 0295 | AI923675.1 |
| 0296 | AI924150.1 |
| 0297 | AI924660.1 |
| 0298 | AI925305.1 |
| 0299 | AI925572.1 |
| 0300 | AI927329.1 |
| 0301 | AI927512.1 |
| 0302 | AI934556.1 |
| 0303 | AI936531.1 |
| 0304 | AI937206.1 |
| 0305 | AI939442.1 |
| 0306 | AI939447.1 |
| 0307 | AI950023.1 |
| 0308 | AI963460.1 |
| 0309 | AI970788.1 |
| 0310 | AI979301.1 |
| 0311 | AI982758.1 |
| 0312 | AI983432.1 |
| 0313 | AJ239383.1 |
| 0314 | AJ275469.1 |
| 0315 | AJ292969.1 |
| 0316 | AK000667.1 |
| 0317 | AK000677.1 |
| 0318 | AK001657.1 |
| 0319 | AK001728.1 |
| 0320 | AK001731.1 |
| 0321 | AK001734.1 |
| 0322 | AK001816.1 |
| 0323 | AK001822.1 |
| 0324 | AK021431.1 |
| 0325 | AK021457.1 |
| 0326 | AK021487.1 |
| 0327 | AK021525.1 |
| 0328 | AK021569.1 |
| 0329 | AK022100.1 |
| 0330 | AK022166.1 |
| 0331 | AK022188.1 |
| 0332 | AK022280.1 |
| 0333 | AK022303.1 |
| 0334 | AK022473.1 |
| 0335 | AK023029.1 |
| 0336 | AK023499.1 |
| 0337 | AK023732.1 |
| 0338 | AK023783.1 |
| 0339 | AK023788.1 |
| 0340 | AK023827.1 |
| 0341 | AK024072.1 |
| 0342 | AK024246.1 |
| 0343 | AK024379.1 |
| 0344 | AK024460.1 |
| 0345 | AK024482.1 |
| 0346 | AK024629.1 |
| 0347 | AK024690.1 |
| 0348 | AK024789.1 |
| 0349 | AK024877.1 |
| 0350 | AK024881.1 |
| 0351 | AK024913.1 |
| 0352 | AK024958.1 |
| 0353 | AK024960.1 |
| 0354 | AK024981.1 |
| 0355 | AK025077.1 |
| 0356 | AK025343.1 |
| 0357 | AK025566.1 |
| 0358 | AK025696.1 |
| 0359 | AK025700.1 |
| 0360 | AK026025.1 |
| 0361 | AK026820.1 |
| 0362 | AK075503.1 |
| 0364 | AK098125.1 |
| 0366 | AL035687.13 |
| 0367 | AL036662.1 |
| 0368 | AL037339.1 |
| 0369 | AL037917.2 |
| 0370 | AL038125.1 |
| 0371 | AL038450.1 |
| 0372 | AL046017.1 |
| 0373 | AL047052.1 |
| 0377 | AL050136.1 |
| 0378 | AL080068.1 |
| 0379 | AL080112.1 |
| 0380 | AL080149.1 |
| 0381 | AL110135.1 |
| 0382 | AL110191.1 |
| 0383 | AL117451.1 |
| 0385 | AL118510.6 |
| 0386 | AL119182.1 |
| 0387 | AL119957.1 |
| 0388 | AL121747.41 |
| 0389 | AL121994.12 |
| 0390 | AL122039.1 |
| 0391 | AL136599.1 |
| 0392 | AL136769.1 |
| 0393 | AL136800.1 |
| 0394 | AL136896.1 |
| 0395 | AL137162.25 |
| 0396 | AL137266.1 |
| 0397 | AL137312.1 |
| 0398 | AL137733.1 |
| 0399 | AL161953.1 |
| 0400 | AL358975.8 |
| 0401 | AL390158.1 |
| 0402 | AL512728.1 |
| 0403 | AL513583.3 |
| 0404 | AL513917.3 |
| 0405 | AL514295.3 |
| 0406 | AL524033.3 |
| 0407 | AL524467.3 |
| 0408 | AL526448.3 |
| 0409 | AL527365.3 |
| 0410 | AL533334.3 |
| 0411 | AL534321.3 |
| 0412 | AL534848.3 |
| 0413 | AL537457.2 |
| 0415 | AL550657.3 |
| 0416 | AL552384.3 |
| 0417 | AL565238.3 |
| 0418 | AL565415.3 |
| 0419 | AL568449.2 |
| 0420 | AL571424.3 |
| 0421 | AU134977.1 |
| 0422 | AU144305.1 |
| 0423 | AU144775.2 |
| 0424 | AU146027.1 |
| 0425 | AU146329.1 |
| 0426 | AU146870.1 |
| 0427 | AU147419.2 |
| 0428 | AU148154.1 |
| 0429 | AU149225.2 |
| 0430 | AU150824.1 |
| 0431 | AU151342.2 |
| 0432 | AU155187.1 |
| 0433 | AU155198.1 |
| 0434 | AU155361.1 |
| 0435 | AU155565.1 |
| 0436 | AU155612.1 |
| 0437 | AU156202.1 |
| 0438 | AU157224.1 |
| 0439 | AU157915.1 |
| 0440 | AU159474.1 |
| 0441 | AU160004.2 |
| 0442 | AV655640.1 |
| 0443 | AV659198.1 |
| 0444 | AV683684.1 |
| 0445 | AV699657.1 |
| 0446 | AV700591.1 |
| 0447 | AV700815.2 |
| 0448 | AV700849.1 |
| 0449 | AV701229.1 |
| 0450 | AV703288.2 |
| 0451 | AV722006.1 |
| 0452 | AV725195.1 |
| 0453 | AV725328.1 |
| 0454 | AV729557.1 |
| 0455 | AV735241.1 |
| 0456 | AV739670.1 |
| 0457 | AV744101.1 |
| 0458 | AV748469.1 |
| 0459 | AV760302.2 |
| 0460 | AW001274.1 |
| 0461 | AW001847.1 |
| 0462 | AW005982.1 |
| 0463 | AW006288.1 |
| 0464 | AW006438.1 |
| 0465 | AW006750.1 |
| 0466 | AW008914.1 |
| 0467 | AW024741.1 |
| 0468 | AW025108.1 |
| 0469 | AW026509.1 |
| 0470 | AW027923.1 |
| 0472 | AW057545.1 |
| 0473 | AW070424.1 |
| 0474 | AW070431.1 |
| 0476 | AW082219.1 |
| 0477 | AW082668.1 |
| 0478 | AW089673.1 |
| 0479 | AW131450.1 |
| 0480 | AW163148.1 |
| 0481 | AW173222.1 |
| 0482 | AW189430.1 |
| 0483 | AW189843.1 |
| 0484 | AW190862.1 |
| 0485 | AW190873.1 |
| 0486 | AW193531.1 |
| 0487 | AW195800.1 |
| 0488 | AW238632.1 |
| 0489 | AW243081.1 |
| 0490 | AW244016.1 |
| 0491 | AW263086.1 |
| 0492 | AW268886.1 |
| 0493 | AW269397.1 |
| 0494 | AW272262.1 |
| 0495 | AW275093.1 |
| 0496 | AW276186.1 |
| 0497 | AW292746.1 |
| 0498 | AW292752.1 |
| 0499 | AW293276.1 |
| 0500 | AW293296.1 |
| 0501 | AW297731.1 |
| 0502 | AW298119.1 |
| 0503 | AW298590.1 |
| 0504 | AW298731.1 |
| 0505 | AW300405.1 |
| 0506 | AW301806.1 |
| 0507 | AW364833.1 |
| 0508 | AW438464.1 |
| 0509 | AW451634.1 |
| 0510 | AW468885.1 |
| 0511 | AW469714.1 |
| 0512 | AW501507.1 |
| 0513 | AW511222.1 |
| 0514 | AW511257.1 |
| 0515 | AW514168.1 |
| 0516 | AW515443.1 |
| 0517 | AW519168.1 |
| 0518 | AW575927.1 |
| 0519 | AW582267.1 |
| 0520 | AW590838.1 |
| 0522 | AW664859.1 |
| 0523 | AW665509.1 |
| 0524 | AW665624.1 |
| 0525 | AW953794.1 |
| 0526 | AW960454.1 |
| 0527 | AW963634.1 |
| 0528 | AW972359.1 |
| 0529 | AW973078.1 |
| 0530 | AW974995.1 |
| 0531 | AW978375.1 |
| 0532 | AY014180.1 |
| 0534 | AY034078.1 |
| 0535 | BC000154.2 |
| 0536 | BC000268.1 |
| 0537 | BC000454.2 |
| 0538 | BC000715.2 |
| 0539 | BC000756.2 |
| 0540 | BC000819.1 |
| 0541 | BC001189.1 |
| 0542 | BC001605.1 |
| 0543 | BC002323.2 |
| 0544 | BC002356.2 |
| 0545 | BC002711.2 |
| 0546 | BC002737.2 |
| 0547 | BC003049.1 |
| 0548 | BC004242.1 |
| 0550 | BC004492.2 |
| 0551 | BC004865.2 |
| 0552 | BC004895.2 |
| 0553 | BC005162.2 |
| 0554 | BC005851.2 |
| 0556 | BC006001.1 |
| 0557 | BC006112.1 |
| 0558 | BC006249.2 |
| 0559 | BC012538.1 |
| 0560 | BC025309.1 |
| 0561 | BC040523.1 |
| 0562 | BC043583.1 |
| 0564 | BE048857.1 |
| 0565 | BE049439.1 |
| 0566 | BE049621.1 |
| 0567 | BE138647.1 |
| 0568 | BE205790.1 |
| 0569 | BE220399.1 |
| 0570 | BE251303.1 |
| 0571 | BE326569.1 |
| 0572 | BE348304.1 |
| 0573 | BE350811.1 |
| 0574 | BE464729.1 |
| 0575 | BE466173.1 |
| 0576 | BE501862.1 |
| 0577 | BE503118.1 |
| 0578 | BE551407.1 |
| 0579 | BE568651.1 |
| 0580 | BE613133.1 |
| 0581 | BE645241.1 |
| 0582 | BE646573.1 |
| 0583 | BE669703.1 |
| 0584 | BE674143.1 |
| 0585 | BE674760.1 |
| 0586 | BE674896.1 |
| 0587 | BE675337.1 |
| 0588 | BE675800.1 |
| 0589 | BE675843.1 |
| 0590 | BE675995.1 |
| 0591 | BE676543.1 |
| 0592 | BE708432.1 |
| 0593 | BE732345.1 |
| 0594 | BE734356.1 |
| 0595 | BE748802.1 |
| 0596 | BE779764.1 |
| 0597 | BE783949.1 |
| 0598 | BE784583.1 |
| 0599 | BE788667.1 |
| 0600 | BE795104.1 |
| 0601 | BE856598.1 |
| 0602 | BE856637.1 |
| 0603 | BE856980.1 |
| 0604 | BE869583.1 |
| 0605 | BE876628.1 |
| 0606 | BE899402.1 |
| 0607 | BE961949.2 |
| 0608 | BE962483.2 |
| 0609 | BE965998.2 |
| 0610 | BE966878.2 |
| 0611 | BE972419.1 |
| 0612 | BF001773.1 |
| 0613 | BF055311.1 |
| 0614 | BF056303.1 |
| 0615 | BF060683.1 |
| 0616 | BF062203.1 |
| 0617 | BF062399.1 |
| 0618 | BF063271.1 |
| 0620 | BF111821.1 |
| 0621 | BF114745.1 |
| 0622 | BF115480.1 |
| 0623 | BF115786.1 |
| 0624 | BF116032.1 |
| 0625 | BF195608.1 |
| 0626 | BF195808.1 |
| 0627 | BF206389.1 |
| 0628 | BF218804.1 |
| 0629 | BF221850.1 |
| 0631 | BF246499.1 |
| 0632 | BF247552.1 |
| 0633 | BF347362.1 |
| 0634 | BF347859.1 |
| 0635 | BF433725.1 |
| 0636 | BF438017.1 |
| 0637 | BF438386.1 |
| 0638 | BF448315.1 |
| 0639 | BF475280.1 |
| 0640 | BF476076.1 |
| 0641 | BF507952.1 |
| 0642 | BF510713.1 |
| 0643 | BF513820.1 |
| 0644 | BF514509.1 |
| 0645 | BF514975.1 |
| 0646 | BF525395.1 |
| 0647 | BF571480.1 |
| 0648 | BF575213.1 |
| 0649 | BF594371.1 |
| 0650 | BF594446.1 |
| 0651 | BF670827.1 |
| 0652 | BF673699.1 |
| 0653 | BF677084.1 |
| 0654 | BF680255.1 |
| 0655 | BF686824.1 |
| 0656 | BF696840.1 |
| 0658 | BF739943.1 |
| 0659 | BF791874.1 |
| 0660 | BF795297.1 |
| 0661 | BF796940.1 |
| 0662 | BF797555.1 |
| 0663 | BF939788.1 |
| 0664 | BF954306.1 |
| 0665 | BF966155.1 |
| 0666 | BF971923.1 |
| 0667 | BF976260.1 |
| 0668 | BF982174.1 |
| 0669 | BG028844.1 |
| 0670 | BG032366.1 |
| 0671 | BG111168.1 |
| 0672 | BG112118.1 |
| 0673 | BG149765.1 |
| 0674 | BG149849.1 |
| 0675 | BG178695.1 |
| 0676 | BG180003.1 |
| 0677 | BG222258.1 |
| 0678 | BG231551.1 |
| 0679 | BG251467.1 |
| 0680 | BG252666.1 |
| 0681 | BG253884.1 |
| 0682 | BG256677.1 |
| 0683 | BG260337.1 |
| 0684 | BG260394.1 |
| 0685 | BG261071.1 |
| 0686 | BG288330.1 |
| 0687 | BG290742.1 |
| 0688 | BG334196.1 |
| 0689 | BG341906.1 |
| 0690 | BG387555.1 |
| 0691 | BG389744.1 |
| 0692 | BG390306.1 |
| 0693 | BG421209.1 |
| 0694 | BG428861.1 |
| 0695 | BG430133.1 |
| 0696 | BG481972.1 |
| 0697 | BG485163.1 |
| 0698 | BG500677.1 |
| 0699 | BG534952.1 |
| 0700 | BG538482.1 |
| 0701 | BG548427.1 |
| 0702 | BQ002274.1 |
| 0703 | BU618741.1 |
| 0704 | D13989.1 |
| 0705 | D25284.1 |
| 0706 | D32039.1 |
| 0707 | D83485.1 |
| 0708 | D84430.1 |
| 0709 | D86043.1 |
| 0710 | D86862.1 |
| 0711 | D87021.1 |
| 0712 | D87858.1 |
| 0713 | H24473.1 |
| 0714 | H49240.1 |
| 0715 | H75391.1 |
| 0719 | L01639.1 |
| 0720 | L12711.1 |
| 0721 | L14599.1 |
| 0723 | L34164.1 |
| 0724 | L36675.1 |
| 0725 | L38951.1 |
| 0726 | L48784.1 |
| 0728 | L78790.1 |
| 0730 | M11167.1 |
| 0731 | M24668.1 |
| 0732 | M25915.1 |
| 0733 | M28590.1 |
| 0734 | M32221.1 |
| 0738 | M80776.1 |
| 0740 | M90686.1 |
| 0741 | M93255.1 |
| 0744 | N21202.1 |
| 0745 | N23033.1 |
| 0746 | N24868.1 |
| 0747 | N51961.1 |
| 0748 | N53555.1 |
| 0749 | N57510.1 |
| 0750 | N63244.1 |
| 0751 | N64578.1 |
| 0752 | N66397.1 |
| 0753 | N90377.1 |
| 0754 | NM_000051.3 |
| 0755 | NM_000099.2 |
| 0758 | NM_000404.2 |
| 0759 | NM_000689.3 |
| 0760 | NM_000697.2 |
| 0761 | NM_000760.2 |
| 0762 | NM_000918.3 |
| 0763 | NM_000923.3 |
| 0764 | NM_000962.2 |
| 0766 | NM_000971.3 |
| 0767 | NM_000978.3 |
| 0768 | NM_001069.2 |
| 0769 | NM_001155.3 |
| 0770 | NM_001228.4 |
| 0772 | NM_001549.3 |
| 0779 | NM_001787.1 |
| 0780 | NM_002000.2 |
| 0781 | NM_002073.2 |
| 0782 | NM_002076.2 |
| 0784 | NM_002339.2 |
| 0785 | NM_002346.2 |
| 0787 | NM_002534.2 |
| 0788 | NM_002541.2 |
| 0790 | NM_002616.1 |
| 0792 | NM_002620.2 |
| 0793 | NM_002624.3 |
| 0794 | NM_002709.2 |
| 0795 | NM_002764.3 |
| 0796 | NM_002858.3 |
| 0797 | NM_003043.3 |
| 0799 | NM_003153.3 |
| 0800 | NM_003189.1 |
| 0802 | NM_003403.3 |
| 0804 | NM_003461.4 |
| 0805 | NM_003563.3 |
| 0807 | NM_003655.2 |
| 0809 | NM_003928.1 |
| 0810 | NM_004030.1 |
| 0812 | NM_004103.3 |
| 0813 | NM_004117.2 |
| 0814 | NM_004126.3 |
| 0815 | NM_004145.2 |
| 0816 | NM_004233.3 |
| 0818 | NM_004309.3 |
| 0819 | NM_004313.3 |
| 0820 | NM_004323.4 |
| 0824 | NM_004500.3 |
| 0825 | NM_004630.2 |
| 0826 | NM_004640.5 |
| 0827 | NM_004648.1 |
| 0828 | NM_004731.3 |
| 0829 | NM_004774.3 |
| 0830 | NM_004902.2 |
| 0832 | NM_004945.2 |
| 0834 | NM_005022.2 |
| 0836 | NM_005184.2 |
| 0837 | NM_005195.3 |
| 0839 | NM_005231.3 |
| 0840 | NM_005238.2 |
| 0841 | NM_005399.3 |
| 0843 | NM_005697.3 |
| 0844 | NM_005775.4 |
| 0845 | NM_005781.4 |
| 0846 | NM_005808.2 |
| 0848 | NM_005911.4 |
| 0849 | NM_006006.4 |
| 0850 | NM_006024.5 |
| 0851 | NM_006026.2 |
| 0852 | NM_006079.3 |
| 0854 | NM_006187.2 |
| 0855 | NM_006265.1 |
| 0856 | NM_006275.4 |
| 0858 | NM_006305.2 |
| 0859 | NM_006348.2 |
| 0860 | NM_006417.4 |
| 0861 | NM_006457.2 |
| 0862 | NM_006459.3 |
| 0863 | NM_006471.2 |
| 0864 | NM_006639.2 |
| 0865 | NM_006640.4 |
| 0867 | NM_006725.2 |
| 0868 | NM_006743.3 |
| 0869 | NM_006754.2 |
| 0871 | NM_006820.2 |
| 0872 | NM_007043.6 |
| 0874 | NM_007107.2 |
| 0875 | NM_007279.2 |
| 0876 | NM_012124.1 |
| 0877 | NM_012179.3 |
| 0878 | NM_012268.2 |
| 0879 | NM_013232.3 |
| 0881 | NM_014048.3 |
| 0882 | NM_014086.1 |
| 0883 | NM_014308.2 |
| 0885 | NM_014358.2 |
| 0886 | NM_014380.1 |
| 0887 | NM_014704.2 |
| 0888 | NM_014709.3 |
| 0889 | NM_014721.2 |
| 0891 | NM_015859.2 |
| 0892 | NM_015995.2 |
| 0893 | NM_016019.2 |
| 0894 | NM_016022.2 |
| 0895 | NM_016127.4 |
| 0896 | NM_016128.3 |
| 0897 | NM_016231.4 |
| 0898 | NM_016285.2 |
| 0900 | NM_016340.4 |
| 0901 | NM_016376.3 |
| 0902 | NM_016509.3 |
| 0903 | NM_016526.4 |
| 0904 | NM_016542.3 |
| 0905 | NM_016592.2 |
| 0906 | NM_016626.3 |
| 0907 | NM_016817.2 |
| 0908 | NM_017523.2 |
| 0909 | NM_017618.1 |
| 0910 | NM_017644.3 |
| 0911 | NM_017654.2 |
| 0912 | NM_017778.2 |
| 0913 | NM_017831.3 |
| 0914 | NM_017905.4 |
| 0915 | NM_017912.3 |
| 0916 | NM_017932.1 |
| 0917 | NM_017936.3 |
| 0918 | NM_017976.1 |
| 0919 | NM_017987.4 |
| 0920 | NM_018037.1 |
| 0921 | NM_018072.4 |
| 0922 | NM_018115.2 |
| 0923 | NM_018263.4 |
| 0924 | NM_018563.1 |
| 0925 | NM_018616.1 |
| 0926 | NM_018986.3 |
| 0927 | NM_019049.1 |
| 0928 | NM_019058.2 |
| 0929 | NM_019067.4 |
| 0930 | NM_019843.2 |
| 0931 | NM_019903.3 |
| 0932 | NM_020152.2 |
| 0933 | NM_021029.4 |
| 0934 | NM_021630.4 |
| 0936 | NM_021978.3 |
| 0937 | NM_022047.3 |
| 0938 | NM_022168.2 |
| 0939 | NM_022367.2 |
| 0940 | NM_022461.2 |
| 0941 | NM_022781.4 |
| 0942 | NM_022873.2 |
| 0943 | NM_023015.3 |
| 0944 | NM_023914.2 |
| 0945 | NM_024021.2 |
| 0946 | NM_024498.1 |
| 0947 | NM_024564.1 |
| 0948 | NM_024693.4 |
| 0949 | NM_024852.2 |
| 0950 | NM_024906.2 |
| 0951 | NM_025134.4 |
| 0952 | NM_025190.2 |
| 0953 | NM_030576.3 |
| 0954 | NM_030664.3 |
| 0955 | NM_030674.3 |
| 0956 | NM_030912.2 |
| 0957 | NM_030917.2 |
| 0958 | NM_030927.1 |
| 0959 | NM_030930.2 |
| 0960 | NM_172313.1 |
| 0961 | R05895.1 |
| 0962 | R44974.1 |
| 0963 | R64130.1 |
| 0964 | R94644.1 |
| 0965 | S55735.1 |
| 0966 | S67779.1 |
| 0968 | S72848.1 |
| 0969 | T70262.1 |
| 0970 | T90760.1 |
| 0971 | T90999.1 |
| 0972 | T92908.1 |
| 0973 | U03891.2 |
| 0977 | U19599.1 |
| 0980 | U33448.1 |
| 0982 | U41070.1 |
| 0983 | U43677.1 |
| 0985 | U53470.1 |
| 0986 | U56237.1 |
| 0988 | U66145.1 |
| 0990 | U77413.1 |
| 0991 | U80737.1 |
| 0992 | U82276.1 |
| 0993 | U82277.1 |
| 0994 | U82278.1 |
| 0996 | U84744.1 |
| 0997 | U92706.1 |
| 0998 | W27419.1 |
| 0999 | W58365.1 |
| 1000 | W74580.1 |
| 1001 | W80418.1 |
| 1002 | W80468.1 |
| 1003 | W86302.1 |
| 1004 | W86659.1 |
| 1006 | X15132.1 |
| 1007 | X52075.1 |
| 1008 | X57812.1 |
| 1011 | X81001.1 |
| 1012 | X83300.1 |
| 1013 | Z25430.1 |
| 1014 | Z39557.1 |

1. A method for evaluating multiple sclerosis (MS) in a patient, comprising:
determining a gene expression profile for a blood sample of a patient having or suspected of having MS, said gene expression profile comprising gene expression values for a plurality of genes that are differentially expressed in MS, said genes being listed in Tables 1-5;
comparing said gene expression profile to an MS- and/or a non-MS profile; and
classifying the gene expression profile as an MS profile or a non-MS profile.

2. (canceled)

3. The method of claim 1, wherein the gene expression profile is determined prior to treatment for MS.

4. The method of claim 1, wherein said patient is being treated with at least one of Beta-interferon, Glatiramer acetate, and Natalizumab.

5. The method of claim 1, wherein said patient is suspected of having MS.

6. (canceled)

7. (canceled)

8. The method of claim 1, wherein the gene expression profile is determined after about 3 months of treatment, after about 6 months of treatment, and/or after about 12 months of treatment for MS.

9. The method of claim 1, wherein classifying the gene expression profile as an MS profile or a non-MS profile includes determining a probability that the patient has MS.

10. The method of claim 1, wherein the non-MS profile and/or the MS profile is matched to the patient by at least one of age, race, gender, and clinical manifestation of MS.

11. The method of claim 1, wherein said MS profile is a relapsing—remitting MS profile.

12. The method of claim 1, wherein the sample is a whole blood sample.

13. The method of claim 11, wherein the genes that are differentially expressed in MS patients are listed in Table 1 or 2.

14. The method of claim 1, wherein the sample is a white blood cell sample.

15. The method of claim 14, wherein the genes that are differentially expressed in MS patients are listed in Table 2.

16. (canceled)

17. The method of claim 1, wherein said gene expression profile comprises the levels of expression for at least 7 genes listed in Table 1 or 2.

18-21. (canceled)

22. The method of claim 1, wherein said levels of expression have been normalized to the MS-profile and non-MS profile based on the level of expression of at least one constitutively expressed gene.

23. The method of claim 1, wherein the sample is a Peripheral Blood Mononuclear Cell (PBMC) sample.

24. The method of claim 1, wherein said gene expression profile is determined using a hybridization-based assay or a polymerase-based assay.

25. The method of claim 1, wherein the gene expression profile is classified as an MS profile or a non-MS profile using one or more classification schemes selected from Naïve Bayes, Support Vector Machine, Nearest Neighbor, Decision Tree, Logistic, Artificial Neural Network, and Rule-based scheme.

26. (canceled)

27. A method for preparing a patient gene expression profile for evaluating MS, comprising, quantifying the level of expression of a plurality of genes listed in one or more of Tables 1-5 in a patient blood sample.

28-44. (canceled)

45. A method for monitoring treatment of a multiple sclerosis patient, comprising:
determining a pre-treatment gene expression profile and at least one post-treatment gene expression profile, said pre-treatment and said post-treatment gene expression profiles comprising gene expression values for a plurality of genes that are differentially expressed upon treatment for MS, said genes being listed in one of Tables 3-5;
comparing said pre-treatment gene expression profile with said post-treatment gene expression profile; and
identifying differences between the pre-treatment gene expression profile and the post-treatment gene expression profile, wherein said differences are indicative of effective treatment for MS.

46-54. (canceled)

55. A computer system comprising a database on one or more computer readable medium, and containing mean gene expression values for at least ten genes in an MS patient population and in a non-MS patient population, said at least ten genes being selected from Table 1 and/or 2.

56-65. (canceled)

66. A kit for evaluating MS, comprising nucleic acid primers and/or probes for determining the level of expression in a patient sample of a plurality of genes listed in Tables 1-5.

67. (canceled)

68. (canceled)
